SEQUENCE LISTING (32 sequences)

SEQ ID NO: 1 - DNA, 53 bp, Synthetic construct
  source 1..53: mol_type = other DNA
catgccatgggctgtcgtacctctggcgctggggttttagagctagaaatagc

SEQ ID NO: 2 - DNA, 18 bp, Synthetic construct
  source 1..18: mol_type = other DNA
gctggacgaacgcaagat

SEQ ID NO: 3 - DNA, 25 bp, Synthetic construct
  source 1..25: mol_type = other DNA
tccagggctggggctacatcaacct

SEQ ID NO: 4 - DNA, 25 bp, Synthetic construct
  source 1..25: mol_type = other DNA
ttcatgcttgtggtcctcgggcact

SEQ ID NO: 5 - DNA, 50 bp, Synthetic construct
  source 1..50: mol_type = other DNA
tcgtcgaaggcactagaaggggccacatgaccaacgcgttcatcctcggc

SEQ ID NO: 6 - DNA, 50 bp, Synthetic construct
  source 1..50: mol_type = other DNA
gcgttcgtccagcggcggggttgcggtgcagcagcgggaagtcatcgggt

SEQ ID NO: 7 - DNA, 30 bp, Synthetic construct
  source 1..30: mol_type = other DNA
ccccgccgctggacgaacgcaagatcctca

SEQ ID NO: 8 - DNA, 50 bp, Synthetic construct
  source 1..50: mol_type = other DNA
ggtcgatccccgcatataggatcagctcggagtggctgcactacggctac

SEQ ID NO: 9 - DNA, 48 bp, Synthetic construct
  source 1..48: mol_type = other DNA
ctggtgccgcgcggcagccatgtgcgggaggccggctccggccggtgt

SEQ ID NO: 10 - DNA, 56 bp, Synthetic construct
  source 1..56: mol_type = other DNA
tggtggtggtgctcgagtgcctaccggctccagccgtgcgggccggcgtcgacccg

SEQ ID NO: 11 - DNA, 47 bp, Synthetic construct
  source 1..47: mol_type = other DNA
ccatggcgatatcggatccggtgcgggaggccggctccggccggtct

SEQ ID NO: 12 - DNA, 56 bp, Synthetic construct
  source 1..56: mol_type = other DNA
tggtggtggtgctcgagtgcccggctccagccgtgcgggccggcgtcgacccgctg

SEQ ID NO: 13 - DNA, 45 bp, Synthetic construct
  source 1..45: mol_type = other DNA
ctggtgccgcgcggcagccatatggcaatcgatctctgcaacaag

SEQ ID NO: 14 - DNA, 39 bp, Synthetic construct
  source 1..39: mol_type = other DNA
tggtggtggtgctcgagtgcttacggcagcagggtggtg

SEQ ID NO: 15 - DNA, 47 bp, Synthetic construct
  source 1..47: mol_type = other DNA
ccacgcggttccatggcgataatggcaatcgatctctgcaacaagtc

SEQ ID NO: 16 - DNA, 38 bp, Synthetic construct
  source 1..38: mol_type = other DNA
tggtggtggtgctcgagtgccggcagcagggtggtgtg

SEQ ID NO: 17 - DNA, 53 bp, Synthetic construct
  source 1..53: mol_type = other DNA
catgccatgggaccggacttgaagaacgtccgggttttagagctagaaatagc

SEQ ID NO: 18 - DNA, 31 bp, Synthetic construct
  source 1..31: mol_type = other DNA
catctcgatgaggttggtgtagacctcggcg

SEQ ID NO: 19 - DNA, 32 bp, Synthetic construct
  source 1..32: mol_type = other DNA
atccggccggcgcagtgcacgatcgcggtggg

SEQ ID NO: 20 - DNA, 50 bp, Synthetic construct
  source 1..50: mol_type = other DNA
tcgtcgaaggcactagaaggggaccagagtgaccactcggcggtgcagca

SEQ ID NO: 21 - DNA, 52 bp, Synthetic construct
  source 1..52: mol_type = other DNA
tcgtcgaaggcactagaaggccgccgcagcagggtgagcatccggcggacgt

SEQ ID NO: 22 - DNA, 42 bp, Synthetic construct
  source 1..42: mol_type = other DNA
gcagggtggtgttgttgcagagatcgattgccattgctgcct

SEQ ID NO: 23 - DNA, 30 bp, Synthetic construct
  source 1..30: mol_type = other DNA
acaccaccctgctgccgtaacgaaggaaga

SEQ ID NO: 24 - DNA, 51 bp, Synthetic construct
  source 1..51: mol_type = other DNA
ggtcgatccccgcatatagggcatgccaacctccgactgtcgtcgctcctt

SEQ ID NO: 25 - protein, 210 aa, Micromonospora chersina
MSTKSVLFGRPVQTEGVPNVYAGAPVVPWTPPEPGIDNLGINSIDTFAVPGVGEYTVAFDGWVRVVRSPSTSGEWADAEVYTNLIEMKMVGECEELGKITVTLNPDCLSAGQIRTPFDPYAGEGPSAKACRMAVGAIFDMPKLGLKLMNREPIILTIDDVRSIPPAGAPGKGQIYRMMPLLDVNDPDGQPVAYLTSLRFNMGGYLKPDQM

SEQ ID NO: 26 - DNA, 50 bp, Synthetic construct
  source 1..50: mol_type = other DNA
tcgtcgaaggcactagaaggccggctggacaaccgcaccaaccaggaact

SEQ ID NO: 27 - DNA, 50 bp, Synthetic construct
  source 1..50: mol_type = other DNA
catctggtcgggcttcaggtgaacgctcttggtgctcatctctggtgtcc

SEQ ID NO: 28 - DNA, 30 bp, Synthetic construct
  source 1..30: mol_type = other DNA
acctgaagcccgaccagatgtgattcccca

SEQ ID NO: 29 - DNA, 50 bp, Synthetic construct
  source 1..50: mol_type = other DNA
ggtcgatccccgcatataggagtagccggtgaggatcttgcgttcgtcca

SEQ ID NO: 30 - DNA, 50 bp, Synthetic construct
  source 1..50: mol_type = other DNA
catgccatggatcttcgacatgccgaagctgttttagagctagaaatagc

SEQ ID NO: 31 - DNA, 27 bp, Synthetic construct
  source 1..27: mol_type = other DNA
acgcgctcaacttctcctaccggacca

SEQ ID NO: 32 - DNA, 26 bp, Synthetic construct
  source 1..26: mol_type = other DNA
ctgaccgttcccggtgccgttcaggt

(19) United States
(12) Patent Application Publication
(10) Pub. No.: US 2026/0216352 A1
(43) Pub. Date: Jul. 30, 2026

(54) DIRECTED BIOSYNTHESIS OF MODIFIED ANTHRAQUINONE-FUSED ENEDIYNES AS POTENTIAL CANCER THERAPEUTICS

(71) Applicant: The Johns Hopkins University, Baltimore, MD (US)

(72) Inventors: Craig A. TOWNSEND, Baltimore, MD (US); Paramita PAL, Baltimore, MD (US); Jamie ALLEY, Baltimore, MD (US)

(21) Appl. No.: 19/144,725

(22) PCT Filed: Jan. 9, 2024
(86) PCT No.: PCT/US2024/010940

§ 371 Date: Jun. 30, 2025

Related U.S. Application Data
(60) Provisional application No. 63/479,083, filed on Jan. 9, 2023.
(60) Provisional application No. 63/481,029, filed on Jan. 23, 2023.

(51) Int. Cl. A61K 47/68; A61K 47/58; A61P 35/00; C07D 333/50; C07D 491/08

(52) U.S. Cl. CPC A61K 47/6803; A61K 47/585; A61P 35/00; C07D 333/50; C07D 491/08

(57) ABSTRACT

Analogues of dynemicin, methods of their preparation, and their use for treating cancer are disclosed.

### FEDERALLY SPONSORED RESEARCH OR DEVELOPMENT

This invention was made with government support under grant ES001670 awarded by the National Institutes of Health. The government has certain rights in the invention.

### BACKGROUND

Unique among the three structural classes of highly cytotoxic enediyne natural products (FIG. 1), dynemicin (3, Dyn) is biosynthesized from two equal-size polyketide-derived building blocks, resulting in “enediyne” and “anthraquinone” halves. Cohen and Townsend, 2018a. An iterative highly-reducing polyketide synthase (HR-PKS) DynE8, having strikingly similar homologs encoded by all known enediyne biosynthetic gene clusters (BGCs) (Gao and Thorson, 2008), generates the acyl carrier protein-bound β-hydroxyhexaene 4 to initiate the synthesis of all three classes of enediyne products (FIG. 2).

Cohen and Townsend, 2018a. In the case of dynemicin, this C16 precursor is modified to a C15 intermediate that bifurcates into two parallel paths to form the structurally dissimilar enediyne and anthraquinone halves (Cohen and Townsend, 2020) of Dyn, which link in the key heterodimerization reaction. Cohen and Townsend, 2020. This heterodimerization reaction, and the biosynthesis of dynemicin and related compounds, however, are not well understood.

### SUMMARY

In some aspects, the presently disclosed subject matter provides a compound of formula (I):

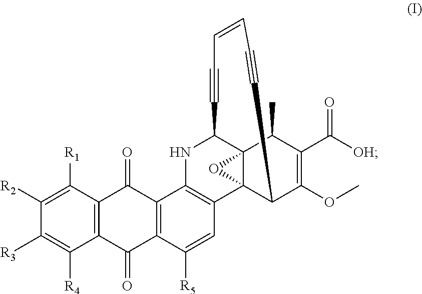

wherein: R₁ is selected from H, hydroxyl, and —O—(C═O)—CH₃; R₂ and R₃ are each independently selected from H, hydroxyl, C₁-C₄ alkyl, C₁-C₄ alkoxyl, halogen, carbonyl, carboxyl, acetyl, cyano, mercapto, nitro, —O—(C═O)—CH₃, and —(CH₂)ₙ—NR₆R₇, wherein n is an integer selected from 0, 1, 2, 3, and 4, R₆ and R₇ are each independently selected from H, C₁-C₄ alkyl, and a protecting group; R₅ is hydroxyl or —O—(C═O)—CH₃; provided that if R₁, R₄, and R₅ are each hydroxyl, then R₂ and R₃ cannot both be H; and; stereoisomers and pharmaceutically acceptable salts thereof.

In certain aspects, the compound of formula (I) is a compound of formula (I′):

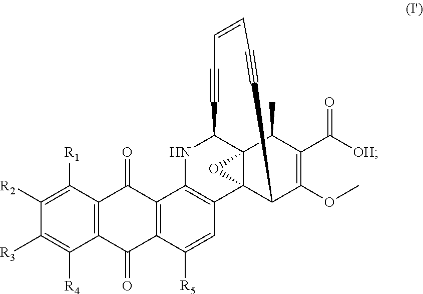

wherein: R₁ is selected from H, hydroxyl, and —O—(C═O)—CH₃; R₂ and R₄ are each H; R₃ is selected from C₁-C₄ alkyl, halogen, —(CH₂)ₙ—NR₆R₇, wherein n is an integer selected from 0, 1, 2, 3, and 4, R₆ and R₇ are each independently selected from H, C₁-C₄ alkyl, and a protecting group; R₅ is hydroxyl or —O—(C═O)—CH₃; and stereoisomers and pharmaceutically acceptable salts thereof.

In certain aspects, the compound is a compound of formula (Ta):

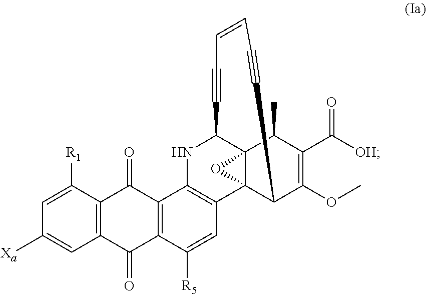

wherein: Xₐ is halogen or cyano; R₁ is selected from H, hydroxyl, and —O—(C═O)—CH₃; and R₅ is hydroxyl or —O—(C═O)—CH₃. In particular aspects, Xₐ is selected from Br, Cl, F, and cyano.

In certain aspects, the compound is a compound of formula (Ib):

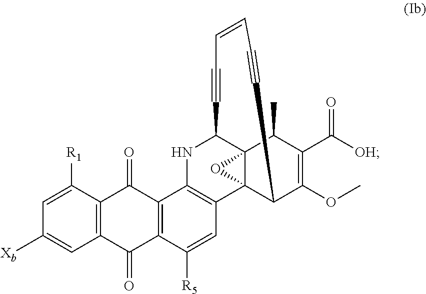

wherein: X_{b} is C₁-C₄ alkyl; R₁ is selected from H, hydroxyl, and —O—(C═O)—CH₃; and R₃ is hydroxyl or —O—(C═O)—CH₃. In particular aspects, X_{b} is methyl or ethyl.

In certain aspects, the compound is a compound of formula (Ic):

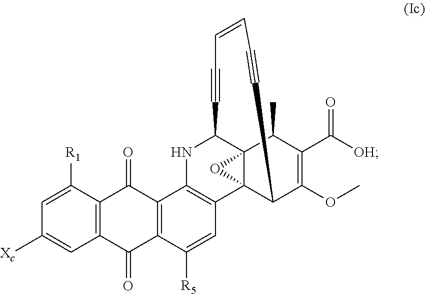

wherein: X_{c} is selected from Br, cyano, and —(CH₂)ₙ—NR₆R₇, wherein n is an integer selected from 0, 1, 2, 3, and 4, R₆ and R₇ are each independently selected from H, C₁-C₄ alkyl, and a protecting group; R₁ is selected from H, hydroxyl, and —O—(C═O)—CH₃; and R₅ is hydroxyl or —O—(C═O)—CH₃. In particular aspects, R₆ and R₇ are each H.

In certain aspects, the compound is a compound of formula (Id):

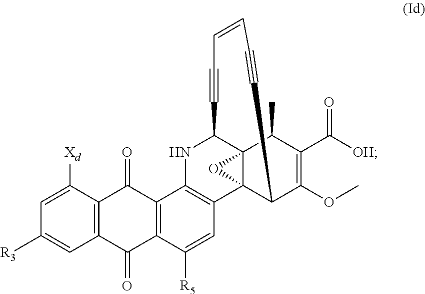

wherein: X_{d} is H; R₃ is selected from C₁-C₄ alkyl, halogen, and —(CH₂)ₙ—NR₆R₇, wherein n is an integer selected from 0, 1, 2, 3, and 4, R₆ and R₇ are each independently selected from H, C₁-C₄ alkyl, and a protecting group; and R₅ is hydroxyl or —O—(C═O)—CH₃.

In certain aspects, the compound is a compound of formula (Ie):

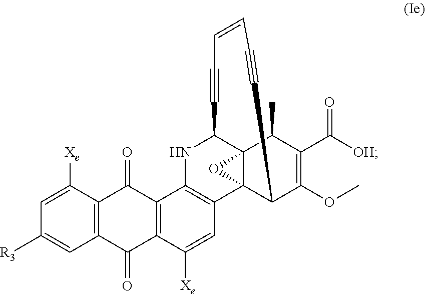

wherein: each Xₑ is hydroxyl; and R₃ is selected from C₁-C₄ alkyl, halogen, and —(CH₂)ₙ—NR₆R₇, wherein n is an integer selected from 0, 1, 2, 3, and 4, R₆ and R₇ are each independently selected from H, C₁-C₄ alkyl, and a protecting group.

In certain aspects, the compound is a compound of formula (If):

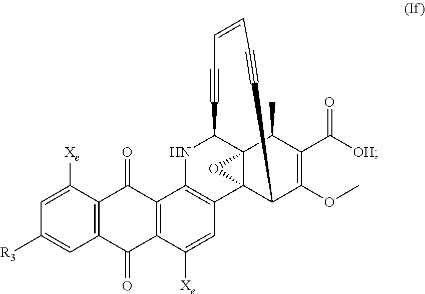

wherein: each Xₑ is —O—(C═O)—CH₃; and R₃ is selected from C₁-C₄ alkyl, halogen, and —(CH₂)ₙ—NR₆R₇, wherein n is an integer selected from 0, 1, 2, 3, and 4, R₆ and R₇ are each independently selected from H, C₁-C₄ alkyl, and a protecting group.

In particular aspects of the compound of formula (I) or formula (I′): R₃ is F and R₁ and R₅ are each independently OH; R₃ is F, R₁ is H, and R₅ is OH; R₃ is F and R₁ and R₅ are each independently —O—(C═O)—CH₃; R₃ is Cl and R₁ and R₅ are each independently —O—(C═O)—CH₃; R₃ is methyl and R₁ and R₅ are each independently OH; R₃ is methyl, R₁ is H, R₅ is OH; R₃ is methyl, and R₁ and R₅ are each independently —O—(C═O)—CH₃; R₃ is Cl and R₁ and R₅ are each independently OH; R₃ is Cl, R₁ is H, and R₅ is OH; R₃ is Cl, R₁ is H, and R₅ is —O—(C═O)—CH₃; R₃ is ethyl, R₁ is H, and R₅ is OH; R₃ is ethyl, R₁ is H, and R₅ is —O—(C═O)—CH₃; R₃ is NH₂ and R₁ and R₅ are each independently OH; or R₃ is NH₂, R₁ is H, and R₅ is OH.

In other aspects, the presently disclosed subject matter provides a pharmaceutical composition comprising a compound of formula (I), formula (I′), or formula (Ia-If) and a pharmaceutically acceptable carrier.

In yet other aspects, the presently disclosed subject matter provides a conjugate comprising a compound of formula (I), formula (I′), or formula (Ia-If) and an antibody.

In certain aspects, the antibody is selected from a CD33 monoclonal antibody (mAB); a CD22 mAB; an mAb directed against type IV collagenase, including MMP-2 and MMP-9; anti-CD19(Fab); an antibody directed against polymorphic epithelial mucin, e.g., hCTM01; an antibody recognizing Lewis^{γ} (Le^{γ}) antigen, e.g., hu3S193; an antibody that recognizes human renal gamma-glutamyltransferase (GGT), e.g., 138H11; an antibody recognizing a tumor-specific antigen, such as the glycoprotein on the cell surface of human colon cancer, e.g., A7; and an antibody recognizing a tumor-specific antigen, such as pancreatic cancer e.g., chA7Fab.

In other aspects, the presently disclosed subject matter provides a conjugate comprising a compound of formula (I), formula (I′), or formula (Ia-If) and a delivery system. In particular aspects, the delivery system comprises a poly(styrene-co-maleic acid).

In other aspects, the presently disclosed subject matter provides a compound of formula (II):

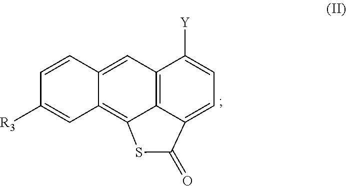

wherein: Y is selected from halogen, hydroxyl, methoxyl, and —O—S(═O)₂—CF₃ (—OTf); and R₃ is selected from halogen, C₁-C₄ alkyl, —C(═O)—CH₃, —NH—C(═O)—CH₃, cyano, and —(CH₂)ₙ—NR₆R₇, wherein n is an integer selected from 0, 1, 2, 3, and 4, R₆ and R₇ are each independently selected from H, C₁-C₄ alkyl, and a protecting group. In particular aspects, Y is iodine. In particular aspects, R₃ is selected from Cl, Br, F, methyl, and ethyl.

In other aspects, the presently disclosed subject matter provides a method for generating dynemicin or an analogue thereof, the method comprising: culturing a plurality of mutant Micromonospora lacking ORF15 in growth media supplemented with an iodoanthracene of formula (II) for a period of time and under conditions sufficient to process the iodoanthracene; and purifying the dynemicin or analogue thereof.

In certain aspects, the iodoanthracene of formula (II) is a modified iodoanthracene. In particular aspects, the modified iodoanthracene comprises a modification at one of more positions selected from C3, C8, and C9. In more particular aspects, the modified iodoanthracene comprises a modification at one of more positions selected from C8 and C9. In yet more particular aspects, the modified iodoanthracene is a 9-substituted iodoanthracene.

In certain aspects, the modified iodoanthracene is a compound of formula (II):

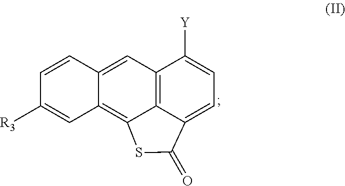

wherein: Y is selected from halogen, hydroxyl, methoxyl, and —O—S(═O)₂—CF₃ (—OTf); and R₃ is selected from halogen, C₁-C₄ alkyl, —C(═O)—CH₃, —NH—C(═O)—CH₃, cyano, and —(CH₂)ₙ—NR₆R₇, wherein n is an integer selected from 0, 1, 2, 3, and 4, R₆ and R₇ are each independently selected from H, C₁-C₄ alkyl, and a protecting group. In particular aspects, Y is iodine. In particular aspects, R₃ is selected from Cl, Br, F, cyano, methyl, and ethyl.

In more particular aspects, the modified iodoanthracene is selected from the group consisting of: 9-fluoro-5-iodoanthracene-γ-thiolactone, 9-chloro-5-iodoanthracene-γ-thiolactone, 9-methyl-5-iodoanthracene-γ-thiolactone, 9-ethyl-5-iodoanthracene-γ-thiolactone, and 9-amino-5-iodoanthracene-γ-thiolactone.

In certain aspects, the modified iodoanthracene is prepared by one or all of: (a) providing a 5-substituted phthalide; (b) formation of an anthracene-γ-thiolactone comprising a masking group capable of being converted to an iodine substituent group; (c) modification of an A-ring substituent group; and (d) introduction of an iodine group in a C-ring of the anthracene-γ-thiolactone.

In certain aspects, the 5-substituted phthalide is prepared by the method provided in FIG. 21. In certain aspects, the modified iodoanthracene is prepared by the method provided in FIG. 22.

In particular aspects, the dynemicin analogue is a methyl-, chloro-, ethyl-, fluoro-, cyano-, or an amino-analogue of dynemicin.

In certain aspects, the period of time is about seven or more days. In certain aspects, the conditions sufficient to process the iodoanthracene include incubation at 28° C. with shaking. In certain aspects, the method further comprises generating a mutant Micromonospora lacking ORF15. In particular aspects, the mutant Micromonospora is Micromonospora chersina.

In yet other aspects, the presently disclosed subject matter provides a method for treating cancer, the method comprising administering a therapeutically effective amount of a compound of formula (I), formula (I′), or formula (Ia-If), or a composition or a conjugate thereof, to a subject in need of treatment thereof.

In certain aspects, the cancer is selected from cervical carcinoma; leukemia; melanoma; hepatocellular carcinoma, also known as hepatoma (including advanced or recurrent hepatocellular carcinoma); acute myeloid leukemia (AML) (including first relapse with CD33-positive AML); B-cell acute lymphoblastic leukemia (ALL) (including CD22-positive B-cell ALL); colorectal carcinoma; sarcoma; breast cancer; ovarian carcinoma; gastric (or stomach) cancer; prostate carcinomas; metastasized renal cell carcinoma (RCC) (including metastasized RCC), and pancreatic carcinoma.

Certain aspects of the presently disclosed subject matter having been stated hereinabove, which are addressed in whole or in part by the presently disclosed subject matter, other aspects will become evident as the description proceeds when taken in connection with the accompanying Examples and Drawings as best described herein below.

### BRIEF DESCRIPTION OF THE FIGURES

The patent or application file contains at least one drawing executed in color. Copies of this patent or patent application publication with color drawing(s) will be provided by the Office upon request and payment of the necessary fee.

Having thus described the presently disclosed subject matter in general terms, reference will now be made to the accompanying Figures, which are not necessarily drawn to scale, and wherein:

FIG. 1 shows representative members of three enediyne structural classes: 9-membered neocarzinostatin (Ncs, 1), 10-membered calicheamicin γ1I (Clm, 2), and anthraquinone-fused enediyne (AFE) dynemicin A (Dyn, 3);

FIG. 2 shows an overview of Dyn biosynthesis and shunt products 6-9 from ΔdynE13;

FIG. 3a, FIG. 3b, FIG. 3c, and FIG. 3d show: (FIG. 3a) Synthesis of iodoanthracene 5 and bromoanthracene 13; (FIG. 3b) HPLC of Dyn 3, reduced anthracene 12 and iodoanthracene 5 production in M. chersina fermentations with and without supplementation with iodoanthracene 5, and (FIG. 3c) bromoanthracene 13; (FIG. 3d) Dyn production by wild type fermentation in the presence of bromoanthracene 13 at increasing concentrations.

FIG. 4a, FIG. 4b, and FIG. 4c shows: (FIG. 4a) Structure of sungeidine B 21 and sungeidine C 22 and potential structural types of the enediyne coupling partner as secondary amine 23 or primary amine 24; (FIG. 4b) Synthesis of aminoanthracene 25; (FIG. 4c) HPLC comparisons of Dyn 3, reduced anthracene 12 and iodoanthracene 5 production in M. chersina fermentations with and without aminoanthracene 25;

FIG. 5a and FIG. 5b show: (FIG. 5a) Synthesis of the anthracene carboxylic acid (28); (FIG. 5b) Small-molecule-protein pull-down assay with the SDS-PAGE gel after 4 h of incubation (L=Ladder, W1, W2=Washes with PBS buffer, E1, E2=Elutions with PBS buffer+0.25 mM and 0.50 mM substrates (5 and 13, 4:1 ratio) respectively;

FIG. 6a and FIG. 6b show: (FIG. 6a) Predicted structure of Orf14 using AlphaFold, N-terminal domain, C-terminal domain and the N-terminal unstructured region is colored in dark purple, dark pink and light pink respectively; (FIG. 6b) Overlay of Orf14 AlphaFold model (dark purple, structured domains only) with the crystal structures of DynU16 (orange) and CalU16 (green);

FIG. 7a and FIG. 7b show: (FIG. 7a) Comparison of the metabolite profile of Δorf14 and Δorf16 strains with wild type M. chersina (with and without NaI); (FIG. 7b) Quantification of Dyn 3, reduced anthracene 12 and iodoanthracene 5 production by wild type M. chersina, Δorf14 and Δorf16 strains;

FIG. 8 is a chemical model reaction for C—N bond formation;

FIG. 9 shows quantification of compound 30 in washes after loading onto BcMag™ magnetic beads and after hydrolysis from the beads;

FIG. 10a and FIG. 10b show: (FIG. 2a) SDS-PAGE gel of the M. chersina Δorf14 and wild type cell-free extract pull-down experiment and (FIG. 2b) the control after 24 h. Bands B1 and B2 correspond to Orf16 and Orf14 respectively from wild type cell-free extract pull down experiment as a control. B3 corresponds to Orf16 pulled down from Δorf14 cell-free extract, whereas B4 is an amino acid adenylation domain-containing protein (accession number SCL46042), a non-specific protein outside of the dynemicin BGC;

FIG. 11a, FIG. 11b, and FIG. 11c show: (FIG. 11a) Predicted structure of Orf16 using AlphaFold, with two domains colored separately, (FIG. 11b) sequence coverage map of Orf14 structural model, and (FIG. 11c) Orf16 structural model;

FIG. 12 is SDS-PAGE gel of small-scale expression tests for a) p28a_Orf14, p29b_Orf14, and b) p28a_Orf16 and p29b_Orf16. U=Uninduced, I=Induced, Ins=Insoluble, Sol=Soluble;

FIG. 13a, FIG. 13b, FIG. 13c, FIG. 13d, FIG. 13e, FIG. 13f, FIG. 13g; FIG. 13h, FIG. 13i, and FIG. 13j show HPLC analysis of in vitro reaction of Orf14+Orf16 with iodoanthracene 5 and (FIG. 13a) 4-methoxybenzylamine (amine 1), (FIG. 13b) α-methoxybenzylamine (amine 2), (FIG. 13c) cyclohexylamine (amine 3), (FIG. 13d) benzylamine (amine 4), (FIG. 13e) butylamine (amine 5), (FIG. 13f) 4-chlorobenzylamine (amine 6), (FIG. 13g) 1-methyl pyrrolidine (amine 7), (FIG. 13h) pyrrolidine (amine 8), (FIG. 13i) piperidine (amine 9), and (FIG. 13j) isoquinolene (amine 10). Amines 3, 5, 7, 8 and 9 have no absorption at 280 nm;

FIG. 14a, FIG. 14b, and FIG. 14c shows HPLC analysis of in vitro reaction of Orf14+Orf16 with iodoanthracene 5 and day 7 extract of (FIG. 14a) wild type, (FIG. 14b) ΔOrf14 and (FIG. 14c) ΔdynE8 fermentations;

FIG. 15 shows representative examples of enediyne natural products;

FIG. 16 shows examples of linker installation on anthraquinone fused enediynes. Multiple sites on the A-ring of both Ucm and Tnm have been used for linker installation. Additionally, linker has been attached on the C-ring hydroxyl as well as D-ring alcohol.

FIG. 17a, FIG. 17b, and FIG. 17c show a comparison of product profile of ΔOrf15 to wild type M. chersina. HPLC traces at two different wavelengths (FIG. 17a) 570 nm and (FIG. 17b) 450 nm have been shown for better visualization of Dyn (3) and iodo- (7) and des-iodoanthracene (8, shunt product), respectively. (FIG. 17c) Plausible biosynthetic route involving Orf15 in the synthesis of the iodoanthracene (7). The anthraquinone core in Dyn (3) originating from 7, is colored in blue. The numbering systems for 3 and 7 also are shown;

FIG. 18 is a combinatorial biosynthesis strategy for the presently disclosed dynemicin analogues;

FIG. 19 shows a mechanism of activation and DNA cleavage by Dyn;

FIG. 20 is a general strategy for the synthesis of the iodoanthracene analogues;

FIG. 21 is a scheme for the synthesis of 5-substituted phthalides;

FIG. 22 is a scheme for the synthesis of the 9-substituted-5-iodoanthracene-γ-thiolactones;

FIG. 23a and FIG. 23b is a product profile of ΔOrf15 fermentation supplemented with 20a. (FIG. 23a) comparison of small and large-scale fermentations of ΔOrf15 are shown along with Dyn 3 standard and 20a standard at 570 nm (except 20a standard is shown at 450 nm for better visualization). The Dyn peak almost overlaps with 22, but both have different UV-Vis spectra. The peaks around 25 min and 29 min in the ΔOrf15 large fermentation arises from break-down of the LH-20 resin beads used in the fermentation. (FIG. 23b) UV-Vis spectra of 21, 22 and Dyn 3 are shown for comparison;

FIG. 24a and FIG. 24b product profile of ΔOrf15 fermentation supplemented with 20b, 20c, 20d. (FIG. 24a) comparison of small-scale fermentations of ΔOrf15 supplemented with 20b-20d are shown along with Dyn 3 standard and 20b-20d standards at 570 nm (except 20b-20d standards are shown at 450 nm for better visualization). (FIG. 24b) UV-Vis spectra of 24, 25, 27, 28 and 29 are shown for comparison;

FIG. 25 shows diversification of the bromo-intermediate to various other functional groups;

FIG. 26a and FIG. 26b show product profile of ΔOrf15 fermentations supplemented with 34 and 36. (FIG. 26a) comparison of small-scale fermentations of ΔOrf15 supplemented with 34 and 36 is shown along with Dyn 3 standard and 34, 36 standards at 570 nm (except 34, 36 standards are shown at 450 nm for better visualization). (FIG. 26b) UV-Vis spectra of 37, 38, 39, 40, 41 and 42 are shown for comparison;

FIG. 27 are representative dynemicin analogues;

FIG. 28a and FIG. 28b show: (FIG. 28a) Representative enediyne structures and clinical examples from the three structural classes; Polymer-conjugated neocarzinostatin (9-membered enediyne) SMANCS (1), Maeda et al., 2001, uncialamycin ADC (10-membered anthraquinone fused enediyne) (2) and calicheamicin ADC (10-membered enediyne, non-anthraquinone fused) Gemtuzumab (3). (FIG. 28b) Comparison of biosynthetic gene clusters of uncialamycin (UCM, 6), tiancimycin (TNM, 7), dynemicin (DYN, 4) and calicheamicin (CAL);

FIG. 29a, FIG. 29b, FIG. 29c, FIG. 29d, FIG. 29e show the product profile of Δorf15 fermentation supplemented with 16a. (FIG. 29a) comparison of fermentation of Δorf15 supplemented with 16a along with DYN (4) standard and 16a standard at 570 nm (except 16a standard is shown at 450 nm for better visualization). The DYN peak almost overlaps with 29, but both have different UV-vis spectra. (FIG. 29b) Structure of compounds 28-30. UV-vis spectra of (FIG. 29c) 28, (FIG. 29d) 29 and (FIG. 29e) DYN (4) are shown for comparison;

FIG. 30a, FIG. 30b, FIG. 30c, FIG. 30d, FIG. 30e, FIG. 30f, and FIG. 30g show product profiles of Δorf15 fermentations supplemented with 16b, 16c, 16d. (FIG. 30a) Comparison of small-scale fermentations of Δorf15 supplemented with 16b-d are shown along with DYN (4) standard and 16b-d standards at 570 nm (except 16b-d standards are shown at 450 nm for better visualization). (FIG. 30b) Structure of compounds 31-38. UV-vis spectra of (FIG. 30c) 31, (FIG. 30d) 32, (FIG. 30e) 34, (FIG. 30f) 35 and (FIG. 30g) 29 are shown for comparison; and

FIG. 31a, FIG. 31b, FIG. 31c, FIG. 31d, FIG. 31e, FIG. 31f, FIG. 31g, and FIG. 31h show product profile of Δorf15 fermentations supplemented with 43 and 45. (a) comparison of small-scale fermentations of Δorf15 supplemented with 43 and 45 is shown along with DYN (4) standard and 43, 45 standards at 570 nm (except 43, 45 standards are shown at 450 nm for better visualization). (FIG. 31b) Structure of compounds 50, 51. UV-vis spectra of (FIG. 31c) 37, (FIG. 31d) 38, (FIG. 31e) 39, (FIG. 31f) 40, (FIG. 31g) 41 and (FIG. 31h) 42 are shown for comparison.

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

There are three main families of enediyne anti-tumor natural products, the anthraquinone-fused being one. Many are potent cleavers of DNA (as low as pM) and can be conjugated to antibodies for selective cell delivery (actual drugs and many in clinical trials). The presently disclosed subject matter, in part, uses synthetic modifications of a natural anthracene biosynthetic intermediate to direct the biosynthetic machinery of the producing organism to synthesize structurally modified anthraquinone-fused enediynes (AFE's) to modulate DNA binding and redox potential of the anthraquinone to “tune” DNA cleavage behavior. The compounds are a new structural type and have bright yellow fluorescence properties.

### A. Dyneinicin Analogues

Accordingly, in some embodiments, the presently disclosed subject matter provides a compound of formula (I):

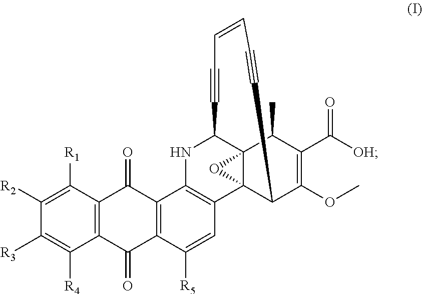

wherein: R₁ is selected from H, hydroxyl, and —O—(C═O)—CH₃; R₂ and R₃ are each independently selected from H, hydroxyl, C₁-C₄ alkyl, C₁-C₄ alkoxyl, halogen, carbonyl, carboxyl, acetyl, cyano, mercapto, nitro, —O—(C═O)—CH₃, and —(CH₂)ₐ—NR₆R₇, wherein n is an integer selected from 0, 1, 2, 3, and 4, R₆ and R₇ are each independently selected from H, C₁-C₄ alkyl, and a protecting group; R₅ is hydroxyl or —O—(C═O)—CH₃; provided that if R₁, R₄, and R₅ are each hydroxyl, then R₂ and R₃ cannot both be H; and; stereoisomers and pharmaceutically acceptable salts thereof.

The term “alkyl” refers to univalent groups derived from alkanes by removal of a hydrogen atom from any carbon atom —CₙH₂ₙ₊₁. The groups derived by removal of a hydrogen atom from a terminal carbon atom of unbranched alkanes form a subclass of normal alkyl (n-alkyl) groups H(CH₂)ₙ. The groups RCH₂, R₂CH (R≠H), and R₃C (R≠H) are primary, secondary and tertiary alkyl groups, respectively.

“C₁-C₄ alkyl” refers to an alkyl group having 1 to 4 carbon atoms, e.g., 1, 2, 3, or 4 carbon atoms. Representative C₁-C₄ alkyl groups include methyl, ethyl, n-propyl, isopropyl, n-butyl, and t-butyl.

“C₁-C₄ alkoxyl” refers to a —OR group, where R is a C₁-C₄ alkyl as defined immediately hereinabove. Representative alkoxyl groups include methoxyl, ethoxyl, and the like.

“Halogen” refers to chlorine (Cl), bromine (Br), fluorine (F), and iodine (I).

In certain embodiments, the compound of formula (I) is a compound of formula (I′):

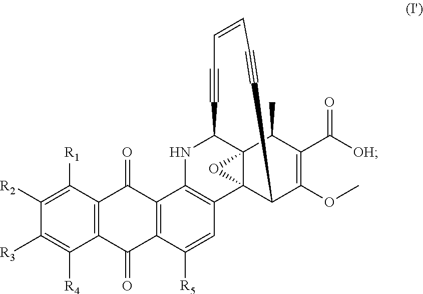

wherein: R₁ is selected from H, hydroxyl, and —O—(C═O)—CH₃; R₂ and R₄ are each H; R₃ is selected from C₁-C₄ alkyl, halogen, —(CH₂)ₙ—NR₆R₇, wherein n is an integer selected from 0, 1, 2, 3, and 4, R₆ and R₇ are each independently selected from H, C₁-C₄ alkyl, and a protecting group; R₅ is hydroxyl or —O—(C═O)—CH₃; and stereoisomers and pharmaceutically acceptable salts thereof.

In certain embodiments, the compound is a compound of formula (Ta):

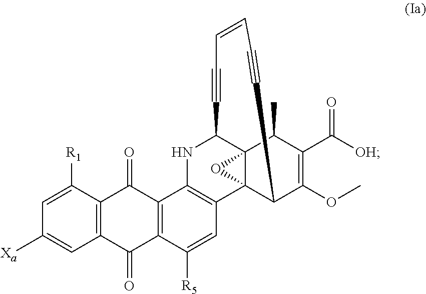

wherein: Xₐ is halogen or cyano; R₁ is selected from H, hydroxyl, and —O—(C═O)—CH₃; and R₅ is hydroxyl or —O—(C═O)—CH₃. In particular embodiments, Xₐ is selected from Br, Cl, F, and cyano.

In certain embodiments, the compound is a compound of formula (Ib):

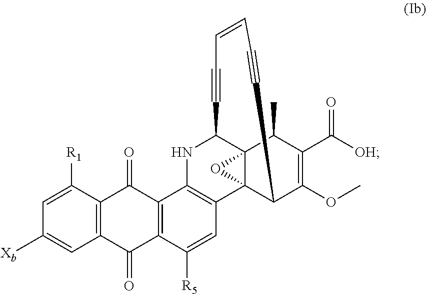

wherein: X_{b} is C1-C₄ alkyl; R₁ is selected from H, hydroxyl, and —O—(C═O)—CH₃; and R₃ is hydroxyl or —O—(C═O)—CH₃. In particular embodiments, X_{b} is methyl or ethyl.

In certain embodiments, the compound is a compound of formula (Ic):

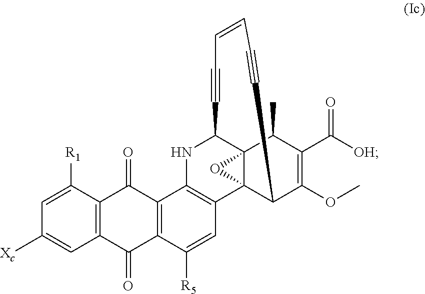

wherein: X_{c} is selected from Br, cyano, and —(CH₂)ₙ—NR₆R₇, wherein n is an integer selected from 0, 1, 2, 3, and 4, R₆ and R₇ are each independently selected from H, C₁-C₄ alkyl, and a protecting group; R₁ is selected from H, hydroxyl, and —O—(C═O)—CH₃; and R₅ is hydroxyl or —O—(C═O)—CH₃. In particular embodiments, R₆ and R₇ are each H.

In certain embodiments, the compound is a compound of formula (Id):

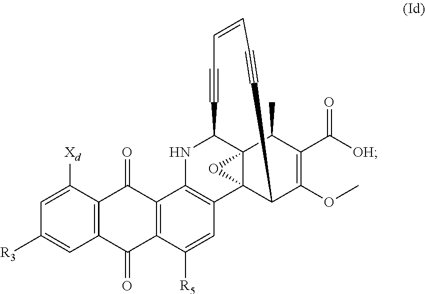

wherein: X_{d} is H; R₃ is selected from C₁-C₄ alkyl, halogen, and —(CH₂)ₙ—NR₆R₇, wherein n is an integer selected from 0, 1, 2, 3, and 4, R₆ and R₇ are each independently selected from H, C₁-C₄ alkyl, and a protecting group; and R₅ is hydroxyl or —O—(C═O)—CH₃.

In certain embodiments, the compound is a compound of formula (Ie):

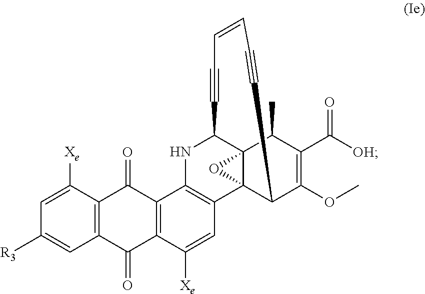

wherein: each Xₑ is hydroxyl; and R₃ is selected from C₁-C₄ alkyl, halogen, and —(CH₂)~—NR₆R₇, wherein n is an integer selected from 0, 1, 2, 3, and 4, R₆ and R₇ are each independently selected from H, C₁-C₄ alkyl, and a protecting group.

In certain embodiments, the compound is a compound of formula (If):

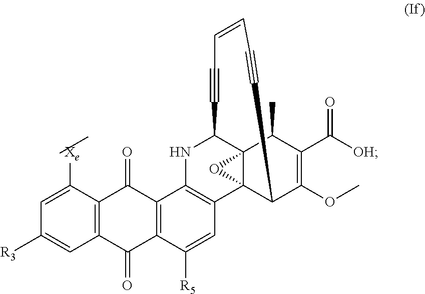

wherein: each Xₑ is —O—(C═O)—CH₃; and R₃ is selected from C₁-C₄ alkyl, halogen, and —(CH₂)ₙ—NR₆R₇, wherein n is an integer selected from 0, 1, 2, 3, and 4, R₆ and R₇ are each independently selected from H, C₁-C₄ alkyl, and a protecting group.

In particular embodiments of the compound of formula (I) or formula (I′): R₃ is F and R₁ and R₅ are each independently OH; R₃ is F, R₁ is H, and R₅ is OH; R₃ is F and R₁ and R₅ are each independently —O—(C═O)—CH₃; R₃ is Cl and R₁ and R₅ are each independently —O—(C═O)—CH₃; R₃ is methyl and R₁ and R₅ are each independently OH; R₃ is methyl, R₁ is H, R₅ is OH; R₃ is methyl, and R₁ and R₅ are each independently —O—(C═O)—CH₃; R₃ is Cl and R₁ and R₅ are each independently OH; R₃ is Cl, Rₜ is H, and R₅ is OH; R₃ is Cl, R₁ is H, and R₅ is —O—(C═O)—CH₃; R₃ is ethyl, R₁ is H, and R₅ is OH; R₃ is ethyl, R₁ is H, and R₅ is —O—(C═O)—CH₃; R₃ is NH₂ and R₁ and R₅ are each independently OH; or R₃ is NH₂, R₁ is H, and R₅ is OH.

In other embodiments, the presently disclosed subject matter provides a pharmaceutical composition comprising a compound of formula (I), formula (I′), or formula (Ia-If) and a pharmaceutically acceptable carrier.

In yet other embodiments, the presently disclosed subject matter provides a conjugate comprising a compound of formula (I), formula (I′), or formula (Ia-If) and an antibody.

In certain embodiments, the antibody is selected from a CD33 monoclonal antibody (mAB); a CD22 mAB; an mAb directed against type IV collagenase, including MMP-2 and MMP-9; anti-CD19(Fab); an antibody directed against polymorphic epithelial mucin, e.g., hCTM01; an antibody recognizing Lewis^{γ} (Le^{γ}) antigen, e.g., hu3S193; an antibody that recognizes human renal gamma-glutamyltransferase (GGT), e.g., 138H11; an antibody recognizing a tumor-specific antigen, such as the glycoprotein on the cell surface of human colon cancer, e.g., A7; and an antibody recognizing a tumor-specific antigen, such as pancreatic cancer e.g., chA7Fab.

In other embodiments, the presently disclosed subject matter provides a conjugate comprising a compound of formula (I), formula (I′), or formula (Ia-If) and a delivery system. In particular embodiments, the delivery system comprises a poly(styrene-co-maleic acid).

### B. Modified Anthracenes of Formula (II)

In other embodiments, the presently disclosed subject matter provides a compound of formula (II):

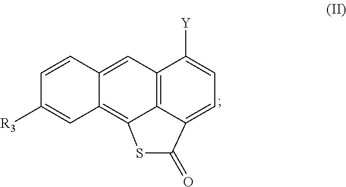

wherein: Y is selected from halogen, hydroxyl, methoxyl, and —O—S(═O)₂—CF₃ (—OTf); and R₃ is selected from halogen, C₁-C₄ alkyl, —C(═O)—CH₃, —NH—C(═O)—CH₃, cyano, and —(CH₂)ₙ—NR₆R₇, wherein n is an integer selected from 0, 1, 2, 3, and 4, R₆ and R₇ are each independently selected from H, C₁-C₄ alkyl, and a protecting group. In particular embodiments, Y is iodine. In particular embodiments, R₃ is selected from Cl, Br, F, methyl, and ethyl.

### B. Methods for Generating Dynemicin or an Analogue Thereof

In other embodiments, the presently disclosed subject matter provides a method for generating dynemicin or an analogue thereof, the method comprising: culturing a plurality of mutant Micromonospora lacking ORF15 in growth media supplemented with an iodoanthracene of formula (II) for a period of time and under conditions sufficient to process the iodoanthracene; and purifying the dynemicin or analogue thereof.

In certain embodiments, the iodoanthracene of formula (II) is a modified iodoanthracene. In particular embodiments, the modified iodoanthracene comprises a modification at one of more positions selected from C3, C8, and C9. In more particular embodiments, the modified iodoanthracene comprises a modification at one of more positions selected from C8 and C9. In yet more particular embodiments, the modified iodoanthracene is a 9-substituted iodoanthracene.

In certain embodiments, the modified iodoanthracene is a compound of formula (II):

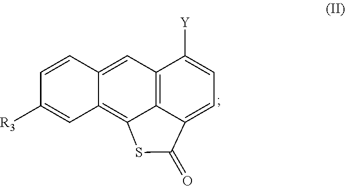

wherein: Y is selected from halogen, hydroxyl, methoxyl, and —O—S(═O)₂—CF₃ (—OTf); and R₃ is selected from halogen, C₁-C₄ alkyl, —C(═O)—CH₃, —NH—C(═O)—CH₃, cyano, and —(CH₂)ₙ—NR₆R₇, wherein n is an integer selected from 0, 1, 2, 3, and 4, R₆ and R₇ are each independently selected from H, C₁-C₄ alkyl, and a protecting group. In particular embodiments, Y is iodine. In particular embodiments, R₃ is selected from Cl, Br, F, cyano, methyl, and ethyl.

In more particular embodiments, the modified iodoanthracene is selected from the group consisting of: 9-fluoro-5-iodoanthracene-γ-thiolactone, 9-chloro-5-iodoanthracene-γ-thiolactone, 9-methyl-5-iodoanthracene-γ-thiolactone, 9-ethyl-5-iodoanthracene-γ-thiolactone, and 9-amino-5-iodoanthracene-γ-thiolactone.

In certain embodiments, the modified iodoanthracene is prepared by one or all of: (a) providing a 5-substituted phthalide; (b) formation of an anthracene-γ-thiolactone comprising a masking group capable of being converted to an iodine substituent group; (c) modification of an A-ring substituent group; and (d) introduction of an iodine group in a C-ring of the anthracene-γ-thiolactone.

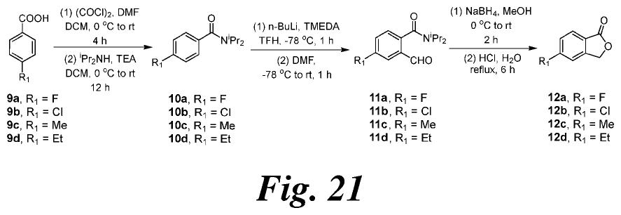

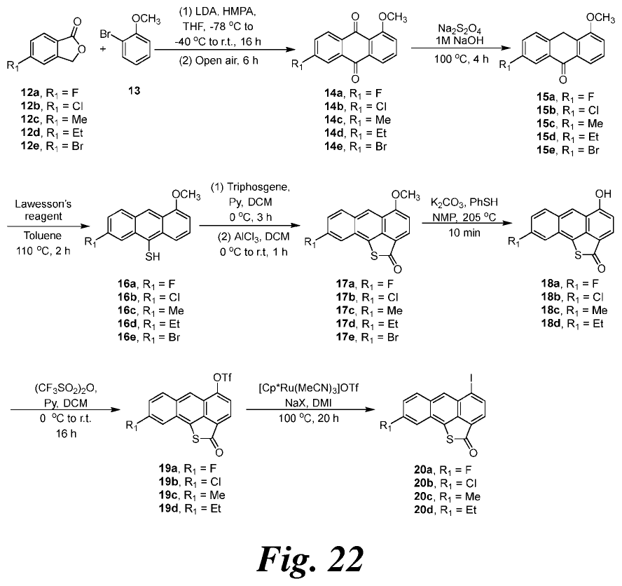

In certain embodiments, the 5-substituted phthalide is prepared by the method provided in FIG. 21. In certain embodiments, the modified iodoanthracene is prepared by the method provided in FIG. 22.

In particular embodiments, the dynemicin analogue is a methyl-, chloro-, ethyl-, fluoro-, cyano-, or an amino-analogue of dynemicin.

In certain embodiments, the period of time is about seven or more days. In certain embodiments, the conditions sufficient to process the iodoanthracene include incubation at 28° C. with shaking. In certain embodiments, the method further comprises generating a mutant Micromonospora lacking ORF15. In particular embodiments, the mutant Micromonospora is Micromonospora chersina.

### D. Methods for Treating Cancer

In yet other embodiments, the presently disclosed subject matter provides a method for treating cancer, the method comprising administering a therapeutically effective amount of a compound of formula (I), formula (I′), or formula (Ia-If), or a composition or a conjugate thereof, to a subject in need of treatment thereof.

In certain embodiments, the cancer is selected from cervical carcinoma; leukemia; melanoma; hepatocellular carcinoma, also known as hepatoma (including advanced or recurrent hepatocellular carcinoma); acute myeloid leukemia (AML) (including first relapse with CD33-positive AML); B-cell acute lymphoblastic leukemia (ALL) (including CD22-positive B-cell ALL); colorectal carcinoma; sarcoma; breast cancer; ovarian carcinoma; gastric (or stomach) cancer; prostate carcinomas; metastasized renal cell carcinoma (RCC) (including metastasized RCC), and pancreatic carcinoma.

As used herein, the term “treating” can include reversing, alleviating, inhibiting the progression of, preventing or reducing the likelihood of the disease, disorder, or condition to which such term applies, or one or more symptoms or manifestations of such disease, disorder or condition. Preventing refers to causing a disease, disorder, condition, or symptom or manifestation of such, or worsening of the severity of such, not to occur. Accordingly, the presently disclosed peptides can be administered prophylactically to prevent or reduce the incidence or recurrence of the disease, disorder, or condition.

In general, the “therapeutically effective amount” of an active agent or drug delivery device refers to the amount necessary to elicit the desired biological response. As will be appreciated by those of ordinary skill in this art, the effective amount of an agent or device may vary depending on such factors as the desired biological endpoint, the agent to be delivered, the makeup of the pharmaceutical composition, the target tissue, and the like.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; poultry, such as domestic fowls including, but not limited to chickens, turkeys, geese, ducks, quail, guinea fowl, and pigeons; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

While the following terms in relation to compounds of formula (I) or formula (I′), are believed to be well understood by one of ordinary skill in the art, the following definitions are set forth to facilitate explanation of the presently disclosed subject matter. These definitions are intended to supplement and illustrate, not preclude, the definitions that would be apparent to one of ordinary skill in the art upon review of the present disclosure.

The terms substituted, whether preceded by the term “optionally” or not, and substituent, as used herein, refer to the ability, as appreciated by one skilled in this art, to change one functional group for another functional group on a molecule, provided that the valency of all atoms is maintained. When more than one position in any given structure may be substituted with more than one substituent selected from a specified group, the substituent may be either the same or different at every position. The substituents also may be further substituted (e.g., an aryl group substituent may have another substituent off it, such as another aryl group, which is further substituted at one or more positions).

Where substituent groups or linking groups are specified by their conventional chemical formulae, written from left to right, they equally encompass the chemically identical substituents that would result from writing the structure from right to left, e.g., —CH₂O— is equivalent to —OCH₂—; —C(═O)O— is equivalent to —OC(═O)—; —OC(═O)NR— is equivalent to —NRC(═O)O—, and the like.

When the term “independently selected” is used, the substituents being referred to (e.g., R groups, such as groups R₁, R₂, and the like, or variables, such as “m” and “n”), can be identical or different. For example, both R₁ and R₂ can be substituted alkyls, or R₁ can be hydrogen and R₂ can be a substituted alkyl, and the like.

The terms “a,” “an,” or “a(n),” when used in reference to a group of substituents herein, mean at least one. For example, where a compound is substituted with “an” alkyl or aryl, the compound is optionally substituted with at least one alkyl and/or at least one aryl. Moreover, where a moiety is substituted with an R substituent, the group may be referred to as “R-substituted.” Where a moiety is R-substituted, the moiety is substituted with at least one R substituent and each R substituent is optionally different.

A named “R” or group will generally have the structure that is recognized in the art as corresponding to a group having that name, unless specified otherwise herein. For the purposes of illustration, certain representative “R” groups as set forth above are defined below.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

Unless otherwise explicitly defined, a “substituent group,” as used herein, includes a functional group selected from one or more of the following moieties, which are defined herein:

The term hydrocarbon, as used herein, refers to any chemical group comprising hydrogen and carbon. The hydrocarbon may be substituted or unsubstituted. As would be known to one skilled in this art, all valencies must be satisfied in making any substitutions. The hydrocarbon may be unsaturated, saturated, branched, unbranched, cyclic, polycyclic, or heterocyclic. Illustrative hydrocarbons are further defined herein below and include, for example, methyl, ethyl, n-propyl, isopropyl, cyclopropyl, allyl, vinyl, n-butyl, tert-butyl, ethynyl, cyclohexyl, and the like.

The term “alkyl,” by itself or as part of another substituent, means, unless otherwise stated, a straight (i.e., unbranched) or branched chain, acyclic or cyclic hydrocarbon group, or combination thereof, which may be fully saturated, mono- or polyunsaturated and can include di- and multivalent groups, having the number of carbon atoms designated (i.e., C₁₋₁₀ means one to ten carbons, including 1, 2, 3, 4, 5, 6, 7, 8, 9, and 10 carbons). In particular embodiments, the term “alkyl” refers to C₁₋₂₀ inclusive, including 1, 2, 3, 4, 5, 6, 7, 8, 9, 10, 11, 12, 13, 14, 15, 16, 17, 18, 19, and 20 carbons, linear (i.e., “straight-chain”), branched, or cyclic, saturated or at least partially and in some cases fully unsaturated (i.e., alkenyl and alkynyl) hydrocarbon radicals derived from a hydrocarbon moiety containing between one and twenty carbon atoms by removal of a single hydrogen atom.

Representative saturated hydrocarbon groups include, but are not limited to, methyl, ethyl, n-propyl, isopropyl, n-butyl, isobutyl, sec-butyl, tert-butyl, n-pentyl, sec-pentyl, isopentyl, neopentyl, n-hexyl, sec-hexyl, n-heptyl, n-octyl, n-decyl, n-undecyl, dodecyl, cyclohexyl, (cyclohexyl)methyl, cyclopropylmethyl, and homologs and isomers thereof.

“Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C1.8 alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to Cis branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, cyano, and mercapto.

The term “heteroalkyl,” by itself or in combination with another term, means, unless otherwise stated, a stable straight or branched chain having from 1 to 20 carbon atoms or heteroatoms or a cyclic hydrocarbon group having from 3 to 10 carbon atoms or heteroatoms, or combinations thereof, consisting of at least one carbon atom and at least one heteroatom selected from the group consisting of O, N, P, Si and S, and wherein the nitrogen, phosphorus, and sulfur atoms may optionally be oxidized and the nitrogen heteroatom may optionally be quaternized. The heteroatom(s) 0, N, P and S and Si may be placed at any interior position of the heteroalkyl group or at the position at which alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, O—CH₃, —O—CH₂—CH₃, and —CN. Up to two or three heteroatoms may be consecutive, such as, for example, —CH₂—NH—OCH₃ and —CH₂—O—Si(CH₃)₃.

As described above, heteroalkyl groups, as used herein, include those groups that are attached to the remainder of the molecule through a heteroatom, such as —C(O)NR′, —NR′R″, —OR′, —SR, —S(O)R, and/or —S(O₂)R′. Where “heteroalkyl” is recited, followed by recitations of specific heteroalkyl groups, such as —NR′R or the like, it will be understood that the terms heteroalkyl and —NR′R″ are not redundant or mutually exclusive.

Rather, the specific heteroalkyl groups are recited to add clarity. Thus, the term “heteroalkyl” should not be interpreted herein as excluding specific heteroalkyl groups, such as —NR′R″ or the like.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. The cycloalkyl group can be optionally partially unsaturated. The cycloalkyl group also can be optionally substituted with an alkyl group substituent as defined herein, oxo, and/or alkylene. There can be optionally inserted along the cyclic alkyl chain one or more oxygen, sulfur or substituted or unsubstituted nitrogen atoms, wherein the nitrogen substituent is hydrogen, unsubstituted alkyl, substituted alkyl, aryl, or substituted aryl, thus providing a heterocyclic group. Representative monocyclic cycloalkyl rings include cyclopentyl, cyclohexyl, and cycloheptyl. Multicyclic cycloalkyl rings include adamantyl, octahydronaphthyl, decalin, camphor, camphane, and noradamantyl, and fused ring systems, such as dihydro- and tetrahydronaphthalene, and the like.

The term “cycloalkylalkyl,” as used herein, refers to a cycloalkyl group as defined hereinabove, which is attached to the parent molecular moiety through an alkylene moiety, also as defined above, e.g., a C₁₋₂₀ alkylene moiety. Examples of cycloalkylalkyl groups include cyclopropylmethyl and cyclopentylethyl.

The terms “cycloheteroalkyl” or “heterocycloalkyl” refer to a non-aromatic ring system, unsaturated or partially unsaturated ring system, such as a 3- to 10-member substituted or unsubstituted cycloalkyl ring system, including one or more heteroatoms, which can be the same or different, and are selected from the group consisting of nitrogen (N), oxygen (O), sulfur (S), phosphorus (P), and silicon (Si), and optionally can include one or more double bonds.

The cycloheteroalkyl ring can be optionally fused to or otherwise attached to other cycloheteroalkyl rings and/or non-aromatic hydrocarbon rings. Heterocyclic rings include those having from one to three heteroatoms independently selected from oxygen, sulfur, and nitrogen, in which the nitrogen and sulfur heteroatoms may optionally be oxidized and the nitrogen heteroatom may optionally be quaternized. In certain embodiments, the term heterocylic refers to a non-aromatic 5-, 6-, or 7-membered ring or a polycyclic group wherein at least one ring atom is a heteroatom selected from O, S, and N (wherein the nitrogen and sulfur heteroatoms may be optionally oxidized), including, but not limited to, a bi- or tri-cyclic group, comprising fused six-membered rings having between one and three heteroatoms independently selected from the oxygen, sulfur, and nitrogen, wherein (i) each 5-membered ring has 0 to 2 double bonds, each 6-membered ring has 0 to 2 double bonds, and each 7-membered ring has 0 to 3 double bonds, (ii) the nitrogen and sulfur heteroatoms may be optionally oxidized, (iii) the nitrogen heteroatom may optionally be quaternized, and (iv) any of the above heterocyclic rings may be fused to an aryl or heteroaryl ring. Representative cycloheteroalkyl ring systems include, but are not limited to pyrrolidinyl, pyrrolinyl, imidazolidinyl, imidazolinyl, pyrazolidinyl, pyrazolinyl, piperidinyl, piperazinyl, indolinyl, quinuclidinyl, morpholinyl, thiomorpholinyl, thiadiazinanyl, tetrahydrofuranyl, and the like.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. The terms “cycloalkylene” and “heterocycloalkylene” refer to the divalent derivatives of cycloalkyl and heterocycloalkyl, respectively.

An unsaturated hydrocarbon has one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. Alkyl groups which are limited to hydrocarbon groups are termed “homoalkyl.”

More particularly, the term “alkenyl” as used herein refers to a monovalent group derived from a C₂₋₂₀ inclusive straight or branched hydrocarbon moiety having at least one carbon-carbon double bond by the removal of a single hydrogen molecule. Alkenyl groups include, for example, ethenyl (i.e., vinyl), propenyl, butenyl, 1-methyl-2-buten-1-yl, pentenyl, hexenyl, octenyl, allenyl, and butadienyl.

The term “cycloalkenyl” as used herein refers to a cyclic hydrocarbon containing at least one carbon-carbon double bond. Examples of cycloalkenyl groups include cyclopropenyl, cyclobutenyl, cyclopentenyl, cyclopentadiene, cyclohexenyl, 1,3-cyclohexadiene, cycloheptenyl, cycloheptatrienyl, and cyclooctenyl.

The term “alkynyl” as used herein refers to a monovalent group derived from a straight or branched C₂₋₂₀ hydrocarbon of a designed number of carbon atoms containing at least one carbon-carbon triple bond. Examples of “alkynyl” include ethynyl, 2-propynyl (propargyl), 1-propynyl, pentynyl, hexynyl, and heptynyl groups, and the like.

The term “alkylene” by itself or a part of another substituent refers to a straight or branched bivalent aliphatic hydrocarbon group derived from an alkyl group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —CH₂CH₂CH₂CH₂—, —CH₂CH═CHCH₂—, —CH₂CsCCH₂—, —CH₂CH₂CH(CH₂CH₂CH₃)CH₂—, —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons. Typically, an alkyl (or alkylene) group will have from 1 to 24 carbon atoms, with those groups having 10 or fewer carbon atoms being some embodiments of the present disclosure. A “lower alkyl” or “lower alkylene” is a shorter chain alkyl or alkylene group, generally having eight or fewer carbon atoms.

The term “heteroalkylene” by itself or as part of another substituent means a divalent group derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms also can occupy either or both of the chain termini (e.g., alkyleneoxo, alkylenedioxo, alkyleneamino, alkylenediamino, and the like). Still further, for alkylene and heteroalkylene linking groups, no orientation of the linking group is implied by the direction in which the formula of the linking group is written. For example, the formula —C(O)OR′— represents both —C(O)OR′— and —R′OC(O)—.

The term “aryl” means, unless otherwise stated, an aromatic hydrocarbon substituent that can be a single ring or multiple rings (such as from 1 to 3 rings), which are fused together or linked covalently. The term “heteroaryl” refers to aryl groups (or rings) that contain from one to four heteroatoms (in each separate ring in the case of multiple rings) selected from N, O, and S, wherein the nitrogen and sulfur atoms are optionally oxidized, and the nitrogen atom(s) are optionally quaternized. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of above noted aryl and heteroaryl ring systems are selected from the group of acceptable substituents described below. The terms “arylene” and “heteroarylene” refer to the divalent forms of aryl and heteroaryl, respectively.

For brevity, the term “aryl” when used in combination with other terms (e.g., aryloxy, arylthioxy, arylalkyl) includes both aryl and heteroaryl rings as defined above. Thus, the terms “arylalkyl” and “heteroarylalkyl” are meant to include those groups in which an aryl or heteroaryl group is attached to an alkyl group (e.g., benzyl, phenethyl, pyridylmethyl, furylmethyl, and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like). However, the term “haloaryl,” as used herein is meant to cover only aryls substituted with one or more halogens.

Where a heteroalkyl, heterocycloalkyl, or heteroaryl includes a specific number of members (e.g. “3 to 7 membered”), the term “member” refers to a carbon or heteroatom.

Further, a structure represented generally by the formula:

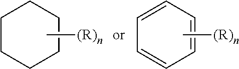

as used herein refers to a ring structure, for example, but not limited to a 3-carbon, a 4-carbon, a 5-carbon, a 6-carbon, a 7-carbon, and the like, aliphatic and/or aromatic cyclic compound, including a saturated ring structure, a partially saturated ring structure, and an unsaturated ring structure, comprising a substituent R group, wherein the R group can be present or absent, and when present, one or more R groups can each be substituted on one or more available carbon atoms of the ring structure. The presence or absence of the R group and number of R groups is determined by the value of the variable “n,” which is an integer generally having a value ranging from 0 to the number of carbon atoms on the ring available for substitution. Each R group, if more than one, is substituted on an available carbon of the ring structure rather than on another R group. For example, the structure above where n is 0 to 2 would comprise compound groups including, but not limited to:

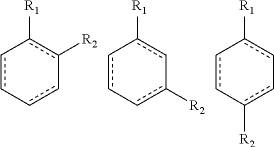

and the like.

A dashed line representing a bond in a cyclic ring structure indicates that the bond can be either present or absent in the ring. That is, a dashed line representing a bond in a cyclic ring structure indicates that the ring structure is selected from the group consisting of a saturated ring structure, a partially saturated ring structure, and an unsaturated ring structure.

The symbol ( [symbol] ) denotes the point of attachment of a moiety to the remainder of the molecule.

When a named atom of an aromatic ring or a heterocyclic aromatic ring is defined as being “absent,” the named atom is replaced by a direct bond.

Each of above terms (e.g., “alkyl,” “heteroalkyl,” “cycloalkyl, and “heterocycloalkyl”, “aryl,” “heteroaryl,” “phosphonate,” and “sulfonate” as well as their divalent derivatives) are meant to include both substituted and unsubstituted forms of the indicated group. Optional substituents for each type of group are provided below.

Substituents for alkyl, heteroalkyl, cycloalkyl, heterocycloalkyl monovalent and divalent derivative groups (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, =NR′, =N—OR′, —NR′R″, —SR′, -halogen, —SiR′R″R″′, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R″)═NR″′, —S(O)R′, —S(O)₂R′, —S(O)₂NR′R″, —NRSO₂R′, —CN, CF₃, fluorinated C₁₋₄ alkyl, and —NO₂ in a number ranging from zero to (2m′+1), where m′ is the total number of carbon atoms in such groups. R′, R″, R″′ and R″″ each may independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups. As used herein, an “alkoxy” group is an alkyl attached to the remainder of the molecule through a divalent oxygen. When a compound of the disclosure includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 4-, 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for alkyl groups above, exemplary substituents for aryl and heteroaryl groups (as well as their divalent derivatives) are varied and are selected from, for example: halogen, —OR′, —NR′R″, —SR′, —SiR′R″R″′, —OC(O)R′, —C(O)R′, —CO₂R′, —C(O)NR′R″, —OC(O)NR′R″, —NR″C(O)R′, —NR′—C(O)NR″R″′, —NR″C(O)OR′, —NR—C(NR′R″R′″)═NR″″, —NR—C(NR′R″)═NR″′—S(O)R′, —S(O)₂R′, —S(O)₂NR′R″, —NRSO₂R′, —CN and —NO₂, —R′, —N₃, —CH(Ph)₂, fluoro(C₁₋₄)alkoxo, and fluoro(C₁₋₄)alkyl, in a number ranging from zero to the total number of open valences on aromatic ring system; and where R′, R″, R″′ and R″″ may be independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound of the disclosure includes more than one R group, for example, each of the R groups is independently selected as are each R′, R″, R″′ and R″″ groups when more than one of these groups is present.

Two of the substituents on adjacent atoms of aryl or heteroaryl ring may optionally form a ring of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4.

One of the single bonds of the new ring so formed may optionally be replaced with a double bond. Alternatively, two of the substituents on adjacent atoms of aryl or heteroaryl ring may optionally be replaced with a substituent of the formula —(CRR′)ₛ—X′—(C″R″′)_{d}—, where s and d are independently integers of from 0 to 3, and X′ is —O—, —NR′—, —S—, —S(O)—, —S(O)₂—, or —S(O)₂NR′—. The substituents R, R′, R″ and R″′ may be independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the term “acyl” refers to an organic acid group wherein the —OH of the carboxyl group has been replaced with another substituent and has the general formula RC(═O)—, wherein R is an alkyl, alkenyl, alkynyl, aryl, carbocylic, heterocyclic, or aromatic heterocyclic group as defined herein). As such, the term “acyl” specifically includes arylacyl groups, such as a 2-(furan-2-yl)acetyl)- and a 2-phenylacetyl group. Specific examples of acyl groups include acetyl and benzoyl. Acyl groups also are intended to include amides, —RC(═O)NR′, esters, —RC(═O)OR′, ketones, —RC(═O)R′, and aldehydes, —RC(═O)H.

The terms “alkoxyl” or “alkoxy” are used interchangeably herein and refer to a saturated (i.e., alkyl-O—) or unsaturated (i.e., alkenyl-O— and alkynyl-O—) group attached to the parent molecular moiety through an oxygen atom, wherein the terms “alkyl,” “alkenyl,” and “alkynyl” are as previously described and can include C₁₋₂₀ inclusive, linear, branched, or cyclic, saturated or unsaturated oxo-hydrocarbon chains, including, for example, methoxyl, ethoxyl, propoxyl, isopropoxyl, n-butoxyl, sec-butoxyl, tert-butoxyl, and n-pentoxyl, neopentoxyl, n-hexoxyl, and the like.

The term “alkoxyalkyl” as used herein refers to an alkyl-O-alkyl ether, for example, a methoxyethyl or an ethoxymethyl group.

“Aryloxyl” refers to an aryl-O— group wherein the aryl group is as previously described, including a substituted aryl. The term “aryloxyl” as used herein can refer to phenyloxyl or hexyloxyl, and alkyl, substituted alkyl, halo, or alkoxyl substituted phenyloxyl or hexyloxyl.

“Aralkyl” refers to an aryl-alkyl-group wherein aryl and alkyl are as previously described, and included substituted aryl and substituted alkyl. Exemplary aralkyl groups include benzyl, phenylethyl, and naphthylmethyl.

“Aralkyloxyl” refers to an aralkyl-O— group wherein the aralkyl group is as previously described. An exemplary aralkyloxyl group is benzyloxyl, i.e., C₆H₅—CH₂—O—. An aralkyloxyl group can optionally be substituted.

“Alkoxycarbonyl” refers to an alkyl-O—C(═O)— group. Exemplary alkoxycarbonyl groups include methoxycarbonyl, ethoxycarbonyl, butyloxycarbonyl, and tert-butyloxycarbonyl.

“Aryloxycarbonyl” refers to an aryl-O—C(═O)— group. Exemplary aryloxycarbonyl groups include phenoxy- and naphthoxy-carbonyl.

“Aralkoxycarbonyl” refers to an aralkyl-O—C(═O)— group. An exemplary aralkoxycarbonyl group is benzyloxycarbonyl.

“Carbamoyl” refers to an amide group of the formula —C(═O)NH₂. “Alkylcarbamoyl” refers to a R′RN—C(═O)— group wherein one of R and R′ is hydrogen and the other of R and R′ is alkyl and/or substituted alkyl as previously described. “Dialkylcarbamoyl” refers to a R′RN—C(═O)— group wherein each of R and R′ is independently alkyl and/or substituted alkyl as previously described.

The term carbonyldioxyl, as used herein, refers to a carbonate group of the formula —O—C(═O)—OR.

“Acyloxyl” refers to an acyl-O— group wherein acyl is as previously described.

The term “amino” refers to the —NH₂ group and also refers to a nitrogen containing group as is known in the art derived from ammonia by the replacement of one or more hydrogen radicals by organic radicals. For example, the terms “acylamino” and “alkylamino” refer to specific N-substituted organic radicals with acyl and alkyl substituent groups respectively.

An “aminoalkyl” as used herein refers to an amino group covalently bound to an alkylene linker. More particularly, the terms alkylamino, dialkylamino, and trialkylamino as used herein refer to one, two, or three, respectively, alkyl groups, as previously defined, attached to the parent molecular moiety through a nitrogen atom. The term alkylamino refers to a group having the structure —NHR′ wherein R′ is an alkyl group, as previously defined; whereas the term dialkylamino refers to a group having the structure —NR′R″, wherein R′ and R″ are each independently selected from the group consisting of alkyl groups. The term trialkylamino refers to a group having the structure —NR′R″R″′, wherein R′, R″, and R′ are each independently selected from the group consisting of alkyl groups. Additionally, R′, R″, and/or R″′ taken together may optionally be —(CH₂)ₖ— where k is an integer from 2 to 6. Examples include, but are not limited to, methylamino, dimethylamino, ethylamino, diethylamino, diethylaminocarbonyl, methylethylamino, isopropylamino, piperidino, trimethylamino, and propylamino.

The amino group is —NR′R″, wherein R′ and R″ are typically selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

The terms alkylthioether and thioalkoxyl refer to a saturated (i.e., alkyl-S—) or unsaturated (i.e., alkenyl-S— and alkynyl-S—) group attached to the parent molecular moiety through a sulfur atom. Examples of thioalkoxyl moieties include, but are not limited to, methylthio, ethylthio, propylthio, isopropylthio, n-butylthio, and the like.

“Acylamino” refers to an acyl-NH— group wherein acyl is as previously described. “Aroylamino” refers to an aroyl-NH— group wherein aroyl is as previously described.

The term “carbonyl” refers to the —C(═O)— group, and can include an aldehyde group represented by the general formula R—C(═O)H.

The term “carboxyl” refers to the —COOH group. Such groups also are referred to herein as a “carboxylic acid” moiety.

The term “cyano” refers to the —C≡N group.

The terms “halo,” “halide,” or “halogen” as used herein refer to fluoro, chloro, bromo, and iodo groups. Additionally, terms such as “haloalkyl,” are meant to include monohaloalkyl and polyhaloalkyl. For example, the term “halo(C14)alkyl” is mean to include, but not be limited to, trifluoromethyl, 2,2,2-trifluoroethyl, 4-chlorobutyl, 3-bromopropyl, and the like.

The term “hydroxyl” refers to the —OH group.

The term “hydroxyalkyl” refers to an alkyl group substituted with an —OH group.

The term “mercapto” refers to the —SH group.

The term “oxo” as used herein means an oxygen atom that is double bonded to a carbon atom or to another element.

The term “nitro” refers to the —NO₂ group.

The term “thio” refers to a compound described previously herein wherein a carbon or oxygen atom is replaced by a sulfur atom.

The term “sulfate” refers to the —SO₄ group.

The term thiohydroxyl or thiol, as used herein, refers to a group of the formula —SH.

More particularly, the term “sulfide” refers to compound having a group of the formula —SR.

The term “sulfone” refers to compound having a sulfonyl group —S(O₂)R.

The term “sulfoxide” refers to a compound having a sulfinyl group —S(O)R The term ureido refers to a urea group of the formula —NH—CO—NH₂.

Throughout the specification and claims, a given chemical formula or name shall encompass all tautomers, congeners, and optical- and stereoisomers, as well as racemic mixtures where such isomers and mixtures exist.

Certain compounds of the present disclosure may possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as D- or L- for amino acids, and individual isomers are encompassed within the scope of the present disclosure. The compounds of the present disclosure do not include those which are known in art to be too unstable to synthesize and/or isolate. The present disclosure is meant to include compounds in racemic, scalemic, and optically pure forms. Optically active (R)- and (S)-, or D- and L-isomers may be prepared using chiral synthons or chiral reagents, or resolved using conventional techniques. When the compounds described herein contain olefenic bonds or other centers of geometric asymmetry, and unless specified otherwise, it is intended that the compounds include both E and Z geometric isomers.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

It will be apparent to one skilled in the art that certain compounds of this disclosure may exist in tautomeric forms, all such tautomeric forms of the compounds being within the scope of the disclosure. The term “tautomer,” as used herein, refers to one of two or more structural isomers which exist in equilibrium and which are readily converted from one isomeric form to another.

Unless otherwise stated, structures depicted herein are also meant to include compounds which differ only in the presence of one or more isotopically enriched atoms. For example, compounds having the present structures with the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present disclosure, whether radioactive or not, are encompassed within the scope of the present disclosure.

The compounds of the present disclosure may exist as salts. The present disclosure includes such salts. Examples of applicable salt forms include hydrochlorides, hydrobromides, sulfates, methanesulfonates, nitrates, maleates, acetates, citrates, fumarates, tartrates (e.g. (+)-tartrates, (−)-tartrates or mixtures thereof including racemic mixtures, succinates, benzoates and salts with amino acids such as glutamic acid. These salts may be prepared by methods known to those skilled in art. Also included are base addition salts such as sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent or by ion exchange. Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. Certain specific compounds of the present disclosure contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

In addition to salt forms, the present disclosure provides compounds, which are in a prodrug form. Prodrugs of the compounds described herein are those compounds that readily undergo chemical changes under physiological conditions to provide the compounds of the present disclosure. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent.

The term “protecting group” refers to chemical moieties that block some or all reactive moieties of a compound and prevent such moieties from participating in chemical reactions until the protective group is removed, for example, those moieties listed and described in T. W. Greene, P. G. M. Wuts, Protective Groups in Organic Synthesis, 3rd ed. John Wiley & Sons (1999). It may be advantageous, where different protecting groups are employed, that each (different) protective group be removable by a different means. Protective groups that are cleaved under totally disparate reaction conditions allow differential removal of such protecting groups. For example, protective groups can be removed by acid, base, and hydrogenolysis. Groups such as trityl, dimethoxytrityl, acetal and tert-butyldimethylsilyl are acid labile and may be used to protect carboxy and hydroxy reactive moieties in the presence of amino groups protected with Cbz groups, which are removable by hydrogenolysis, and Fmoc groups, which are base labile. Carboxylic acid and hydroxy reactive moieties may be blocked with base labile groups such as, without limitation, methyl, ethyl, and acetyl in the presence of amines blocked with acid labile groups such as tert-butyl carbamate or with carbamates that are both acid and base stable but hydrolytically removable.

Carboxylic acid and hydroxy reactive moieties may also be blocked with hydrolytically removable protective groups such as the benzyl group, while amine groups capable of hydrogen bonding with acids may be blocked with base labile groups such as Fmoc. Carboxylic acid reactive moieties may be blocked with oxidatively-removable protective groups such as 2,4-dimethoxybenzyl, while co-existing amino groups may be blocked with fluoride labile silyl carbamates.

Allyl blocking groups are useful in the presence of acid- and base-protecting groups since the former are stable and can be subsequently removed by metal or pi-acid catalysts. For example, an allyl-blocked carboxylic acid can be deprotected with a palladium(O)-catalyzed reaction in the presence of acid labile t-butyl carbamate or base-labile acetate amine protecting groups. Yet another form of protecting group is a resin to which a compound or intermediate may be attached. As long as the residue is attached to the resin, that functional group is blocked and cannot react. Once released from the resin, the functional group is available to react.

Typical blocking/protecting groups include, but are not limited to the following moieties:

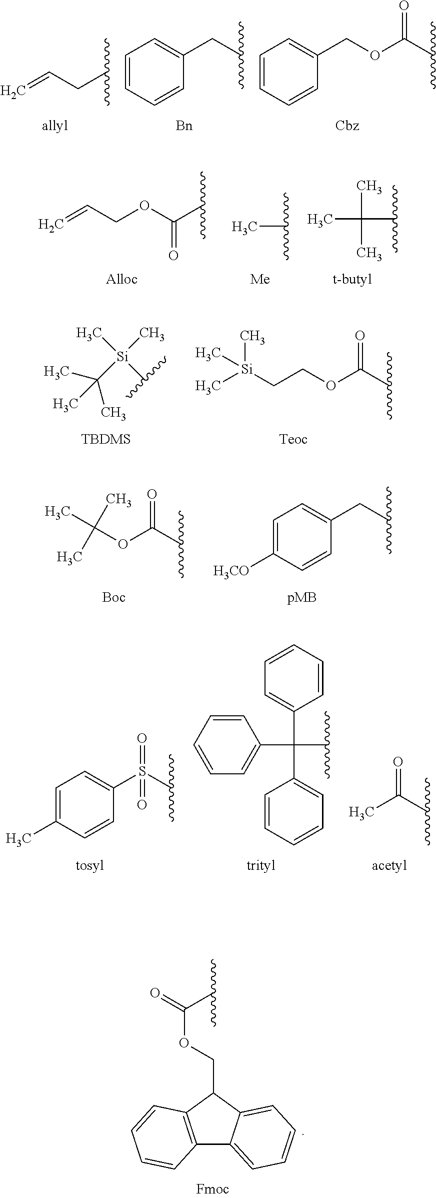

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Examining Heterodimerization by Aryl C—N Coupling in Dynemicin Biosynthesis

### 1.1 Overview

Distinct among the enediyne antitumor antibiotics, the dynemicin subgroup is comprised of two discrete halves, an enediyne and an anthraquinone, but each is ultimately derived from the same linear β-hydroxyhexaene intermediate. The linkage of these two halves by an aryl C—N bond is examined here using a variety of experimental approaches.

In this example, we demonstrate that this heterodimerization is specific for an aryl iodide as the corresponding bromo- and amino-substituted anthracenes failed to support dynemicin biosynthesis. Furthermore, biochemical experiments and chemical model reactions suggest an S_{RN}1 mechanism for the aryl C—N coupling in which electron transfer occurs to the iodoanthracene followed by loss of iodide and partition of the resulting aryl radical between C—N coupling and reduction by hydrogen abstraction.

An enzyme pull-down experiment aiming to capture the proteins involved in the coupling reaction is described in which two proteins, Orf14 and Orf16, encoded by the dynemicin biosynthetic gene cluster, are specifically isolated. Deletion of orf14 from the genome abolished dynemicin production accompanied by a 3-fold increased accumulation of the iodoanthracene coupling partner, indicating the plausible involvement of this protein in the heterodimerization process.

On the other hand, the deletion of orf16 only reduced dynemicin production by 55%, implying a non-catalytic, auxiliary role for the protein. Structural comparisons using AlphaFold imply key similarities between Orf14 and x-ray crystal structures of several proteins from enediyne BGCs, believed to bind hydrophobic polyene or enediyne motifs, suggest Orf14 templates aryl C—N bond formation during the central heterodimerization in dynemicin biosynthesis.

### 1.2 Background

An important advance in understanding Dyn biosynthesis was the isolation and characterization of the iodoanthracene 5 bearing a fused γ-thiolactone ring, which accompanies Dyn in the wild type producer, Micromonospora chersina. Cohen and Townsend, 2018b. ¹³C-Labeled 5 was subsequently shown to incorporate its isotopic label into only the anthraquinone portion of Dyn and established its role as one probable component in the heterodimerization reaction. The presence of iodine and sulfur in 5 was surprising as the Dyn BGC2 contains neither a predicted halogenase, a family of well-characterized and annotated proteins, Neumann et al., 2008, nor any evident sulfur-metabolizing enzyme(s).

Further clues about the heterodimerization came with the discovery of four shunt products 6-9 isolated from a CRISPR-Cas9 knockout of the single apparent flavin-dependent oxidoreductase in the Dyn BGC, dynE13; and also, for example, sungeidines B (21) and C (22) isolated from Micromonospora sp. MD118, which contains a truncated dynemicin-like BGC that lacks a dynE13 paralog. Low et al., 2020. Striking in all these heterodimeric structures, however, is a single aryl C—N bond, which we take to represent the first coupling step in a more complex linkage process that connects the two halves of Dyn. Cohen and Townsend, 2020.

### 1.3 Results and Discussion

### 1.3.1 Specificity of Iodine in Biosynthesis

Iodide added in minute amounts to fermentation media has a profound effect on dynemicin production. In our hands, Dyn titers in M. chersina were barely detectable in its absence but approximately 300-times greater in its presence (NaI, 0.5 mg/L). Cohen and Townsend, 2018b. Interestingly, supplementing iodoanthracene 5 without added NaI fully restored Dyn production, but also partitioned to the reduced anthracene 12. Cohen and Townsend, 2018b. We interpreted these findings to support a radical nucleophilic substitution (S_{RN}1) mechanism initiated by single-electron transfer to the iodoanthracene, loss of iodide leaving an aryl radical, which partitions between C—N coupling or H-atom abstraction to form the reduced anthracene 12. Cohen and Townsend, 2018b. In contrast, NaCl and NaBr, even in 100-fold higher concentration, did not lead to recovery of Dyn synthesis.

To test the specificity of iodine and gain more insight into the enzyme(s) involved, we wanted to see the effect of bromoanthracene 13 supplementations on wild type M. chersina Dyn fermentation. Natural production of bromoanthracene 13 by wild type M. chersina was not observed even supplementing NaBr instead of NaI, Cohen and Townsend, 2018b, but that does not prove that it could not substitute for the iodoanthracene in the aryl C—N coupling step. We, therefore, synthesized 13, a new anthracene structural class containing a fused γ-thiolactone ring to test whether the heterodimerization itself could still occur.

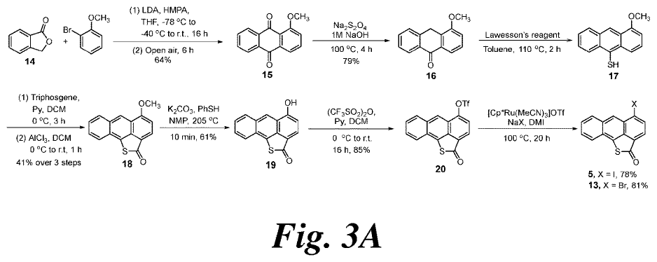

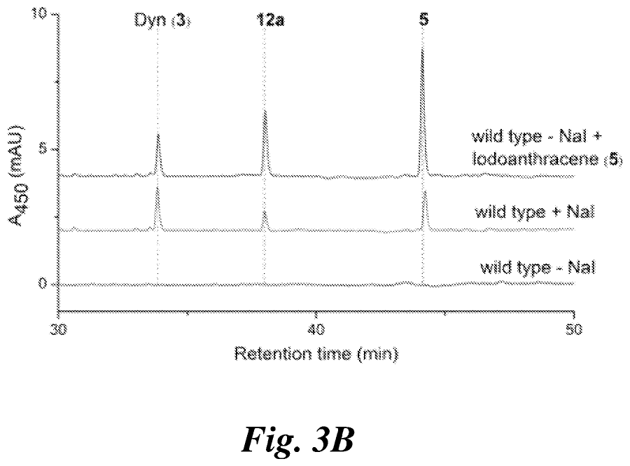

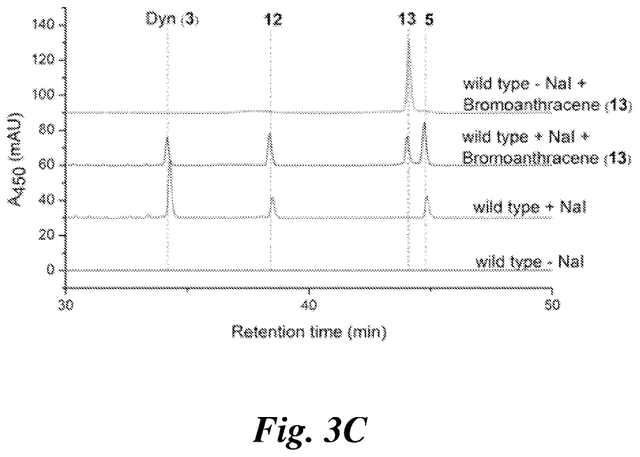

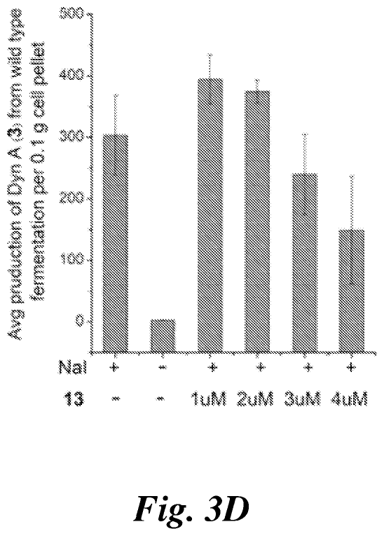

The anthracene skeleton can be assembled by either starting with a naphthalene core and building the flanking C-ring, Cover and Noyes, 1921, or taking two separate benzene rings and constructing the middle B-ring. Finar, 1959. Both approaches generate either a dihydroanthracene or anthraquinone as an intermediate, where either oxidation or reduction, respectively, leads to the anthracene skeleton. Owing to the specific pattern of the three substituents in 13, we elected to construct the tricyclic aromatic core starting from separate precisely substituted benzene precursors and proceed by way of an anthraquinone intermediate where a B-ring carbonyl could be used for sulfur introduction. Regiospecific addition of the phthalide 14 anion to the benzyne derived from 2-bromoanisole was governed by the inductive effect of the methoxy group to give 1-methoxyanthraquinone 15, Khanapure et al., 1987, where the peri-methoxyl additionally serves as a masked equivalent of the halide to be introduced later (FIG. 3a). This peri-substituent further directed the reduction of the adjacent anthraquinone carbonyl by alkaline sodium dithionite to produce anthrone 16 as a single regioisomer. Prinz et al., 1996; Conradt, 2015.

The position of the anthrone carbonyl allowed the efficient introduction of sulfur as desired by reaction with Lawesson's reagent, Roger et al., 2015, to give the anthracene thiol 17 after spontaneous tautomerization of the initially formed thioketone. The reaction of the aryl thiol with triphosgene followed by Friedel-Crafts acylation created the unique γ-thiolactone 18 bridging the B- and C-rings dictated by the activating C-ring methoxy group in its fourth role in the synthesis. Mitsudo et al., 2016. Final steps involved the conversion of the methoxyl to triflate 20 and a Ru(II)-catalyzed substitution by iodine to 5, or other halogens, such as bromine 13. Imazaki et al., 2012.

Synthetic bromoanthracene 13 was administered to the wild type M. chersina without added sodium halide in the fermentation medium. Dyn production was not restored. Furthermore, no reduced anthracene 12 was observed unlike the behavior of the on-pathway intermediate, iodoanthracene 5 (FIG. 3c). Cohen and Townsend, 2018b.

In a further test of the bromide, the addition of 13 to M. chersina fermentation medium with added sodium iodide was shown to decrease Dyn production with increasing concentrations of 13 (FIG. 3d). These results suggest that 13, being structurally similar to 5, can compete for the active site of the enzyme/enzyme complex and inhibit Dyn production without turnover. The key C—N coupling reaction in Dyn biosynthesis is specific for the iodoanthracene 5. Installation of the C—N bond by deiodination points to an unprecedented biosynthetic strategy observed in natural product biochemistry.

### 1.3.2 Introduction of Nitrogen

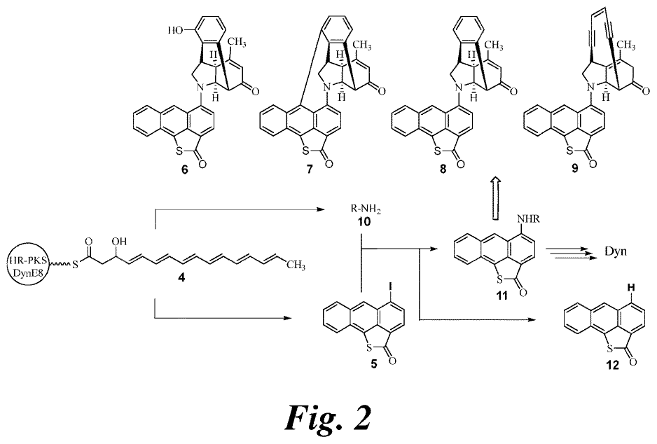

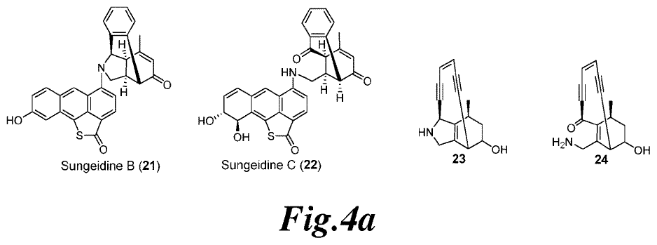

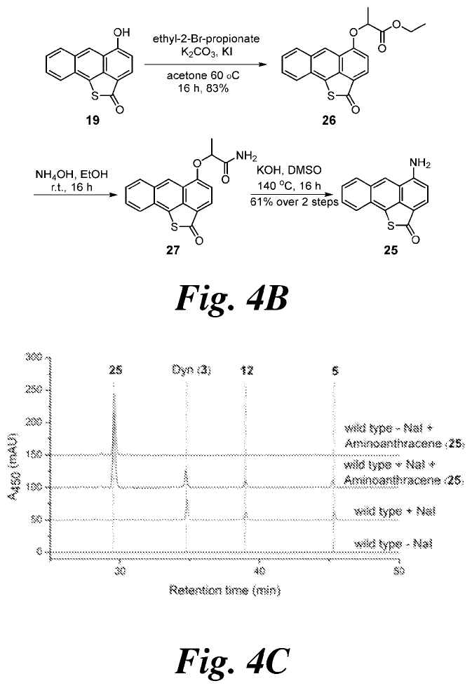

As noted above, shunt products 6-9 isolated from a variant of M. chersina in which a putative flavin-monooxygenase, DynE13, was deleted, Cohen and Townsend, 2020, and notably similar secondary metabolites, sungeidines B 21 and C 22 (FIG. 4a), arising from a truncated Dyn-like BGC lacking a homolog of DynE13, Low et al., 2020, all show a direct aryl C—N single bond connection. These heterodimerization products imply three important things. First, conversion of the intermediate anthracene-γ-thiolactone to the anthraquinone of the final product owes, at least in part, to the action of DynE13. Cohen and Townsend, 2020. Second, the preservation of the anthracene-thiolactone in both sets of metabolites suggests that aryl C—N bond formation occurs first in the overall sequence of linkage events that join the anthracene/anthraquinone half of Dyn to its enediyne-containing partner. Third, while all enediynes derive ultimately from the C16-hexaene 4, the heterodimeric shunt products 6-9, 21, and 22 each contain two C15 halves suggesting the possibility of a shared pathway from 4 to a common C15 intermediate before differentiation to iodoanthracene 5 and aminoenediyne 10 (FIG. 2). One can logically visualize the C—N coupling between an enediyne containing a secondary alkylamine, such as 23, or a primary amine like 24, as the structure of sungeidine C 22 would support. Alternatively, a simpler process involving an ammonia (NH₃) donor would give initially aminoanthracene 25, which can readily undergo reductive amination to achieve heterodimerization in a familiar manner commonly observed in all branches of alkaloid biosynthesis. Org Zeigler and Facchini, 2008; Lichman, 2021. Without any structural knowledge about the enediyne half, both pathways seem equally possible. To distinguish between these two global possibilities, aminoanthracene-γ-thiolactone 25 was synthesized for direct experimental test.

The desired amine 25 was synthesized starting with the free alcohol 19. The reaction of the aryl hydroxyl with ethyl 2-bromopropionate under mildly basic conditions to 26 followed by treatment with ammonium hydroxide generated the aryloxyamide 27. This compound undergoes ipso nucleophilic attack by the aryloxyamide anion produced in presence of hydroxide, resulting in C—N bond formation and C—O bond cleavage, and finally intramolecular acyl transfer to release the aryl amine. Like the bromoanthracene 13, aminoanthracene 25 failed to rescue Dyn production in wild type M. chersina in the absence of added NaI (FIG. 4c), thus bringing the options to only one possibility; the nitrogen comes preinstalled on the enediyne coupling partner.

### 1.3.4 Identification of Proteins Involved in C—N Bond Formation by Substrate Mimic Pull-Down Assay

Clear positive or negative results from in vivo incorporation experiments with iodo-, bromo- and aminoanthracenes 5, 13, and 25, respectively, were sufficient to infer the presence of a dedicated enzyme in the C—N bond-forming reaction catalyzing the dehalogenative C—N coupling reaction. Unable to identify any candidate protein(s) with this proposed activity in the BGC by bioinformatics, we decided to perform a biochemical pull-down experiment assuming strong, selective binding to the co-substrate iodoanthracene 5. Proteins involved in enzymatic transformations typically have a strong affinity for their substrate(s), even in the absence of covalent interactions. Therefore, close substrate mimics having a suitable linker to a stationary phase (resin bead) can be used to isolate bound protein(s) for their identification. For our purpose, we chose to synthesize the substrate-like 28 having a hydroxyl in place of the iodine and a carboxylic acid on the A-ring. We replaced the iodine with hydroxyl so that 28 can still fit in the active site having overall structural similarity, but the interaction with protein is presumed weaker compared to the native substrate to therefore allow selective elution with iodoanthracene 5. The A-ring was functionalized with a carboxylic acid to attach a suitable linker at a position furthest from the locus of C—N coupling and, it was hoped, to interfere minimally with active site recognition.

Formation of the required anthracene 28 bearing the γ-thiolactone utilized the same synthetic strategy as before but substituted the commercially available 5-bromophthalide 31 as starting material. To introduce the carboxyl group, the bromide 33 was first converted to the alkyne 34 using a tetrakis(triphenylphosphine)palladium(0) catalyzed Sonogashira reaction with trimethylsilylacetylene in the presence of triethylamine and catalytic copper(I) iodide. Wang et al., 2014. Hydration of the alkyne gave the methyl ketone 35, which upon reaction with molecular oxygen in the presence of catalytic iron(III) nitrate and iodine afforded the carboxylic acid 36. Xu et al., 2018. The final step involved removal of the O-methyl cap utilizing potassium carbonate (catalytic) and thiophenol in N-methyl-2-pyrrolidone at 210° C. to give the free hydroxyl 28. Chakraborti et al., 2002.

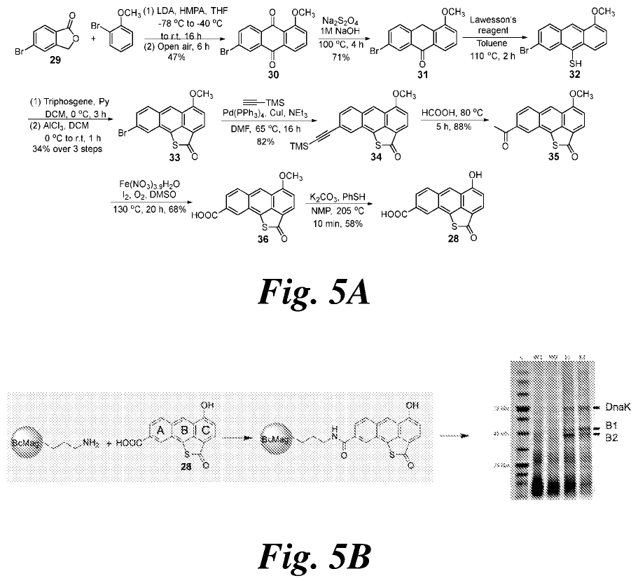

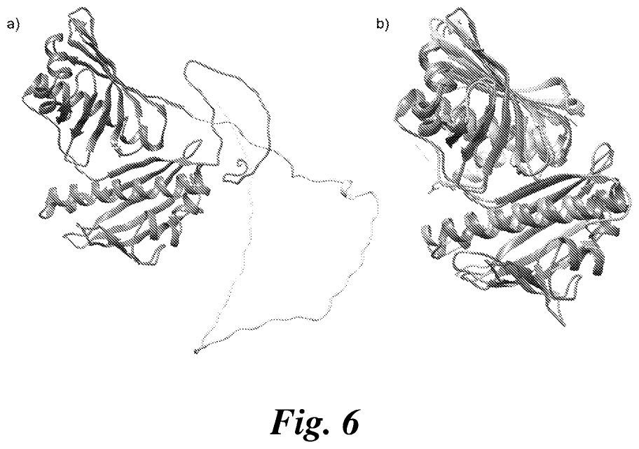

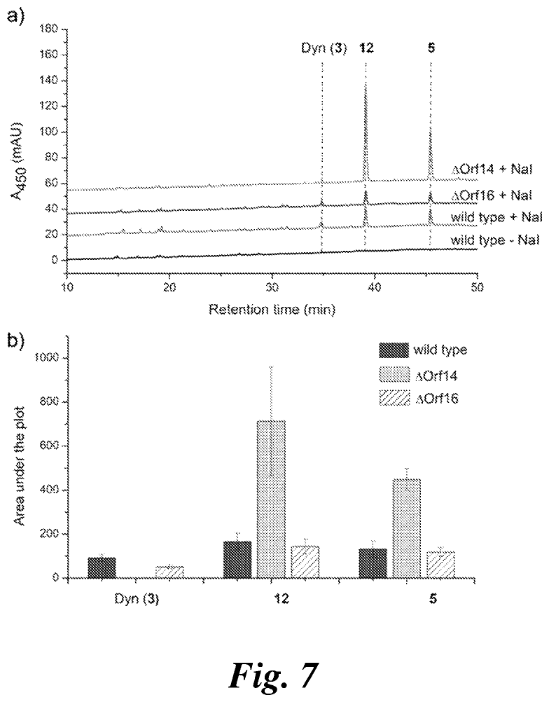

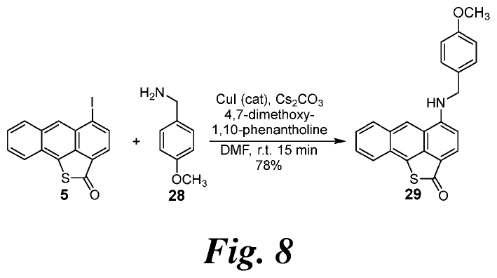

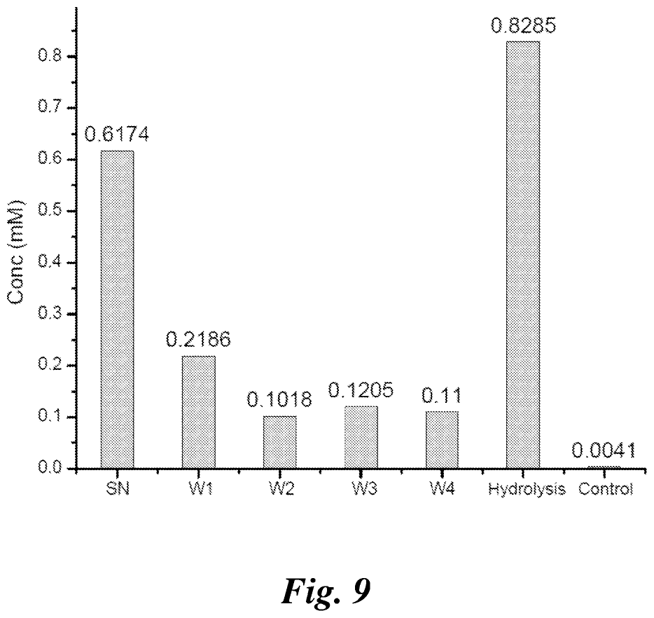

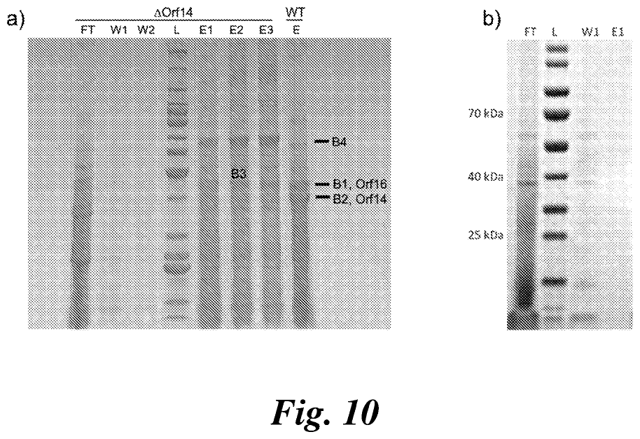

The anthracene carboxylic acid 28 was coupled to BcMag amine-terminated magnetic beads through a three-carbon linker. The loaded beads were incubated with M. chersina cell-free extract (CFE) at 4° C. for 4 h. The beads were washed, and bound proteins were eluted with the native iodoanthracene 5 and bromoanthracene 13 (4:1) in the same buffer. The proteins were concentrated and analyzed by denaturing PAGE. Two prominent bands in the haloanthracene elutions (B1 and B2, FIG. 5b and FIG. 9) were subjected to trypsinolysis and the proteolytic fragments were separated by HPLC and directly analyzed by electrospray-ionization tandem mass spectrometry (LC/ESI-MS/MS). Unbiased comparisons of peptide sequence information to the entire M. chersina proteome unambiguously identified B1 as Orf16 (68% coverage) and B2 as Orf14 (64% coverage) encoded by the Dyn BGC along with a varying amounts of the universal chaperone DnaK (58% coverage). The remarkably clear and specific identification of two proteins from the Dyn BGC, Orf14 and Orf16, suggests that this enzyme pair may be required to carry out aryl C—N bond formation, as previously speculated. Chakraborti et al., 2002. A control experiment with resin beads bearing the short amino linker but no bound ligand, when eluted as previously gave no protein binding above the background (FIG. 10).

### 1.3.4 in Silico Characterization of Orf14 and Orf16

The sequences for orf14 and orf16 from the BGC were subjected to BLASTx for translated nucleotide alignment to associate potential functions as both genes are retained in all ACE BGCs. Bioinformatics only revealed low similarity of Orf14 to proteins containing an SRPBCC (START/RHO_alpha_C/PITP/Bet_v1/CoxG/CalC) ligand-binding domain. This broad, loosely defined domain is characterized by its deep hydrophobic pocket thought to bind a wide variety of ligands, such as an enediyne to confer resistance (CalC, 28% identity), Singh et al., 2006, Orf16 contains a HEAT-repeat structural motif made up of alpha helices connected by short loops. This widely-occurring motif has been observed in chromosome regulatory proteins to apoptosis inhibitors. Singh et al., 2006; Marchler-Baer et al., 2017; Kobe et al., 1999; Altchul et al., 1997.

In the absence of sufficient information to suggest function from sequence homology, 3D models of the proteins were developed using AlphaFold, Jumper et al., 2021, and SWISS-MODEL, Bertoni et al., 2017. In line with the BLAST results, the predicted structure of Orf14 consists of two SRPBCC domains forming a heterodimeric hydrophobic core, with a predicted unstructured N-terminal region of about 75 amino acids. A structure-based similarity search of Orf14 using Dalilite, Holm et al., 2010, revealed more than 500 hits with close structural similarity but very low sequence identity. Overlaying these protein structures with the Orf14 predicted structure revealed the prominent conservation of the core fold that binds a structurally diverse class of hydrophobic ligands ranging from polyketide, polyene, and lipids to small aromatic rings, steroids, and hormones; for example, the type II PKS ARO/CYL BexL (PDB:4XRW) involved in the regiospecific cyclization of the precursor of anticancer agent BE-7585A, Caldara-Festin et al., 2015, abscisic acid receptor PYL (PDB:7MLD), Vaidya et al., 2021, a pathogenesis-related class 10 protein LIPR10.2B (PDB:5MXB) that binds melatonin, Sliwiak et al., 2018, thebaine synthase 2 (PDB:6KA3) that converts (7S)-salutaridinol 7-O-acetate to thebaine, the key component in codeine and morphine biosynthesis, Chen et al., 2020, the isomerase LFS (PDB:5GTG) involved in the natural production of the lachrymatory agent syn-propanethial S-oxide, Arakawa et al., 2020, and plant allergen Pru av 1 (PDB:1E09). Neudecker et al., 2001. However, more interesting is the structural homology to two proteins, the proposed self-sacrifice resistance protein CalU16 (PDB:4FPW) from calicheamicin 2 biosynthesis, Elshahawi et al., 2014, and the biosynthetic protein DynU16 (PDB:6V04) from the Dyn 3 BGC. Alvarado et al., 2021. CalU16 is a small monodomain protein containing the conserved core SRBPCC fold, that is proposed to bind the enediyne skeleton of 2. DynU16 is a hetero-didomain protein, strikingly similar to the organized portion of the Orf14 model. DynU16 is hypothesized to bind polyene/enediyne-like molecules involved in the upstream part of Dyn 3 biosynthesis using the hydrophobic binding pocket at the domain junction. All these results suggest Orf14 may similarly be involved in the binding of hydrophobic polyene/eneyne-like molecules.

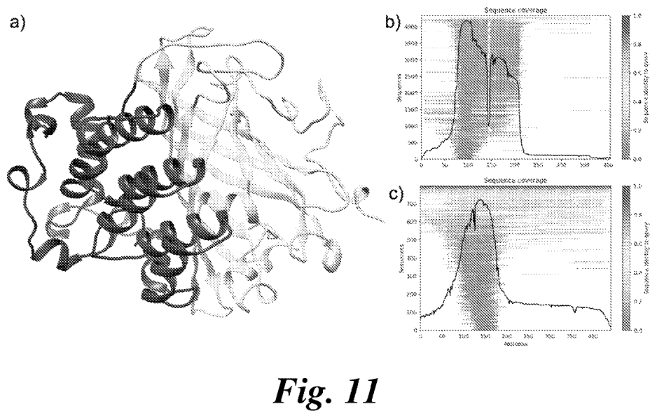

The 3D model of Orf16 predicted by AlphaFold24 shows a didomain architecture (FIG. 11), with one domain containing the HEAT-repeat motif at high confidence and another domain of partial ß-barrel structure with low confidence. An analogous structure-based similarity search using Dalilite returned a large group of proteins mostly with the conserved HEAT-repeat motif and involved in cellular functions like intracellular transport, Campanacci et al., 2022, protein-protein interaction, Tiouajni et al., 2014, and protein binding. Xiang et al., 2010. Whether Orf16 is involved in similar cellular processes is the subject of further biochemical experiments.

### 1.3.5 Biochemical Experiments to Establish the Role of Orf14 and Orf16 in Biosynthesis.

To investigate the functions of the gratifyingly unambiguous identification of only two Dyn biosynthetic proteins from the cell-free pull-down experiments, it was decided to delete orf14 and orf16 individually from the Dyn BGC in M. chersina. Orf14 was removed first from the genome using CRISPR-Cas9. Tong et al., 2015. Dyn production was completely abolished, but iodoanthracene 5 was not. In fact, compared to wild type an approximately 3-fold higher production of iodoanthracene 5 was observed along with a further increased proportion of the reduced anthracene 12. The implications of these observations are three-fold. First, Orf14 is not involved in the previously observed reduction of the iodoanthracene to 12. Cohen and Townsend, 2018a. Second, the increased accumulation of anthracene products is consistent with blocking aryl C—N coupling in the heterodimerization process. Third, no new product, as, for example, the enediyne coupling partner could be detected by sensitive UV-vis/HPLC assay. Polyene, eneyne or conceivable Bergman rearrangement products, Cohen and Townsend, 2020, would be expected to be readily detected in a stoichiometry commensurate with observed titers of Dyn. They were not.

Next, orf16 was similarly deleted from the M. chersina genome. Unexpectedly, the removal of Orf16 did not eliminate Dyn production but reduced it to slightly more than one-half (approximately 55%) compared to the wild type. Production of the co-occurring anthracenes 5 and 12 was correspondingly reduced. One can imagine that Orf16 might have been pulled down in this experiment by strong association with Orf14 and not owing to direct affinity for the resin-bound probe 5. To test for this possibility, the pull-down experiment was repeated identically from the fermentation of the Δorf14 strain. A strong band correlating to Orf16 (76% sequence coverage) appeared in the iodo/bromoanthracene elution clearly indicating a direct association of Orf16 with the iodoanthracene 5 affinity ligand in the absence of Orf14 (FIG. 10).

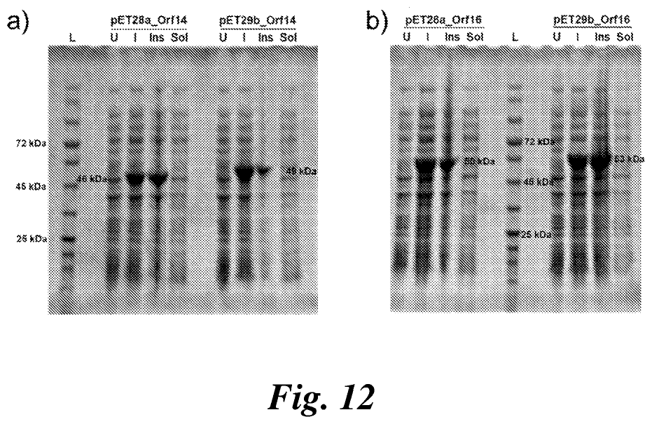

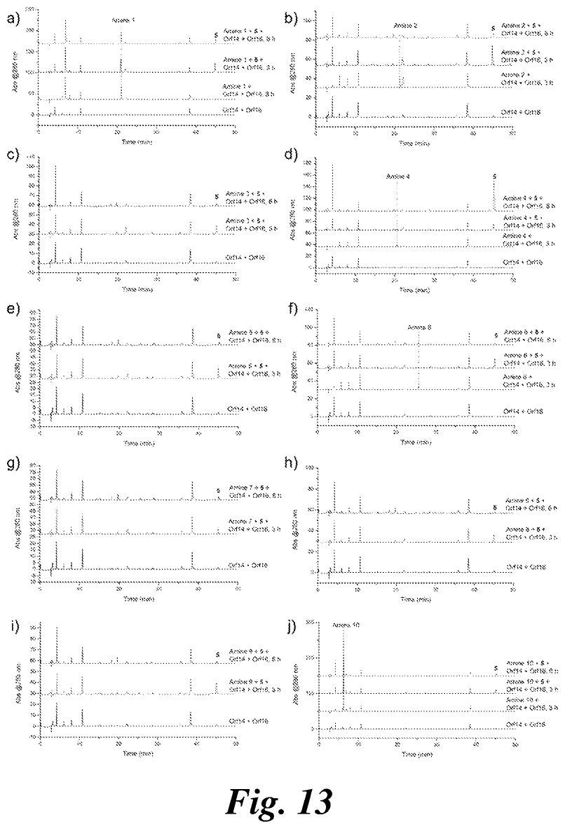

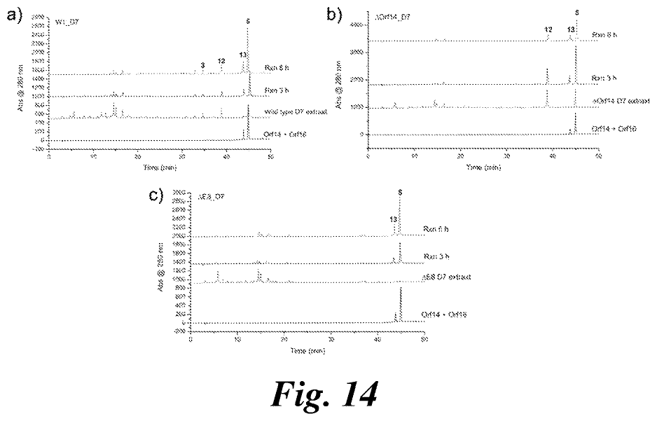

Multiple attempts were made to express Orf14 and Orf16. Thus far, only insoluble proteins have been observed (FIG. 12). However, even if we had been successful in obtaining soluble, and, one hopes, functional, protein(s), we were faced with the impossibility of developing an assay without knowing the structure of the enediyne coupling partner to iodoanthracene 5. Realizing that the Δorf14 strain produces no Dyn, but accumulates super-physiological amounts of the iodoanthracene compared to wild type, we anticipated that the cells would be “enriched” in the enediyne coupling partner or earlier intermediates and, therefore, we designed an experiment to expose a cell-free extract (CFE) of this M. chersina variant to both Orf14 and Orf16 that had been isolated as above by affinity chromatography and concentrated from a wild type fermentation in the hope of finally seeing the elusive aryl C—N heterodimerization product. To our disappointment, no such coupling product was observed (FIG. 13). In a set of final attempts, 10 primary and secondary model amines (4-methoxybenzylamine, α-methylbenzylamine, cyclohexylamine, benzylamine, butylamine, 4-chlorobenzylamine, 1-methyl pyrrolidine, pyrrolidine, piperidine, isoquinoline) were incubated (10 μM) with Orf14, Orf16 and iodoanthracene 5 (100 μM), but no new products were detected by HPLC analysis at 3-, and 6-hour time points (FIG. 14).

### 1.3.6 Chemical Model Reactions of Aryl C—N Bond Formation

As discussed previously, both chemical precedent and observation of the reduction product 12 of iodoanthracene 5 point to an S_{RN}1 radical nucleophilic substitution mechanism to account for the central aryl C—N bond formation to initiate the heterodimerization process in Dyn biosynthesis. Cohen and Townsend, 2018a. Such a reaction is favored by the presence of iodine (which would be lost as iodide ion) as opposed to fluorine (which would favor more classical nucleophilic aromatic substitution, SNAr. One might think having the unusual fused thiolactone places a carbonyl para to the leaving group (iodine) and constrains it in the plane of the anthracene would favor the formation of the Meisenheimer complex. A chemical model of an SNAr substitution reaction, therefore, was employed to test the intrinsic reactivity of the iodoanthracene 5. To maximize the chance for success, we chose the primary amine p-methoxybenzylamine 37 in the presence of a variety of bases; for example, cesium carbonate in N,N-dimethylformamide, and prolonged heating at elevated temperature gave no indication of aryl C—N bond formation. In marked contrast, treatment in the presence of catalytic copper(I) iodide and 4,7-dimethoxy-1,10-phenanthroline, Shafir and Buchwald, 2006, gave the expected Ullman/Goldberg product in under 15 min at room temperature. Reaction of the corresponding bromoanthracene 13 proceeded at room temperature as well, but approximately three times more slowly. The same amino substitution product 38 was formed in both cases. While these copper-mediated reactions are probably not a perfect model of an enzymatic S_{RN}1 reaction, the inherent properties of the iodoanthracene-γ-thiolactone do not uniquely favor an SNAr reaction path.

### 1.4 Summary

Paralogs of Orf14 and Orf16 are common to all Dyn family anthraquinone-containing enediyne natural product BGCs, but not to the other 9- or 10-membered families (Table 2).

TABLE 2
Orf14 and Orf16 from Dynemicin BGC and top homologs in the uncialamycin (UCM),
tiancimycin (TNM), yangpumicin (YPM), and calicheamicin (CLM) clusters.

| | Putative | | % CV/ | | % CV/ | | % CV/ | | % CV/ |
|---|---|---|---|---|---|---|---|---|---|
| Gene | Function | UCM | % ID | YNM | % ID | YPM | % ID | CLM | % ID |
| Orf14 | SRPBCC | ucmG | 91/54 | tnmG | 86/55 | ypmG | 94/70 | calU16 | 33/29 |
| | domain- | | | | | | | | |
| | containing | | | | | | | | |
| | protein | | | | | | | | |
| Orf16 | HEAT | ucmD | 99/79 | tnmD | 99/82 | ypmD | 100/87 | N/A | N/A |
| | repeat | | | | | | | | |
| | domain- | | | | | | | | |
| | containing | | | | | | | | |
| | protein | | | | | | | | |

Biochemical experiments described herein, supported by in silico analyses, strongly suggest that Orf14 is intimately involved in the central heterodimerization of iodoanthracene 5 and its still unknown aminoenediyne coupling partner, whereas Orf16 takes part in an apparently non-catalytic but auxiliary manner with iodoanthracene 5. It has been a long-standing frustration throughout our investigation of the Dyn biosynthetic pathway that multiple seeming opportunities to identify this intermediate or post-hexaene 4 precursors have been thwarted at every turn. In particular, in the present investigation Δorf14, which produces no Dyn, yields approximately 3-times the wild type concentration of the iodoanthracene heterodimerization co-reactant, yet accumulates no detectable polyene, polyeneyne or aryl products, and none having diagnostic mass signatures reflecting the presence of a single (or odd number of) nitrogen atom(s). Direct queries of the aryl C—N bond-forming process with the bromo- and aminoanthracenes 13 and 25 establish that the coupling is strictly dependent on the presence of the unusual anthracenyl iodide and that the enediyne partner must contain an amine. The co-production of the reduced anthracene 12 and chemical model reactions support an S_{RN}1 heterodimerization mechanism but do not unambiguously prove it.

Given the 1:1 stoichiometry of the anthracene and aminoenediyne building blocks in Dyn and its production in sufficient titers, one would expect to readily observe the enediyne partner or some earlier intermediate(s). That we do not suggests that they are (1) very unstable and decompose readily, and/or (2) are sequestered and protected in not only Dyn biosynthesis but also in all enediyne biosynthetic pathways. For reference, the 9-membered enediyne chromophore of neocarzinostatin (NCS, 1) is stabilized by noncovalent interaction with an apoprotein, Ncs A, but is rapidly degraded by heat, thiols, light and pH values above 4.8 when separated from its apoprotein (t_{1/2} is 12 s at pH 8 and 25° C.). Kappen and Goldberg, 1980; Porvik and Goldberg, 1980. Analogous stabilization of a labile enediyne coupling partner in the aryl C—N heterodimerization can be readily envisioned to involve a protein complex of Orf14 and Orf16.

The resistance protein CalU16 from calicheamicin (CLM, 2) BGC is known to bind the enediyne warhead in a hydrophobic cavity at the core of its SRPBCC domain. Elshahawi et al., 2014. Superimposition of the CalU16 monodomain with the predicted N-terminal domain of Orf14 reveals a remarkably good alignment (RMSD 1.907 Å) strongly suggestive of a similar enediyne binding ability. By comparison, the C-terminal domain of Orf14 only poorly overlays with the enediyne binding fold of CalU16 (RMSD 15.584 Å). Bearing in mind the biochemical pull-down experiments, however, that led to the original isolation of Orf14, these structural comparisons are best accommodated by differential binding by the N- and C-terminal domains of Orf14 to bring the proposed aminoenediyne and iodoanthracene 5 into proximity to enable aryl C—N linkage to take place.

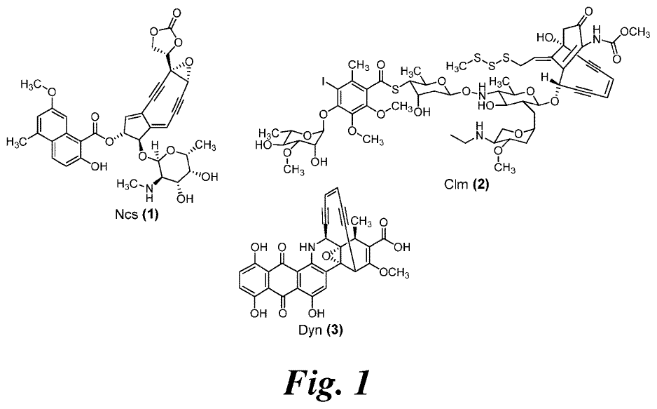

The BGC's that encode the synthesis of each of the three main structural groups of enediyne antitumor antibiotics (FIG. 1) are formidably large (50-75 genes) and comprised of many proteins of unknown function. Gao and Thorson, 2008; Liu et al., 2005. The biosyntheses of these natural products remains largely unsolved and experimentally inaccessible, but from the numbers of proteins involved, they are certainly complex. Cohen and Townsend, 2018a. In each of these pathways representative proteins here and in other recent work have been identified as enediyne binding and often thought to be self-resistance proteins. Elshahawi et al., 2014. Biggins et al., 2003. We suggest the same properties of enediyne/hydrophobic substrate binding, intermediate stabilization and protection have and will be attributed to BGC proteins having large hydrophobic binding pockets that not only confer resistance but also deliver labile intermediates in the biosynthesis of enediyne products. Unambiguous functional assignment of these proteins is a complex technical challenge requiring improved biochemical and spectroscopic approaches to overcome the barriers to understanding both arms of Dyn biosynthesis where the insights gained can be applied to the other structural classes.

### 1.5 Methods

1.5.1 Fermentation and Supplementation of M. chersina

Samples cut from medium 53 agar were used to inoculate 50 mL liquid medium 53 cultures. The cultures were shaken at 28° C. for seven days. Then, samples from these vegetative cultures were used to inoculate H881 media (10 g/L soluble starch, 5 g/L Pharmamedia, 1 g/L CaCO₃, 0.05 g/L CuSO₄·5 H₂O and 0.0005 g/L NaI) for fermentation. These cultures were then shaken at 28° C. for 7 days.

### 1.5.2 High-Performance Liquid Chromatographic Analysis of Whole-Cell Metabolite Extracts

Samples (5 mL) of fermentations were extracted with 5 mL of ethyl acetate by vortexing the emulsion for one minute and separating the layers by centrifugation at 4000×g. The ethyl acetate layer (4 mL) was isolated and dried by SpeedVac at room temperature. The residue was then resuspended in 200 μL of dimethyl sulfoxide and filtered through 0.2 μm polytetrafluoroethylene filters for analysis on an Agilent 1200 HPLC equipped with a Phenomenex Prodigy ODS3 100 Å, 5 μm, 250×4.6 mm column. Injections were 30 μL and separated with a gradient method increasing from 5% to 95% acetonitrile supplemented with 0.1% formic acid over 40 min, followed by a 10 min hold at 95% acetonitrile before re-equilibration. The method was run at 1 mL/min and monitored at 280, 375, 450 and 570 nm.

### 1.5.3 Small Molecule Baited Pull-Down Assay

BcMag™ amine-terminated magnetic beads were coupled to compound 30 using EDC·HCl and 4-DMAP (see SI for detailed procedure). Activated beads (5 mL) were mixed with 40 mL of the M. chersina CFE and gently rotated at 4° C. for 4 h. As a control, 20 mL of CFE was incubated with 1 mL of unactivated beads. After 4 h, 20 mL of the reaction mixture was transferred to a 50 mL tube, placed on the magnetic separator for 5 min at 4° C. and the supernatant was removed as the flow-through (FT). The beads were resuspended with 2×10 mL of wash buffer (lx PBS buffer, 0.1 mM DTT, 10% (v/v) glycerol) by gentle mixing and iced for 5 min. The supernatant was removed using the magnetic separator and stored on ice as wash 1 and 2 respectively. The proteins of interest were then eluted by washing the beads twice with 3×5 mL wash buffer with added iodoanthracene 5 at increasing concentrations (E1 and E2 with 0.25 mM and 0.5 mM 5, respectively). All the washes and elutions were further concentrated by centrifugation at 4000×g using 3 k MWCO Amicon Ultra centrifugal filter (Millipore) at 4° C. to 1 mL final volume. A portion (20 mL) of the control reaction was washed once with 10 mL of wash buffer and eluted once with 5 mL of wash buffer+0.5 mM iodoanthracene 5 after 24 h. A sample of 100 μL for each was mixed with 25 μL of 5×SDS-PAGE loading dye, heated at 95° C. for 5 min, and centrifuged for 5 min. The supernatant (25 μL) from each sample was run on a 12% SDS-PAGE gel, stained with SimplyBlue™ SafeStain.

### 1.5.4 CRISPR-Cas9 Mediated Deletion Mutants

CRISPR-Cas9 plasmids with homologous recombination templates and sgRNAs were delivered to E. coli GM2929 hsdS::Tn10 (pUB307::Tn7) by electroporation and cultured to transfer the CRISPR-Cas9 plasmids to M. chersina by conjugation. Kieser et al., 2000. The resulting suspension was then incubated on medium 5346 with 2% agar and 10 mM MgCl₂ at 28° C. overnight and then overlaid to a final concentration of 20 μg/mL nalidixic acid and 50 μg/mL apramycin. The plates were then incubated until colonies were visible (4-7 days).

The healthy ex-conjugants were re-streaked on medium 53 agar containing nalidixic acid, apramycin and thiostrepton (1 μg/mL) to induce CRISPR-Cas9 activity. The plates were incubated for 6-7 days at 28° C. and colonies were re-streaked on fresh medium 53 agar. The resulting mutants were screened by colony PCR. Deletions were confirmed by gDNA isolation, PCR and sequencing. See Table 1 for primer sequences and genetic confirmation.

TABLE 1
Primers used for the assembly of CRISPR-Cas9 and heterologous expression
plasmids.

| Primer Name | Sequence |
|---|---|
| Orf14_sgRNA_ | CATGCCATGGGCTGTCGTACCTCTGGCGCTGGGGTTTT |
| 1F_180719 | AGAGCTAGAAATAGC (SEQ ID NO: 1) |
| Orf14_seq1_ | GCTGGACGAACGCAAGAT (SEQ ID NO: 2) |
| 180719 | |
| Orf14_KOtestF_ | TCCAGGGCTGGGGCTACATCAACCT (SEQ ID NO: 3) |
| 180719 | |
| Orf14_KOtestR_ | TTCATGCTTGTGGTCCTCGGGCACT (SEQ ID NO: 4) |
| 180719 | |
| Orf14_5F1_ | TCGTCGAAGGCACTAGAAGGGGCCACATGACCAACGC |
| 181227 | GTTCATCCTCGGC (SEQ ID NO: 5) |
| Orf14_5R_ | GCGTTCGTCCAGCGGCGGGGTTGCGGTGCAGCAGCGGGAA |
| 181231 | GTCATCGGGT (SEQ ID NO: 6) |
| Orf14_3F1_ | CCCCGCCGCTGGACGAACGCAAGATCCTCA (SEQ ID |
| 181227 | NO: 7) |
| Orf14_3R_ | GGTCGATCCCCGCATATAGGATCAGCTCGGAGTGGCT |
| 180719 | GCACTACGGCTAC (SEQ ID NO: 8) |
| pET28a_Orf14_ | CTGGTGCCGCGCGGCAGCCATGTGCGGGAGGCCGGCT |
| full_Fwd | CCGGCCGGTGT (SEQ ID NO: 9) |
| pET28a_Orf14_ | TGGTGGTGGTGCTCGAGTGCCTACCGGCTCCAGCCGT |
| rev | GCGGGCCGGCGTCGACCCG (SEQ ID NO: 10) |
| pET29b_Orf14_ | CCATGGCGATATCGGATCCGGTGCGGGAGGCCGGCTCCGG |
| full_fwd | CCGGTCT (SEQ ID NO: 11) |
| pET29b_Orf14_ | TGGTGGTGGTGCTCGAGTGCCCGGCTCCAGCCGTGCGGGCC |
| rev | GGCGTCGACCCGCTG (SEQ ID NO: 12) |
| pET28a_Orf16_ | CTGGTGCCGCGCGGCAGCCATATGGCAATCGATCTCTGCAA |
| fwd | CAAG (SEQ ID NO: 13) |
| pET28a_Orf16_ | TGGTGGTGGTGCTCGAGTGCTTACGGCAGCAGGGTGGTG |
| rev | (SEQ ID NO: 14) |
| pET29b_Orf16_ | CCACGCGGTTCCATGGCGATAATGGCAATCGATCTCTGCAA |
| fwd | CAAGTC (SEQ ID NO: 15) |
| pET29b_Orf16_ | TGGTGGTGGTGCTCGAGTGCCGGCAGCAGGGTGGTGTG |
| rev | (SEQ ID NO: 16) |
| Orf16_sgRNA_ | CATGCCATGGgaccggacttgaagaacgtccggGTTTTAGAGCTA |
| 2F_220103 | GAAATAGC (SEQ ID NO: 17) |
| Orf16_seq5_ | Catctcgatgaggttggtgtagaccteggcg (SEQ ID NO: 18) |
| 20220323 | |
| Orf16_seq4_ | Atccggccggcgcagtgcacgatcgcggtggg (SEQ ID NO: 19) |
| 20220220 | |
| Orf16_5F_ | TCGTCGAAGGCACTAGAAGGggaccagagtgaccactcggcggtg |
| 180711 | cagca (SEQ ID NO: 20) |
| Orf16_5F1_ | TCGTCGAAGGCACTAGAAGGccgccgcagcagggtgagcatccg |
| 211230 | gcggacgt (SEQ ID NO: 21) |
| Orf16_5R2_ | Gcagggtggtgttgttgcagagatcgattgccattgctgcct (SEQ ID NO: 22) |
| 211230 | |
| Orf16_3F1_ | Acaccaccctgctgccgtaacgaaggaaga (SEQ ID NO: 23) |
| 211230 | |
| Orf16_3R_ | GGTCGATCCCCGCATATAGGgcatgccaacctccgactgtcgtcgct |
| 20190115 | cctt (SEQ ID NO: 24) |

### 1.5.5 General Information

All chemicals and reagents were purchased from Sigma Aldrich, AK Scientific, Fischer Scientific or Oakwood Chemical unless otherwise noted and used without further purification. All solvents were distilled before use using an SPBT-1 benchtop solvent purification system from LC Technology Solutions Inc. Anhydrous DMF and anhydrous NMP over molecular sieves were purchased from ACROS Organic and used without further purification. Anhydrous DMI was purchased from Sigma Aldrich. DMSO was dried over heat-activated 4 Å molecular sieves. Nitrogenous bases were purified immediately before use by distillation over CaH₂.

Synthetic compounds were purified using a CombiFlash EZ Prep from Teledyne ISCO equipped with RediSep Rf normal-phase silica flash columns.

Analytical HPLC methods were carried out on an Agilent 1200 series HPLC equipped with a multi-wavelength UV-vis detector using a Prodigy ODS3 100 Å, 5μ, 250×4.6 mm column (Phenomenex). Preparative HPLC methods were carried out on a CombiFlash EZ Prep from Teledyne ISCO using Luna C18(2) 100 Å 10μ 250×21.20 mm preparative column (Phenomenex) equipped with a multi-wavelength UV-vis detector.

UPLC-HRMS experiments to determine exact masses and purity levels of organic compounds were carried out on a Waters Acquity/Xevo-G2 UPLC-MS system at the Johns Hopkins Mass Spectrometry Facility.

NMR spectra were recorded on either a 400 MHz Bruker Advance or 600 MHz Bruker Avance II spectrometer. Chemical shifts are reported relative to the reference shift for the solvent used relative to TMS. ¹³C NMR spectra were recorded on a 400 MHz Bruker Advance operating at 100 MHz or 600 MHz Bruker Avance II operating at 150 MHz. Chemical shifts are reported relative to the reference chemical shift of the NMR solvent.

### 1.5.6 Synthesis of Substrates for In Vivo Supplementation Assays

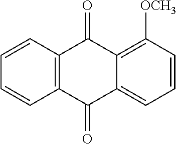 [SMILES: COc1cccc2c1C(=O)c1ccccc1C2=O]

Khanapure et al., 1987; Townsend et al., 1981. In a flame-dried round-bottomed flask N,N-diisopropylamine (11 mL, 78.3 mmol) was dissolved in 120 mL dry THE and cooled to −78° C. in an acetone/dry ice bath. To this n-BuLi (29.5 mL of 2.50 M in hexane, 73.8 mmol) was added dropwise and stirred at −78° C. for 20 min. Phthalide 14 (3.00 g, 22.4 mmol) was added to the reaction mixture at the same temperature, followed by immediate addition of HMPA (13.6 mL, 78.3 mmol). After stirring at −78° C. for another 10 min, the reaction mixture was warmed to −40° C. using an ACN/dry ice bath and 2-bromoanisole (6.1 mL, 49.2 mmol) was added. The reaction mixture was allowed to warm up to room temperature over 16 h and stirred open to the air for at least another 6 h. After completion, the reaction was quenched with sat. NH₄Cl solution and extracted with EtOAc. The organic layer was washed twice with a large volume of water, dried over anhydrous Na₂SO₄ and the solvent was removed in vacuo. The crude compound was purified by silica column chromatography using 50% EtOAc in hexanes to give the product as a yellow solid (3.40 g, 64% yield). ¹H NMR (400 MHz CDCl₃) δ ppm 8.19 (d, J=7.7 Hz, 1H), 8.14 (d, J=7.4 Hz, 1H), 7.85 (d, J=7.6, 1H), 7.73-7.61 (m, 3H), 7.27 (d, J=8.2 Hz, 1H), 3.98 (s, 3H); ¹³C (100 MHz CDCl₃) δ ppm 183.3, 182.3, 160.3, 135.6, 135.0, 134.9, 134.2, 133.2, 132.3, 127.1, 126.4, 121.3, 119.7, 117.9, 56.5; UPLC-ESI-HMS: calculated exact mass for C₁₅H₁₁O₃⁺ [M+H⁺]: 239.0703, found [M+H⁺]: 239.0705.

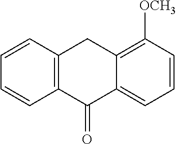 [SMILES: COc1cccc2c1Cc1ccccc1C2=O]

4-Methoxyanthracen-9(10H)-one (16). Prinz et al., 1996; Rogers et al., 2015. 1-Methoxy anthracene-9,10-dione (15, 2.00 g, 8.39 mmol) was dissolved in 56 mL of 1M aqueous NaOH to a final concentration of 0.15 M. To this solution Na₂S₂O₄ (5.04 g, 28.9 mmol) was added and the reaction mixture was heated to reflux for 4 h. After cooling the reaction to room temperature, the precipitate was filtered and washed with 50 mL of 1M NaOH, followed by H₂O until the filtrate became neutral. The precipitate was dried under vacuum. The product was further purified on silica gel with 10% EtOAc in hexanes to yield a pale-yellow solid (1.48 g, 79% yield). ¹H NMR (400 MHz CDCl₃) δ ppm 8.31 (d, J=7.9 Hz, 1H), 7.91 (d, J=7.9 Hz, 1H), 7.52 (t, J=7.3 Hz, 1H), 7.39 (t, J=6.0 Hz, 2H), 7.31 (t, J=8.0 Hz, 1H), 6.95 (d, J=8.0, 1H), 3.99 (s, 2H), 3.82 (s, 3H); ¹³C (100 MHz CDCl₃) δ ppm 184.1, 156.5, 140.6, 132.6, 132.6, 131.6, 129.6, 128.8, 127.2, 127.2, 126.7, 119.0, 113.0, 55.5, 27.2; UPLC-ESI-HMS: calculated exact mass for C₁₅H₁₃O₂⁺ [M+H⁺]: 225.0910, found [M+H⁺]: 225.0923.

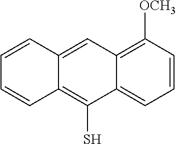 [SMILES: COc1cccc2c(S)c3ccccc3cc12]

4-Methoxyanthracen-9-thiol (17). Rogers et al., 2015. In a flame-dried round-bottomed flask 4-methoxyanthracen-9(10H)-one (16, 1.30 g, 5.8 mmol) was dissolved in 100 mL dry toluene and degassed by bubbling Ar though the solution for 15 min. To this mixture Lawesson's reagent (1.41 g, 3.48 mmol) was added and heated to reflux for 2 h. After completion of the reaction, the solution was cooled to room temperature and filtered through a small silica plug washing with 80% DCM in hexane until the filtrate became colorless. The filtrate was dried in vacuo to obtain the crude thiol as a yellow oil. No further purification was possible due to the very fast oxidation of the thiol to disulfide.

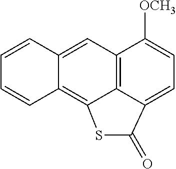 [SMILES: COc1ccc2c3c(c4ccccc4cc13)SC2=O]

5-Methoxy-2H-anthra[9,1-bc]thiophen-2-one (18). Mitsudo et al., 2016. The crude 4-methoxyanthracene-9-thiol (17, 1.40 g, 5.83 mmol) was dissolved in 100 mL dry DCM and cooled to 0° C. To this solution triphosgene (5.19 g, 17.49 mmol) and pyridine (1.88 mL, 23.3 mmol) were added and stirred at 0° C. for 3 h. After completion, the reaction was quenched very slowly by addition of ice-cold water, extracted with DCM and dried in vacuo. The metastable carbonochloridothioate intermediate was dissolved in 100 mL dry DCM and cooled to 0° C. To this solution AlCl₃ (1.17 g, 8.74 mmol) was added and stirred for 1 h, while allowing it to warm up to room temperature. After completion of the reaction, DCM was removed in vacuo, the crude product was dissolved in EtOAc and washed twice with 1N HCl. The organic layer was dried over anhydrous Na₂SO₄ and the solvent was removed in vacuo. The product was purified using silica gel chromatography with 20% EtOAc in hexanes to afford an orange solid (0.63 g, 41% yield over three steps). ¹H NMR (400 MHz CDCl₃) δ ppm 8.52 (s, 1H), 8.12 (d, J=7.8, 1H), 7.99 (d, J=8.5, 1H), 7.83 (d, J=8.3, 1H), 7.57 (t, J=7.3 Hz, 1H), 7.50 (t, J=7.5, 1H), 6.90 (d, J=7.8, 1H), 4.16 (s, 3H); ¹³C (100 MHz CDCl₃) S ppm 191.3, 162.0, 132.7, 130.1, 130.1, 129.8, 128.6, 127.5, 127.5, 126.0, 124.8, 125.7, 121.4, 112.0, 104.0, 56.6; UPLC-ESI-HMS: calculated exact mass for C₁₆H₁₁O₂S⁺ [M+H⁺]: 267.0474, found [M+H⁺]: 267.0472.

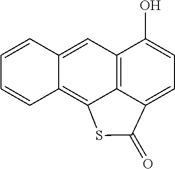 [SMILES: O=C1Sc2c3ccccc3cc3c(O)ccc1c23]

5-hydroxy-2H-anthra[9,1-bc]thiophen-2-one (19). Chakraborti et al., 2002. In a flame-dried round-bottomed flask, 5-methoxy-2H-anthra[9,1-bc]thiophen-2-one (18, 0.50 g, 1.88 mmol) was dissolved in 5 mL of dry N-methyl-2-pyrrolidone (NMP). To this solution K₂CO₃ (13 mg, 0.09 mmol) and thiophenol (0.19 mL, 1.88 mmol) were added and the reaction mixture was heated in a pre-heated oil bath to 205° C. for 10 min. After cooling to room temperature, the reaction mixture was acidified with 1N HCl and extracted with EtOAc. The organic layer was washed once with 1N HCl and twice with a large volume of H₂O, dried over anhydrous Na₂SO₄ and the solvent was removed in vacuo. The product was purified by silica gel column using 20% EtOAc in hexanes with 1% acetic acid to give an orange solid (0.29 g, 61% yield). ¹H NMR (400 MHz DMSO-d₆) δ ppm 12.39 (s, br, 1H), 8.69 (s, 1H), 8.19-8.15 (m, 2H), 7.77 (d, J=8.5, 1H), 7.60 (ddd, J=8.5, 6.7, 1.0 Hz, 1H), 7.53 (ddd, J=8.4, 6.7, 1.0 Hz, 1H), 7.13 (d, J=7.9 Hz, 1H); ¹³C (100 MHz DMSO-d₆) δ ppm 189.6, 162.9, 132.3, 131.0, 131.0, 130.8, 128.6, 128.6, 127.4, 126.3, 124.5, 121.9, 121.1, 121.1, 109.7; UPLC-ESI-HMS: calculated exact mass for C₁₅H₉O₂S⁺ [M+H⁺]: 253.0318, found [M+H⁺]: 253.0309.

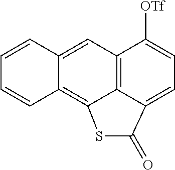 [SMILES: O=C1Sc2c3ccccc3cc3c(OS(=O)(=O)C(F)(F)F)ccc1c23]

2-Oxo-2H-anthra[9,1-bc]thiophen-5-yl trifluoromethane sulfonate (20). In a flame-dried round-bottomed flask, 5-hydroxy-2H-anthra[9,1-bc]thiophen-2-one (19, 0.20 g, 0.79 mmol) was dissolved in 10 mL dry DCM and cooled to 0° C. To this solution triflic anhydride (0.20 mL, 1.19 mmol) and pyridine (0.10 mL, 1.19 mmol) were added in succession and left to stir overnight at room temperature. After completion of the reaction, DCM was removed in vacuo. The crude mixture was dissolved in EtOAc and washed with H₂O. After drying down in vacuo, the remaining pyridine was removed by azeotropic distillation with toluene. The product was purified by silica gel chromatography with 10% EtOAc in hexanes to yield an orange solid (0.26 g, 85% yield). ¹H NMR (400 MHz CDCl₃) δ ppm 8.52 (s, 1H), 8.24 (d, J=7.6 Hz, 1H), 8.14 (dd, J=8.1, 1.3 Hz, 1H), 7.91 (dd, J=8.3, 1.1 Hz, 1H), 7.74 (d, J=7.6, 1H), 7.67 (quintd, J=6.6, 1.3 Hz, 2H); ¹³C (100 MHz CDCl₃) δ ppm 190.6, 149.6, 134.3, 132.5, 131.6, 130.1, 129.5, 128.7, 127.9, 127.7, 125.4, 125.0, 122.1, 118.8, 118.5, 117.2; UPLC-ESI-HMS: calculated exact mass for C₁₆H₈F₃O₄S₂⁺ [M+H⁺]: 384.9811, found [M+H⁺]: 384.9801.

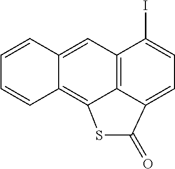 [SMILES: O=C1Sc2c3ccccc3cc3c(I)ccc1c23]

5-Iodo-2H-anthra[9,1-bc]thiophen-2-one (5). Imazaki et al., 2012. In a flame-dried round-bottomed flask, 2-oxo-2H-anthra[9,1-bc]thiophen-5-yl trifluoromethanesulfonate (20, 0.10 g, 0.26 mmol) was dissolved in 3 mL of dry 1,3-dimethyl-2-imidazolidinone (DMI). To this [Cp*Ru(MeCN)₃]OTf (7 mg, 0.01 mmol) and NaI (58 mg, 0.39 mmol) were added and the mixture heated at 100° C. for 20 h. After cooling to room temperature, the reaction mixture was diluted with EtOAc, filtered through Celite and the solvent was removed in vacuo. The crude product was purified by silica gel column with 10% EtOAc in hexanes to give the desired product as an orange solid (73 mg, 78% yield). For in vivo feeding experiments, the compound was further purified by preparative HPLC (Phenomenex Luna 10μ C18(2) 100 Å preparatory column, 250×21.20 mm ID) using water and acetonitrile with 0.1% TFA as mobile phase. ¹H NMR (400 MHz CDCl₃) δ ppm 8.43 (s, 1H), 8.33 (d, J=7.3 Hz, 1H), 8.12 (d, J=8.4 Hz, 1H), 7.88 (d, J=8.9 Hz, 1H), 7.87 (d, J=7.3 Hz, 1H), 7.62 (quintd, J=6.6, 1.4 Hz, 2H); ¹³C (100 MHz CDCl₃) δ ppm 191.9, 137.9, 134.3, 133.2, 131.4, 130.9, 130.0, 128.9, 128.1, 128.1, 127.6, 127.0, 126.0, 124.8, 108.0; UPLC-ESI-HMS: calculated exact mass for C₁₅H₈IOS⁺ [M+H⁺]: 362.9335, found [M+H⁺]: 362.9330.

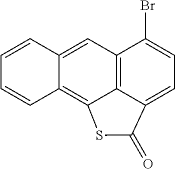 [SMILES: O=C1Sc2c3ccccc3cc3c(Br)ccc1c23]

5-Bromo-2H-anthra[9,1-bc]thiophen-2-one (13). Imazaki et al. 2012. In a flame-dried round-bottomed flask, 2-oxo-2H-anthra[9,1-bc]thiophen-5-yl trifluoromethanesulfonate (20, 0.10 g, 0.26 mmol) was dissolved in 3 mL of dry 1,3-dimethyl-2-imidazolidinone (DMI). To this [Cp*Ru(MeCN)₃]OTf (7 mg, 0.01 mmol) and NaBr (40 mg, 0.39 mmol) were added and the mixture heated at 100° C. for 20 h. After cooling to room temperature, the reaction mixture was diluted with EtOAc, filtered through Celite and the solvent was removed in vacuo. The crude product was purified by silica gel column with 10% EtOAc in hexanes to give the desired bromide as an orange solid (66 mg, 81% yield). For in vivo feeding experiments, the compound was further purified by preparative HPLC (Phenomenex Luna 10μ C18(2) 100 Å preparatory column, 250×21.20 mm ID) using water and acetonitrile with 0.1% TFA as mobile phase. ¹H NMR (600 MHz DMSO-d₆) δ ppm 8.83 (s, 1H), 8.44 (d, J=8.5 Hz, 1H), 8.30 (d, J=7.3 Hz, 1H), 8.22 (d, J=7.3 Hz, 1H), 8.00 (d, J=8.5 Hz, 1H), 7.80 (t, J=7.0 Hz, 1H), 7.75 (t, J=6.9, 1H); ¹³C (150 MHz DMSO-d₆) δ ppm 191.1, 134.3, 131.8, 131.7, 130.8, 130.6, 130.5, 129.6, 129.5, 128.8, 128.1, 128.0, 127.7, 127.5, 124.9; UPLC-ESI-HMS: calculated exact mass for C₁₅H₈BrOS⁺ [M+H⁺]: 314.9474, found [M+H⁺]: 314.9480.

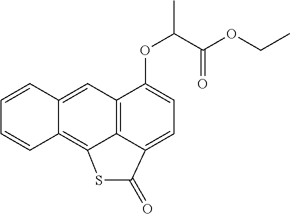 [SMILES: CCOC(=O)C(C)Oc1ccc2c3c(c4ccccc4cc13)SC2=O]

Ethyl 2-((2-oxo-2H-anthra[9,1-bc]thiophen-5-yl)oxy) propanoate (26). Yu et al., 2013. In a flame-dried round-bottomed flask 5-hydroxy-2H-anthra[9,1-bc]thiophen-2-one (19, 0.20 g, 0.79 mmol) was dissolved in 20 mL dry acetone. To this mixture anhydrous K₂CO₃ (0.22 g, 1.58 mmol), KI (13 mg, 0.08 mmol) and ethyl 2-bromopropionate (0.12 mL, 0.95 mmol) were added sequentially and heated to reflux for 16 h. After cooling to room temperature, the reaction mixture was filtered through Celite and concentrated in vacuo. The product was purified by silica gel chromatography using 20% EtOAc in hexanes to obtain a yellowish orange oil (0.23 g, 83% yield). ¹H NMR (400 MHz CDCl₃) δ ppm 8.62 (s, 1H), 8.06 (d, J=7.8 Hz, 1H), 8.00 (d×quint, J=8.5, 0.6 Hz, 1H), 7.79 (dq, J=8.5, 1.0 Hz, 1H), 7.54 (ddd, J=8.5, 7.9, 1.2 Hz, 1H), 7.48 (ddd, J=8.4, 6.7, 1.3 Hz, 1H), 6.75 (d, J=8.0, 1H), 5.10 (q, J=6.8 Hz, 1H), 4.28 (q, J=7.1 Hz, 2H), 1.89 (d, J=6.8 Hz, 3H), 1.27 (t, J=7.1 Hz, 3H); ¹³C (100 MHz CDCl₃) δ ppm 191.2, 170.9, 159.8, 132.8, 130.2, 130.1, 129.7, 128.1, 127.6, 127.6, 126.0, 125.4, 124.7, 121.4, 120.3, 105.2, 73.6, 61.8, 18.5, 14.1; UPLC-ESI-HMS: calculated exact mass for C₂₀H₁₇O₄S⁺ [M+H⁺]: 353.0842, found [M+H⁺]: 353.0839.

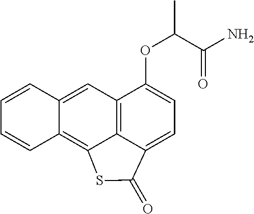 [SMILES: CC(Oc1ccc2c3c(c4ccccc4cc13)SC2=O)C(N)=O]

((2-Oxo-2H-anthra[9,1-bc]thiophen-5-yl)oxy)propanamide (27). Yu et al., 2013. Ethyl 2-((2-oxo-2H-anthra[9,1-bc]thiophen-5-yl)oxy) propanoate (26, 0.20 g, 0.57 mmol) was dissolved the minimum amount of THF. To this solution 15 mL of 1:1 EtOH and 30% NH₄OH solution were added and stirred at room temperature for 16 h. After completion of the reaction, the solvent was removed in vacuo. The crude amide was used directly for the next step without further purification.

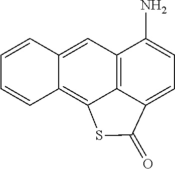 [SMILES: Nc1ccc2c3c(c4ccccc4cc13)SC2=O]

5-Amino-2H-anthra[9,1-bc]thiophen-2-one (25). Yu et al., 2013. The crude 2-((2-oxo-2H-anthra[9,1-bc]thiophen-5-yl)oxy) propanamide 27 was dissolved in 3 mL dry DMSO and to this solution solid KOH (32 mg, 0.57 mmol) was added and the mixture was heated to 140° C. overnight. After completion of the reaction, the mixture was cooled, diluted with brine and extracted with EtOAc. The combined organic layers were washed again with brine and water, dried over anhydrous Na₂SO₄ and concentrated in vacuo. The crude product was purified by preparative HPLC (Phenomenex Luna 10μ C18(2) 100 Å preparatory column, 250×21.20 mm ID) using water and acetonitrile with 0.1% TFA as mobile phase and obtained as an orange solid (87 mg, 61% yield). ¹H NMR (400 MHz DMSO-d₆) 5 ppm 8.92 (s, 1H), 8.07 (d, J=8.5 Hz, 1H), 8.03 (d, J=8.2 Hz, 1H), 7.83 (d, J=8.5 Hz, 1H), 7.64 (ddd, J=8.5, 6.6, 1.1 Hz, 1H), 7.55 (ddd, J=8.4, 6.8, 1.0 Hz, 1H), 6.83 (d, J=8.2 Hz, 1H); ¹³C (100 MHz DMSO-d₆) δ ppm 187.7, 155.4, 132.1, 131.9, 131.3, 130.6, 128.6, 128.5, 127.6, 125.9, 124.4, 121.3, 118.8, 116.2, 107.7; UPLC-ESI-HMS: calculated exact mass for C₁₅H₁₀NOS⁺ [M+H⁺]: 252.0478, found [M+H⁺]: 252.0477.

### 1.5.7 Synthesis of Substrate Mimic for Cell-Free Protein Pull-Down Experiment

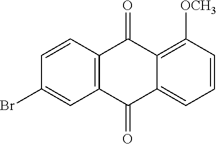 [SMILES: COc1cccc2c1C(=O)c1ccc(Br)cc1C2=O]

6-Bromo-1-methoxyanthracene-9,10-dione (30). In a flame-dried round-bottomed flask N,N-diisopropylamine (6.91 mL, 49.3 mmol) was dissolved in 120 mL dry THE and cooled to −78° C. in an acetone/dry ice bath. To this solution n-BuLi (18.6 mL of 2.50 M in hexane, 46.5 mmol) was added dropwise and stirred at −78° C. for 20 min. 5-Bromophthalide (29, 3.00 g, 14.1 mmol) was added to the reaction mixture at the same temperature, followed by immediate addition of HMPA (8.6 mL, 49.3 mmol). After stirring at −78° C. for another 10 min, the reaction mixture was warmed to −40° C. using an ACN/dry ice bath and 2-bromoanisole (3.9 mL, 31.0 mmol) was added. The reaction mixture was allowed to warm to room temperature over 4 h and stirred open to the air for at least another 6 h. After completion, the reaction was quenched with sat. NH₄Cl solution and extracted with EtOAc. The organic layer was washed twice with a large volume of water, dried over anhydrous Na₂SO₄ and the solvent was removed in vacuo. The crude product was purified by silica gel chromatography using 50% EtOAc in hexanes to give a yellow solid (2.10 g, 47% yield). ¹H NMR (400 MHz CDCl₃) δ ppm 8.32 (dd, J=2.1, 0.4 Hz, 1H), 8.12 (dd, J=8.3, 0.4 Hz, 1H), 7.93 (dd, J=7.7, 1.1 Hz, 1H), 7.88 (dd, J=8.3, 2.0 Hz, 1H), 7.73 (dd, J=8.4, 7.7 Hz, 1H), 7.36 (dd, J=8.4, 1.0 Hz, 1H), 4.05 (s, 3H); ¹³C (100.0 MHz CDCl₃) δ ppm 182.3, 181.6, 160.5, 137.2, 135.3, 135.3, 133.5, 133.5, 129.4, 129.1, 128.7, 121.1, 119.9, 118.3, 56.6; UPLC-ESI-HMS: calculated exact mass for C₁₅H₁₀BrO₃⁺ [M+H⁺]: 316.9808, found [M+H⁺]: 316.9809.

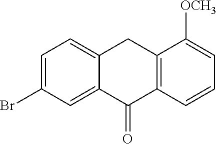 [SMILES: COc1cccc2c1Cc1ccc(Br)cc1C2=O]

2-Bromo-5-methoxyanthracene-9(10H)-one (31). 6-Bromo-1-methoxyanthracene-9,10-dione (30, 2.00 g, 6.31 mmol) was dissolved in 42 mL of 1M aqueous NaOH to a final concentration of 0.15 M. To this solution Na₂S₂O₄ (3.78 g, 21.7 mmol) was added and the reaction mixture was heated to reflux for 4 h. After cooling the reaction to room temperature, the accumulated precipitate was filtered and washed with 50 mL of 1M NaOH, followed by H₂O until the filtrate became neutral. The precipitate was dried under vacuum. The product was further purified on a silica gel column with 10% EtOAc in hexanes to afford the product as a pale-yellow solid (1.35 g, 71% yield). ¹H NMR (400 MHz CDCl₃) δ ppm 8.31 (dd, J=2.2, 0.3 Hz, 1H), 7.80 (dd, J=7.9, 1.1 Hz, 1H), 7.53 (dd, J=8.2, 2.2 Hz, 1H), 7.29 (t, J=8.0 Hz, 1H), 7.20 (dd, J=8.3, 0.4 Hz, 1H), 6.95 (dd, J=8.1, 1.0 Hz, 1H), 3.84 (s, 3H), 3.83 (s, 2H); ¹³C (100 MHz CDCl₃) δ ppm 182.7, 156.4, 139.1, 135.3, 132.9, 131.9, 130.6, 129.9, 129.2, 127.4, 120.9, 119.0, 113.3, 55.6, 26.8; UPLC-ESI-HMS: calculated exact mass for C₁₅H₁₂BrO₂⁺ [M+H⁺]: 303.0015, found [M+H⁺]: 303.0012.

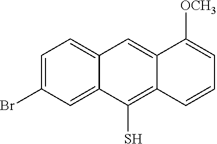 [SMILES: COc1cccc2c(S)c3cc(Br)ccc3cc12]

2-Bromo-5-methoxyanthracene-9-thiol (32). In a flame-dried round-bottomed flask 2-bromo-5-methoxyanthracen-9(10H)-one (31, 1.20 g, 3.96 mmol) was dissolved in 100 mL dry toluene and degassed by bubbling Ar though the solution for 15 min. To this solution Lawesson's reagent (0.96 g, 2.37 mmol) was added and heated to reflux for 2 h. After completion of the reaction, the solution was cooled to room temperature and filtered through a small silica plug washing with 80% DCM in hexanes until the filtrate became colorless. The filtrate was dried in vacuo to obtain the crude thiol as a yellow oil. No further purification was possible due to fast oxidation to the di-sulfide.

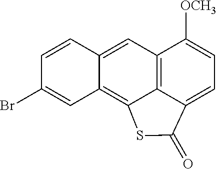 [SMILES: COc1ccc2c3c(c4cc(Br)ccc4cc13)SC2=O]

9-Bromo-5-methoxy-2H-anthra[9,1-bc]thiophen-2-one (33). The crude 2-bromo-5-methoxyanthracene-9-thiol (32, 1.26 g, 3.96 mmol) was dissolved in 100 mL dry DCM and cooled to 0° C. To this mixture triphosgene (3.53 g, 11.9 mmol) and pyridine (1.28 mL, 15.8 mmol) were added and stirred at 0° C. for 3 h. After completion, the reaction was quenched by the slow addition of ice-cold water, extracted with DCM and dried in vacuo. The metastable carbonochloridothioate intermediate obtained was dissolved in 100 mL dry DCM and cooled down to 0° C. To this AlCl₃ (0.79 g, 5.94 mmol) was added and the mixture stirred for 1 h while allowing it to warm up to room temperature. After completion of the reaction, DCM was removed in vacuo, the crude product was dissolved in EtOAc and washed twice with 1N HCl. The organic layer was dried over anhydrous Na₂SO₄ and the solvent was removed in vacuo. The product was purified using silica column chromatography with 20% EtOAc in hexanes to afford an orange solid (0.46 g, 34% yield over three steps). ¹H NMR (400 MHz CDCl₃) δ ppm 8.58 (s, 1H), 8.21 (d, J=7.8 Hz, 1H), 8.02 (s, 1H), 7.90 (d, J=9.0 Hz, 1H), 7.55 (dd, J=9.0, 1.6 Hz, 1H), 7.00 (d, J=7.8 Hz, 1H), 4.21 (s, 3H); ¹³C (100 MHz CDCl₃) δ ppm 190.7, 162.2, 131.7, 130.9, 130.7, 129.6, 129.5, 129.2, 128.1, 126.7, 124.8, 122.0, 121.6, 120.4, 104.5, 56.7; UPLC-ESI-HMS: calculated exact mass for C₁₆H₁₀BrO₂S⁺ [M+H⁺]: 344.9579, found [M+H⁺]: 344.9577.

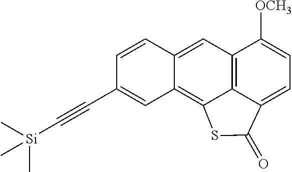 [SMILES: COc1ccc2c3c(c4cc(C#C[Si](C)(C)C)ccc4cc13)SC2=O]

5-Methoxy-9-((trimethylsilyl)ethynyl)-2H-anthra [9,1-bc]thiophen-2-one (34). Wang et al., 2014. In a flame-dried round-bottomed flask 9-bromo-5-methoxy-2H-anthra[9,1-bc]thiophen-2-one (33, 0.50 g, 1.45 mmol), Pd(PPh₃)₄ (0.17 g, 0.14 mmol) and CuI (83 mg, 0.43 mmol,) were added and flushed with Ar. To this mixture 10 mL of 1:1 anhydrous DMF and freshly distilled NEt₃ were added followed by TMS-acetylene (0.82 mL, 5.8 mmol). The reaction mixture was heated to 65° C. and stirred at that temperature for 16 h. After allowing the reaction to cool, the mixture was poured into ice-cold water and extracted with EtOAc. The organic layer was washed with water and brine, dried over anhydrous Na₂SO₄ and evaporated in vacuo. The crude product was purified by silica gel column using 20% EtOAc in hexanes to give the product as an orange solid (0.43 g, 82% yield). ¹H NMR (400 MHz CDCl₃) δ ppm 8.34 (s, 1H), 8.04 (d, J=7.9 Hz, 1H), 7.89 (quint, J=0.8 Hz, 1H), 7.83 (t, J=0.6 Hz, 1H), 7.81 (t, J=0.6 Hz, 1H), 7.41 (dd, J=8.7, 1.5 Hz, 1H), 6.84 (d, J=7.9 Hz, 1H), 4.13 (s, 3H), 0.36 (s, 9H); ¹³C (100 MHz CDCl₃) δ ppm 190.8, 161.9, 131.5, 130.4, 129.9, 129.9, 128.9, 128.5, 128.2, 126.7, 124.6, 122.0, 121.5, 119.7, 104.9, 104.4, 97.3, 56.6, −0.01; UPLC-ESI-HMS: calculated exact mass for C21H₁₉O₂SSi⁺ [M+H⁺]: 363.0870, found [M+H⁺]: 363.0869.

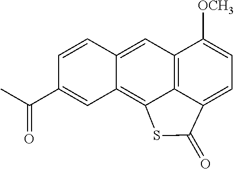 [SMILES: COc1ccc2c3c(c4cc(C(C)=O)ccc4cc13)SC2=O]

9-Acetyl-5-methoxy-2H-anthra[9,1-bc]thiophen-2-one (35). Menashe and Shvo, 1993. 5-Methoxy-9-((trimethylsilyl)ethynyl)-2H-anthra-[9,1-bc]thiophen-2-one (34, 0.40 g, 1.30 mmol) was dissolved in 10 mL neat HCOOH and refluxed for 5 h. After cooling the reaction, HCOOH was removed by azeotropic distillation with CHCl₃. The crude mixture was purified by silica column with 25% EtOAc in hexanes to a yellowish orange solid (0.35 g, 88% yield). ¹H NMR (400 MHz CDCl₃) δ ppm 8.69 (s, 1H), 8.49 (quint, J=0.8 Hz, 1H), 8.27 (d, J=7.9 Hz, 1H), 8.13 (d, J=8.9 Hz, 1H), 8.06 (dd, J=8.9, 1.6 Hz, 1H), 7.08 (d, J=7.9 Hz, 1H), 4.24 (s, 3H), 2.81 (s, 3H); ¹³C (100 MHz CDCl₃) δ ppm 197.5, 190.4, 162.1, 135.4, 134.0, 133.3, 130.9, 130.8, 129.5, 127.6, 126.6, 125.0, 123.5, 122.9, 119.9, 105.2, 56.8, 26.7; UPLC-ESI-HMS: calculated exact mass for C₁₅H₁O₃S⁺ [M+H⁺]: 309.0580, found [M+H⁺]: 309.0571.

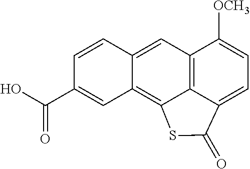 [SMILES: COc1ccc2c3c(c4cc(C(=O)O)ccc4cc13)SC2=O]

5-Methoxy-2-oxo-2H-anthra [9,1-bc]thiophene-9-carboxylic acid (36). Xu et al., 2018. In a flame-dried round-bottomed flask 9-acetyl-5-methoxy-2H-anthra[9,1-bc]thiophen-2-one (35, 0.10 g, 0.32 mmol), Fe(NO₃)₃·9H₂O (13 mg, 0.03 mmol) and 12 (8 mg, 0.03 mmol) were add and flushed with O₂. DMSO (5 mL) was added and the solution was degassed by bubbling with 02 for 15 min. The reaction mixture was heated at 130° C. for 20 h under O₂. After completion, the reaction was quenched with H₂O, the pH adjusted to 11.0 with 1M NaOH and the solution washed with EtOAc (3×). The pH of the aqueous phase was adjusted to 2.0 with 1N HCl and extracted with EtOAc. This organic layer was dried over anhydrous Na₂SO₄ and dried in vacuo. The product was purified by preparative HPLC (Phenomenex Luna 10μ C18(2) 100 Å preparatory column, 250×21.20 mm ID) using water and acetonitrile with 0.1% TFA as mobile phase to yield an orange solid (67 mg, 68% yield). ¹H NMR (400 MHz DMSO-d₆) δ ppm 8.55 (s, 1H), 8.24 (s, 1H), 8.21 (d, J=6.9 Hz, 1H), 8.18 (d, J=9.2 Hz, 1H), 7.88 (d, J=8.9 Hz, 1H), 7.23 (d, J=8.0 Hz, 1H), 4.18 (s, 3H); ¹³C (100 MHz DMSO-d₆) δ ppm 189.3, 167.1, 162.3, 133.5, 131.4, 131.2, 130.9, 130.1, 130.0, 127.1, 125.9, 125.0, 123.6, 122.2, 120.3, 106.7, 57.6; UPLC-ESI-HMS: calculated exact mass for C₁₇H₉O₄S⁻ [M−H⁺]: 309.0227, found [M+H⁺]: 309.0239.

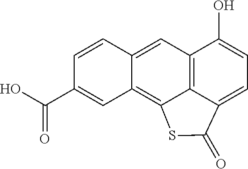 [SMILES: O=C(O)c1ccc2cc3c(O)ccc4c3c(c2c1)SC4=O]

5-Hydroxy-2-oxo-2H-anthra[9,1-bc]thiophene-9-carboxylic acid (28). In a flame-dried round-bottomed flask, 5-methoxy-2-oxo-2H-anthra[9,1-bc]thiophene-9-carboxylic acid (36, 0.10 g, 0.32 mmol) was dissolved in 5 mL of dry N-methyl-2-pyrrolidone (NMP), and to this solution K₂CO₃ (47 mg, 0.34 mmol) and PhSH (0.32 mL of 1M solution in NMP, 0.32 mmol) were added and the reaction mixture was placed in a pre-heated oil bath at 205° C. for 10 min. After cooling to room temperature, the reaction mixture was acidified with 1N HCl and extracted with EtOAc. The organic layer was washed once with 1N HCl and twice with a large volume of H₂O, dried over anhydrous Na₂SO₄ and the solvent was removed in vacuo. The product was purified by preparative HPLC (Phenomenex Luna 10μ C18(2) 100 Å preparatory column, 250×21.20 mm ID) using water and acetonitrile with 0.1% TFA as mobile phase as an orange solid (55 mg, 58% yield). ¹H NMR (400 MHz DMSO-d₆) δ ppm 8.78 (s, 1H), 8.41 (s, 1H), 8.30 (d, J=8.8 Hz, 1H), 8.21 (d, J=7.9 Hz, 1H), 7.93 (d, J=8.8 Hz, 1H), 7.31 (s, 1H), 7.14 (d, J=7.8 Hz, 1H); ¹³C (100 MHz DMSO-d₆) δ ppm 188.8, 167.3, 133.2, 131.8, 131.7, 131.5, 131.3, 130.0, 129.5, 128.5, 127.2, 126.3, 124.7, 122.6, 121.1, 110.7; UPLC-ESI-HMS: calculated exact mass for C₁₆H₇O₄S⁻ [M−H⁺]: 295.0071, found [M+H⁺]: 295.0095.

### 1.5.8 Procedure for Model C—N Coupling Reactions.

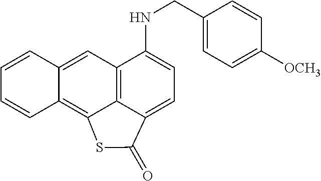 [SMILES: COc1ccc(CNc2ccc3c4c(c5ccccc5cc24)SC3=O)cc1]

5-((4-Methoxybenzyl)amino)-2H-anthra[9,1-bc]thiophen-2-one (38). Shafir and Buchwald, 2006; Altman and Buchwald, 2006. In a flame-dried round-bottomed flask 5-iodo-2H-anthra[9,1-bc]thiophen-2-one (5, 0.10 g, 0.28 mmol), CuI (3 mg, 0.01 mmol), CS₂CO₃ (0.18 g, 0.55 mmol) were added and the flask was evacuated and backfilled with Ar three times. Under a flow of Ar, 5 mL anhydrous DMF was added followed by 4-methoxybenzylamine (37, 0.41 mL of a 1M solution in dry DMF, 0.41 mmol). Finally, 4,7-dimethoxy-1,10-phenantholine (13 mg, 0.06 mmol) was added and the reaction was stirred at room temperature for 30 min. After completion, the reaction mixture was diluted with EtOAc, filtered through Celite and dried in vacuo. The crude product was purified by silica gel column using 15% EtOAc in hexanes to give an orange solid (81 mg, 78% yield). ¹H NMR (400 MHz CD₂Cl₂) δ ppm 8.54 (s, 1H), 8.29 (d, J=7.6 Hz, 1H), 8.13 (dd, J=7.2, 1.8 Hz, 1H), 8.10 (d, J=7.6 Hz, 1H), 7.88 (d, J=8.2 Hz, 1H), 7.66-7.59 (m, 2H), 7.49 (d, J=8.6 Hz, 2H), 6.94 (d, J=8.7 Hz, 2H), 5.01 (s, 2H), 3.82 (s, 3H).

When the ¹H NMR sample is freshly prepared and immediately recorded, mainly the single species above is detected, although a small amount of another can be seen in the baseline. In the longer acquisition of the ¹³C NMR spectrum the number of expected resonances approximately doubles, indicating two species are now present in roughly equal amounts. Re-recording the ¹H NMR spectrum of this “aged” sample revealed that the minor peaks noted above had now grown to approximately equal intensity with those of the original spectrum. We take these observations to suggest a monomer-dimer equilibrium in CD₂Cl₂ solution. Set out below are the ¹³C spectra of the mixture and the ¹H NMR spectral data for just the new species in addition to that noted above.

¹³C (150 MHz CD₂Cl₂) δ ppm 162.7, 159.6, 159.4, 138.3, 138.3, 138.0, 137.9, 133.3, 132.3, 131.8, 131.6, 131.1, 130.9, 130.3, 130.0, 129.9, 129.8, 129.3, 128.9, 128.5, 128.0, 127.5, 127.2, 127.0, 126.9, 125.6, 125.6, 125.1, 125.1, 124.0, 123.7, 122.6, 122.2, 121.6, 114.0, 113.9, 108.6, 105.1, 55.2, 55.2, 29.7, 29.6; ¹H NMR (600 MHz CD₂Cl₂) δ ppm 9.12 (s, 1H), 8.73 (d, J=8.6 Hz, 1H), 8.46 (dd, J=22.4, 7.6 Hz, 2H), 8.30 (d, J=8.2 Hz, 1H), 7.88 (d, J=8.8 Hz, 1H), 7.76 (dd, J=8.0, 7.1 Hz, 1H), 7.46 (d, J=8.7, 2H), 6.90 (d, J=8.7, 2H), 5.55 (d, J=15.1 Hz, 1H), 5.06 (d, J=15.1 Hz, 1H), 3.80 (s, 3H); UPLC-ESI-HMS: calculated exact mass for C₂₃H₁₈NO₂S⁺ [M+H⁺]: 372.1053, found [M+H⁺]: 372.1045.

1.5.9 Small-Scale Supplementation with 5, 13 and 25 in Wild Type M. chersina Fermentation and Analysis of Dynemicin a (3) Production.

Wild type M. chersina mycelial stock (100 L) was plated on med 53, Lam et al., 1995, with 2% agar and grown at 28° C. After 7 d, mycelia from the plate were inoculated into 50 mL med 53 liquid fermentation medium in a 125 mL Erlenmeyer flask and shaken at 250 rpm at 28° C. for another 7 d. This starter culture (2 mL) was inoculated into 50-mL H881 liquid medium, Lam et al., 1995, with or without added NaI (0.5 mg/L) in 250-mL Erlenmeyer flask and shaken at 250 rpm and 28° C. After 24 h, 250 L of 2-mM stock solutions of 5, 13 and 25 in DMSO (sterile filtered using 0.2-μm PTFE filters) were added to the respective flasks with or without NaI supplementation to provide a final concentration of the added compounds of 1 μM. After shaking at 250 rpm and 28° C. for another 2 d, Diaion® HP-20 (0.50 g, sterilized by autoclave) was added to each flask and shaking was continued for another 4 d. After a total 7 d of fermentation, 5 mL from each sample was extracted with 5 mL of EtOAc by vortexing for 1 min. Extracts were centrifuged at 4000×g and 4° C. for 5 min, and 4 mL of each EtOAc layer was dried by SpeedVac without heating. Samples were dissolved in 200 μL DMSO, filtered through 0.2 μm PTFE filters and analyzed on an Agilent 1200 HPLC using a Prodigy ODS3 100 Å, 5 μM, 250×4.6 mm column (Phenomenex). A gradient method of 5-95% ACN+0.1% (v/v) formic acid over 40 min, followed by 10 min hold at 95% ACN before column re-equilibration was used at a 1 mL/min flow rate to achieve separation of metabolites. Production of dynemicin and other related metabolites was monitored at 280 nm, 450 nm, and 570 nm. Each experiment was performed in triplicate.

1.5.10 Small-Scale Supplementation with CuSO₄ in Wild Type M. chersina Fermentation and Analysis of Dynemicin a (3) Production

Wild type M. chersina mycelial stock (100 μL) was plated on med 53 with 2% agar and grown at 28° C. After 7 d, mycelia from the plate were inoculated into 50-mL med 53 liquid fermentation medium in a 125-mL Erlenmeyer flask and shaken at 250 rpm at 28° C. for another 7 d. A 2-mL sample of this starter culture was inoculated into 50-mL H881 liquid medium in 250 mL Erlenmeyer flasks prepared with and without CuSO₄·5H₂O (0.05 g/L), each in triplicate, and shaken at 250 rpm and 28° C. On day 3 of fermentation, Diaion® HP-20 (0.50 g. sterilized by autoclave) was added to each flask and shaking was continued for another 4 d. After a total 7 d of fermentation, 5 mL from each culture were individually extracted with 5 mL of EtOAc by vortexing for 1 min. Extracts were centrifuged at 4000×g and 4° C. for 5 min, and 4 mL of each EtOAc layer were dried by SpeedVac without heating. Samples were dissolved in 200-μL DMSO, filtered through 0.2 μm PTFE filters and analyzed on an Agilent 1200 HPLC using a Prodigy ODS3 100 Å, 5 μM, 250×4.6 mm column (Phenomenex). A gradient method of 5-95% ACN+0.1% (v/v) formic acid over 40 min, followed by 10 min hold at 95% ACN before column re-equilibration was used with 1 mL/min flow rate to achieve separation of metabolites. Production of dynemicin and other related metabolites was monitored at 280, 450 and 570 nm. Area under the peaks at 280 nm was calculated in ChemStation for dynemicin A 3, iodoanthracene 5 and des-iodoanthracene 12 to determine their relative production amounts. Each experiment was performed in triplicate.

1.5.11 M. chersina Cell-Free Extract Protein Pull-Down Experiment
1.5.11.1 Large Scale Fermentation of Wild Type M. chersina and ΔOrf14 Mutant

Mycelial stock of the wild type M. chersina or the Δorf14 mutant was plated on med 53 with 2% agar and incubated at 28° C. for 7 d. Then mycelia from the plate were inoculated into 4×50 mL med 53 liquid culture in 125 mL Erlenmeyer flasks and shaken at 250 rpm at 28° C. After 7 d, seed cultures were combined, and 6×25 mL was inoculated into 6×1 L H881 liquid medium in 2.8 L shake flasks and rotated at 180 rpm and 28° C. for 5 d. After 5 d, the cultures were centrifuged at 5000×g for 15 min and the cell pellets were frozen in liq nitrogen for future use.

### 1.5.11.2 Cell Lysis and Preparation of Cell Free Extract (CFE)

Frozen cell pellets (100 g) were washed by resuspending in 400 mL CFE buffer (50 mM Tris-HCl, 0.1 mM DTT, 10% glycerol (v/v), pH 7.4) and re-pelleting by centrifugation at 5000×g for 15 min at 4° C. The supernatant was discarded, and the pellet was finally suspended in 150 mL CFE buffer. To this solution, 250 mg lysozyme was added (2.50 mg per 1 g of cell pellet) and incubated on ice for 45 min. Lysozyme-treated cells were lysed by sonication on ice for 10 min (9.9 sec on, 9.9 sec off) at 40% amplitude (Vibra-Cell Ultrasonic Processor, Sonics & Materials, Inc.). The lysate was cleared by centrifugation at 27000×g at 4° C. for 30 min. The supernatant was separated and further concentrated to 60 mL final volume by centrifugation at 4000×g using 3 k MWCO Amicon Ultra centrifugal filter (Millipore) at 4° C.

### 1.5.11.3 Magnetic Bead Preparation

BcMag™ amine-terminated magnetic beads (5 mL, silica-based superparamagnetic beads coated with a high density of primary amine functional groups with an R₃ linker on the surface, approximately 1 μm diameter, approximately 1.7×10⁸ beads/mg, effective density 2.5 g/mL, functional group density approximately 250 mol/g of beads) were transferred to a 50-mL tube and resuspended in 30 mL coupling buffer (10 mM K/PO4, 0.15 M NaCl, 40% dioxane (v/v), pH 5.5) by vortex. The tube was left at room temperature for 5 min, placed on the magnetic separator (BcMag Separator-50, Cat. #MS-04) for another 5 min and the supernatant was removed while the tube remained on the separator. The wash steps were performed twice more and finally the beads were resuspended in 5 mL coupling buffer and stored on ice.

### 1.5.11.4 Coupling Substrate Mimic to Magnetic Beads

Compound 30 (15 mg, 0.05 mmol) was dissolved in 2 mL DMSO and added to the 5 mL of prepared beads in a 15 mL tube. To this mixture 0.55 mL of EDC·HCl stock solution (0.10 M in coupling buffer, 0.05 mmol) and 25 μL 4-DMAP stock solution (0.10 M in coupling buffer, 25 mol) were added and the tube was gently rotated at room temperature for 24 h. The tube was placed on the magnetic separator for 5 min and the supernatant was removed while the tube remained on the separator. The beads were resuspended in 10 mL of 1:4:5 DMSO:dioxane:water and mixed by vortex. The tube was left at room temperature for 5 min and the supernatant was removed using the magnetic separator. The above washing step was repeated once with the DMSO:dioxane:water mixture and then twice with the CFE buffer. Finally, the activated beads were suspended in 6 mL of CFE buffer and stored on ice. To confirm the coupling, 0.5 mL of the activated beads was transferred to a 1.5 mL Eppendorf tube and to this a few drops of conc. H₂SO₄ were added, and the tube was heated at 95° C. for 16 h. The supernatant was removed using the magnetic separator and the absorbance at 450 nm (selective absorption of the affinity label 30) was measured and compared to the previous washes. An increased absorbance of the acid-treated supernatant compared to the washes confirmed the release of 30 from the activated beads upon acid treatment. As a control, 0.50 mL of unactivated beads as treated the same way with cone. H₂SO₄ and the absorbance of the supernatant at 450 nm was measured; no increase in absorbance was observed after acid treatment.

### 1.5.11.5 Cell-Free Extract Protein Pull-Down

Activated beads (5 mL) were mixed with 40 mL of the M. chersina CFE prepared above and gently rotated at 4° C. for 24 h. As a control, 20 mL of CFE was mixed with 1 mL of unactivated beads and gently rotated at 4° C. for 24 h. After 24 h, 20 mL of the reaction mixture was transferred to a 50-mL tube, placed on the magnetic separator for 5 min at 4° C. and the supernatant was removed as the flow-though (FT). The beads were resuspended with 10 mL of wash buffer (1×PBS buffer, 0.1 mM DTT, 10% (v/v) glycerol) by gentle mixing and left on ice for 5 min. The supernatant was removed using the magnetic separator and stored on ice as wash 1 (W1). The beads were washed again with another 10 mL of wash buffer and the supernatant was collected as wash 2 (W2). The proteins of interest were then eluted by washing the beads twice with 3×5 mL wash buffer with added substrates (mixture of iodoanthracene 5 and bromoanthracene 13 in 4:1 ratio) at increasing concentrations (E1 and E2 with 0.25-mM and 0.5-mM final concentration of substrate mixtures, respectively). All the washes and elutions were further concentrated by centrifugation at 4000×g using 3 k MWCO Amicon Ultra centrifugal filter (Millipore) at 4° C. to 1 mL final volume. A portion (20 mL) of the control reaction was washed once with 10 mL of wash buffer and eluted once with 5 mL of wash buffer+0.5 mM iodoanthracene 5 after 24 h. A 100 μL sample of each was mixed with 25 μL of 5×SDS-PAGE loading dye, heated at 95° C. for 5 min and centrifuged for 5 min. Supernatant (25 L) from each sample was run on a 12% SDS-PAGE gel, stained with SimplyBlue™ SafeStain from Thermo Fisher.

### 1.5.11.6 Mass Spectrometry Methods for Protein Identification

Proteins in gel bands were proteolyzed with trypsin (Promega) as previously described. Shevchenko et al., 1996. Peptides were analyzed by liquid chromatography interfaced with electrospray ionization tandem mass spectrometry (LC/ESI-MSMS) using an Easy-LC 1100 HPLC system (Thermo Fisher Scientific) interfaced with an Orbitrap Q-Exactive Plus Mass Spectrometer (Thermo Fisher). Peptides were resuspended in 20 L loading buffer (2% acetonitrile in 0.1% formic acid) and loaded onto a C18 trap (S-10 μM, 120A, 75 μm×2 cm, YMC, Japan), subsequently separated on an in-house packed PicoFrit column (75 μm×200 mm, 15 u, +/−1 um tip, New Objective) with C18 phase (ReproSil-Pur C18-AQ, 3 μm, 120A, www.dr-maisch.com) using a 2-90% acetonitrile gradient at 300 nL/min over 90 min. Eluting peptides were sprayed at 2.2 kV directly into the Q-Exactive.

Survey scans (full MS) were acquired from m/z=350-1700 with data-dependent monitoring with top 15 loop count. Each precursor was individually isolated in a 1.6 Da window and fragmented using HCD activation collision energy of 27 and 20 sec dynamic exclusion, first mass being 120 m/z. Precursor and the fragment ions were analyzed at resolutions 140,000 and 35,000, respectively, with automatic gain control (AGC) target values at 3e6 with 60 ms maximum injection time (IT) and 1e5 with 200 ms maximum IT, respectively.

Proteome Discoverer (Version 1.4, Thermo Fisher). Charge state deconvolution and deisotoping were not performed. All MS/MS samples were analyzed using Mascot (version 1.4.0.288, Matrix Science, London, UK). Mascot was set up to search the 190923_TownsendC_PP_Mchersina_20190923 database (11701 entries) assuming the digestion enzyme trypsin. Mascot was searched with a fragment ion mass tolerance of 0.030 Da and a parent ion tolerance of 5.0 PPM. Deamidated of asparagine and glutamine, oxidation of methionine and carbamidomethyl of cysteine were specified in Mascot as variable modifications.

Scaffold (version Scaffold_5.0.1, Proteome Software Inc., Portland, OR) was used to validate MS/MS based peptide and protein identifications. Peptide identifications were accepted if they could be established at greater than 85.0% probability to achieve an FDR less than 1.0% by the Scaffold Local FDR algorithm. Protein identifications were accepted if they could be established at greater than 95.0% probability and contained at least 1 identified peptide. Protein probabilities were assigned by the Protein Prophet algorithm. Nesvizhskii et al., 2003. Proteins that contained similar peptides and could not be differentiated based on MS/MS analysis alone were grouped to satisfy the principles of parsimony.

1.5.11.7 Protein Coverage for Band 1 for Pull-Down Experiment with Wild Type Fermentation CFE

| ACB47059.1 (100%), 48,841.8 Da | | | |
|---|---|---|---|
| PBS lyase HEAT-like repeat protein [Micromonospora chersina] | | | |
| 36 exclusive unique peptides, 92 exclusive unique spectra, 283 total spectra, | | | |
| 301/441 amino acids (68% coverage) | | | |
| MAIDLCNKSP | GALAEGAQGA | WEEAVPADAA | PVNLDALTAD |
| QRDALNFSYR | TTLPAMDPRF | VAGDPAAWAS | DFGYALNKVA |
| VRLDNRTNQE | LRDAALNHPD | AAMREQALFE | YADRDLPDAI |
| ELLGQAVLHD | PNREVRWDAL | WAIEKLGGAH | AVASLSKFAK |
| DADPEIAEWS | HLFRSELQTG | DPAFDGRAGK | FTPGRTFDET |
| IYLLIHCDLY | VRLDDSNQHW | GKISLAPQGL | ARIYGQAHAC |
| PNVATRERQL | VIAKTIDGLH | ADGSPHCDNY | LFRGFTDRTR |
| RDRGNFFFES | LVPRTFFKSG | HADDPSEGTR | QANIGFARYG |
| TWHLDPKFQI | HDEAAIRYVR | GRFQGQGYIN | LARIAGRSME |
| EILQPGNGVL | STLHDPEIGH | MTNAFILGTF | KGKLNDWDGD |
| GVIDMNSRDV | YSTVDGEIDM | NQDGVADQPG | LTCCDHTTLL |
| P | | | |

1.5.11.8 Protein Coverage for Band 2 for Pull-Down Experiment with Wild Type Fermentation CFE

| ACB47057.1 (100%), 44,576.1 Da | | | |
|---|---|---|---|
| unkown protein [Micromanospora chersina] | | | |
| 20 exclusive unique peptides, 64 exclusive unique spectra, 449 total spectra, | | | |
| 258/405 amino acids (64% coverage) | | | |
| MREAGSGRSP | APARPPRPPR | PGRGNPKERR | QSEVGMPVDD |
| RSAPAPRLEP | RPGFTGVAPD | DFPLLHRNGG | YPLAQDRWER |
| LVSQVHIPAP | ADRVWSALTD | PEQVAQWLAV | CRGGWATADG |
| EAILDFEDGE | FFFCRIRDVR | EPASGGEGRL | SYLWRWVGVG |
| PATEVTWQLT | ATPHGTQVTV | VEEAFNPPSD | WRSWNGMGWP |
| GILDQLAQFI | RTGVPWRWPW | RRMGPYVQTE | LPGTTFEVWG |
| LLTSTPATQF | WLSRTSGSLD | AGSEVGLILG | DASGVATMTV |
| TRHVEAYQQF | PSYLPRLEFR | LGRPGWPGEL | EGHLWIEPAG |
| LGGSIIQVFH | SNWEALSTIA | PPLDERKILT | GYWVGAFGRA |
| ALLCQRAFGD | SFPAAPPGRD | LDIPADLNGT | GNGQRVDAGP |
| HGWSR | | | |

1.5.11.9 Protein Coverage for Band 3 for Pull-Down Experiment with ΔOrf14 Fermentation CFE

| ACB47059.1 (100%), 48,841.8 Da | | | |
|---|---|---|---|
| PBS lyase HEAT-Ilke repeat protein [Micromonospora chersina] | | | |
| 31 exclusive unique peptides, 67 exclusive unique spectra, 293 total spectra, | | | |
| 337/441 amino acids (76% coverage) | | | |
| MAIDLCNKSP | GALAEGAQGA | WEEAVPADAA | PVNLDALTAD |
| QRDALNFSYR | TTLPAMDPRF | VAGDPAAWAS | DFGYALNKVA |
| VRLDNRTNQE | LRDAALNHPD | AAMREQALFE | YADRDLPDAI |
| ELLGQAVLHD | PNREVRWDAL | WAIEKLGGAH | AVASLSKFAK |
| DADPEIAEWS | HLFRSELQTG | DPAFDGRAGK | FTPGRTFDET |
| IYLLIHCDLY | VRLDDSNQHW | GKISLAPQGL | ARIYGQAHAC |
| PNVATRERQL | VIAKTIDGLH | ADGSPHCDNY | LFRGFTDRTR |
| RDRGNFFFES | LVPRTFFKSG | HADDPSEGTR | QANIGFARYG |
| TWHLDPKFQI | HDEAAIRYVR | GRFQGQGYIN | LARIAGRSME |
| EILQPGNGVL | STLHDPEIGH | MTNAFILGTF | KGKLNDWDGD |
| GVIDMNSRDV | YSTVDGEIDM | NQDGVADQPG | LTCCDHTTLL |
| P | | | |

### 1.5.12 Protein Modeling and Function Prediction

The 3D-model of Orf14 and Orf16 was generated using AlphaFold. Jumper et al., 2021. Structural alignment of Orf14 with DynU16 (PDB:6V04) and CalU16 (PDB:4FPW) was performed using PyMOL. Structure based similarity search was made using DaliLite server. Holm et al., 2022.

### 1.5.13 Generation of ΔOrf14 and ΔOrf16 Mutants

CRISPR-Cas9 plasmids with HRTs and sgRNAs were delivered to M. chersina by conjugation as described previously. Cohen and Townsend, 2018a; Kieser et al., 2000. The CRISPR-Cas9 plasmids were conjugated into electrocompetent cells, E. coli GM2929 hsdS::Tn10 (pUB307::Tn7). The colonies were selected by growing on LB agar with apramycin and streptomycin (100 and 50 g/mL respectively). Positive clones were grown overnight in LB at 37° C. with apramycin and streptomycin. 500 L of each overnight starter culture was added to separate 50 mL LB with the same drug concentrations were inoculated. Once the cells reached OD₆₀₀ of approximately 0.4 at 37° C., the cells were pelleted by centrifugation at 6500×g at 4° C. The pellets were washed twice with approximately 40 mL sterile LB. After washing, the cells were resuspended in approximately 2.5 mL LB. 500 μL of the pCRISPR-Cas9 containing E. coli was added to germinated M. chersina spores. The spores, 50 μL, were germinated by diluting with 500 μL of 2×YT and warmed at 50° C. for 10 min. After concentration of the spore/E. coli mixture, 75 μL of the mixture was plated on medium 53 with 2% agar plates supplemented with 10-mM MgCl₂ in duplicate. The plates were incubated at 28° C. overnight. Afterwards, nalidixic acid and apramycin in sterile ddH₂O (20 and 50 g/mL respectively) were overlaid on the plates. The plates were then incubated at 28° C. for 7 days.

Ex-conjugates were restreaked 3 times: first on to medium 53 agar with nalidixic acid and apramyicin plates, next on medium 53 agar with nalidixic acid, apramycin, and thiostrepton (1 μg/mL) plates to induce the CRISPR-Cas9, and lastly, on medium 53 agar plates without drug to recover. After each restreak, the plates were incubated at 28° C. for 7 days. Mutants were finally screened by colony PCR and gene deletions confirmed by isolation, PCR and sequencing of the gDNA. Cohen and Townsend, 2018a.

### 1.5.13 Small-Scale Fermentation of ΔOrf14 and ΔOrf16 Mutants and Analysis of Dynemicin a (3), Iodoanthracene (5) and Reduced Anthracene (12) Productions

M. chersina Δorf14 and Δorf16 mutant mycelial stock (100 μL) was plated on med 53, Lam et al. 1995, with 2% agar and grown at 28° C. After 7 d, mycelia from the plate were inoculated into 50 mL med 53 liquid fermentation medium in a 125 mL Erlenmeyer flask and shaken at 250 rpm at 28° C. for another 7 d. This starter culture (2 mL) was inoculated into 50 mL H881 liquid medium, Lam et al. 1995, in 250-mL Erlenmeyer and shaken at 250 rpm and 28° C. After a total 7 d of fermentation, 5 mL from each sample was extracted with 5 mL of EtOAc by vortexing for 1 min. Extracts were centrifuged at 4000×g and 4° C. for 5 min, and 4 mL of each EtOAc layer was dried by SpeedVac without heating. Samples were dissolved in 200 μL DMSO, filtered through 0.2 μm PTFE filters and analyzed on an Agilent 1200 HPLC using a Prodigy ODS3 100 Å, μM, 250×4.6 mm column (Phenomenex). A gradient method of 5-95% ACN+0.1% (v/v) formic acid over 40 min, followed by 10 min hold at 95% ACN before column re-equilibration was used at a 1 mL/min flow rate to achieve separation of metabolites. Production of dynemicin and other related metabolites was monitored at 280, 450 and 570 nm. Each experiment was performed in quadruplecates.

1.5.14 Heterologous Expression of Orf14 and Orf16 in E. coli

Orf14 and orf16 sequences were isolated from M. chersina wild type gDNA using PCR following a standard protocol. Herbst et al., 2018. Primers were used to ligate in both pET-28a and pET-29b having appropriate restriction cut sites. The pET-28a vector was digested with NdeI and NotI to ligate with both orf14 and orf16. The pET-29b vector was digested with EcoRI and NotI for ligation with orf14 and with EcoRV and NotI for orf16 ligation. The digested vectors were gel purified. Amplified orf14 and orf16 were ligated to appropriately digested vectors using the Gibson assembly protocol. The newly constructed vectors were transferred into electrocompetent NEB-5a E. coli cells using the manufactures high-efficiency transformation protocol and plated on LB agar plates with 25 μg/mL kanamycin for selection. Constructed plasmids were first screened using restriction digestion to verify successful ligation into the vectors and finally confirmed by sequencing at the Johns Hopkins University Sequencing and Synthesis Facility or Genewiz (South Plainfield, New Jersey).

The vectors were finally transformed into Rosetta 2 cells by electroporation. The resulting Rosetta cells were grown in 100 mL LB with 25 μg/mL kanamycin at 37° C. until OD₆₀₀ reached 0.6 and then cold shocked in ice water for 30 min. A 1 mL sample was centrifuged at 14000×rpm for 5 min and the pellet was resuspended in 250-μL ddH₂O and frozen down as the uninduced sample. Protein expressions were induced in the remaining cultures by addition of 1 mM IPTG and shaken at 16° C. overnight. A 1 mL sample of induced culture was saved as described above for later analysis. The remaining culture was harvested by centrifugation at 4000×g for 10 min. The cell pellet was resuspended in 20 mL lysis buffer (50-mM potassium phosphate buffer at pH 8.0 with 300-mM NaCl and 10% glycerol) and lysed by sonication. The resulting lysate was cleared by centrifugation at 27000×g for 20 min. The supernatant served as the soluble protein component. A small portion of the pellet was resuspended in 250 μL ddH₂O to check for insoluble proteins. All fractions were run on SDS-PAGE gel to check for the expression and solubility of the desired protein.

1.5.15 Extraction of ΔOrf14, Wild Type and ΔdynE8 Fermentations for In Vitro Experiments

Mycelial stocks of the Δorf14 mutant, wild type and ΔdynE8 mutant as control were plated on med 53 with 2% agar and incubated at 28 C for 7 d. Then mycelia from each plate were inoculated into three separate 50 mL med 53 liquid culture in 125-mL Erlenmeyer flasks and shaken at 250 rpm at 28° C. After 7 d, 3 mL of seed cultures were inoculated into 2×50 mL H881 liquid medium in 250-mL shake flasks for each fermentation and rotated at 250 rpm and 28° C. After 2 d, Diaion® HP-20 (10 g, sterilized by autoclave) was added to each flask and shaking was continued. After 7 d of fermentation, each 50-mL cultures were combined with 50 mL of EtOAc, stirred at room temperature for 2 h, covering the flasks with aluminum foil to minimize exposure to light. The combined mixture was centrifuged at 5000×g for 15 min, the supernatant was filtered through Celite, and the organic layer was separated. The combined organic layer was dried with anhydrous Na₂SO₄ and concentrated in vacuo. The dried extracts were dissolved in 50 μL of DMSO and used as a source of metabolites in in vitro reaction with Orf14+Orf16 mixtures.

1.5.16 In Vitro Reaction Attempts Between Iodoanthracene (5) and Commercially Available Amines or Metabolites from Fermentations in Presence of Orf14+Orf16 Mixture

5-mM stock solutions of iodoanthracene 5 and the amines used were made in DMSO, 5 mg/mL CuSO₄ solution was made in CFE reaction buffer (50 mM Tris-HCl, 0.1 mM DTT, 10% glycerol, pH 7.4). Reactions were performed in CFE reaction buffer by diluting Orf14+Orf16 mixtures to 1.0 μM final concentration (measured by Bradford absorbance), followed by addition of 5 (0.1 mM final concentration) and CuSO₄ (0.05 mg/mL final concentration). Reactions were initiated on ice by adding the amines (0.1 mM final concentration) or 50 μL DMSO solutions of metabolites from Δorf14 (day 5 and 7), wild type (day 5 and 7) and ΔdynE8 (day 7) fermentations. The reactions were allowed to run for 3 h on ice followed by another 3 h at room temperature in the dark. Samples were taken at 3 h and 6 h time points, quenched with 1N HCl (5 μL to 250 μL reaction), filtered through 0.2-μm PTFE filters and analyzed by HPLC using a Prodigy ODS3 100 Å, μM, 250×4.6 mm column (Phenomenex). A gradient method of 5-95% ACN+0.1% (v/v) formic acid over 40 min, followed by 10 min hold at 95% ACN before column re-equilibration was used with 1 mL/min flow rate to achieve separation of metabolites. Production of coupled products was monitored at 280 nm.

### Example 2

### Combinatorial Biosynthesis of Dynemicin Analogues

### 2.1 Overview

Nature offers a plethora of structurally and functionally fascinating molecules that present opportunities and challenges to the scientific community. Enediyne anticancer antibiotics, derived from bacterial sources, are a class of such compounds with utmost cytotoxicity observed among natural products and have a rapidly developing interest as cancer chemotherapeutics. Soon after the establishment of their unprecedented molecular architecture and exceptional mode of action, they quickly moved to center stage, eliciting extensive chemical, biological and biochemical research activities.

Enediynes are characterized by the presence of bicyclo[7.3.0]dodecadienediyne or bicyclo[7.3.1]-tridecadiynene core; or more simply, a carbocycle containing an alkene flanked by two alkynes. Based on the size of the carbocycle, all known enediynes can be categorized as 9- or 10-membered: 9-membered enediynes are usually present as a 1:1 complex of an apoprotein and a chemically unstable chromophore. 10-Membered enediynes are stable small molecules with either a calicheamicin-like structure or an anthraquinone fused structure. Shen et al., 2005.

Most of the known enediynes exhibit rapid and much higher activity against cancer cells relative to the commonly used anticancer drugs, such as Adriamycin. Zhen et al., 1989. IC₅₀ values from in vitro experiments for enediynes range approximately 1-100 pg mL⁻¹. In vivo they are also extremely potent against transplantable cervical carcinoma HeLa, leukemia P388, L-1210 and melanoma B16 (exceptions include NCS) (see Table 3).

TABLE 3
In vivo and in vitro activity of well-studied
enediyne anticancer antibiotics. Shao, 2010.

| | | | In vivo |
|---|---|---|---|
| | | In vitro | ID50 |
| Enediyne | Producing strain | IC50 (nM) | (μg/kg) |
| Neocarzinostatin | Streptomyces | 225-900 | 380 |
| Meada et al., 1978; | carzinostaticus | | |
| Shimoyama et al., | | | |
| 1979; Kimura, 1978 | | | |
| Lidamycin | Streptomyces | 0.01-0.05 | 0.25-0.5 |
| Zhen et al., 1989; | globisporus C-1027 | | |
| Zhen et al., 1988 | | | |
| Kedarcidin | Actinomycete | 1 | 2-3.3 |
| Lam et al., 1991; | strain L585-6 | | |
| Leet et al., 1992 | | | |
| Calicheamicins | Micromonospora | 6-9 | 0.5-1.5 |
| Zein et al., 1988; | echinospora ssp | | |
| Maise et al., 1989 | | | |
| Esperamicins | Actinomadura | 0.3-8.3 | 0.1-0.2 |
| Konishi et al., | verrucosospora | | |
| 1985; Batchelder | | | |
| et al., 1996 | | | |
| Dynemicins | Micromonospora | 0.9-10 | 30-60 |
| Konishi et al., | chersina M956-1 | | |
| 1989; Konishi et | | | |
| al., 1991 | | | |

The unparalleled cytotoxic activity of enediynes stems from their ability to cleave DNA. Nicolaou et al., 1991; Nicolaou et al., 1993. Upon in vivo activation, the enediyne moiety undergoes a cycloaromatization, similar to the Bergman cyclization, Jones and Bergman, 1972, or related Myers-Saito cyclization, Nagata et al., 1989; Myers et al., 1989, to generate a benzenoid diradical. This highly reactive intermediate abstracts hydrogen atoms from the DNA sugar backbone leading to DNA interstrand crosslinking and single- or double-strand cleavage. The molecular architecture of enediynes is comprised of (1) the warhead that consists of the enediyne moiety, (2) a DNA interaction unit (either minor groove binders, such as calicheamicin and neocarzinostatin or DNA intercalators, such as dynemicin), and (3) a triggering unit that upon proper activation initiates the cascade of reactions that ultimately result in the generation of the reactive radical species that initiate DNA damage. However, indiscriminate cytotoxicity has limited their clinical use. But when linked to proper delivery systems, enediynes have shown high potential as chemotherapeutic agents; two of the thirteen known enediynes are currently on the FDA approved drug lists for cancer treatment. The poly(styrene-co-maleic acid)-conjugated neocarzinostatin was the first to obtain approval in Japan in 1993 and has been used ever since under the brand name SMANCS as one of the practical treatments for advanced or recurrent hepatocellular carcinoma. Maeda, 2001; Maeda, 1994. A CD33 monoclonal antibody (mAB) conjugated with calicheamicin was approved by the FDA in 2000 with the brand name Mylotarg (CMA-676, gemtuzumab ozogamicin). Hamann et al., 2002; Boghaert et al., 2006. This was the first of the antibody-drug conjugate (ADC) class for the treatment of first relapse with CD33-positive acute myeloid leukemia in patients 60 years and older. More recently in 2017, FDA has approved another calicheamicin-based ADC with the CD22 mAB, as a first-in-class medication for CD22-positive B-cell acute lymphoblastic leukemia under the trade name Besponsa (CMC-544, Inotuzumab ozogamicin). Wynne et al., 2019; Dijoseph et al, 2006; Dijoseph et al., 2005; Dijoseph et al., 2004. There are several other calicheamicin-, neocarzinostatin- and C-1027-based ADCs under development and clinical trials with promising outcomes. See Table 4.

TABLE 4
Derivative and conjugate of enediynes in development. Shao, 2010.

| Enediyne derivatives | Molecular target | Tumor type |
|---|---|---|
| 3G11-LDM | type IV collagenase | hepatoma, colorectal |
| Feng et al., 2007; | | carcinoma |
| Li et al., 2005 | | |
| Fab-LDM | tumor-specific | hepatoma BEL-7402 |
| Li et al., 1994. | antigen | |
| VH-LDP-AE | type IV | sarcoma HT-1080 |
| Miao et al., 2007 | collagenase | |
| hCTM01-calicheamicin-γ | MUCI antigen | breast, ovarian |
| Hamann et al., 2005a; | | carcinoma |
| Hamann et al., 2005b | | |
| hu3S193-CalichDMH | Lewis^{γ} antigen | gastric, colon, |
| Boghaert et al., 2004 | | prostate carcinomas |
| 138H11-Camtheta | γ- | metastasized renal |
| Knoll et al., 2000. | glutamyltransferase | cell carcinoma |
| A7-NCS | tumor-specific | human gastric, |
| Okamoto et al., 1998. | antigen | colorectal, |
| | | pancreatic carcinoma |
| chA7Fab-NCS | tumor-specific | human pancreatic |
| Okamoto et al., 1998; | antigen | carcinoma |
| Otsuji et al., 1996. | | |

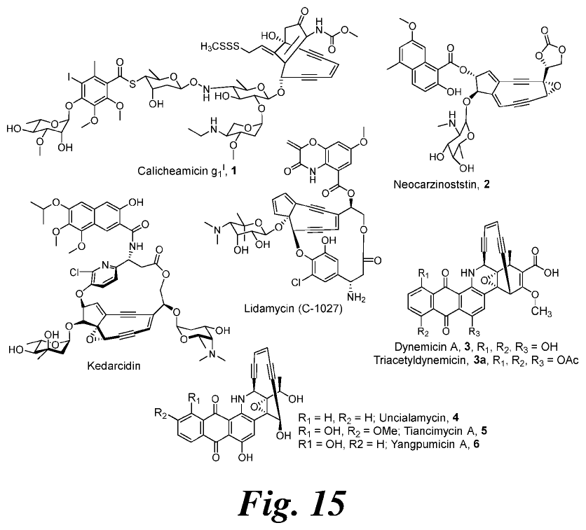

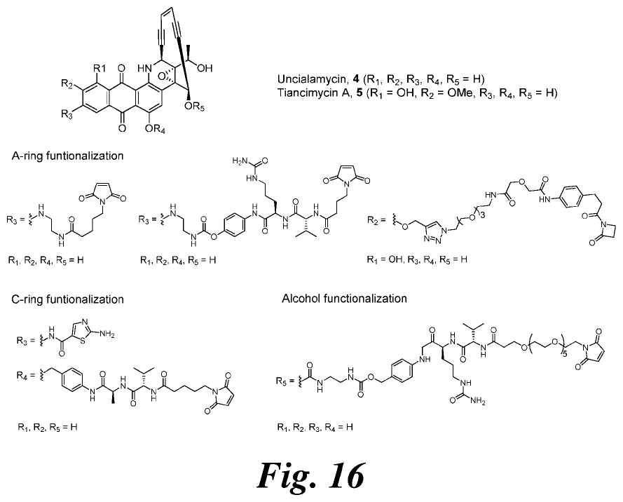

These examples illustrate the potential of utilizing enediynes as powerful rugs by controlling their cytotoxicity and in combination with suitable target-specific delivery systems. On the other hand, they also present the huge challenge of developing new enediyne analogues for mechanistic and pharmaceutical studies. Total syntheses of enediynes and their analogues with their very complicated architecture, while a monumental task, have already been achieved for some members of this family. In 1992, Nicolaou et al. first reported the total synthesis of enantiopure calicheamicin (Cal). Nicolaou et al., 1992. Soon after, total syntheses of dynemicin A (Dyn A), Shair et al., 1995; Shair et al., 1996; Myers et al., 1995, uncialamycin (Ucm), Nicolaou et al., 2016, tiancimycin (Tnm), yangpumicin (Ypm) and related anthraquinone-fused enediynes have been published. Nicolaou et al., 2020. More recently, various analogues of Ucm, Tnm and Ypm have been synthesized by Nicolaou et al., some of which have shown remarkably high activity compared to the naturally-occurring enediynes against various cancer cell lines including multidrug resistant cell lines. Nicolaou et al., 2016; Nicolaou et al., 2020. Two Ucm analogues with an —NH₂ and —NH—CH₂—CH₂—NH₂ group at the analogous A-ring position in Dyn-C16, synthesized by Chowdari et al., have shown much higher potency than MMAE, the payload used in the FDA approved ADC Adcetris. Chowdari et al., 2019. Extensive research is ongoing to develop ADCs based on these anthraquinone fused enediynes and their analogues, Wang et al., 2018; Adhikari et al., 2021, by attaching target specific antibodies via a linker to the newly installed substituents on the A-ring or the native hydroxyl group on the C-ring (FIG. 16). Poudel et al., 2020. Some ADCs developed with Ucm have shown highly selective and strong bystander killing effect both in vitro and in vivo. Nicolaou et al., 2021.

Similar efforts with Dyn are limited to total synthesis of Dyn A, dideoxydynemicin (without the A ring hydroxyls) and simpler models for SAR studies. Myers et al., 1995; Nicolaou et al., 1990. However, the total synthesis of this large and intricate enantiopure molecule is long and complex, posing a formidable challenge to generate a library of analogues for SAR studies. On the other hand, combinatorial or directed biosynthesis provides an alternative way to produce natural product analogues with a far smaller burden of organic synthesis. The prerequisite for this approach is to find a proper genetic system by manipulation of the native producer or in a recombinant host, to administer modified substrates or early on-pathway intermediates to generate the modified final metabolites. With some exciting new discoveries about the Dyn biosynthetic pathway made by our lab, we sought to explore the possibility of generating Dyn analogues using combinatorial or directed biosynthetic methods.

### 2.2 Results

### 2.2.1 Identification of Orf15 as the Key to Combinatorial Biosynthesis

To gain more insight into the biosynthesis of the anthraquinone half of Dyn, we looked for genes common to the anthraquinone-fused enediynes (AFEs), uncialamycin (Ucm), tiancimycin (Tnm) and yangpumicin (Ypm). Among other genes targeted for genetic manipulation that are conserved across the AFEs, Orf15 particularly turned out to be interesting and useful for our purposes. Orf15, also known as “DynF,” is a protein of unknown function with highly conserved homologues in Ucm (ucmF, 78% ID), Tnm (tnmF, 80% ID), Ypm (ypmF, 92% ID).

The amino acid sequence for Orf15 (NCBI protein ID: ACB47058.1) is:

(SEQ ID NO: 25)
MSTKSVLFGRPVQTEGVPNVYAGAPVVPWTPPEPGIDNLGINSIDTFAVPGVGEYTV
AFDGWVRVVRSPSTSGEWADAEVYTNLIEMKMVGECEELGKITVTLNPDCLSAGQI
RTPFDPYAGEGPSAKACRMAVGAIFDMPKLGLKLMNREPIILTIDDVRSIPPAGAPG
KGQIYRMMPLLDVNDPDGQPVAYLTSLRFNMGGYLKPDQM*

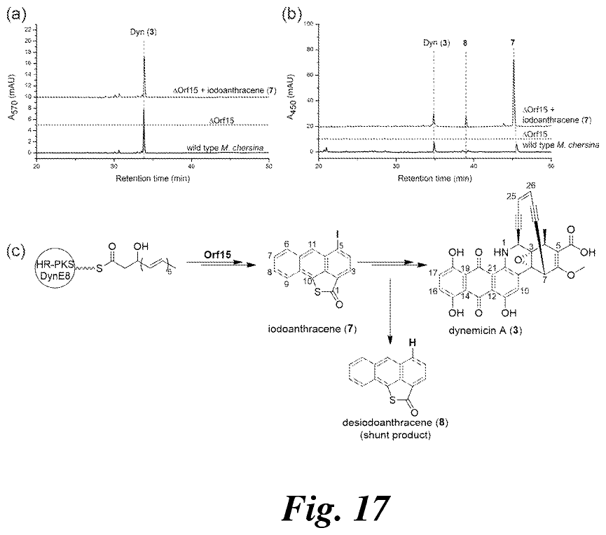

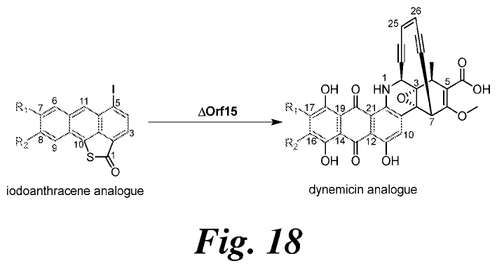

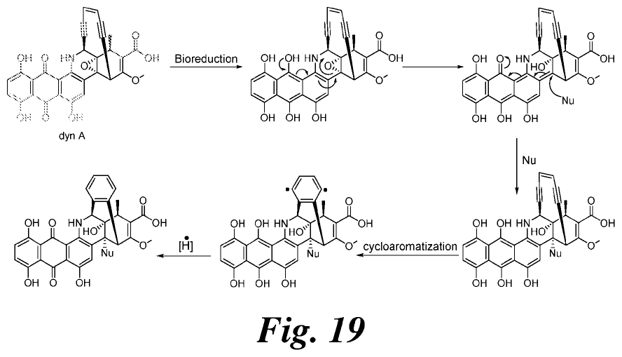

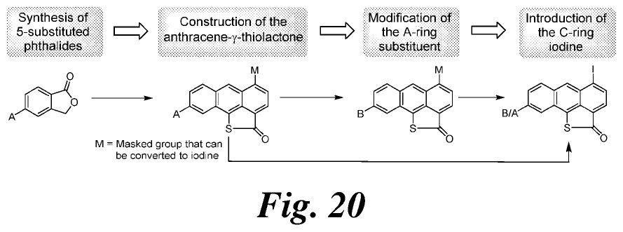

Deletion of Orf15 from the M. chersina wild type strain using CRISPR-Cas 9 abolished production of both Dyn 3 and the on-pathway intermediate iodoanthracene 7 without significant production of any new metabolites. Interestingly, when the iodoanthracene 7 is added to the fermentation medium of ΔOrf15, Dyn production resumed (FIG. 17), indicating that Orf15 may be involved in the early steps of iodoanthracene 7 biosynthesis, and the unstable nature of putative early intermediates made it impossible to detect their presence in the ΔOrf15 fermentation. This observation, however, presented us with the opportunity to incorporate modified iodoanthracenes into the ΔOrf15 fermentations to possibly generate Dyn analogues.

### 2.2.2 Design Strategy for the Iodoanthracene Analogues

With the goal to explore a combinatorial biosynthesis strategy, and considering the fact that the iodoanthracene finally ends up as the hydroxylated anthraquinone moiety in Dyn, we were left with C3 (C-ring), C8 and C9 (A-ring) to be modified by the incorporation of other substituents. These modified iodoanthracenes, if they are accepted as an on-pathway intermediate by the biosynthetic machinery, will produce C10 (C-ring), C16 and C17 (A-ring) modified Dyn analogues. However, if in addition the cytochrome P450 enzymes Orf19 and E10, which specifically hydroxylate C6 and C9, respectively, (analogous to C18 and C15 positions of Dyn), were removed (as has already been done), Cohen and Townsend, 2018a, any position(s) 6-9 could in principle be substituted. After further consideration, we disregarded modifying C3 of iodoanthracene, as this position is too close to the centers of major chemical transformations that take place to couple the two “halves” of Dyn and subsequent steps in the biosynthesis and could have adverse electronic or steric effects on the successful generation of the Dyn analogues.

In applying this strategy, we had three main goals in mind; first, introducing substituents having varying electronic effects on the A-ring of Dyn, we will be able to tune the activity of the analogues. Mode of action of Dyn is initiated by the bioreduction of the anthraquinone core to anthraquinol which leads to epoxide opening. Sugiura et al., 1990; Langley et al., 1991. The conformational change and strain relief introduced by this process facilitates the cycloaromatization of the enediyne with the generation of reactive benzenoid diradicals for DNA cleavage. Therefore, introducing proper substituents on the A-ring, it will be possible to tune the redox potential of the anthraquinone moiety, that will eventually translate into the activity of the molecule itself. Secondly, although the anthraquinone core can intercalate into DNA, Langley et al., 1991; Sugiura et al., 1990, mechanistic experiments performed by Myers et al. have demonstrated that substituents that favor strong intercalative binding, lead to diminished DNA cleaving activity. Myers et al., 1995; Myers et al., 1997. Hence, they proposed that bioreduction and hydrogen atom abstraction from DNA happens in a transient edge-wise minor-groove inserted state. Tuttle et al., 2007a; Tuttle et al., 2007b. In this conformation, the N-substituted side of the anthraquinone (colored in blue) faces towards the rim, and the other side (colored in red) points toward the floor of the minor groove (FIG. 19).

Therefore, by introducing substituents on the A-ring, it is possible to alter the equilibrium between the intercalated state and the triggerable minor-groove inserted state of the molecule to favor higher DNA cleaving activity. Finally, the substituents can be used as a handle to attach the mABs to develop novel ADCs for potential clinical use. Because of commercial availability or ease of synthesis of the starting materials, we first focused on synthesizing C9-modified iodoanthracene analogues.

### 2.2.3 Synthesis of the C9-Substituted Iodoanthracenes

Anthracenes can be synthesized following two major strategies: (a) starting from naphthalene and building the C-ring as dihydroanthracene, which upon aromatization leads to anthracene; Colver and Noyes, 1921; or (b) starting with two separate benzene rings and constructing the middle B-ring as an anthraquinone, which will lead to anthracene upon reduction. Finar, 1959. Due to the location of the substituents on our desired molecule, we chose the second strategy, which would provide greater flexibility to derivatize a benzene ring over a naphthalene required in the first strategy. Moreover, the carbonyl of the anthraquinone can be repurposed to install the sulfur regiospecifically on the B-ring in subsequent steps.

Our streamlined synthesis of the iodoanthracene analogues can be broken down into four steps: (1) synthesis of the 5-substituted phthalide; (2) construction of the anthraquinone, subsequent reduction and derivatization to generate the γ-thiolactone fused anthracene; (3) modification of the initial A-ring substituent to other possible functional groups; and (4) introduction of the C-ring iodine.

Synthesis of the 5-substituted phthalides that are not commercially available was readily achieved starting from 4-substituted benzoic acid 9 and converting it to a diisopropylbenzamide 10. Bisht et al., 2018. Diisopropylbenzamides are well known to direct ortho-lithiation upon reaction with alkyl-lithium reagents by virtue of their ability to coordinate with the metal ion. Therefore, treatment of the benzamide with n-BuLi and quenching the reaction with DMF introduces the aldehyde ortho to the amide, which results owing to the symmetry of the 4-substituted amides in a single product 11. Subsequent reduction of the aldehyde to alcohol and acid-catalyzed lactonization produces the desired 5-substituted phthalides 12. Faigl et al., 2010.

Construction of the anthraquinone moiety was achieved by reacting the phthalide 12 with 2-bromoanisole 13 in the presence of LDA, which generates the phthalide anion as well as benzyne intermediate from the 2-bromoanisole. The reaction of the phthalide anion with the benzyne resulted in a single regioisomer of the anthraquinone 14 by virtue of the directing effect of the methoxyl group. Khanapure et al., 1987. Instead of reducing the anthraquinone entirely to the anthracene, we took advantage of the peri-effect of the 1-methoxyl group to partially reduce the anthraquinone to anthrone 15 by selectively reducing only the carbonyl peri-to the methoxyl. Prinz et al., 1996. Treatment of the anthrone 15 with Lawesson's reagent generated the oxidatively unstable B-ring thiol 16 after tautomerization of the initially formed thioketone. Rogers et al., 2015. The thiol, upon reacting with triphosgene and subsequent AlCl₃-mediated Friedel-Crafts cyclization, created the γ-thiolactone 17, guided by the activating effect of the C-ring methoxyl group. Mitsudo et al., 2016. The iodine was introduced as the final modification by converting the methoxyl to triflate 18, followed by reaction of the aryl triflate with NaI in presence of catalytic Ru[Cp*(MeCN)₃]OTf. Imazaki et al., 2012.

### 2.2.4 Production of a Fluoro-Analogue of Dyn

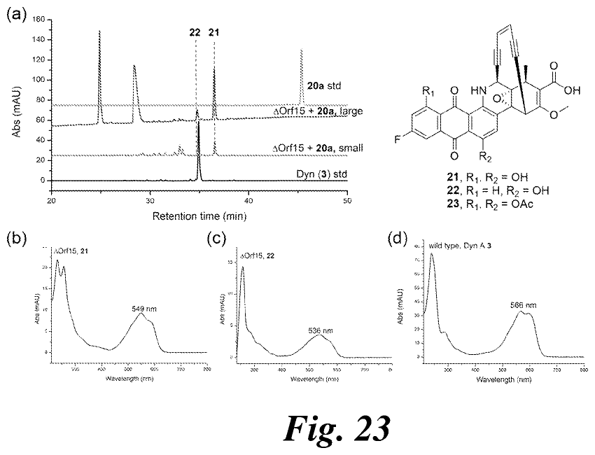

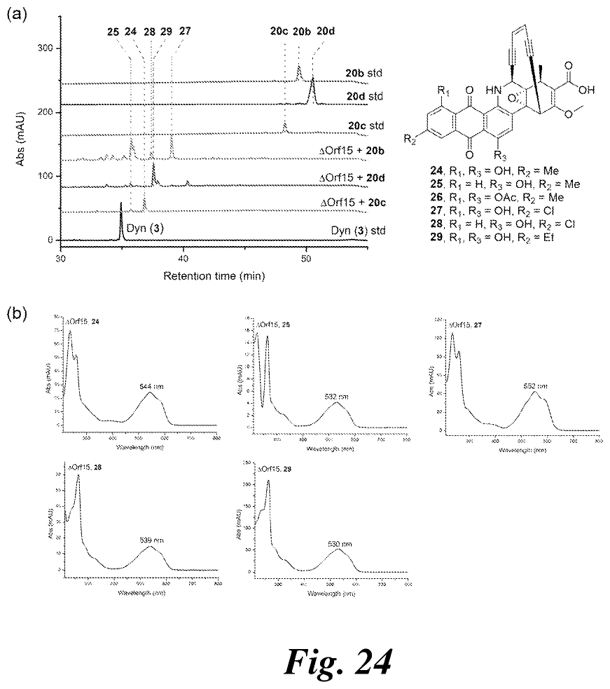

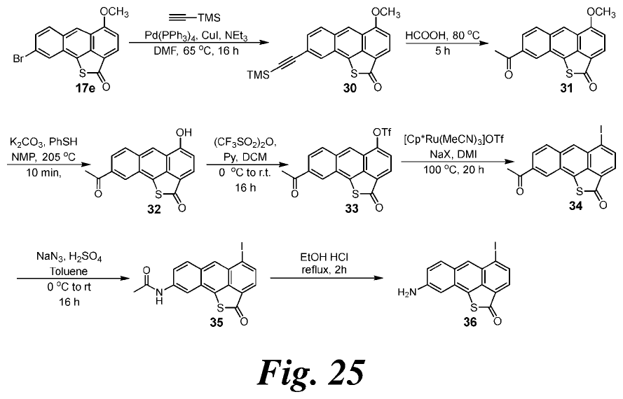

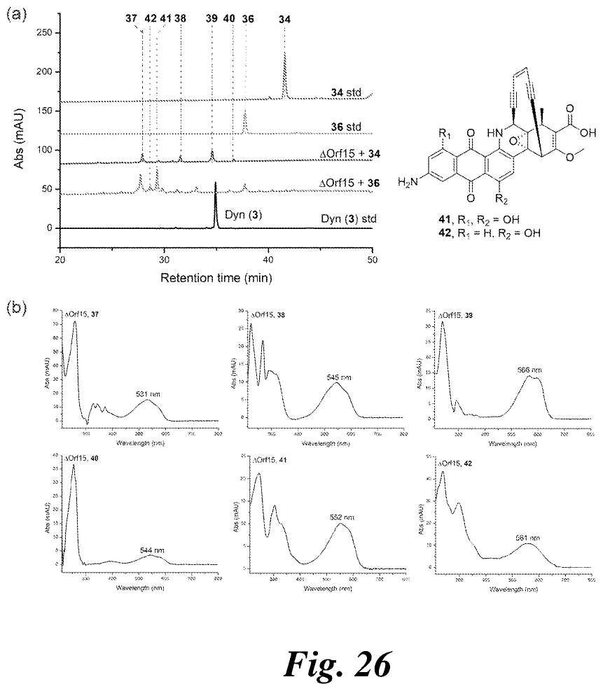

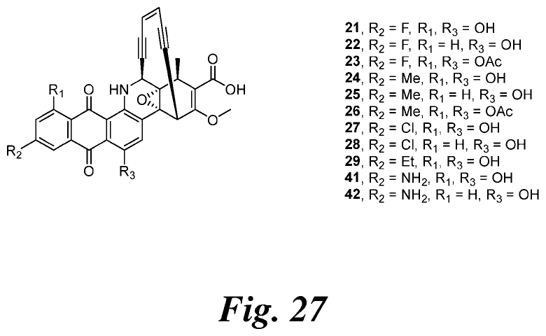

Without any detailed knowledge about the functions and mechanisms of any proteins involved in the biosynthesis or prior information about the steric bulk of the substituents that could be tolerated in the entire process, we first attempted the smallest possible substituent, fluorine. Synthesis of the 5-fluoro phthalide 12a was achieved starting from 4-fluorobenzoic acid 9a, following the general strategy described above. Due to the inert nature of the fluorine to all the reactions involved in the synthesis of the 9-substituted iodoanthracene, 9-fluoro-5-iodoanthracene-γ-thiolactone 20a was obtained in good overall yield. To our delight, incorporation of 20a into the ΔOrf15 strain produced one major and one minor product, 21 and 22, respectively, both with different HPLC-retention times but with similar UV-Vis spectra compared to Dyn (enediynes can be easily detected by their characteristic UV-Vis spectra with maxima in the 500-600 nm range) (FIG. 23). The major product 21 was consistent with the mass of the desired F-analogue of Dyn, with only two hydroxylations, and the minor 22 was similar with only one hydroxylation (compared to Dyn A, which has a total three hydroxylations). To characterize the analogues, 20a was incorporated in 12×1 L fermentations of ΔOrf15, and the products were isolated and acetylated following the standard Dyn purification protocol. Cohen and Townsend, 2018a. Whereas the major dihydroxylated product was successfully purified and characterized as the diacetylated derivative 23 by NMR spectroscopy, isolation of the minor monohydroxylated product was not successful due to very low production and was only detected by LCMS.

Both the ¹H and ¹³C NMR spectra of 23 were very similar to triacetylated Dyn, Cohen and Townsend, 2018a, with all characteristic hydrogens in both compounds almost overlapping with each other, except the aryl hydrogens, as expected. The singlet at δ=8.02 ppm in 23 is consistence with the C-ring hydrogen of triacetyldynemicin at δ=7.99 ppm, confirming the presence of C-ring hydroxylation in 23. The d×d signal at 7.62 (J=9.2, 8.8 Hz) corresponding to the two ortho-hydrogens on the A-ring of triacetylated Dyn is missing from the NMR of 23. Instead, two sets of d×d at δ=7.77 ppm (J=8.5, 2.7 Hz) and 7.64 ppm (J=8.8, 2.8 Hz), each corresponding to single hydrogen, indicated a different substitution pattern on the A-ring of 23 compared to triacetyldynemicin. With the prior knowledge that C16 is substituted with F, this splitting pattern with equal J values (equal J coupling of both hydrogens by the F-atom) places the second hydroxyl group at C18 instead of C15. Therefore, we can conclude the final structure of 21 and 23 as shown in FIG. 23. The structure of monohydroxylated 22 is predicted based upon the information that the A-ring hydroxylations are very late-stage phenomenon catalyzed by two separate P450s DynE10 and Orf19, Cohen and Townsend, 2018a, and the only reactions that can be affected by the strong electron withdrawal of the substituent on the A-ring.

### 2.2.5 Production of Methyl-, Chloro- and Ethyl-Analogues of Dyn

Encouraged by the success of our initial attempt, we wanted to test the tolerance of the system for increased steric bulk of the A-ring substituent. For this purpose, we synthesized 9-chloro and 9-methyl-5-iodoanthracene-γ-thiolactone 20b and 20c starting from 4-chloro and 4-methyl benzoic acids 9b and 9c, respectively, following the general streamlined synthesis. Supplementation with 20b and 20c to ΔOrf15 fermentations resulted in similar observations, where dihydroxylated Dyn analogues 24 and 27, respectively, were the major products with minor production of monohydroxylated Dyn analogues 25 and 28, respectively. The major dihydroxylated methyl analogue of Dyn 24 was isolated and purified as its diacetylated derivative 26 from 12 L of fermentation, following the same procedure as above, and characterized using NMR spectroscopy.

¹H NMR spectrum of 26 was very similar to that of both 23 and triacetyldynemicin 3a, except for the A-ring hydrogens as anticipated. The two hydrogens on the A-ring of 26 are present as two separate d×d at δ=7.85 ppm (J=1.7, 0.8 Hz), and 7.44 ppm (J=1.7, 0.8 Hz), resembling more the splitting pattern seen for 23. The small (J=1.7 Hz) coupling constant between the two A-ring hydrogens is consistent with meta coupling; thus, we concluded that the structure of 24 and 26 are as shown in FIG. 24. The structure of monohydroxylated analogue 25 was assigned following the same logic as the C16-fluoro analogue 22 above. Large-scale fermentation of the chloro analogue is ongoing for NMR characterization of the final metabolites.

Next, we attempted to increase the steric bulk from methyl or chloro to an ethyl substituent. Following the same synthetic procedure starting from 4-ethyl benzoic acid 9d, 9-methyl-5-iodoanthracene-γ-thiolactone 20d was synthesized. Incorporation of 20d in the ΔOrf15 fermentation also successfully produced the dihydroxylated ethyl-analogue of Dyn 29 as the major product.

2.2.6 Diversification of the Synthetic Route to Access to More 9-Substituted Iodoanthracenes from a Common Synthetic Intermediate

With the goal to generate an analogue library, it is always desirable to have a divergent synthesis rather than a linear, albeit streamlined one, where a substituent on a common advanced intermediate can be converted to various other functional groups to efficiently expand the library. It would be ideal to have a functional group that can tolerate the reactions involved in the general synthesis, as well as give access to modified groups. Amines are often the preferred substituent for this purpose, but it's high reactivity also interferes with many other reactions. Our initial attempt to synthesize the protected 6-amino-1-methoxyanthraquinone starting from protected 5-aminophthalide resulted in a very low yield of the desired product. On the other hand, commercially available 5-bromophthalide 12e was more successful. Synthesis of the 6-bromo-1-methoxyanthraquinone 17e was achieved is good yield from 5-bromo phthalide 12e and 2-bromoanisole 13. Reduction of the anthraquinone 14e to anthrone 15e required little optimization owing to dehalogenation in presence of higher equivalents of reductant or prolonged reaction time. Nevertheless, 9-bromo-5-methoxyanthracene was synthesized in good overall yield. Compound 17e could not be converted to the final 9-bromo-5-iodoanthracene-γ-thiolactone because of the lack of chemical discrimination between bromine and the O-triflate group in the last step of Ru catalyzed iodination. This difficulty was circumvented as follows.

After construction of the basic anthracene-γ-thiolactone skeleton, we attempted to convert the bromine to other functional groups. Reaction of 17e with alkyl lithium reagents in an effort to perform lithium halogen exchange followed by quenching with various electrophiles to access carbonyl or alkyl functionality was not fruitful; with n-BuLi, the initially formed aryl anion reacted with the butyl bromide formed in the reaction to install butyl at C9 of the anthracene, and t-BuLi mostly generated polymerized products. The bromine was successfully substituted to alkyne 30 in a Pd(PPh₃)₄ and CuI catalyzed reaction with TMS-acetylene. Wang et al., 2014. Although, the alkyne was not a suitable functional group for the final Ru catalyzed iodination reaction, due to higher affinity of the Ru for the alkyne than insertion into the C—X bond. The alkyne was eventually hydrated by refluxing in formic acid to the methyl ketone 31. Xu et al., 2018. The O-methoxyl of 31 was eventually converted to iodine to generate the iodoanthracene-γ-thiolactone 34 with a methyl ketone substituent at C9.

Administration of 34 to the ΔOrf15 fermentation did not produce the desired result. Instead, a multitude of very small peaks (37, 38, 39, 40) was observed by HPLC. Analysis of the peaks by LCMS confirmed no production of a Dyn analogue. The masses of the peaks and the UV-Vis spectra (with absorption maxima in the 500-600 nm range) were indicative of probable dimerized products (i.e., after the coupling of the anthracene and the enediyne half). Due to very small production of the metabolites, isolation and purification to determine their structures was not possible. Nevertheless, this result shed some lights upon the tolerance of the biosynthetic machinery to steric bulk of the C9 substituent.

With the indication that any substituent larger than two carbons probably cannot be tolerated for combinatorial biosynthesis, we focused on repurposing the methyl ketone to generate other smaller substituents. Schmidt reaction, Wang and Dong, 2018, of the methyl ketone of 34 in presence of NaN₃ and catalytic H₂SO₄ resulted in nitrogen insertion between the carbonyl and the aromatic ring, producing the N-acetylated amine 35. Removal of the acetyl protecting group by refluxing in EtOH-HCl provided the 9-amino-5-iodoanthracene-γ-thiolactone 36. Incorporation of 36 to a ΔOrf15 fermentation was again successful, as hoped, producing dihydroxylated 16-amino-Dyn analogue 41 as the major product, accompanied by the small production of the mono-hydroxylated analogue 42. Characterization of the final analogues is under way.

With the five examples in hand, there is no correlation that can be deduced between the yield of the final metabolite and the electronic nature of the substituents. Despite opposing electronic properties among fluoro-, chloro-, and amino-groups, Dyn analogues containing all three of these functional groups have all shown low production in small (50 mL), seven-day fermentations. On the other hand, the yields for methyl- and ethyl-substituted analogues are much higher for the same scale fermentation and time frame. Interestingly, comparing the small (50 mL) seven-day fermentation of the fluoro-analogue to the large (1 L) at ten days (FIG. 23), it was evident that the rates of production for all the analogues are not the same. The production of the fluoro-analogue after ten days can increase to be comparable to that of the methyl- and ethyl-analogues production after only seven days. However, the factors that affect the small vs large cultures are not known, but possibly the higher aeration in large culture acts in favor of better cell growth and higher metabolite production.

### 2.3 Summary

In summary, we can successfully generate analogues of Dyn by applying our combinatorial biosynthetic method. This approach has several advantages over total synthesis requiring, by comparison, only the shorter and more straightforward synthesis of simpler, achiral anthracene molecules to reach the same structurally complex and enantiopure final Dyn derivative. With an efficient, streamlined procedure to build the anthracene core starting from two separate benzene building blocks, installation of the thiolactone ring and introduction of the iodine as common steps, structural differentiation reduces ultimately to synthesizing differently substituted phthalides to generate all the different anthracene analogues. Later stage diversification of the synthesis also provides flexibility to introduce different functional groups efficiently towards the end of the synthesis. As exemplified, the alkyne can be directed to generate a carboxylic acid or aldehyde.

No direct connection between the electronic nature of the substituents and the final yields of the metabolites eliminates any apparent restrictions on the choice of substituents apart from steric bulk. This observation can be rationalized by the distance of the substituents (on the A-ring) from the epicenter of the chemical transformations (mainly the C-ring). Nevertheless, the substituent at C16 of the Dyn analogues definitely has an influence on the hydroxylation at C15, the observation of dihydroxy-Dyn analogues as the major products (missing the hydroxylation at C15 present in Dyn) are attributed solely to steric hindrance of the cytochrome P450 E10. Cohen and Townsend, 2018a.

Although, the monohydroxylated analogues could not be isolated and characterized owing to their low production, these metabolites could be generated, if desired, as the major product by removal of the encoding genes. With the already established fact that the two P450 enzymes, Orf19 and E10 are responsible for hydroxylation of C18 and C15, respectively, Cohen and Townsend, 2018a, it is possible to use the double deletion mutant ΔOrf19ΔOrf15 to further eliminate the C18 hydroxylation in these analogues, thus extending the analogue library in a programmable manner.

Orf15 is a highly conserved gene among all the AFEs, and from the results presented here, it can be stated with certainty that Orf15 is involved in the biosynthesis of the iodoanthracene-γ-thiolactone 7. Combining other work from the Townsend lab and recent reports from Liang et al., it is quite certain that 7 is a common intermediate for all AFE biosynthetic pathways where it serves as the precursor for the anthraquinone core of these natural products. It is differently hydroxylated in late-stage modifications by cytochrome P450 hydroxylases present in the individual BGCs. Finally, the amine-containing analogue 36 can be utilized to develop ADCs by attaching mABs to the amine as a handle.

### 2.4 Methods

### 2.4.1 General Information

General information is provided in section 1.5.5.

### 2.4.2 General Synthetic Procedures

2.4.2.1 General Procedure for the Synthesis of Diisopropyl Amides from Carboxylic Acids. Faigl et al., 2010.

In a flame-dried round-bottomed flask, benzoic acid (1 eq) was dissolved in dry DCM and cooled to 0° C. in an ice-water bath. To this solution catalytic DMF (0.1 eq) was added followed by the dropwise addition of oxalyl chloride (1.3 eq). After stirring at the same temperature for 15 min, the ice-bath was removed and the reaction mixture was stirred at room temperature for 4 h. After removal of the solvent in vacuo, the crude acid chloride was redissolved in minimum amount of dry DCM.

In a separate flame-dried round-bottomed flask, dry DCM was cooled to 0° C. in an ice-bath. To this solution, DIPA (1.3 eq) and TEA (1.5 eq) were added sequentially and stirred at the same temperature for 15 min. The crude acid chloride solution was added to this reaction mixture dropwise at 0° C. and stirred for 12 h at room temperature. After completion, the reaction was quenched with water and the mixture was extracted with DCM. The combined organic layer was washed once with sat. NaHCO₃ solution, followed by brine, dried over anhydrous Na₂SO₄ and the solvent was removed in vacuo. The crude compound was purified by silica column chromatography using 20-30% EtOAc in hexanes to give the pure amide.

2.4.2.2 General Procedure for Ortho-Formylation of the Amides. Faigl et al., 2010.

In a flame-dried round-bottomed flask the amide (1 eq) was dissolved in dry THF and cooled to −78° C. using a dry ice-acetone bath. To this solution TMEDA (2 eq) was added followed by slow dropwise addition of n-BuLi (1.2 eq). The reaction mixture was stirred at the same temperature for 1 h. At this time N,N-dimethylformamide (1.4 eq) was added, and the reaction mixture was stirred for another hour while allowing it to warm to room temperature. After completion, the reaction was quenched with sat. NH₄Cl solution and extracted with EtOAc. The combined organic layer was washed with brine, dried over anhydrous Na₂SO₄ and the solvent was removed in vacuo. The crude product was purified by silica column chromatography using 20-30% EtOAc in hexanes to yield the pure aldehyde.

2.4.2.3 General Procedure for the Synthesis of the 5-Substituted Phthalides. Faigl et al., 2010.

The aldehyde (1 eq) was dissolved in MeOH and cooled to 0° C. in an ice-water bath. To this solution NaBH₄ (1.3 eq) was added very slowly to avoid any vigorous effervescence. After stirring at that temperature for 15 min, the ice-bath was removed and the reaction mixture was allowed to warm to room temperature and stirred for another 2 h. After consumption of all starting material, the reaction was quenched by adding an equal volume of water and the MeOH was removed in vacuo. To the remaining aqueous solution, 15 mL conc. HCl was added and refluxed for 6-8 h. After completion, the reaction was cooled to room temperature and extracted with EtOAc. The combined organic layer was washed with brine, dried over anhydrous Na₂SO₄ and the solvent was removed in vacuo. The crude product was purified by silica column chromatography using 50-70% EtOAc in hexanes to afford the pure phthalide.

2.4.2.4 General Procedure for the Synthesis of the 1-Methoxyanthraquinones. Khanapure et al., 1987; Townsend et al., 1981.

In a flame-dried round-bottomed flask, N,N-diisopropylamine (3.5 eq) was dissolved in dry THF and cooled to −78° C. in a dry ice-acetone bath. To this solution n-BuLi (3.3 eq) was added dropwise and stirred at −78° C. for 20 min. Phthalide (1 eq) was added to the reaction mixture at the same temperature, followed by immediate addition of HMPA (3.5 eq). After stirring at −78° C. for another 10 min, the reaction mixture was warmed to −40° C. using a dry ice-ACN bath and 2-bromoanisole (13, 2.2 eq) was added. The reaction mixture was allowed to warm up to room temperature over 16 h and stirred open to the air for at least another 6 h. After completion, the reaction was quenched with sat. NH₄Cl solution and extracted with EtOAc. The organic layer was washed twice with a large volume of water, dried over anhydrous Na₂SO₄ and the solvent was removed in vacuo. The crude compound was purified by silica column chromatography using 50-70% EtOAc in hexanes to give the anthraquinone.

2.4.2.5 General Procedure for Reduction of 1-Methoxyanthraquinones to Anthrones. Prinz et al., 1996; Rogers et al., 2015.

1-Methoxyanthraquinone (1 eq) was dissolved in 1M aqueous NaOH solution to a final concentration of 0.15 M. To this solution Na₂S₂O₄ (1 g/mL of solvent) was added and the reaction mixture was heated to reflux until complete consumption of the starting material was observed. After cooling the reaction to room temperature, the precipitate was filtered and washed with 50 mL of 1M NaOH, followed by H₂O until the filtrate became neutral. The precipitate was dried under vacuum. The product was further purified on silica gel with 5-10% EtOAc in hexanes to yield the pure anthrone.

2.4.2.6 General Procedure for Thionation of the Anthrones. Rogers et al., 2015.

In a flame-dried round-bottomed flask the anthrone (1 eq) was dissolved in dry toluene and degassed by bubbling Ar though the solution for 15 min. To this solution Lawesson's reagent (0.6 eq) was added and heated to reflux for 2 h. After completion of the reaction, the solution was cooled to room temperature and filtered through a small silica plug washing using 80% DCM in hexanes until the filtrate became colorless. The filtrate was dried in vacuo to obtain the crude thiol as a yellow oil. No further purification was possible due to very fast oxidation of the thiol to the disulfide.

2.4.2.7 General Procedure for Thiolactonization. Mitsudo et al., 2016.

The crude 4-methoxyanthracene-9-thiol (1 eq) was dissolved in dry DCM and cooled to 0° C. in an ice-water bath. To this solution triphosgene (3 eq) and pyridine (4 eq) were added and stirred at 0° C. for 3 h. After completion, the reaction was quenched by the very slow addition of ice-cold water, extracted with DCM and dried in vacuo. The metastable carbonochloridothioate intermediate was dissolved in dry DCM and cooled to 0° C. using an ice-water bath. To this solution AlCl₃ (1.5 eq) was added and stirred for 1 h, while allowing it to warn to room temperature. After completion of the reaction, the DCM was removed in vacuo, the crude product was dissolved in EtOAc and washed twice with 1N HCl. The organic layer was dried over anhydrous Na₂SO₄ and the solvent was removed in vacuo. The product was purified by silica gel chromatography eluting with 20% EtOAc in hexanes to afford pure 5-methoxyanthracene-γ-thiolactone.

2.4.2.8 General Procedure for Demethylation. Chakraborti et al., 2002.

In a flame-dried round-bottomed flask, 5-methoxyanthracene-γ-thiolactone (1 eq) was dissolved in a small volume of dry N-methyl-2-pyrrolidone (NMP). To this mixture K₂CO₃ (0.05 eq) and thiophenol (1 eq) were added and the reaction mixture was heated in a pre-heated oil bath to 205° C. for 10 min. After cooling to room temperature, the reaction mixture was acidified with 1N HCl and extracted with EtOAc. The organic layer was washed once with 1N HCl and twice with a large volume of H₂O, dried over anhydrous Na₂SO₄ and the solvent was removed in vacuo. The product was purified by silica gel column using 20% EtOAc in hexanes with 1% acetic acid to give the free 5-hydroxyanthracene-γ-thiolactone

### 2.4.2.9 General Procedure for Triflate Formation

In a flame-dried round-bottomed flask, 5-hydroxyanthracene-γ-thiolactone (1 eq) was dissolved in 10 mL dry DCM and cooled to 0° C. To this solution triflic anhydride (1.5 eq) and pyridine (1.5 eq) were added in succession and allowed to stir for 1 h at room temperature. After completion of the reaction, DCM was removed in vacuo. The crude mixture was dissolved in EtOAc and washed with H₂O. After drying in vacuo, the remaining pyridine was removed by azeotropic distillation with toluene. The product was purified by silica gel column chromatography with 10% EtOAc in hexanes to yield the corresponding triflate.

2.4.2.10 General Procedure for Iodination. Imazaki et al., 2012.

In a flame-dried round-bottomed flask the triflate (1 eq) was dissolved in a small volume of dry 1,3-dimethyl-2-imidazolidinone (DMI). To this [Cp*Ru(MeCN)₃]OTf (0.05 eq) and NaI (1.5 eq) were added and the mixture was heated at 100° C. for 20 h. After cooling to room temperature, the reaction mixture was diluted with EtOAc, filtered through Celite and the solvent was removed in vacuo. The crude product was purified by silica gel column chromatography with 10% EtOAc in hexanes to give the desired iodides. For in vitro feeding experiments, the compound was further purified by preparative HPLC (Phenomenex Luna 10 m C18(2) 100 Å preparatory column, 250×21.20 mm ID) using water and acetonitrile with 0.1% TFA as mobile phase.

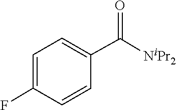

4-Fluoro-N,N-diisopropylbenzamide (10a). This compound was synthesized from 4-fluorobenzoic acid (9a) using the general procedure for the synthesis of diisopropyl amides from carboxylic acids described above in 88% yield as a white solid. ¹H NMR (400 MHz CDCl₃) δ ppm 6.97-6.93 (m, 2H), 6.67 (t, J=8.7 Hz, 2H), 3.30 (s, br, 2H), 0.96 (s, br, 12H); ¹³C NMR (100 MHz CDCl₃) δ ppm 169.4 (s), 162.3 (d, J=246.2 Hz), 134.9 (d, J=3.6 Hz), 127.4 (d, J=8.2 Hz), 115.0 (d, J=21.5 Hz), 20.2 (s); UPLC-ESI-HMS: calculated exact mass for C₃H₁₉FNO⁺ [M+H⁺]: 224.1445, found [M+H⁺]: 224.1461.

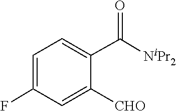

4-Fluoro-2-formyl-N,N-diisopropylbenzamide (11a). This compound was synthesized from 10a using the general procedure for ortho-formylation of the amides discussed above in 95% yield as a white solid. ¹H NMR (400 MHz CDCl₃) δ ppm 9.80 (d, J=2.2 Hz, 1H), 7.35 (dt, J=8.4, 1.4 Hz, 1H), 7.10 (dd, J=6.6, 1.4 Hz, 2H), 3.36 (octet, J=6.6 Hz, 2H), 1.33 (d, J=6.8 Hz. 6H), 0.87 (d, J=6.7 Hx, 6H); ¹³C NMR (100 MHz CDCl₃) δ ppm 188.8 (d, J=1.52 Hz), 167.1 (s), 162.1 (d, J=248.7 Hz), 137.4 (d, J=3.6 Hz), 134.3 (d, J=6.1 Hz), 128.0 (d, J=7.5 Hz), 121.1 (d, J=22.0 Hz), 115.1 (d, J=22.1 Hz), 51.2 (s), 45.9 (s), 20.2; UPLC-ESI-HMS: calculated exact mass for C₁₄H₁₉FNO₂⁺ [M+H⁺]: 252.1394, found [M+H⁺]: 252.1402.

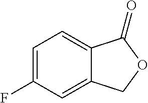 [SMILES: O=C1OCc2cc(F)ccc21]

5-Fluoroisobenzofuran-1(3H)-one (12a). This compound was synthesized starting from 11a following the above general procedure for the synthesis of the 5-substituted phthalides in 83% yield as a white solid. ¹H NMR (400 MHz CDCl₃) δ ppm 7.93 (dd, J=8.4, 4.8 Hz, 1H), 7.27-7.19 (m, 2H), 5.32 (s, 2H); ¹³C NMR (100 MHz CDCl₃) δ ppm 169.9 (s), 166.5 (d, J=254.1 Hz), 149.4 (d, J=10.6 Hz), 127.9 (d, J=10.4 Hz), 121.7 (d, J=1.9 Hz), 117.2 (d, J=24 Hz), 109.6 (d, J=24.4 Hz), 69.1 (d, J=2.9 Hz); UPLC-ESI-HMS: calculated exact mass for C₈H₆FO₂⁺ [M+H⁺]: 153.0346, found [M+H⁺]: 153.0349.

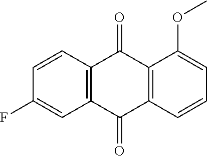 [SMILES: COc1cccc2c1C(=O)c1ccc(F)cc1C2=O]

6-Fluoro-1-methoxyanthracene-9,10-dione (14a). This compound was synthesized from 12a and 13 using the general procedure for the synthesis of the 1-methoxy anthraquinones in 67% yield as a yellow solid. ¹H NMR (400 MHz CDCl₃) δ ppm 8.27 (dd, J=8.7, 5.3 Hz, 1H), 7.90 (d, J=7.7 Hz, 1H), 7.80 (dd, J=8.6, 2.7 Hz, 1H), 7.70 (t, J=8.0 Hz, 1H), 7.41 (td, J=8.2, 2.7 Hz, 1H), 7.34 (d, J=8.3 Hz, 1H), 4.03 (s, 3H); ¹³C NMR (100 MHz CDCl₃) δ ppm 182.4 (d, J=1.8 Hz), 181.1 (d, J=0.6 Hz), 165.7 (d, J=254.7 Hz), 160.5 (s), 135.5 (d, J=1.6 Hz), 135.1 (s), 134.9 (d, J=7.8 Hz), 131.5 (d, J=3.1 Hz), 130.6 (d, J=8.7 Hz), 121.5 (d, J=22.4 Hz), 121.1 (s), 119.9 (s), 118.3 (s), 112.8 (d, J=23.0 Hz), 56.6 (d, J=0.9 Hz); UPLC-ESI-HMS: calculated exact mass for C₁₅H₁₀FO₃⁺ [M+H⁺]: 257.0608, found [M+H⁺]: 257.0604.

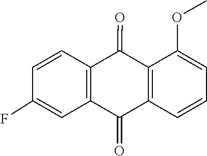 [SMILES: COc1cccc2c1C(=O)c1ccc(F)cc1C2=O]

2-Fluoro-5-methoxyanthracen-9(10H)-one (15a). This compound was synthesized from 14a following the general procedure for reduction of 1-methoxyanthraquinones to anthrones in 76% yield as a pale-yellow solid. ¹H NMR (400 MHz CDCl₃) δ ppm 7.85 (dd, J=9.3, 2.6 Hz, 1H), 7.80, (d, J=7.9 Hz, 1H), 7.31-7.25 (m, 2H), 7.16 (td, J=8.3, 2.7 Hz, 1H), 6.92 (d, J=8.2 Hz, 1H), 3.81 (s, 5H); ¹³C NMR (100 MHz CDCl₃) δ ppm 183.1 (d, J=2.2 Hz), 161.5 (d, J=244.6 Hz), 156.4 (s), 136.2 (d, J=2.8 Hz), 133.1 (d, J=6.5 Hz), 131.8 (d, J=0.9 Hz), 130.7 (d, J=7.2 Hz), 129.5 (s), 127.3 (s), 120.3 (d, J=22.5 Hz), 118.9 (s), 113.2 (s), 112.6 (d, J=21.8 Hz), 55.5 (s), 26.6 (s); UPLC-ESI-HMS: calculated exact mass for C₁₅H₁₂FO₂⁺ [M+H⁺]: 243.0816 found [M+H⁺]: 243.0618.

9-Fluoro-5-methoxy-2H-anthra[9,1-bc]thiophen-2-one (17a). This compound was

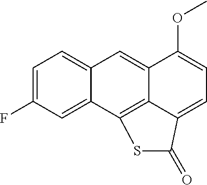 [SMILES: COc1ccc2c3c(c4cc(F)ccc4cc13)SC2=O]

synthesized from 15a following the general procedure for thionation of the anthrones and general procedure for thiolactonization in 49% yield over three steps as an orange solid. ¹H NMR (400 MHz CDCl₃) δ ppm 8.56 (s, 1H), 8.17 (d, J=7.8 Hz, 1H), 8.01 (dd, J=9.1, 5.8 Hz, 1H), 7.39 (d, J=9.5 Hz, 1H), 7.32-7.27 (m, 1H), 6.94 (d, J=7.8 Hz, 1H), 4.18 (s, 3H); ¹³C NMR (100 MHz CDCl₃) δ ppm 190.7 (s), 162.3 (d, J=10.8 Hz), 159.8 (s), 133.1 (d, J=9.4 Hz), 130.5 (s), 129.9 (s), 129.4 (s), 129.0 (d, J=7.6 Hz), 128.0 (d, J=9.8 Hz), 124.7 (s), 120.9 (d, J=2.2 Hz), 120.4 (d, J=1.9 Hz), 117.8 (d, J=27.2 Hz), 107.1 (d, J=22.0 Hz), 104.1 (s), 56.7; UPLC-ESI-HMS: calculated exact mass for C₁₆H₁₀FO₂S⁺ [M+H⁺]: 285.0380 found [M+H⁺]: 285.0375.

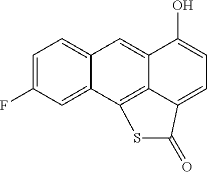 [SMILES: O=C1Sc2c3cc(F)ccc3cc3c(O)ccc1c23]

9-Fluoro-5-hydroxy-2H-anthra[9,1-bc]thiophen-2-one (18a). This compound was synthesized following the general procedure for demethylation from 17a in 87% yield as an orange solid. ¹H NMR (400 MHz DMSO-d₆) δ ppm 8.89 (s, 1H), 8.42 (dd, J=9.2, 5.9 Hz, 1H), 8.26 (d, J=7.9 Hz, 1H), 7.62 (dd, J=10.2, 2.2 Hz, 1H), 7.55 (td, J=8.8, 2.2 Hz, 1H), 7.18 (d, J=7.9 Hz, 1H); ¹³C NMR (100 MHz DMSO-d₆) δ ppm 189.4 (s), 163.3 (s), 161.4 (d, J=248.8 Hz), 134.7 (d, J=9.8 Hz), 131.8 (s), 131.6 (s), 129.9 (s), 128.1 (s), 128.0 (d, J=10.3 Hz), 122.0 (d, J=2.0 Hz), 121.7 (s), 120.8 (d, J=2.1 Hz), 118.0 (d, J=27.2 Hz), 109.8 (s), 107.0 (d, J=21.1 Hz); UPLC-ESI-HMS: calculated exact mass for C₁₅H₈FO₂S⁺ [M+H⁺]: 271.0224 found [M+H⁺]: 271.0225.

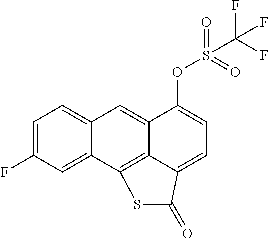 [SMILES: O=C1Sc2c3cc(F)ccc3cc3c(OS(=O)(=O)C(F)(F)F)ccc1c23]

9-Fluoro-2-oxo-2H-anthra[9,1-bc]thiophen-5-yl trifluoromethanesulfonate (19a). This compound was synthesized from 18a following the general procedure for triflate formation in 94% yield as an orange solid. ¹H NMR (400 MHz CDCl₃) δ ppm 8.54 (s, 1H), 8.27 (d, J=7.7 Hz, 1H), 8.19-8.15 (m, 1H), 7.74 (d, J=7.6 Hz, 1H), 7.49-7.44 (m, 2H); ¹³C NMR (100 MHz CDCl₃) δ ppm 189.9 (s), 161.6 (d, J=253.7 Hz), 149.7 (d, J=1.3 Hz), 133.2 (d, J=9.3 Hz), 132.3 (s), 131.6 (s), 130.8 (d, J=8.3 Hz), 129.8 (s), 128.4 (d, J=9.9 Hz), 126.2 (s), 121.7 (d, J=2.1 Hz), 119.9 (d, J=27.6 Hz), 119.3 (d, J=2.1 Hz), 118.5 (s), 117.2 (s), 107.5 (d, J=22.4 Hz); UPLC-ESI-HMS: calculated exact mass for C₁₆H₇F₄O₄S₂⁺ [M+H⁺]: 402.9716, found [M+H⁺]: 402.9715.

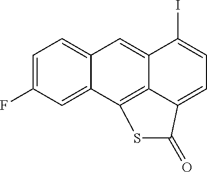 [SMILES: O=C1Sc2c3cc(F)ccc3cc3c(I)ccc1c23]

9-Fluoro-5-iodo-2H-anthra[9,1-bc]thiophen-2-one (20a). This compound was synthesized from 19a using the general procedure for iodination in 84% yield as an orange solid. ¹H NMR (600 MHz CDCl₃) δ ppm 8.47 (s, 1H), 8.35 (d, J=7.3 Hz, 1H), 8.17 (dd, J=9.3, 5.7 Hz, 1H), 7.91 (d, J=7.3 Hz, 1H), 7.47 (dd, J=9.5, 2.0 Hz, 1H), 7.43 (m, 1H); ¹³C NMR (150 MHz CDCl₃) δ ppm 191.4 (s), 161.3 (d, J=252.4 Hz), 137.9 (s), 133.1 (s), 133.1 (s), 131.7 (s), 130.6 (s), 129.4 (s), 128.6 (s), 128.1 (d, J=9.6 Hz), 126.7 (s), 119.2 (d, J=27.6 Hz), 108.3 (s), 107.3 (d, J=22.2 Hz); UPLC-ESI-HMS: calculated exact mass for C₁₅H₇FIOS⁺ [M+H⁺]: 380.9241, found [M+H⁺]: 380.9237.

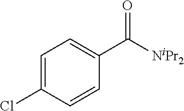

4-Chloro-N,N-diisopropylbenzamide (10b). This compound was synthesized from 4-chlorobenzoic acid (9b) using the general procedure for the synthesis of diisopropyl amides from carboxylic acids described above in 90% yield as a white solid. ¹H NMR (400 MHz CDCl₃) δ ppm 7.18-7.15 (m, 2H), 7.10-7.07 (m, 2H), 3.47 (s, br, 2H), 1.13 (s, br, 12H); ¹³C NMR (100 MHz CDCl₃) δ ppm 169.5, 137.2, 134.3, 128.5, 127.0, 127.0, 20.5; UPLC-ESI-HMS: calculated exact mass for C₁₃H₁₉ClNO⁺ [M+H⁺]: 240.1150, found [M+H⁺]: 240.1156.

4-Chloro-2-formyl-N,N-diisopropylbenzamide (11b). This compound was synthesized

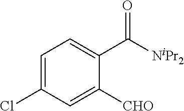

from 10b following the above general procedure for ortho-formylation of the amides discussed above in 91% yield as a white solid. ¹H NMR (400 MHz CDCl₃) δ ppm 9.81 (s, 1H), 7.66 (d, J=2.2 Hz, 1H), 7.37 (d, J=8.1, 2.2 Hz, 1H), 7.07 (d, J=8.1 Hz, 1H), 3.41-3.33 (m, 2H), 1.36 (d, J=6.8 Hz, 6H), 0.89 (d, J=6.7, 6H); ¹³C NMR (100 MHz CDCl₃) δ ppm 188.9, 167.1, 139.0, 134.7, 133.9, 133.6, 129.2, 127.4, 51.2, 46.0, 20.4, 20.2, 20.1; UPLC-ESI-HMS: calculated exact mass for C₁₄H₁₉ClNO₂⁺ [M+H⁺]: 268.1099, found [M+H⁺]: 268.1102.

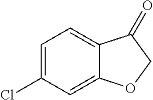 [SMILES: O=C1COc2cc(Cl)ccc21]

5-Chloroisobenzofuran-1(3H)-one (12b). This compound was synthesized starting from 11b following the above general procedure for the synthesis of the 5-substituted phthalides in 79% yield as a white solid. 1H NMR (400 MHz CDCl₃) δ ppm 7.83 (d, J=8.6 Hz, 1H), 7.51-7.49 (m, 2H), 5.30 (s, 2H); ¹³C NMR (100 MHz CDCl₃) δ ppm 169.9, 148.2, 140.8, 129.8, 126.9, 124.3, 122.6, 69.0; UPLC-ESI-HMS: calculated exact mass for C₈H₆ClO₂⁺ [M+H⁺]: 169.0051, found [M+H⁺]: 169.0056.

6-Chloro-1-methoxyanthracene-9,10-dione (14b). This compound was synthesized from

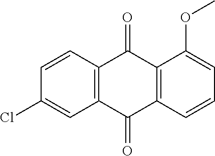 [SMILES: COc1cccc2c1C(=O)c1ccc(Cl)cc1C2=O]

12b and 13 following the above general procedure for the 1-methoxy anthraquinones in 62% yield as a yellow solid. ¹H NMR (400 MHz CDCl₃) δ ppm 8.17 (d, J=8.4 Hz, 1H), 8.13 (d, J=1.6 Hz, 1H), 7.90 (d, J=7.6 Hz, 1H), 7.73-7.68 (m, 2H), 7.34 (d, J=8.4 Hz, 1H), 4.04 (s, 3H); ¹³C NMR (100 MHz CDCl₃) δ ppm 182.3, 181.3, 160.5, 140.1, 135.4, 135.2, 134.2, 133.5, 133.1, 129.1, 126.3, 121.1, 119.9, 118.2, 56.6; UPLC-ESI-HMS: calculated exact mass for C₁₅H₁₀ClO₃⁺ [M+H⁺]: 273.0313, found [M+H⁺]: 271.0320.

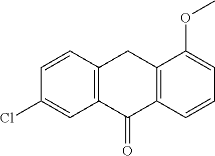 [SMILES: COc1cccc2c1Cc1ccc(Cl)cc1C2=O]

2-Chloro-5-methoxyanthracen-9(10H)-one (15b). This compound was synthesized from 14b following the general procedure for reduction of 1-methoxyanthraquinones to anthrones in 68% yield as a pale-yellow solid. ¹H NMR (400 MHz CDCl₃) δ ppm 8.15 (d, J=2.2 Hz, 1H), 7.80 (d, J=7.6 Hz, 1H), 7.38 (dd, J=8.2, 2.3 Hz, 1H), 7.31-7.24 (m, 2H), 6.95 (d, J=7.8 Hz, 1H), 3.84 (s, 5H); ¹³C NMR (100 MHz CDCl₃) δ ppm 182.9, 156.4, 138.7, 132.9, 132.7, 132.5, 132.0, 130.4, 129.3, 127.4, 126.8, 119.0, 113.3, 55.4, 26.7; UPLC-ESI-HMS: calculated exact mass for C₁₅H₁₂ClO₂⁺ [M+H⁺]: 259.0520, found [M+H⁺]: 259.0533.

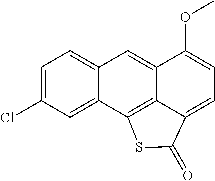 [SMILES: COc1ccc2c3c(c4cc(Cl)ccc4cc13)SC2=O]

9-Chloro-5-methoxy-2H-anthra[9,1-bc]thiophen-2-one (17b). This compound was synthesized from 15b following the general procedure for thionation of the anthrone and general procedure for thiolactonization in 44% yield over three steps as an orange solid. ¹H NMR (400 MHz CDCl₃) δ ppm 8.58 (s, 1H), 8.20 (d, J=7.9 Hz, 1H), 7.96 (d, J=9.1 Hz, 1H), 7.82-7.81 (m, 1H), 7.42 (dd, J=9.0, 2.0 Hz, 1H), 6.99 (d, J=7.9 Hz, 1H), 4.21 (s, 3H); ¹³C NMR (100 MHz CDCl₃) δ ppm 190.76, 162.16, 133.5, 131.8, 130.8, 130.7, 129.5, 129.2, 127.7, 127.3, 124.8, 123.2, 121.5, 120.3, 104.5, 56.7; UPLC-ESI-HMS: calculated exact mass for C₁₆H₁₀ClO₂S⁺ [M+H⁺]: 301.0085, found [M+H⁺]: 301.0081.

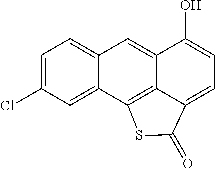 [SMILES: O=C1Sc2c3cc(Cl)ccc3cc3c(O)ccc1c23]

9-Chloro-5-hydroxy-2H-anthra[9,1-bc]thiophen-2-one (18b). This compound was synthesized following the general procedure for demethylation from 17b in 83% yield as an orange solid. ¹H NMR (400 MHz DMSO-d₆) δ ppm 8.86 (s, 1H), 8.35 (d, J=9.1 Hz, 1H), 8.26 (d, J=7.9 Hz, 1H), 7.94-7.93 (m, 1H), 7.59 (d, J=8.8 Hz, 1H), 7.19 (d, J=7.9 Hz, 1H); UPLC-ESI-HMS: calculated exact mass for C₁₅H₈ClO₂S⁺ [M+H⁺]: 286.9928, found [M+H⁺]: 286.9930.

9-Chloro-2-oxo-2H-anthra[9,1-bc]thiophen-5-yl trifluoromethanesulfonate (19b). This

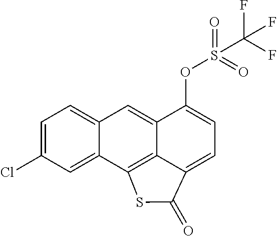 [SMILES: O=C1Sc2c3cc(Cl)ccc3cc3c(OS(=O)(=O)C(F)(F)F)ccc1c23]

compound was synthesized from 18b following the general procedure for triflate formation in 92% yield as an orange solid. ¹H NMR (600 MHz CDCl₃) δ ppm 8.56 (s, 1H), 8.30 (d, J=7.6 Hz, 1H), 8.13 (d, J=9.1 Hz, 1H), 7.94 (dd, J=1.7, 0.8 Hz, 1H), 7.78 (d, J=7.6 Hz, 1H), 7.61 (dd, J=9.1, 1.9 Hz, 1H); ¹³C NMR (150 MHz CDCl₃) δ ppm 190.0, 149.7, 134.9, 132.4, 131.8, 130.9, 130.1, 129.2, 128.1, 126.3, 123.6, 122.3, 119.8, 119.1, 118.9, 117.7; UPLC-ESI-HMS: calculated exact mass for C₁₆H₇ClF₃O₄S₂⁺ [M+H⁺]: 418.9421, found [M+H⁺]: 418.9424.

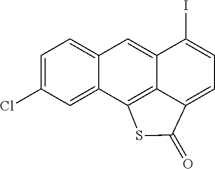 [SMILES: O=C1Sc2c3cc(Cl)ccc3cc3c(I)ccc1c23]

9-Chloro-5-iodo-2H-anthra[9,1-bc]thiophen-2-one (20b). This compound was synthesized from 19b using the general procedure for iodination in 78% yield as an orange solid. ¹H NMR (600 MHz CDCl₃) δ ppm 8.49 (s, 1H), 8.39 (d, J=7.1 Hz, 1H), 8.13 (d, J=8.9 Hz, 1H), 7.94 (d, J=7.2 Hz, 1H), 7.91 (s, 1H), 7.73 (t, 8.0 Hz, 1H); ¹³C NMR (150 MHz CDCl₃) δ ppm 191.4, 167.8, 138.3, 134.3, 133.3, 132.5, 132.4, 131.7, 130.9, 121.3, 128.5, 127.8, 126.8, 123.4, 108.2; UPLC-ESI-HMS: calculated exact mass for C₁₅H₇ClIOS⁺ [M+H⁺]: 396.8945, found [M+H⁺]: 396.8928.

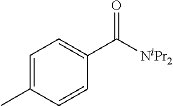

N,N-diisopropyl-4-methylbenzamide (19b). This compound was synthesized synthesized from 4-methylbenzoic acid (9c) using the general procedure for the synthesis of diisopropyl amides from carboxylic acids described above in 92% yield as a white solid. ¹H NMR (400 MHz CDCl₃) δ ppm 6.87 (d, J=8.1 Hz, 2H), 6.80 (d, J=7.9 Hz, 2H), 3.55 (s, br, 2H), 1.97 (s, 3H), 0.98 (s, br, 12H); ¹³C NMR (100 MHz CDCl₃) δ ppm 170.6, 138.0, 135.9, 128.7, 125.3, 20.9, 20.4; UPLC-ESI-HMS: calculated exact mass for C₁₄H₂₂NO⁺ [M+H⁺]: 220.1696, found [M+H⁺]: 220.1705.

2-Formyl-N,N-diisopropyl-4-methylbenzamide (11c). This compound was synthesized

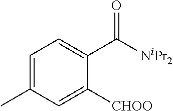

from 10c using the general procedure for ortho-formylation of the amides discussed above in 93% yield as a white solid. ¹H NMR (400 MHz CDCl₃) δ ppm 9.69 (s, 1H), 7.34 (s, 1H), 7.05 (dd, J=7.6, 1.0 Hz, 1H), 6.83 (d, J=7.7 Hz, 1H), 3.28-3.16 (m, 2H), 2.00 (s, 3H), 1.22 (d, J=6.8 Hz, 6H), 0.72 (d, J=6.7 Hz, 6H); ¹³C NMR (100 MHz CDCl₃) δ ppm 190.1, 168.1, 138.3, 138.2, 134.6, 132.0, 129.7, 125.7, 50.8, 45.5, 20.6, 20.0; UPLC-ESI-HMS: calculated exact mass for C₁₅H₂₂NO₂⁺ [M+H⁺]: 248.1645, found [M+H⁺]: 248.1650.

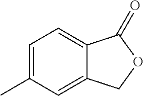

5-Methylisobenzofuran-1(3H)-one (12c). This compound was synthesized starting from 11c following the above general procedure for the synthesis of the 5-substituted phthalides in 83% yield as a white solid. ¹H NMR (400 MHz CDCl₃) δ ppm 7.59 (d, J=8.2 Hz, 1H), 7.20-7.18 (m, 2H), 5.12 (s, 2H), 2.36 (s, 3H); ¹³C NMR (100 MHz CDCl₃) δ ppm 171.1, 147.2, 145.2, 130.0, 125.0, 122.9, 122.5, 69.4, 21.9; UPLC-ESI-HMS: calculated exact mass for C₉H₉O₂⁺ [M+H⁺]: 149.0597, found [M+H⁺]: 149.0608.

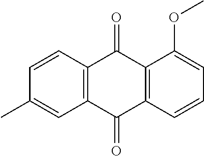

1-Methoxy-6-methylanthracene-9,10-dione (14c). This compound was synthesized from 12c and 13 using the general procedure for the synthesis of the 1-methoxy anthraquinones in 68% yield as a yellow solid. ¹H NMR (400 MHz CDCl₃) δ ppm 7.98 (d, J=7.9 Hz, 1H), 7.81 (dd, J=1.1, 0.5 Hz, 1H), 7.75 (dd, J=7.7, 1.1 Hz, 1H), 7.53 (dd, J=8.4, 7.7 Hz, 1H), 7.42 (ddd, J=8.0, 1.8, 0.7 Hz, 1H), 7.19 (dd, J=8.5, 0.8 Hz, 1H), 3.92 (s, 3H), 2.36 (s, 3H); ¹³C NMR (100 MHz CDCl₃) b ppm 183.3, 182.1, 160.1, 144.0, 135.6, 134.9, 134.7, 132.6, 132.1, 127.2, 126.5, 121.2, 119.5, 117.8, 56.4, 21.6; UPLC-ESI-HMS: calculated exact mass for C16H₁₃O₃⁺ [M+H⁺]: 253.0859, found [M+H⁺]: 253.0864.

5-Methoxy-2-methylanthracen-9(10H)-one (15c). This compound was synthesized from

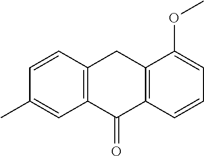

14c following the general procedure for reduction of 1-methoxyanthraquinones to anthrones in 76% yield as a pale-yellow solid. ¹H NMR (400 MHz CDCl₃) δ ppm 8.09 (s, 1H), 7.91 (dd, J=7.9, 0.9 Hz, 1H), 7.34-7.24 (m, 3H), 6.95 (dd, J=8.0, 0.7 Hz, 1H), 3.92 (s, 2H), 3.82 (s, 3H), 2.39 (s, 3H); ¹³C NMR (100 MHz CDCl₃) δ ppm 184.3, 156.5, 137.7, 136.4, 133.7, 132.8, 131.3, 129.8, 128.3, 127.1, 127.1, 119.0, 112.9, 55.5, 26.8, 21.1; UPLC-ESI-HMS: calculated exact mass for C₁₆H₁₅O₂⁺ [M+H⁺]: 239.1067, found [M+H⁺]: 239.1067.

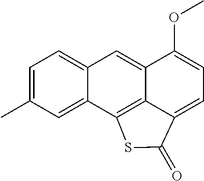

5-Methoxy-9-methyl-2H-anthra[9,1-bc]thiophen-2-one (17c). This compound was synthesized from 15c following the general procedure for thionation of the anthrones and general procedure for thiolactonization in 47% yield over three steps as an orange solid. ¹H NMR (400 MHz CDCl₃) δ ppm 8.57 (s, 1H), 8.18 (d, J=7.9 Hz, 1H), 7.95 (d, J=8.7 Hz, 1H), 7.61 (s, 1H), 7.36 (d, J=8.3 Hz, 1H), 6.95 (d, J=7.8 Hz, 1H), 4.19 (s, 3H), 2.59 (s, 3H); ¹³C NMR (100 MHz CDCl₃) δ ppm 191.6, 162.1, 137.8, 131.4, 130.4, 129.9, 128.9, 128.6, 128.5, 128.0, 125.0, 123.1, 121.1, 119.8, 103.9, 56.6, 22.2; UPLC-ESI-HMS: calculated exact mass for C₁₇HuO2S⁺ [M+H⁺]: 281.0631, found [M+H⁺]: 281.0627.

5-Hydroxy-9-methyl-2H-anthra[9,1-bc]thiophen-2-one (18c). This compound was

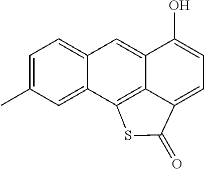 [SMILES: Cc1ccc2cc3c(O)ccc4c3c(c2c1)SC4=O]

synthesized following the general procedure for demethylation from 17c in 87% yield as an orange solid. ¹H NMR (400 MHz DMSO-d₆) δ ppm 8.71 (s, 1H), 8.18 (d, J=7.9, 1H), 8.14 (d, J=8.8 Hz, 1H), 7.59 (s, 1H), 7.41 (dd, J=8.8, 1.4 Hz, 1H), 7.13 (d, J=7.9 Hz, 1H), 2.53 (s, 3H); ¹³C NMR (100 MHz DMSO-d₆) δ ppm 189.8, 163.0, 138.7, 131.3, 131.0, 130.9, 130.7, 129.2, 127.8, 127.3, 122.7, 121.9, 121.0, 120.7, 109.5, 22.1; UPLC-ESI-HMS: calculated exact mass for C₁₆H₁₁O₂S⁺ [M+H⁺]: 267.0474, found [M+H⁺]: 267.0468.

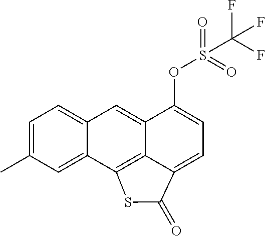 [SMILES: Cc1ccc2cc3c(OS(=O)(=O)C(F)(F)F)ccc4c3c(c2c1)SC4=O]

9-Methyl-2-oxo-2H-anthra[9,1-bc]thiophen-5-yl trifluoromethanesulfonate (19a). This compound was synthesized from 18c following the general procedure for triflate formation in 92% yield as an orange solid. ¹H NMR (400 MHz CDCl₃) δ ppm 8.51 (s, 1H), 8.24 (d, J=7.6 Hz, 1H), 8.07 (d, J=8.7 Hz, 1H), 2.64 (s, 3H); ¹³C NMR (100 MHz CDCl₃) δ ppm 190.9, 149.7, 139.3, 133.0, 132.5, 130.9, 130.7, 130.1, 129.9, 129.7, 128.8, 128.3, 125.3, 123.3, 121.8, 118.6, 118.2, 22.3; UPLC-ESI-HMS: calculated exact mass for C₁₇H₁₀F₃O₄S₂⁺ [M+H⁺]: 398.9967, found [M+H⁺]: 398.9964.

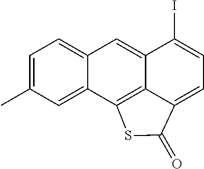 [SMILES: Cc1ccc2cc3c(I)ccc4c3c(c2c1)SC4=O]

5-Iodo-9-methyl-2H-anthra[9,1-bc]thiophen-2-one (20c). This compound was synthesized from 19c using the general procedure for iodination in 82% yield as an orange solid. ¹H NMR (600 MHz CDCl₃) δ ppm 8.43 (s, 1H), 8.33 (d, J=7.3 Hz, 1H), 8.06 (d, J=8.6 Hz, 1H), 7.89 (d, J=7.2 Hz, 1H), 7.65 (s, 1H), 7.46 (d, J=8.5 Hz, 1H), 2.63 (s, 3H); ¹³C NMR (150 MHz CDCl₃) δ ppm 192.3, 138.7, 137.5, 133.2, 133.0, 130.6, 130.0, 129.9, 129.8, 128.8, 128.4, 128.0, 126.0, 123.1, 108.1, 22.3; UPLC-ESI-HMS: calculated exact mass for C₁₆H₁₀IOS⁺ [M+H⁺]: 376.9492, found [M+H⁺]: 376.9497.

4-Ethyl-N,N-diisopropylbenzamide (10d). This compound was synthesized from 4-

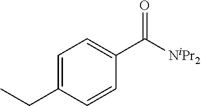

ethylbenzoic acid (9d) using the general procedure for the synthesis of diisopropyl amides from carboxylic acids described above in 90% yield as a white solid. ¹H NMR (400 MHz CDCl₃) δ ppm 7.12-7.07 (m, 4H), 3.58 (s, br, 2H), 2.54-2.50 (m, 2H), 1.23 (s, br, 12H), 1.14-1.03 (m, 3H); ¹³C NMR (100 MHz CDCl₃) δ ppm 171.0, 144.6, 136.3, 127.7, 125.6, 28.6, 28.5, 20.6, 15.3; UPLC-ESI-HMS: calculated exact mass for C₁₅H₂₄NO₂⁺ [M+H⁺]: 234.1852, found [M+H⁺]: 234.1858.

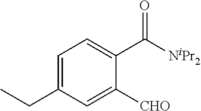

4-Ethyl-2-formyl-N,N-diisopropylbenzamide (11d). This compound was synthesized from 10d using the general procedure for ortho-formylation of the amides discussed above in 95% yield as a white solid. ¹H NMR (400 MHz CDCl₃) δ ppm 9.79 (s, 1H), 7.47 (d, J=1.4 Hz, 1H), 7.16 (dd, J=7.4, 1.8 Hz, 1H), 6.93 (d, J=7.7 Hz, 1H), 3.38-3.24 (m, 2H), 2.40 (q, J=7.6 Hz, 2H), 1.31 (d, J=6.8 Hz, 6H), 0.94 (t, J=7.6 Hz, 3H), 0.80 (d, J=6.7 Hz, 6H); ¹³C NMR (100 MHz CDCl₃) δ ppm 190.3, 168.2, 144.6, 138.6, 133.6, 132.2, 128.5, 125.8, 50.9, 45.7, 28.1, 20.2, 20.1, 14.9; UPLC-ESI-HMS: calculated exact mass for C₁₆H₂₄NO₂⁺ [M+H⁺]: 262.1802, found [M+H⁺]: 262.1806.

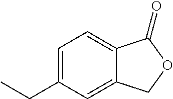

5-Ethylisobenzofuran-1(3H)-one (12d). This compound was synthesized starting from 11d following the above general procedure for the synthesis of the 5-substituted phthalides in 87% yield as a white solid. ¹H NMR (400 MHz CDCl₃) δ ppm 7.58 (d, J=7.7 Hz, 1H), 7.20-7.18 (m, 2H), 5.11 (s, 2H), 2.62 (q, J=7.6 Hz, 2H), 1.12 (t, J=7.6 Hz, 3H); ¹³C NMR (100 MHz CDCl₃) δ ppm 171.0, 151.4, 147.2, 129.0, 125.1, 123.1, 121.3, 69.5, 29.1, 15.2; UPLC-ESI-HMS: calculated exact mass for C₁₀H₁₁O₂⁺ [M+H⁺]: 163.0754, found [M+H⁺]: 163.0762.

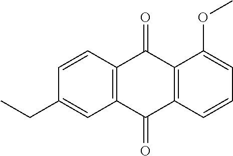 [SMILES: CCc1ccc2c(c1)C(=O)c1cccc(OC)c1C2=O]

6-Ethyl-1-methoxyanthracene-9,10-dione (14d). This compound was synthesized from 12d and 13 using the general procedure for the synthesis of the 1-methoxyanthraquinones in 58% yield as a yellow solid. ¹H NMR (400 MHz CDCl₃) δ ppm 8.03 (d, J=8.0 Hz, 1H), 7.88-7.87 (m, 1H), 7.76 (dd, J=7.7, 1.1 Hz, 1H), 7.53 (dd, J=8.4, 7.7 Hz, 1H), 7.46 (tdd, J=8.0, 1.9, 0.5 Hz, 1H), 7.19 (dd, J=8.5, 1.0 Hz, 1H), 3.92 (s, 3H), 2.67 (q, J=7.6 Hz, 2H), 1.20 (t, J=7.6 Hz, 3H); ¹³C NMR (100 MHz CDCl₃) δ ppm 183.4, 182.1, 160.1, 150.1, 135.6, 134.7, 133.9, 132.8, 132.3, 127.4, 125.4, 121.3, 119.5, 117.8, 56.4, 28.9, 14.9; UPLC-ESI-HMS: calculated exact mass for C₁₇H₁₅O₃⁺ [M+H⁺]: 267.1016, found [M+H⁺]: 267.1020.

2-Ethyl-5-methoxyanthracen-9(10H)-one (15d). This compound was synthesized from

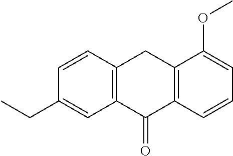 [SMILES: CCc1ccc2c(c1)C(=O)c1cccc(OC)c1C2]

14d following the general procedure for reduction of 1-methoxyanthraquinones to anthrones in 72% yield as a pale-yellow solid. ¹H NMR (400 MHz CDCl₃) δ ppm 8.12 (d, J=1.5 Hz, 1H), 7.88 (dd, J=7.9, 1.0 Hz, 1H), 7.20-7.22 (m, 3H), 6.86 (dd, J=8.1, 0.9 Hz, 1H), 3.85 (s, 2H), 3.74 (s, 3H), 2.67 (q, J=7.6 Hz, 2H), 1.26 (t, J=7.6 Hz, 3H); ¹³C NMR (100 MHz CDCl₃) δ ppm 184.2, 156.4, 142.7, 138.0, 132.7, 132.6, 131.4, 129.7, 128.8, 127.0, 125.9, 118.9, 112.8, 55.4, 28.5, 26.8, 15.5; UPLC-ESI-HMS: calculated exact mass for C17H₁₇₀₂⁺ [M+H⁺]: 253.1223, found [M+H⁺]: 253.1228.

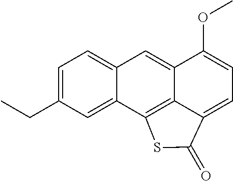 [SMILES: CCc1ccc2cc3c(OC)ccc4c3c(c2c1)SC4=O]

9-Ethyl-5-methoxy-2H-anthra[9,1-bc]thiophen-2-one (17d). This compound was synthesized from 15d following the general procedure for thionation of the anthrones and general procedure for thiolactonization in 52% yield over three steps as an orange solid. ¹H NMR (600 MHz CDCl₃) δ ppm 8.59 (s, 1H), 8.19 (d, J=7.8 Hz, 1H), 7.98 (d, J=8.7 Hz, 1H), 7.62 (s, 1H), 7.41 (dd, J=8.7, 1.4 Hz, 1H), 6.96 (d, J=7.9 Hz, 1H), 4.19 (s, 3H), 2.89 (q, J=7.6 Hz, 2H), 1.40 (t, J=7.6 Hz, 3H); ¹³C NMR (150 MHz CDCl₃) δ ppm 191.7, 162.2, 144.0, 131.6, 130.4, 130.1, 128.7, 128.1, 127.9, 124.9, 121.7, 121.1, 119.8, 103.9, 56.6, 29.4, 15.1; UPLC-ESI-HMS: calculated exact mass for C₁₈H₁₅O₂S⁺ [M+H⁺]: 295.0787, found [M+H⁺]: 295.0793.

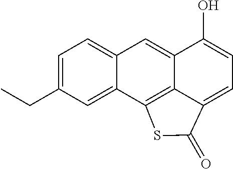 [SMILES: CCc1ccc2cc3c(O)ccc4c3c(c2c1)SC4=O]

9-Ethyl-5-hydroxy-2H-anthra[9,1-bc]thiophen-2-one (18d). This compound was synthesized following the general procedure for demethylation from 17d in 92% yield as an orange solid. ¹H NMR (400 MHz DMSO-d₆) δ ppm 8.72 (s, 1H), 8.18 (t, J=8.0 Hz, 1H), 7.57 (s, 1H), 7.47 (d, J=8.6 Hz, 1H), 7.13 (d, J=7.8 Hz, 1H), 2.83 (q, J=7.3 Hz, 2H), 1.29 (t, J=7.3 Hz, 3H); ¹³C NMR (100 MHz DMSO-d₆) δ ppm 189.8, 162.9, 144.7, 131.2, 130.9, 128.2, 127.8, 127.6, 121.9, 121.4, 121.0, 120.8, 109.5, 29.1, 15.4; UPLC-ESI-HMS: calculated exact mass for C₁₇H₁₃O₂S⁺ [M+H⁺]: 281.0631, found [M+H⁺]: 281.0637.

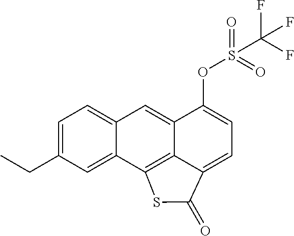 [SMILES: CCc1ccc2cc3c(OS(=O)(=O)C(F)(F)F)ccc4c3c(c2c1)SC4=O]

9-Ethyl-2-oxo-2H-anthra[9,1-bc]thiophen-5-yl trifluoromethanesulfonate (19d). This compound was synthesized from 18d following the general procedure for triflate formation in 90% yield as an orange solid. ¹H NMR (400 MHz CDCl₃) δ ppm 8.42 (s, 1H), 8.19 (d, J=7.6 Hz, 1H), 8.03 (d, J=8.8 Hz, 1H), 7.67 (d, J=7.6 Hz, 1H), 7.60 (s, 1H), 7.51 (d, J=8.8 Hz, 1H), 2.91 (q, J=7.4 Hz, 2H), 1.41 (t, J=7.4 Hz, 3H); ¹³C NMR (100 MHz CDCl₃) δ ppm 190.8, 149.6, 145.3, 133.1, 132.3, 130.2, 129.9, 129.7, 129.6, 128.2, 125.2, 121.8, 121.6, 118.5, 118.1, 29.4, 14.8; UPLC-ESI-HMS: calculated exact mass for C₁₈H₁₂F₃O₄S₂⁺ [M+H⁺]: 413.0124, found [M+H⁺]: 413.0127.

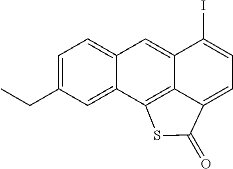 [SMILES: CCc1ccc2cc3c(I)ccc4c3c(c2c1)SC4=O]

9-Ethyl-5-iodo-2H-anthra[9,1-bc]thiophen-2-one (20d). This compound was synthesized from 19d using the general procedure for iodination in 87% yield as an orange solid. ¹H NMR (400 MHz CDCl₃) δ ppm 8.38 (s, 1H), 8.29 (d, J=7.3 Hz, 1H), 8.05 (d, J=8.8 Hz, 1H), 7.85 (d, J=7.3 Hz, 1H), 7.61 (quint, J=0.8 Hz, 1H), 7.48 (dd, J=8.8, 1.5 Hz, 1H), 2.91 (dq, J=7.5, 0.7 Hz, 2H), 1.41 (t, J=7.5 Hz, 3H); ¹³C NMR (100 MHz CDCl₃) δ ppm 192.2, 144.7, 137.5, 133.3, 133.2, 130.6, 130.1, 129.9, 129.0, 128.7, 128.2, 128.0, 125.8, 121.7, 108.0, 29.4, 14.9; UPLC-ESI-HMS: calculated exact mass for C₁₇H₁₂IOS⁺ [M+H⁺]: 390.9648, found [M+H⁺]: 390.9643.

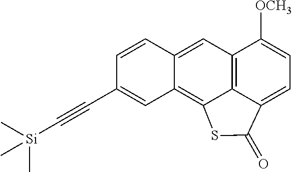 [SMILES: COc1ccc2c3c(c4cc(C#C[Si](C)(C)C)ccc4cc13)SC2=O]

5-Methoxy-9-((trimethylsilyl)ethynyl)-2H-anthra [9,1-bc]thiophen-2-one (30). In a flame-dried round-bottomed flask 9-bromo-5-methoxy-2H-anthra[9,1-bc]thiophen-2-one (17e, 0.50 g, 1.45 mmol), Pd(PPh₃)₄ (0.17 g, 0.14 mmol) and CuI (83 mg, 0.43 mmol,) were added and flushed with Ar. To this 10 mL of 1:1 anhydrous DMF and freshly distilled NEt₃ were added followed by TMS-acetylene (0.82 mL, 5.8 mmol). The reaction mixture was heated to 65° C. and stirred at that temperature for 16 h. After allowing the reaction to cool, the mixture was poured into ice-cold water and extracted with EtOAc. The organic layer was washed with water and brine, dried over anhydrous Na₂SO₄ and evaporated in vacuo. The crude product was purified by silica gel column chromatography using 20% EtOAc in hexanes to give the product as an orange solid (0.43 g, 82% yield). ¹H NMR (400 MHz CDCl₃) δ ppm 8.34 (s, 1H), 8.04 (d, J=7.9 Hz, 1H), 7.89 (quint, J=0.8 Hz, 1H), 7.83 (t, J=0.6 Hz, 1H), 7.81 (t, J=0.6 Hz, 1H), 7.41 (dd, J=8.7, 1.5 Hz, 1H), 6.84 (d, J=7.9 Hz, 1H), 4.13 (s, 3H), 0.36 (s, 9H); ¹³C (100 MHz CDCl₃) δ ppm 190.8, 161.9, 131.5, 130.4, 129.9, 129.9, 128.9, 128.5, 128.2, 126.7, 124.6, 122.0, 121.5, 119.7, 104.9, 104.4, 97.3, 56.6, −0.01; UPLC-ESI-HMS: calculated exact mass for C₂₁H₉O₂SSi⁺ [M+H⁺]: 363.0870, found [M+H⁺]: 363.0869.

9-Acetyl-5-methoxy-2H-anthra[9,1-bc]thiophen-2-one (31). 5-Methoxy-9-

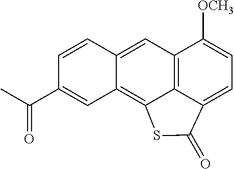 [SMILES: COc1ccc2c3c(c4cc(C(C)=O)ccc4cc13)SC2=O]

((trimethylsilyl)ethynyl)-2H-anthra[9,1-bc]thiophen-2-one (30, 0.40 g, 1.30 mmol) was dissolved in 10 mL neat HCOOH and heated to reflux for 5 h. After cooling the reaction mixture, HCOOH was removed by azeotropic distillation with CHCl₃. The crude mixture was purified by silica column chromatography with 25% EtOAc in hexanes as a yellowish orange solid (0.35 g, 88% yield). ¹H NMR (400 MHz CDCl₃) δ ppm 8.69 (s, 1H), 8.49 (quint, J=0.8 Hz, 1H), 8.27 (d, J=7.9 Hz, 1H), 8.13 (d, J=8.9 Hz, 1H), 8.06 (dd, J=8.9, 1.6 Hz, 1H), 7.08 (d, J=7.9 Hz, 1H), 4.24 (s, 3H), 2.81 (s, 3H); ¹³C (100 MHz CDCl₃) δ ppm 197.5, 190.4, 162.1, 135.4, 134.0, 133.3, 130.9, 130.8, 129.5, 127.6, 126.6, 125.0, 123.5, 122.9, 119.9, 105.2, 56.8, 26.7; UPLC-ESI-HMS: calculated exact mass for C₁₅H₁₃O₃S⁺ [M+H⁺]: 309.0580, found [M+H⁺]: 309.0571.

9-Acetyl-5-hydroxy-2H-anthra[9,1-bc]thiophen-2-one (32). This compound was

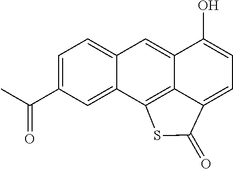 [SMILES: CC(=O)c1ccc2cc3c(O)ccc4c3c(c2c1)SC4=O]

synthesized following the general procedure for demethylation from 31 in 72% yield as an orange solid. ¹H NMR (400 MHz DMSO-d₆) δ ppm 8.65 (s, 1H), 8.28 (t, J 0.8 Hz, 1H), 8.20 (d, J=8.9 Hz, 1H), 8.17 (d, J=7.9 Hz, 1H), 7.87 (dd, J=8.8, 1.6 Hz, 1H), 7.14 (d, J=7.9 Hz, 1H), 2.73 (s, 3H); ¹³C (100 MHz DMSO-d₆) δ ppm 197.9, 189.2, 162.9, 135.5, 133.2, 131.9, 131.5, 131.4, 131.3, 127.0, 126.2, 123.2, 122.3, 121.8, 120.9, 110.5, 27.3; UPLC-ESI-HMS: calculated exact mass for C₁₇H₁₁O₃S⁺ [M+H⁺]: 295.0423, found [M+H⁺]: 295.0428.

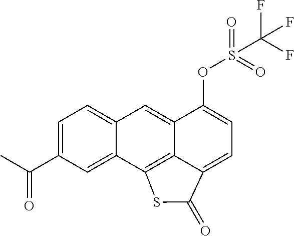 [SMILES: CC(=O)c1ccc2cc3c(OS(=O)(=O)C(F)(F)F)ccc4c3c(c2c1)SC4=O]

9-Acetyl-2-oxo-2H-anthra[9,1-bc]thiophen-5-yl trifluoromethanesulfonate (33). This compound was synthesized from 32 following the general procedure for triflate formation in 81% yield as an orange solid. ¹H NMR (600 MHz CDCl₃) δ ppm 8.59 (s, 1H), 8.53 (s, 1H), 8.32 (dd, J=7.6, 1.6 Hz, 1H), 8.24 (d, J=8.9 Hz, 1H), 8.19 (d, J=8.9 Hz, 1H), 7.84 (dd, J=7.6, 1.6 Hz, 1H), 2.84 (s, 3H); ¹³C (150 MHz CDCl₃) δ ppm 197.0, 189.7, 149.6, 136.2, 135.5, 134.9, 132.6, 130.8, 130.1, 127.4, 127.0, 126.2, 125.3, 123.5, 119.7, 118.7, 26.8; UPLC-ESI-HMS: calculated exact mass for C15H₁₀F₃O₅S₂⁺ [M+H⁺]: 426.9916, found [M+H⁺]: 426.9898.

9-Acetyl-5-iodo-2H-anthra[9,1-bc]thiophen-2-one (34). This compound was synthesized

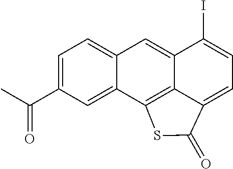 [SMILES: CC(=O)c1ccc2cc3c(I)ccc4c3c(c2c1)SC4=O]

from 33 using the general procedure for iodination in 72% yield as an orange solid. ¹H NMR (600 MHz CDCl₃) δ ppm 8.52 (s, 1H), 8.51 (s, 1H), 8.44 (d, J=7.3 Hz, 1H), 8.23 (d, J=8.9 Hz, 1H), 8.15 (dd, J=8.9, 1.5 Hz, 1H), 7.95 (d, J=7.3 Hz, 1H), 2.83 (s, 3H); ¹³C (150 MHz CDCl₃) δ ppm 197.3, 191.1, 139.1, 135.8, 135.6, 134.8, 133.3, 132.3, 130.7, 128.9, 128.8, 127.5, 126.7, 124.5, 108.2, 26.8; UPLC-ESI-HMS: calculated exact mass for C₁₇H₁₀IO₂S⁺ [M+H⁺]: 404.9441, found [M+H⁺]: 404.9420.

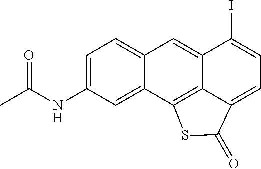 [SMILES: CC(=O)Nc1ccc2cc3c(I)ccc4c3c(c2c1)SC4=O]

N-(5-iodo-2-oxo-2H-anthra[9,1-bc]thiophen-9-yl) acetamide (35). In a flame-dried round-bottomed flask 9-acetyl-5-iodo-2H-anthra[9,1-bc]thiophen-2-one (34, 10 mg, 0.02 mmol) was dissolved in 5 mL dry toluene and cooled to 0° C. in an ice-water bath. To this solution, NaN₃ (6 mg, 0.10 mmol) was added followed by a few drops of conc. H₂SO₄. The solution was stirred for 16 h while allowing the reaction to warm to room temperature. After completion, the reaction was quenched cautiously with sat. NaHCO₃ solution and the reaction mixture was extracted with EtOAc. The combined organic layer was washed with brine, dried over anhydrous Na₂SO₄ and evaporated in vacuo. The crude product was purified by silica gel column chromatography using 50% EtOAc in hexanes with 1% AcOH to give the product as an orange solid (7 mg, 68%). ¹H NMR (400 MHz DMSO-d₆) δ ppm 8.65 (s, 1H), 8.42 (s, 1H), 8.38 (d, J=7.4 Hz, 1H), 8.19 (d, J=7.5 Hz, 1H), 7.94 (d, J=7.6 Hz, 1H), 7.91 (d, J=7.3 Hz, 1H), 2.16 (s, 3H); UPLC-ESI-HMS: calculated exact mass for C₁₇H₁₁INO₂S⁺ [M+H⁺]: 419.9550, found [M+H⁺]: 419.9547.

9-Amino-5-iodo-2H-anthra[9,1-bc]thiophen-2-one (36). N-(5-iodo-2-oxo-2H-anthra[9,1-

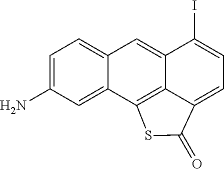 [SMILES: Nc1ccc2cc3c(I)ccc4c3c(c2c1)SC4=O]

bc]thiophen-9-yl)acetamide (35, 5 mg) was dissolved in 2 mL EtOH, to this solution 1 drop of conc. HCl was added and heated to 80° C. for 2 h. After completion, the reaction mixture was cooled to room temperature, diluted with water and extracted with EtOAc. Due to instability of the compound, the reaction was performed right before the in vitro feeding and confirmed only by LCMS. UPLC-ESI-HMS: calculated exact mass for C₁₅H₉INOS⁺ [M+H⁺]: 377.9444, found [M+H⁺]: 377.9440.
2.4.3 Small-Scale Supplementation with 9-Substituted-5-Iodoanthracene-γ-Lactones and Analysis of Dynemicin A-Analogue Production

M. chersina ΔOrf15 mycelial stock (100 μL) was plated on med 53 with 2% agar and grown at 28° C. After 7 d, mycelia from the plate were inoculated into 50 mL med 53 liquid fermentation medium in a 125 mL Erlenmeyer flask and shaken at 250 rpm at 28° C. for another 7 d. This starter culture (2 mL) was inoculated into 50 mL H881 liquid medium in 250 mL Erlenmeyer flasks prepared with or without NaI depending on the experiment and shaken at 250 rpm and 28° C. After 24 h, 250 μL of 2 mM stock solutions of 20a-d, 34 and 36 in DMSO (sterile filtered using 0.2 μm PTFE filters) were added to the respective flasks to provide a final concentration of the added compounds of 10 μM. After shaking at 250 rpm and 28° C. for another 2 d, Diaion® HP-20 (0.50 g, sterilized by autoclave) was added to each flask and shaking was continued for another 4 d. After total 7 d of fermentation, 5 mL from each sample was extracted with 5 mL of EtOAc by vortexing for 1 min. Extracts were centrifuged at 4000×g and 4° C. for 5 min, and 4 mL of each EtOAc layer was dried by SpeedVac without heating. Samples were dissolved in 200 μL DMSO, filtered through 0.2 μm PTFE filters and analyzed on an Agilent 1200 HPLC using a Prodigy ODS3 100 Å, 5 μM, 250×4.6 mm column (Phenomenex). A gradient method of 5-95% ACN+0.1% (v/v) formic acid over 40 min, followed by 10 min hold at 95% ACN before column re-equilibration was used at a 1 mL/min flow rate to achieve separation of metabolites. Production of dynemicin and other related metabolites was monitored at 280, 450 and 570 nm.

2.4.4 UPLC-ESI-MS Analysis of Products from ΔOrf15 Fermentations Supplemented with Synthetic Iodoanthracenes

For UPLC-ESI-MS analysis of metabolites, a 50-mL day 7 fermentation culture was extracted as described before. The concentrated EtOAc layer was resuspended in 2.5-mL MeOH and filtered through a 0.2-μm polytetrafluoroethylene (PTFE) filter. The extract was analyzed on an Agilent 1100 HPLC using a Kinetex 100 Å, 5 μm, 250×10.0 mm C18 LC column (Phenomenex). Injections of 400-500 μL were separated using a gradient method of 50-95% ACN+0.1% (v/v) formic acid over 20 min, followed by a 5-min hold at 95% ACN before column re-equilibration (4 mL min⁻¹ flow rate). Production of the metabolites was monitored at 280 and 570 nm. Peaks were manually collected, frozen at −80° C., and lyophilized. Lyophilized samples were re-dissolved in 200-μL MeOH and analyzed on a Waters ACQUITY/Xevo G-2 UPLC-ESI-MS with positive mode ESI ionization.

2.4.6 Extraction of Compounds from Large Scale Fermentations

100 μL of M. chersina ΔOrf15 mycelia was plated on medium 53 with 2% agar plates and grown for 7 days at 28° C. Afterward, equally sized squares of mycelia were used to inoculate 3×50 mL medium 53 in 125-mL shake flasks. The cultures were incubated at 28° C. at 250 rpm for 7 days. On the seventh day 6×20 mL vegetative cultures were used to inoculate 6×1 L H881 in 2.8 L flasks. After 24 h, 1 mL of 5 mM stock solutions of 20a-d, 34 and 36 in DMSO (sterile filtered using 0.2-μm PTFE filters) were added to the respective flasks to provide a final concentration of the added compounds of 10 μM. After shaking at 250 rpm and 28° C. for another 2 d, Diaion® HP-20 (10 g, sterilized by autoclave) was added to each flask and shaking was continued for another 7 d.

Metabolites were extracted on the tenth day. In a 5 L flask, 3 L of fermentation medium was combined with 1 L EtOAc. The mixture was left stirring on a magnetic stir plate for 2 h. Using 500 mL centrifuge tubes, the mixture was centrifuged 2 L at a time at 6,100×g, 4° C. for 12 min. Once pelleted, the supernatant was filtered through filter paper using a Buchner funnel. A 2 L separatory funnel was then used to separate the organic and aqueous phases. After the first extraction, secondary washes were done with both the aqueous layer, and organic layer with EtOAc and brine, respectively. The organic layers were then filtered through a bed of Celite to remove any residual Diaion® HP-20 resin or particulates. The organic layers were dried over anhydrous Na₂SO₄, and filtered into a 1 L round bottom flask. The EtOAc was removed in vacuo. The residue was then transferred to a tared vial and frozen at −80° C.

2.4.8 Purification of Metabolites from the ΔOrf15 Mutant Supplemented with Synthetic Iodoanthracenes

The concentrated extract from 12 L fermentations supplemented with synthetic compounds was adsorbed to Celite and dry-loaded onto a silica gel flash chromatography column. Using a 0-10% methanol in CHCl₃ gradient, the dark blue band was collected. The crude extract was acetylated using excess acetic anhydride and pyridine (1:1 molar equivalents) at room temperature for 16 h, during which the reaction turned from dark blue to orange. After completion, the reaction mixture was diluted with EtOAc and washed twice with water and once with sat. NH₄Cl solution. The EtOAc layer was dried over anhydrous Na₂SO₄ and concentrated in vacuo. The crude acetylated products were again adsorbed on Celite and dry-loaded onto a silica gel. Using a gradient of 80:20 EtOAc:hexane to 100% EtOAc to 95:5 EtOAc:MeOH, the pink-orange material that eluted at 95:5 EtOAc:MeOH was collected and concentrated in vacuo. The partially pure acetylated compound was further purified using a Sephadex LH-20 (GE Healthcare Life Sciences) resin column. A 1×25 cm column was pre-equilibrated with 1:1 CHCl₃:MeOH and the compound was loaded dissolved in the same solvent system and the pink-orange band was collected. The compound was re-purified using the same column regenerated with MeOH, using MeOH as mobile phase, and the orange band was collected. The MeOH was removed in vacuo to yield the acetylated Dyn analogues.

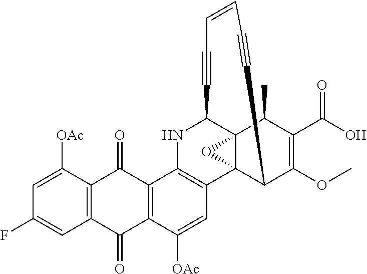 [SMILES: COC1=C(C(=O)O)[C@H](C)[C@@]23O[C@]24c2cc(OC(C)=O)c5c(c2N[C@H]3C#C/C=C\C#C[C@H]14)C(=O)c1c(OC(C)=O)cc(F)cc1C5=O]

16-Fluorodynemicin diacetate (23). The compound was isolated, acetylated and purified following the general protocol above from 12 L fermentation. Pure diacetylated compound (2.3 mg) was isolated as an orange solid. ¹H NMR (400 MHz DMSO-d₆) δ ppm 9.77 (d, J=4.4 Hz, 1H), 8.02 (s, 1H), 7.77 (dd, J=8.5, 2.7 Hz, 1H), 7.64 (dd, J=8.8, 2.8 Hz, 1H), 6.05 (dq, J=9.9, 1.3 Hz, 2H), 5.03 (d, J=4.2 Hz, 1H), 4.53 (s, 1H), 3.73 (s, 3H), 3.51 (q, J=7.0 Hz), 2.46 (s, 3H), 2.38 (s, 3H), 1.25 (d, J=7.0 Hz, 3H); ¹³C (150 MHz DMSO-d₆) δ ppm 183.1 (s), 180.3 (s), 169.7 (s), 169.4 (s), 164.9 (d, J=254.2 Hz), 152.4 (d, J=11.5 Hz) 145.3 (s), 141.0 (s), 137.5 (d, J=8.2 Hz), 132.4 (s), 130.6 (s), 124.9 (s), 124.5 (s), 123.5 (s), 122.7 (s), 118.3 (d, J=24.2 Hz), 115.1 (s), 111.7 (d, J=22.8 Hz), 101.5 (s), 98.0 (s), 90.1 (s), 88.8 (s), 72.1 (s), 63.7 (s), 58.4 (s), 44.4 (s), 36.7 (s), 32.5 (s), 21.5 (s), 21.4 (s), 18.9 (s), 14.6 (s); UPLC-ESI-HMS: calculated exact mass for C₃₄H₂₃FNO₁₀⁺ [M+H⁺]: 624.1301, found [M+H⁺]: 624.1313.

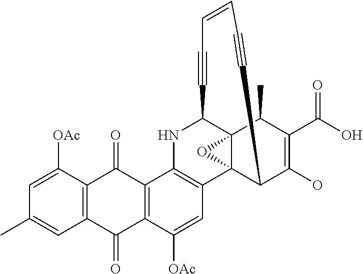 [SMILES: CC(=O)Oc1cc(C)cc2c1C(=O)c1c3c(cc(OC(C)=O)c1C2=O)[C@@]12O[C@@]14[C@H](C#C/C=C\C#C[C@@H]2C(O)=C(C(=O)O)[C@@H]4C)N3]

16-Methyldynemicin diacetate (28). The compound was isolated, acetylated and purified following the general protocol above from 12 L fermentation. Pure diacetylated compound (5.2 mg) was isolated as an orange solid. ¹H NMR (600 MHz DMSO-d₆) δ ppm 9.78 (d, J=4.4 Hz, 1H), 8.05 (s, 1H), 7.85 (dd, J=1.7, 0.8 Hz, 1H), 7.44 (dd, J=1.7, 0.8 Hz, 1H), 6.08 (dq, J=9.9, 1.5 Hz, 2H), 5.08 (dd, J 4.4, 1.3 Hz, 1H), 4.82 (s, 1H), 3.82 (s, 3H), 3.57 (q, J=7.3 Hz, 1H), 2.48 (s, 3H), 2.44 (s, 3H), 2.39 (s, 3H), 1.27 (d, J=7.3 Hz, 3H); ¹³C (150 MHz DMSO-d₆) δ ppm 184.0, 181.6, 169.7, 169.7, 167.9, 153.9, 150.0, 146.8, 145.1, 140.7, 134.9, 131.4, 131.0, 130.5, 125.5, 125.0, 124.7, 123.8, 122.8, 115.6, 115.2, 100.0, 98.0, 90.1, 89.4, 79.7, 71.9, 63.6, 58.3, 44.3, 36.1, 32.0, 21.6, 21.5, 21.4, 19.1; UPLC-ESI-HMS: calculated exact mass for C35H₂₆NO₁₀⁺ [M+H⁺]: 620.1551, found [M+H⁺]: 620.1550.

TABLE 5
Primers used in Example 2.

| Primer | Sequence |
|---|---|
| ORF15_5F | TCGTCGAAGGCACTAGAAGGCCGGCTGGACAACCGCACC |
| | AACCAG GAACT (SEQ ID NO: 26) |
| ORF15_5R | CATCTGGTCGGGCTTCAGGTGAACGCTCTTGGTGCTCAT |
| | CTCTGGT GTCC (SEQ ID NO: 27) |
| ORF15_3F | ACCTGAAGCCCGACCAGATGTGATTCCCCA (SEQ ID |
| | NO: 28) |
| ORF15_3R | GGTCGATCCCCGCATATAGGAGTAGCCGGTGAGGATCTT |
| | GCGTTCG TCCA (SEQ ID NO: 29) |
| ORF15_sgRNA_ | CATGCCATGGATCTTCGACATGCCGAAGCTGTTTTAGAGC |
| 1F | TAGAAAT AGC (SEQ ID NO: 30) |
| ORF15_KOtestF | ACGCGCTCAACTTCTCCTACCGGACCA (SEQ ID NO: 31) |
| ORF15_KOtestR | CTGACCGTTCCCGGTGCCGTTCAGGT (SEQ ID NO: 32) |

TABLE 6
UPLC-ESI-MS and UV-visible analysis of metabolites isolated from
M. chersina ΔOrf15 supplemented with 20a-d, 34 and 36.

| | | | Predicted Mol. |
|---|---|---|---|
| Compound | UV max (nm) | [M + H]⁺ | Formula |
| 21 | 232, 257, 328 (sh), | 540.1089 | C₃₀H₁₉FNO₈⁺ |
| | 549, 585 (sh) | | (Calcd [M + H]⁺ = |
| | | | 540.1089) |
| 22 | 259, 286 (sh), 318 | 524.1138 | C₃₀H₁₉FNO₇⁺ |
| | (sh), 536, 571 (s) | | (Calcd [M + H]⁺ = |
| | | | 524.1140) |
| 24 | 237, 260 (sh), 292 | 536.1339 | C₃₁H₂₂NO₈⁺ |
| | (sh), 544, 582 | | (Calcd [M + H]⁺ = |
| | | | 536.1340) |
| 25 | 223, 265, 288 (sh), | 520.1387 | C₃₁H₂₂NO₇⁺ |
| | 330 (sh), 532 | | (Calcd [M + H]⁺ = |
| | | | 520.1391) |
| 27 | 237, 162, 291, (sh), | 556.0798 | C₃₀H₁₉ClNO₈⁺ |
| | 383 (sh), 552, 590 (sh) | | (Calcd [M + H]⁺ = |
| | | | 556.0794) |
| 28 | 232 (sh0, 262, 288 | 540.0830 | C₃₀H₁₉ClNO₇⁺ |
| | (sh), 324 (sh), 539 | | (Calcd [M + H]⁺ = |
| | | | 540.0845) |
| 29 | 237 (sh), 263, 285 | 550.1484 | C₃₂H₂₄NO₈⁺ |
| | (sh), 330 (sh), 530 | | (Calcd [M + H]⁺ = |
| | | | 550.1496) |
| 41 | 244, 303, 330 (sh), | 537.1279 | C₃₀H₂₁N₂O₈⁺ |
| | 379 (sh), 552 | | (Calcd [M + H]⁺ = |
| | | | 537.1292) |
| 42 | 238, 297, 353(sh), | 521.1325 | C₃₀H₂₁N₂O₇⁺ |
| | 561 | | (Calcd [M + H]⁺ = |
| | | | 521.1343) |

### Example 3

### Dynemicin A Derivatives as Potential Cancer Chemotherapeutics by Mutasynthesis

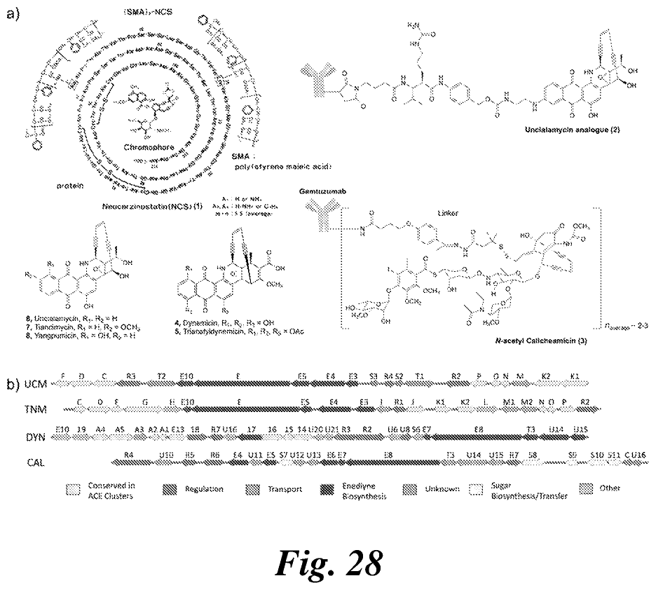

Throughout Example 3, including Schemes 1-5 and FIGS. 28-31, the following chart is used to correlate the compounds numbers presented in Example 3 with those presented in Example 2. Unless note otherwise, the experimental procedures are the same as those provided in Example 1 and Example 2.

TABLE 7
Correlation Chart for Cross Referencing Compounds
of Example 3 (Ex3) and Example 2 (Ex 2)^{†}

| Ex 3 | Ex 2 | Ex 3 | Ex 2 | Ex 3 | Ex 2 | Ex 3 | Ex 2 | Ex 3 | Ex 2 |
|---|---|---|---|---|---|---|---|---|---|
| 16a | 20a | 16b | 20b | 16c | 20c | 16d | 20d | | |
| 17a | 9a | 17b | 9b | 17c | 9c | 17d | 9d | | |
| 18a | 10a | 18b | 10b | 18c | 10c | 18d | 10d | | |
| 19a | 11a | 19b | 11b | 19c | 11c | 19d | 11d | | |
| 20a | 12a | 20b | 12b | 20c | 12c | 20d | 12d | 20e | 12e |
| 21 | 13 | | | | | | | | |
| 22a | 14a | 22b | 14b | 22c | 14c | 22d | 14d | 22e | 14e |
| 23a | 15a | 23b | 15b | 23c | 15c | 23d | 15d | 23e | 15e |
| 24a | 16a | 24b | 16b | 24c | 16c | 24d | 16d | 24e | 16e |
| 25a | 17a | 25b | 17b | 25c | 17c | 25d | 17d | 25e | 17e |
| 26a | 18a | 26b | 18b | 26c | 18c | 26d | 18d | | |
| 27a | 19a | 27b | 19b | 27c | 19c | 27d | 19d | | |
| 28 | 21 | | | | | | | | |
| 29 | 22 | | | | | | | | |
| 30 | 23 | | | | | | | | |
| 31 | 27 | | | | | | | | |
| 32 | 28 | | | | | | | | |
| 33 | — | | | | | | | | |
| 34 | 24 | | | | | | | | |
| 35 | 25 | | | | | | | | |
| 36 | 26 | | | | | | | | |
| 39 | 30 | | | | | | | | |
| 40 | 31 | | | | | | | | |
| 41 | 32 | | | | | | | | |
| 42 | 33 | | | | | | | | |
| 43 | 34 | | | | | | | | |
| 44 | 35 | | | | | | | | |
| 45 | 36 | | | | | | | | |
| 50 | 41 | | | | | | | | |
| 51 | 42 | | | | | | | | |
| Dyn | Dyn | | | | | | | | |
| (4) | (3) | | | | | | | | |

### 3.1 Overview

The enediyne antitumor antibiotics have remarkable structures and exhibit potent DNA cleavage properties that have inspired growing interest as cancer therapeutics. Their complex structures and high reactivity, however, pose formidable challenges to their production and development in the clinic. We report here proof-of-concept studies using a mutasynthesis strategy to combine chemical synthesis of selected modifications to a key iodoanthracene-g-thiolactone intermediate in dynemicin A biosynthesis with complementation of a mutant bacterial producer incapable of synthesizing this essential building block. In the absence of competition from native production of this intermediate, the most efficient utilization of these externally-supplied structural analogues for precursor-directed biosynthesis becomes possible. To achieve this goal, we describe the required Δorf15 blocked mutant and a general synthetic route to a library of iodoanthracene structural variants. Their successful incorporation opens the door to enhancing DNA binding and tuning the bioreductive activation of the product enediynes for DNA cleavage.

### 3.2 Background

The enediyne anticancer antibiotics are derived from bacterial sources and exhibit some of the greatest cytotoxicity observed among natural products. As a consequence, they rapidly stimulated interest as cancer chemotherapeutics. Soon after the establishment of their unprecedented molecular architecture and exceptional modes of action, Borders and Doyle, 1995; Goldberg, 1999; Lee et al., 1987a; Lee et al., 1987b; Konishi et al., 1990; Kim et al., 1979; De Voss et al., 1990; Adhikari et al., 2020, they quickly moved to center stage eliciting extensive synthetic, mechanistic and biomedical research. Most of the known enediynes exhibit rapid and much higher activity against cancer cells relative to the commonly used anticancer drugs such as Adriamycin. Zhen et al., 1989. Indiscriminate cytotoxicity, however, has limited their clinical application. When linked to proper delivery systems, however, enediynes have shown high potential as chemotherapeutic agents. Sievers and Senter, 2013; Shao, 2010.

Two of the 14 known enediynes are currently on the FDA-approved drug list for cancer treatment, with calicheamicin-based Mylotarg 1 (FIG. 28a) (CMA-676, gemtuzumab ozogamicin) being the first-in-class antibody-drug conjugate (ADC) approved by the FDA for the treatment of first relapse of CD33-positive acute myeloid leukemia. Hamann et al., 2002; Norsworthy et al., 2018; Appelbaum and Bernstein, 2017; Fenwarth et al., 2020. More recently in 2017 the FDA approved another calicheamicin-based ADC with the CD22 mAb, as a first-in-class medication for CD22-positive B-cell acute lymphoblastic leukemia (CMC-544, Inotuzumab ozogamicin). Li et al., 2021; De Vries et al., 2012; Takeshita et al., 2009; Wynne et al., 2019. Several other calicheamicin-, neocarzinostatin- and C-1027-based ADCs are under development and in clinical trials with promising outcomes. Shao, 2010; Maeda, 2001; Tsuchiya et al., 2000; Meada and Konno, 1997; Beerman et al., 2009; Zhen et al., 2009; Kennedy et al., 2007.

Of the three principal classes of enediynes that have already shown clinical promise (1-3, FIG. 28a), Adhikari et al., 2021; Nicolaou et al., 2021, the anthraquinone-fused enediynes (AFEs) are recognized and explored here in an especially attractive application of precursor-directed biosynthetic methods to prepare derivatives of dynemicin A (DYN, 4) and other AFEs bearing rationally modified anthraquinones (6-8, FIG. 28a). The anthraquinone of AFEs both intercalates into DNA to confer a key helix-binding element and serves as the site of bioreductive activation in the cell to initiate a cascade of reactions that result in DNA scission. Simple modifications to the anthraquinone A-ring have been demonstrated to enhance cytotoxicity relative to unmodified controls. Nicolaou et al., 2016; 2020. More subtly it is known that the redox potential of anthraquinones can be altered by substituent effects (e.g. electron-withdrawing or -donating). Schwan et al., 2020. That is, in addition to improved DNA-binding effects on account of added substituents, it should be possible to tune the bioreduction, hence in vivo activation of AFEs, for DNA cleavage to optimize physical and cytotoxic properties.

Although the landmark task of synthesizing the AFEs and their analogues has already been achieved for some members of this family, the long and complex total syntheses of these large and intricate molecules in enantiopure form poses a formidable challenge to generate a library of analogues for SAR studies and clinical use. With the goal to prepare practicable quantities of one or more AFEs, precursor-directed fermentation methods, Weissman, 2007; Kennedy, 2008; Claridge, 1983, are recognized here in the case of the AFEs to be especially promising. Examples of this strategy, Wang et al., 2011; Galm et al., 2004; Weist et al., 2004; Moss et al., 2006; Boddy et al., 2004, illustrate the combined power of flexible and scalable organic synthesis, the complex synthetic capabilities of biosynthetic enzymes and precision fermentation technology. The biosynthesis of the AFEs has been explored in the greatest detail for DYN (4), Cohen and Townsend, 2018; Cohen and Townsend, 2018; Pal et al., 2023a; Pal et al., 2023; Ma et al., 2021; Gui et al., 2022; Bhardwaj et al., 2023, but basic observations have been made for DYN that apply to all members. At the heart of every AFE biosynthetic pathway are highly-reducing polyketide synthases that are strongly conserved (FIG. 28b) and produce a common C₁₅ heptaene 9 (Scheme 1). Horsman et al., 2010; Belecki and Townsend, 2013.

Among the known enediyne natural products, the AFEs are unique for utilizing 9 to synthesize both the iodoanthracene 10 bearing a fused g-thiolactone ring (anthracene branch, Scheme 1) and an enediyne precursor 11 thought to contain an amine, but still of unknown structure. Pal et al., 2023a. The available evidence suggests that these two building blocks are coupled to form an aryl C—N bond. Pal et al., 2023a. The heterodimerization product 12 then proceeds through several more steps to the individual members of the AFEs. Pal et al., 2023b.

Shared by all of these pathways is the iodoanthracene 10, which has been demonstrated to be the direct precursor of the anthraquinone core, Cohen and Townsend, 2018b, and can undergo further tailoring reactions by pathway cytochrome P450 (CYP) oxidations of the A-ring observed in the final structures DYN, tiancimycin (TNM) uncialamycin (UCM) and yangpumicin (YPM) (FIG. 28a). Cohen and Townsend, 2018a; Yang et al., 2022.

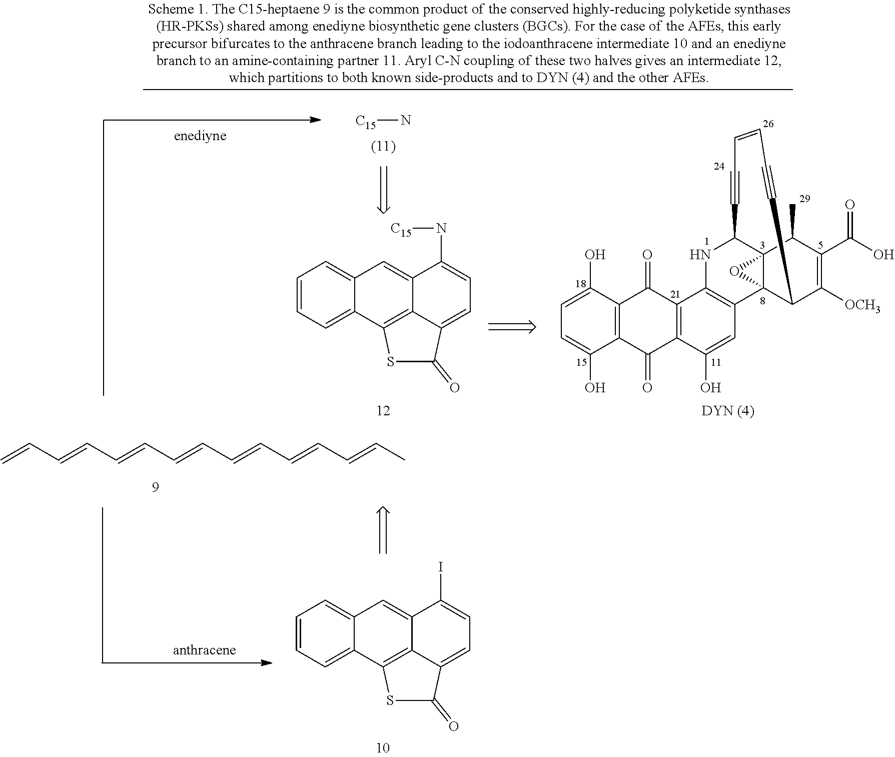

To achieve a proof-of-concept set of experiments, the mutasynthetic creation of a small illustrative library of DYN derivatives variously modified in the A-ring was envisioned. We report here the discovery and implementation of a CRISPR-cas9 deletion mutant in the DYN producer, Micromonospora chersina, specifically in the anthracene arm of the DYN biosynthetic pathway (Scheme 1). AFE production was completely abolished but could be fully restored by the addition of the on-pathway intermediate, iodoanthracene 10, to the fermentation medium. Thus, the optimal condition for mutasynthesis. Weissman, 2007; Kennedy, 2008, was met to synthesize A-ring modified derivatives of iodoanthracene 10 to chemically complement the mutant, enabling precursor-directed biosynthesis of DYN structural analogues in the absence of competition from natively-produced iodoanthracene 10 and, therefore, the production of any unmodified DYN.

### 3.3 Results and Discussion

### 3.3.1 Identification of Orf15 as Key to the Mutasynthetic Approach

In our broader quest to gain insight into the biosynthetic steps upstream of the common intermediate iodoanthracene 10, we looked for conserved genes across all the known AFE biosynthetic gene clusters (BGCs). Among several genes targeted for genetic manipulation, orf15, which encodes a protein of unknown function and is well conserved in the uncialamycin 6 (UcmF, 78% ID), tiancimycin 7 (TnmF, 80% ID) and yangpumicin 8 (YpmF, 92% ID) BGCs (FIG. 28b), but not in calicheamicin, proved to be the most useful deletion for our purpose. Removal of orf15 from the M. chersina wild-type strain using CRISPR-Cas9 halted the production of both DYN and iodoanthracene 10. Of central importance, when synthetic iodoanthracene was added to a culture of Δorf15, DYN production fully resumed, indicating that orf15 is directly or indirectly involved in the early/middle steps of iodoanthracene biosynthesis from the common HR-PKS intermediate 9. This observation presented the opportunity to incorporate modified iodoanthracenes into Δorf15 fermentations to generate DYN structural analogues.

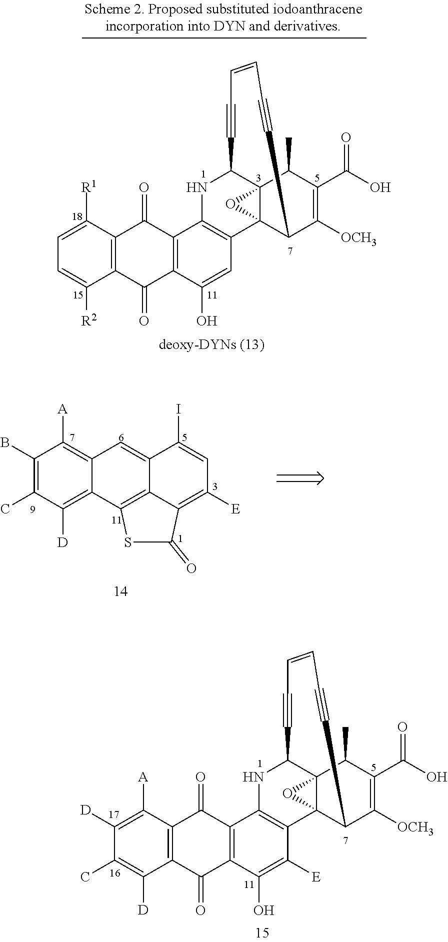

It was known from earlier work that two cytochromes P450 (CYP) encoded in the DYN BGC specifically introduce the two hydroxy groups in the A-ring of DYN in late-stage tailoring steps; Orf18 gives the C18 oxidation and DynE10 hydroxylates C15. Cohen and Townsend, 2018a. A CRISPR-cas9 double knockout of these two genes yields the simplified dideoxy-DYN 13 in unchanged titer from wild-type production of DYN. Cohen and Townsend, 2018a.

One can visualize any number or combination of substituents A-E constituting a library of iodoanthracenes 14 that could potentially incorporate into DYN derivatives 15 using this double knockout mutant in conjunction with Δorf15 (Scheme 2). In opposition to such an ambitious plan, almost all of the biosynthetic transformations that connect the iodoanthracene 10 to DYN or the steric and binding effects that substituents A-E might have on these multiple steps, were unknown. Pal et al., 2023b.

We elected a more circumscribed path to initiate our studies and focused on substitution at position C16 (in 15 or C9 in 14, substituent C, Scheme 2) in a set of demonstrative experiments. This site had been selected in a previous investigation to attach a carboxylic acid to link in an amide spacer to resin beads for the purpose of a classical pull-down experiment of biosynthetic proteins(s) involved in coupling the iodoanthracene 10 to the N-C15 enediyne half 11 (Scheme 1), a heterodimerization process that lies at the center of all AFE biosynthetic pathways. Pal et al., 2023a.

Further encouragement for functionalization at this locus derives from the extensive efforts of the Nicolaou group to synthesize structural analogues of tiancimycin 7 and yangpumicin 8 bearing aminomethyl (NH₂CH₂—) and methylaminomethyl (CH₃NHCH₂—) groups at the carbon corresponding to C16 in DYN. Nicolaou et al., 2016; Nicolaou et al., 2020. These simple structural alterations under physiological conditions place a positively charged substituent on the periphery of the A-ring that leads to enhanced cytotoxicity. Nicolaou et al., 2021; Nicolaou et al., 2020. In addition to an amine substituent at C16, published work with related AFEs also demonstrated that an adjacent fluorine atom at C17 further enhanced cytotoxicity to a small but reproducible extent across all cancer cell lines tested. Nicolaou et al., 2016. We elected to begin our exploration of the viability of a mutasynthetic strategy for the preparation of DYN analogues by targeting the introduction of fluorine owing to its small size, favorable effect on cytotoxicity and absence of competing in vivo reactivity.

### 3.3.2 Synthesis of 9-Substituted Iodoanthracenes

To achieve the synthesis of 9-substituted-5-iodoanthracene-g-thiolactones 16a-d, our previously developed synthesis of the on-pathway intermediate 10, Pal et al., 2023a, was adapted as summarized in Scheme 2 and only required simple modification of the phthalide starting material. Synthesis of 5-substituted phthalides that are not commercially available was achieved starting from 4-substituted benzoic acids 17a-d and converting them to the corresponding diisopropylbenzamides 18a-d. Bisht et al., 2018.

Diisopropylbenzamides are known to direct ortho-lithiation upon reaction with alkyllithium reagents by virtue of their inductive withdrawal and ability to coordinate the metal counterion. Therefore, treatment of the benzamides with n-BuLi and quenching the reaction with DMF introduces an aldehyde ortho to the amide, which, owing to the symmetry of the starting amides, yields a single product 19a-d. Subsequent reduction of the aldehyde and acid-catalyzed lactonization produces the desired 5-substituted phthalides 20a-d (Scheme 3). Faigl et al., 2010.

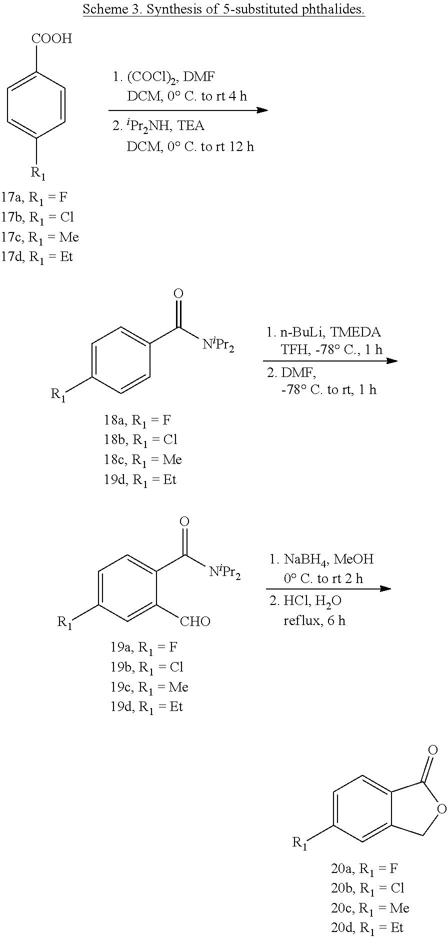

Construction of the anthraquinone moiety was achieved by reacting the phthalide 20a-d with 2-bromoanisole 21 in the presence of lithium diisopropylamide, which generates the phthalide anion as well as a benzyne from the 2-bromoanisole. The reaction of the phthalide anion with the benzyne results in a single regioisomer of the anthraquinone 22a-d by virtue of the inductive effect of the methoxy group. Rather than reducing the anthraquinone entirely to the anthracene, we took advantage of the peri-effect of the 1-methoxy group to partially reduce the anthraquinone to anthrone 23a-d by selectively reducing only the adjacent carbonyl. Treatment of the anthrone 23a-d with Lawesson's reagent generated the readily oxidized B-ring thiol 24a-d after tautomerization of the initially formed thioketone. The thiol, upon reaction with triphosgene and subsequent aluminum chloride-mediated Friedel-Crafts cyclization, created the g-thiolactone 25a-d, guided by the activating effect of the C-ring methoxy group. The iodine was introduced as the final modification by converting the methoxy to triflate 27a-d, followed by reaction of the aryl triflate with sodium iodide in the presence of catalytic Ru[Cp*(MeCN)₃]OTf to generate the desired 9-substituted-5-iodoanthracene-g-thiolactone products 16a-d (Scheme 4). See also FIG. 22.

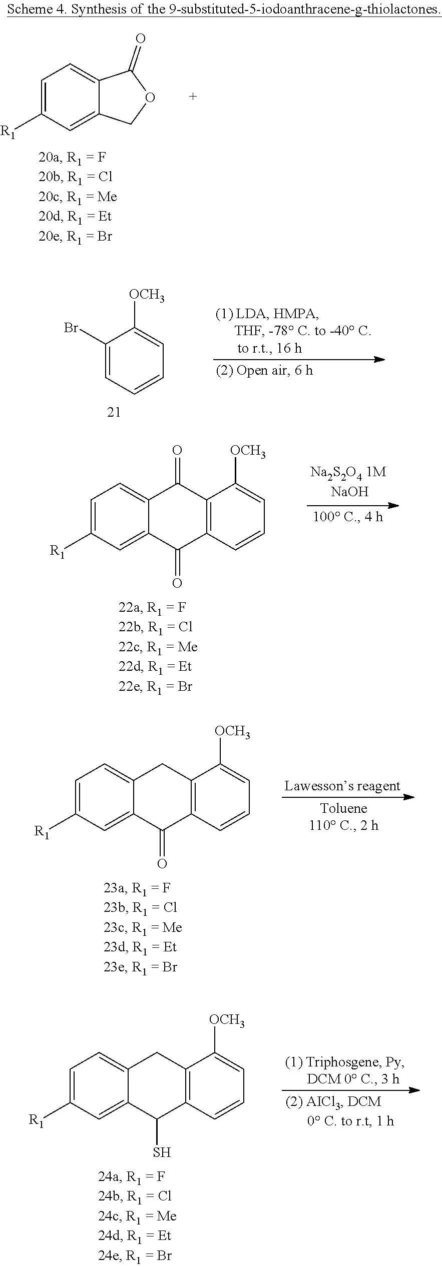

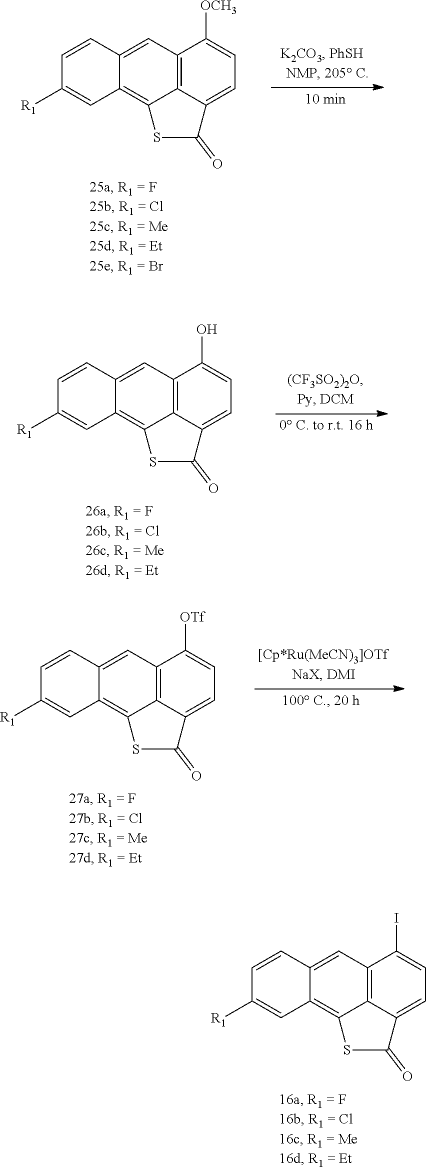

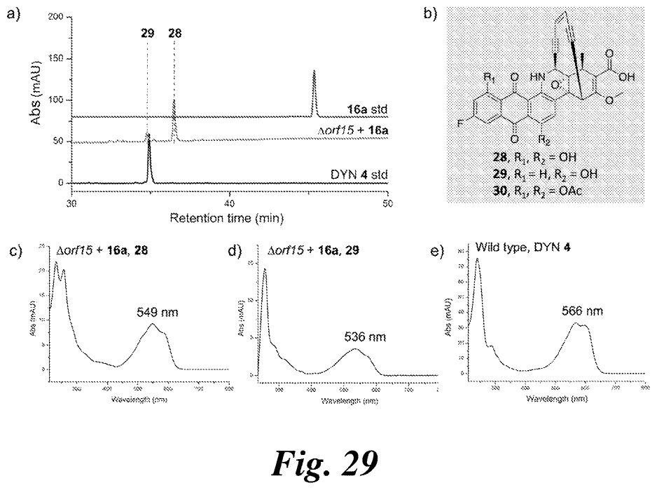

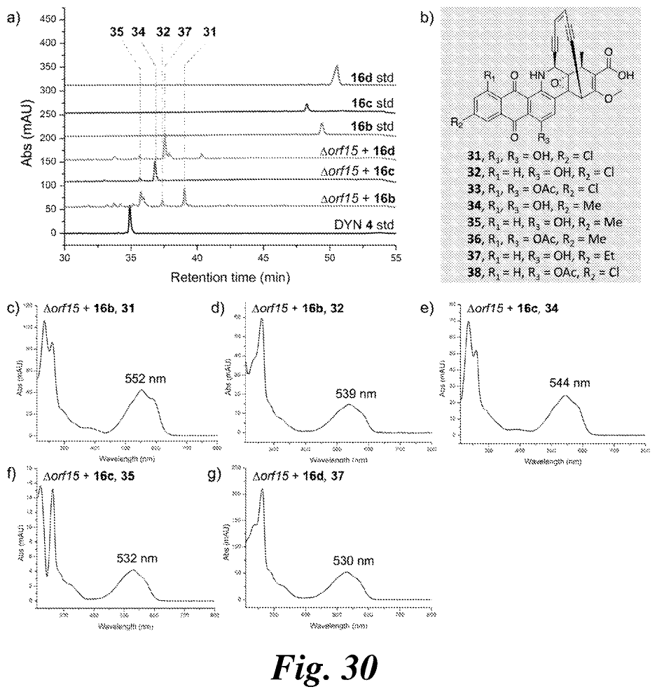

As outlined in Schemes 3 and 4, 9-fluoro-5-iodoanthracene-g-thiolactone 16a was obtained in good overall yield. To our delight, incorporation in the Δorf15 strain produced one major and one minor product, 28 and 29, respectively, each with a different HPLC-retention time but with highly similar UV-vis spectra compared to DYN itself (AFEs can be easily detected by their characteristic UV-vis absorption behavior with maxima in the 500-600 nm range) (FIG. 30c). The major product 28 was consistent with the mass of the desired F-analog as determined by high-resolution ESI-MS, with only two hydroxylations, and the minor 29 with no hydroxyl group in the A-ring (compared to DYN (4), which has three total aryl hydroxy groups). To characterize the new metabolites, 16a was incorporated into larger scale 12×1 L fermentations of Δorf15 enriched with sodium [1-¹³C]- and [2-¹³C]-acetate to improve ¹³C NMR sensitivity for the non-anthraquinone portions of the fermentation products (1-2% ¹³C-enrichment/site over natural abundance). The products were isolated and acetylated following the standard DYN purification protocol. Cohen and Townsend, 2018a. Whereas the major deshydroxy DYN analogue 28 was successfully purified and characterized as its diacetylated derivative 30 by NMR spectroscopy, NMR analysis of the minor desdihydroxy DYN analogue 29 was not possible owing to its very low production, but its structure could be ascertained by its nearly identical UV-vis signature in comparison (FIG. 29d) to dideoxy-DYN (Scheme 2, 13, R₁=R₂=H, lₘₐₓ=532 nm) and exact mass (Table 2). Analogous UV-vis and MS behavior was seen for 28 and 13.

The ¹H-NMR spectrum of 30 was very similar to tri-O-acetylated DYN (5) (FIG. 28a, Table 3), with all characteristic hydrogen resonances in both compounds almost overlapping with each other, except the A-ring hydrogens, as expected. The singlet at 8.02 ppm in 30 is consistent with the C-ring hydrogen (H10) of triacetylated DYN (5) at 7.99 ppm, confirming the presence of C-ring hydroxylation in 30. The apparent singlet at d7.17 (ABq, J=8.8 Hz, 2H) corresponding to the two nearly isochronous ortho-hydrogens on the A-ring of triacetylated DYN (5) was missing from the spectrum of 30. Instead, two sets of d×d at 7.77 ppm (J=2.7, ³J_{H-F}=8.5 Hz) and 7.64 ppm (J=2.7, ³J_{H-F}=8.8 Hz), each corresponding to a single hydrogen flanking a fluorine, indicated a different substitution pattern on the A-ring of 30 compared to triacetylated DYN (5). This splitting pattern with similar ³J values can only be satisfied by placing the second hydroxyl group at C18 instead of C15. Therefore, we can conclude the final structures of 28 and 29 are as shown in FIG. 29b. A-ring hydroxylations are late-stage phenomena catalyzed separately by two CYPs, DynE10 and Orf19, and reactions that can be affected by the bulk and electronic properties of the substituent on the A-ring. Moreover, oxidation by DynE10 is known to be less favorable than Orf19, which is completely suppressed here. Cohen and Townsend, 2018a.

### 3.3.3 Production of C16-Methyl, -Chloro—and -Ethyl Analogues of DYN

Encouraged by the success of our initial experiment, we wanted to test the tolerance of the biosynthetic system for increased steric bulk of the A-ring substituent and the effect on Cyp hydroxylation by electron withdrawal or donation. For this purpose, we synthesized 9-chloro and 9-methyl-5-iodoanthracene-g-thiolactones 16b and 16c starting from 4-chloro and 4-methyl benzoic acids 17b and 17c, respectively. Supplementation of Δorf15 M. chersina fermentations with 16b and 16c resulted in similar observations, where now deshydroxy DYN analogues 31 and 34, respectively, were the major products accompanied by lesser amounts of desdihydroxy DYN analogues 32 and 35, respectively (FIG. 30). The major deshydroxy methyl- and chloro-analogues of DYN 31 and 34 were isolated and purified as their diacetylated derivatives 33 and 36, respectively, from each of 12×1 L fermentations also enriched with ¹³C-labelled acetate following the same procedure as above and characterization using NMR spectroscopy. Next, we attempted to increase the steric bulk from methyl- or chloro- to an ethyl substituent. Following the same synthetic procedures and starting from 4-ethyl benzoic acid 17d, 9-methyl-5-iodoanthracene-g-thiolactone 16d was synthesized. Incorporation of 16d in the Δorf15 fermentation, on the other hand, produced only the desdihydroxy ethyl-analogue of DYN 37 as the major product, confirmed by the LC-MS and the presence of 4-aromatic hydrogens and single acetyl group in the ¹H-NMR of the purified acetylated derivative 38. Table 3). Now the increased bulk of the added substituent suppressed both E10 and orf19 hydroxylation, but not the remainder of the biosynthetic pathway.

3.3.4 Diversification of the Synthetic Route to Access More 9-Substituted Iodoanthracenes from a Common Synthetic Intermediate

With the goal to generate an analogue library, it is always desirable to have a divergent synthesis rather than a linear, even if a streamlined one, where a substituent on a common advanced intermediate can be converted to various other functional groups to efficiently expand the library. It would be ideal to have a functional group that can tolerate the reactions involved in the general synthesis, as well as giving access to modified groups. Amines are often a preferred substituent for this purpose, but their high reactivity also interferes with many reactions. Our initial attempt to synthesize the protected 6-amino-1-methoxyanthraquinone-g-thiolactone starting from protected 5-aminophthalide resulted in a very low yield of the desired product. On the other hand, commercially available 5-bromophthalide 20e was more successful. Synthesis of the 6-bromo-1-methoxyanthraquinone 22e was achieved is good yield from 5-bromophthalide 20e and 2-bromoanisole 21. Reduction of the anthraquinone 22e to anthrone 23e required some optimization owing to dehalogenation in the presence of higher equivalents of reductant or prolonged reaction time. Nevertheless, 9-bromo-5-methoxyanthracene 25e was synthesized in good overall yield (Scheme 4). Pal et al., 2023a. Compound 25e could not be converted to the final 9-bromo-5-iodoanthracene-g-thiolactone, however, because of the lack of chemical discrimination between bromine and the O-triflate group in the last step of the ruthenium-catalyzed iodination. This difficulty was circumvented as follows.

After construction of the basic anthracene-g-thiolactone skeleton, we attempted to convert the bromine to other functional groups. Reaction of 25e with alkyl lithium reagents to perform lithium-halogen exchange followed by quenching with various electrophiles to access carbonyl or alkyl functionality was not fruitful. The bromine, however, was successfully substituted to alkyne 39 in a Tetrakis(triphenylphosphine)palladium(0)and copper(I) iodide catalyzed reaction with TMS-acetylene. Wang et al., 2014.

Although, the alkyne was not a suitable functional group for the final ruthenium catalyzed iodination reaction, due to higher affinity of the ruthenium for the alkyne than insertion into the C—X bond. The alkyne was eventually hydrated by refluxing in formic acid to the methyl ketone 40. The O-methyl group of 40 was then converted to iodine to generate the iodoanthracene-g-thiolactone 43 with a methyl ketone substituent at C9 (Scheme 5).

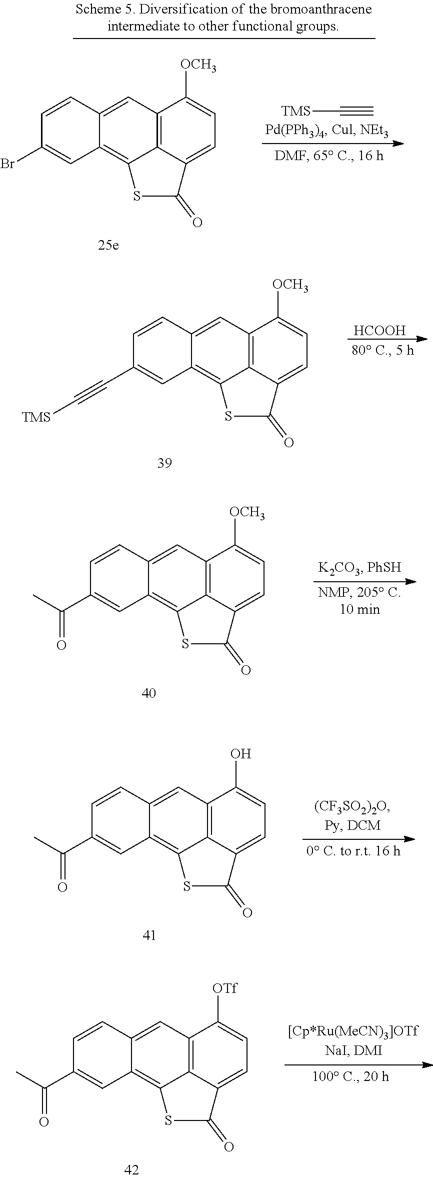

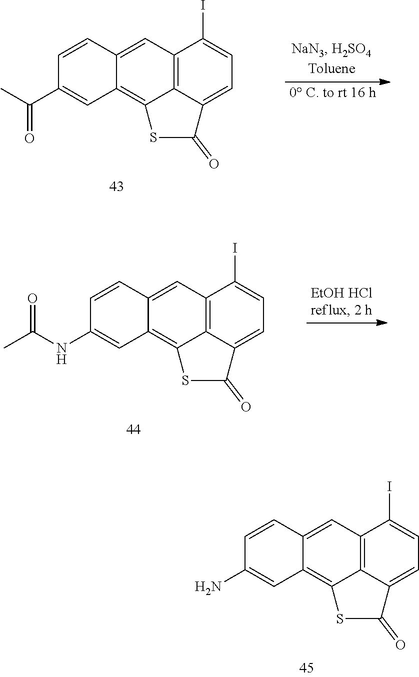 [SMILES: CC(=O)c1ccc2cc3c(I)ccc4c3c(c2c1)SC4=O ; CC(=O)Nc1ccc2cc3c(I)ccc4c3c(c2c1)SC4=O ; Nc1ccc2cc3c(I)ccc4c3c(c2c1)SC4=O]

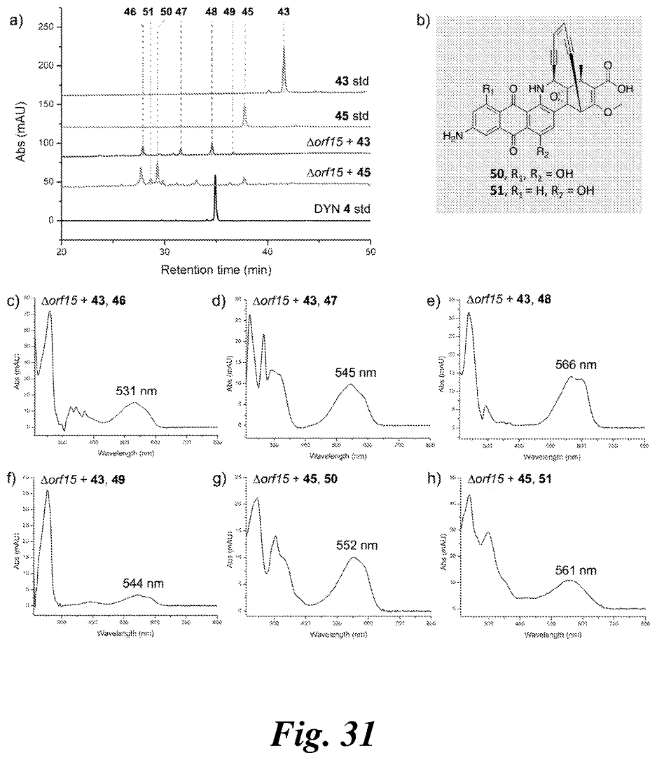

Administration of 43 to the Δorf15 fermentation did not produce the desired outcome. Instead, a multitude of very small peaks (46-49) was observed by HPLC (FIG. 31). Analysis of the peaks by LC-MS confirmed no production of a DYN analogue. The masses of the peaks and the UV-vis spectra (with absorption maxima in the 500-600 nm range) were indicative of probable heterodimerized products (i.e., after the coupling of the anthracene and the enediyne halves). Due to very small production of the metabolites, isolation, and purification to determine their structures was not possible. Nevertheless, this result demonstrates that the biosynthetic machinery can tolerate some steric bulk at C9.

With the indication that substituents larger than two carbons probably are not tolerated by the mutasynthetic approach, we focused on repurposing the methyl ketone to generate other smaller substituents. Schmidt reaction of the methyl ketone of 43 in presence of sodium azide and catalytic sulfuric acid resulted in nitrogen insertion between the carbonyl and the aromatic ring, producing the N-acetylated amine 44. Removal of the acetyl protecting group in refluxing HCl-ethanol solution provided the 9-amino-5-iodoanthracene-g-thiolactone 45 (Scheme 4). Incorporation of 45 in a Δorf15 fermentation was again successful, as hoped, producing deshydroxy amino-DYN analogue 50 as the major product, accompanied by the low production of the desdihydroxy DYN analogue 51 (FIG. 31). Unfortunately, in spite of multiple attempts, the final DYN analogues could not be characterized by NMR spectroscopy owing to the oxidatively unstable nature of the 9-amino-5-iodoanthracene-g-thiolactone 45 precursor and corresponding DYN analogue 50/51 as demonstrated by the observation of higher molecular weight species by LC-MS over time formed presumably via radical polymerization.

### 3.4 Summary

The enediynes calicheamicin and more recently the anthraquinone-fused enediynes (AFEs) have taken their place among important ADC developments for cancer chemotherapy. The pivotal role of iodoanthracene-g-thiolactone (10) as a common intermediate to all AFE biosynthetic pathways opened the way to precursor-directed strategies for the preparation of DYN and other AFE structural analogues by fermentation. With the discovery of the Δorf15 deletion mutant specific to the anthracene arm of the overall biosynthetic pathway, generation of this essential intermediate could be blocked and mutasynthesis successfully demonstrated for a set of synthetic iodoanthracene-g-thiolactones modified at C9 in the A-ring. Two features of the last half of the DYN biosynthetic pathway could be examined. First, tolerance for the steric and electronic properties of anthracene A-ring substituents could be evaluated, and second, the ability of two regiospecific CYPs to carry out late-stage A-ring hydroxylations could be monitored. Such late-stage oxidations are common to all known AFEs but only directly affect the A-ring. Based on the sum of our limited observations above, generalizations about the combined effects of the steric and electronic properties of anthracene C9 substituents on the largely uncharacterized enzymic steps to the final DYN biosynthetic products can be only provisionally made. Steric effects, whether the substituent is electron-donating (—NH₂, -Me or -Et) or. electron-withdrawing (—F, —Cl), E10 CYP oxidation at C15 of the anthraquinone-containing product is essentially blocked while Orf19 hydroxylation at C18 by Orf19 proceeds nearly to completion. When the size of this substituent is three carbons, e.g. 43, (and presumably larger), at least some late transformations in the biosynthesis appear to be derailed and synthesis of DYN analogues is suppressed. Interestingly, both weak electron donation (-Me, -Et) and intermediate electron withdrawal (—F, —Cl) affect overall DYN analogue yield by less than a factor of two owing mostly to run-to-run experimental deviations. Therefore, all subsequent biosynthetic transformations occurring distant from the A-ring, still take place and the downstream biosynthetic steps remain intact to DYN products, which is highly encouraging. Clearly there are many A-ring substituents and combinations of them that remain to be explored as proposed in FIG. 29. The proof-of-concept experiments reported here illustrate the power of partnering organic synthesis of highly functionalized iodoanthracene-g-thiolactones with the prowess and substrate tolerance of biosynthetic enzymes to assemble highly valuable, structurally complex products in a programmable manner.

### 3.5 Experimental Section

3.5.1 Fermentation and Supplementation of M. chersina ΔOrf15

CRISPR-Cas9 mediated deletion of orf15 was conducted as previously described Cohen and Townsend, 2018a; Kieser et al., 2000. Samples of Δorf15 lawns cut from medium 53 xx agar were used to inoculate 50 mL liquid medium 53 cultures. Lam et al., 1995. The cultures were shaken at 250 rpm., 28° C. for seven days. Then, samples (2 mL/50 mL, 25 mL/1 L fermentations) from these vegetative cultures were used to inoculate H881 medium, Lam et al., 1995, for fermentation. NaI was excluded from media for certain control fermentations. Supplementation using 2 mM stock solutions of the iodoanthracene analogues in DMSO (sterile filtered using 0.2 μm PTFE filters) were added to the respective flasks to provide a final concentration of 1 μM 24 h after inoculation. After shaking at 250 rpm and 28° C. for another 2 days, Diaion® HP-20 (0.50 g/L, sterilized by autoclave) was added to each flask and shaking was continued for a total of 7-10 days.

3.5.2 Purification of Metabolites from the ΔOrf15 Mutant Supplemented with Synthetic Iodoanthracenes

Metabolites were extracted on the tenth day of fermentation using 3:1 ratio of broth:EtOAc followed by centrifugation to pellet insoluble cell debris. The supernatant was then filtered and separated into organic and aqueous phases. After the first extraction, secondary washes were done with both the aqueous layer and organic layer with EtOAc and brine, respectively. The organic layers were then filtered through a bed of Celite to remove any residual Diaion® HP-20 resin or particulates. The organic layers were dried over anhydrous Na₂SO₄ and filtered. The EtOAc was removed in vacuo and the residue was then transferred to a tared vial and frozen at −80° C.

The concentrated extract from 12×1 L fermentations supplemented with synthetic compounds was adsorbed to Celite and purified by silica gel flash chromatography using a 0-10% methanol in CHCl₃ gradient. The dark blue band was collected and acetylated using excess acetic anhydride and pyridine (1:1 molar equivalents) at room temperature for 16 h. After completion, the reaction mixture was diluted with EtOAc and washed twice with water and once with sat. NH₄Cl solution. The EtOAc layer was dried over anhydrous Na₂SO₄ and concentrated in vacuo.

The crude acetylated products were further purified by silica gel chromatography using a gradient of 80:20 EtOAc:hexane to 100% EtOAc to 95:5 EtOAc:MeOH. The pink-orange material that eluted at 95:5 EtOAc:MeOH was collected and concentrated in vacuo. The acetylated compound was further purified using a Sephadex LH-20 (GE Healthcare Life Sciences) resin column pre-equilibrated with 1:1 CHCl₃:MeOH. The compound was loaded dissolved in the same solvent system and the pink-orange band was collected. The compound was re-purified over the same column and the MeOH was removed in vacuo to yield the acetylated Dyn analogues.

### 3.5.3 Synthesis of Example 3 Compounds 33 and 38

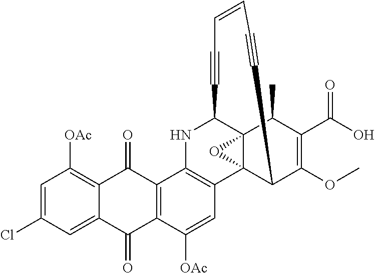 [SMILES: COC1=C(C(=O)O)[C@H](C)[C@@]23O[C@]24c2cc(OC(C)=O)c5c(c2N[C@H]3C#C/C=C\C#C[C@H]14)C(=O)c1c(OC(C)=O)cc(Cl)cc1C5=O]

16-Chloro-deshydroxy dynemicin diacetate (33). The compound was isolated, acetylated and purified from 12 L fermentation. Pure diacetylated compound (3.3 mg) was isolated as an orange solid. ¹H NMR (600 MHz DMSO-d₆) δ ppm 9.77 (d, J=4.4 Hz, 1H), 8.05 (s, 1H), 7.97 (d, J=2.2 Hz, 1H), 7.82 (d, J=2.2 Hz, 1H), 6.06 (dq, J=9.9, 1.5 Hz, 2H), 5.06 (d, J=4.4 Hz, 1H), 4.67 (s, 1H), 3.77 (s, 3H), 3.54 (q, J=7.1 Hz, 1H), 2.45 (s, 3H), 2.38 (s, 3H), 1.26 (d, J=7.1 Hz, 3H); ¹³C (150 MHz DMSO-d₆) δ ppm 183.3, 180.3, 169.7, 169.4, 151.1, 145.2, 140.9, 136.4, 132.1, 130.7, 130.3, 130.1, 124.8, 124.6, 124.3, 123.5, 115.3, 100.8, 98.0, 90.1, 89.2, 72.0, 63.6, 58.3, 44.4, 36.4, 32.2, 21.5, 21.4, 19.0; UPLC-ESI-HMS: calculated exact mass for C₃₄H₂₃ClNO₁₀+ [M+H⁺]: 640.1005, found [M+H⁺]: 640.1001.

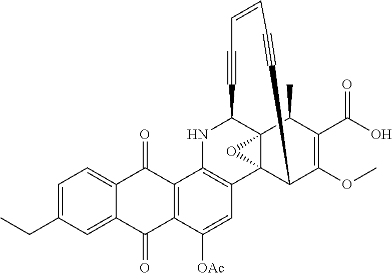 [SMILES: CCc1ccc2c(c1)C(=O)c1c(OC(C)=O)cc3c(c1C2=O)N[C@H]1C#C/C=C\C#C[C@@H]2C(OC)=C(C(=O)O)[C@H](C)[C@]14O[C@@]324]

16-Ethyl-deshydroxy dynemicin monoacetate (38). The compound was isolated, acetylated and purified from 12 L fermentation. Pure monoacetylated compound (4.8 mg) was isolated as an orange solid. ¹H NMR (600 MHz DMSO-d₆) δ ppm 9.99 (d, J=4.3 Hz, 1H), 8.11 (d, J=8.0 Hz, 1H), 8.04 (s, 1H), 7.91 (d, J=1.2 Hz, 1H), 7.76 (dd, J=8.0, 1.4 Hz, 1H), 6.06 (dq, J=10.8, 1.3 Hz, 2H), 5.08 (d, J=4.3 Hz, 1H), 4.71 (s, 1H), 3.78 (s, 3H), 3.54 (q, J=7.2 Hz, 1H), 2.81 (q, J=7.6 Hz, 2H), 2.39 (s, 3H), 1.25 (m, 6H); ¹³C (150 MHz DMSO-d₆) δ ppm 184.9, 182.1, 169.8, 168.3, 151.2, 145.5, 141.2, 134.6, 133.5, 131.8, 131.6, 130.8, 127.2, 125.7, 124.7, 124.3, 114.9, 98.1, 90.0, 89.1, 79.6, 71.8, 63.6, 58.3, 44.4, 36.4, 32.2, 29.5, 28.7, 21.4, 19.0, 15.4; UPLC-ESI-HMS: calculated exact mass for C₃₄H₂₆NO₈+ [M+H⁺]: 576.1653, found [M+H⁺]: 576.1659.

TABLE 8
Comparison of proton chemical shifts of 30, 33, 36 and 38 with triacetyldynemicin (5). Cohen and Townsend, 2018.

| # of C | | | | | |
|---|---|---|---|---|---|
| with H | Dyn (5) | 30 | 33 | 36 | 38 |
| 1 (N—H) | 9.41 (d, 1H) | 9.77 (d, 1H) | 9.77 (d, 1H) | 9.78 (d, 1H) | 9.99 (d, 1H) |
| 2 | 5.00 (d, 1H) | 5.03 (d, 1H) | 5.06 (d, 1H) | 5.08 (dd, 1H) | 5.08 (dd, 1H) |
| 4 | 3.51 (q, 1H) | 3.51 (q, 1H) | 3.54 (q, 1H) | 3.57 (q, 1H) | 3.54 (q, 1H) |
| 7 | 4.57 (s, 1H) | 4.53 (s, 1H) | 4.67 (s, 1H) | 4.82 (s, 1H) | 4.71 (s, 1H) |
| 10 | 7.99 (s, 1H) | 8.02 (s, 1H) | 8.05 (s, 1H) | 8.05 (s, 1H) | 8.04 (s, 1H) |
| 11 | 2.33 (s, 3H) - OAc | 2.38 (s, 3H) - OAc | 2.38 (s, 3H) - OAc | 2.39 (s, 3H) - OAc | 2.39 (s, 3H) - OAc |
| 15 | 2.36 (s, 3H) - OAc | 7.77 (dd, 1H) | 7.97 (d, 1H) | 7.85 (dd, 1H) | 7.91 (d, 1H) |
| 16 | 7.63 (d, 1H) | — | — | 2.48 (s, 3H) | 2.81 (q, 2H) |
| | | | | Me substituent | 1.25 (m, 3H) |
| | | | | | Et substituent |
| 17 | 7.61 (d, 1H) | 7.64 (dd, 1H) | 7.82 (d, 1H) | 7.44 (dd, 1H) | 7.76 (dd, 1H) |
| 18 | 2.44 (s, 3H) - OAc | 2.45 (s, 3H) - OAc | 2.45 (s, 3H) - OAc | 2.44 (s, 3H) - OAc | 8.11 (d, 1H) |
| 25 | 6.03 (dq, 1H) | 6.05 (dq, 1H) | 6.06 (dq, 1H) | 6.08 (dq, 1H) | 6.06 (dq, 1H) |
| 26 | 6.03 (dq, 1H) | 6.05 (dq, 1H) | 6.06 (dq, 1H) | 6.08 (dq, 1H) | 6.06 (dq, 1H) |
| 29 | 1.24 (d, 3H) | 1.25 (d, 3H) | 1.25 (d, 3H) | 1.27 (d, 3H) | 1.25 (m, 3H) |
| 31 | 3.73 (s, 3H) | 3.73 (s, 3H) | 3.77 (s, 3H) | 3.82 (s, 3H) | 3.78 (s, 3H) |

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents form part of the common general knowledge in the art.
Cohen, D. R.; Townsend, C. A. A Dual Role for a Polyketide Synthase in Dynemicin Enediyne and Anthraquinone Biosynthesis. Nat Chem 2018a, 10, 231-236.
Gao, Q.; Thorson, J. S. The Biosynthetic Genes Encoding for the Production of the Dynemicin Enediyne Core in Micromonospora chersina ATCC53710. FEMS Microbiol. Lett. 2008, 282 (1), 105-114.
Cohen, D. R.; Townsend, C. A. C—N-Coupled Metabolites Yield Insights into Dynemicin A Biosynthesis. ChemBioChem 2020, 21 (15), 2137-2142.
Cohen, D. R.; Townsend, C. A. Characterization of an Anthracene Intermediate in Dynemicin Biosynthesis. Angewandte Chemie—International Edition 2018b, 57 (20), 5650-5654.
Neumann, C. S.; Fujimori, D. G.; Walsh, C. T. Halogenation Strategies In Natural Product Biosynthesis. Chem. Biol. 2008, 15 (2), 99-109.
Low, Z. J.; Ma, G. L.; Tran, H. T.; Zou, Y.; Xiong, J.; Pang, L.; Nuryyeva, S.; Ye, H.; Hu, J. F.; Houk, K. N.; Liang, Z. X. Sungeidines from a Non-Canonical Enediyne Biosynthetic Pathway. J. Am. Chem. Soc. 2020, 142 (4), 1673-1679.
Colver, C. W.; Noyes, W. A. Synthesis of Anthracene from Naphthalene. J. Am. Chem. Soc. 1921, 43 (4), 898-905.
Finar, I. Organic Chemistry, Volume 2: Stereochemistry And The Chemistry of Natural Products. Organic Chemistry. 1959, pp 484-541.
Khanapure, S. P.; Reddy, R. T.; Biehl, E. R. The Preparation of Anthraquinones and Anthracyclinones via the Reaction of Haloarenes and Cyanophthalides under Aryne-Forming Conditions. J. Org. Chem. 1987, 52 (26), 5685-5690.
Prinz, H.; Wiegrebe, W.; Müller, K. Syntheses of Anthraeenones. 1. Sodium Dithionite Reduction of Peri-Substituted Anthracenediones. J. Org. Chem. 1996, 61 (8), 2853-2856.
Conradt, D.; Schatzle, M. A.; Haas, J.; Townsend, C. A.; Müller, M. New Insights into the Conversion of Versicolorin A in the Biosynthesis of Aflatoxin B1. J. Am. Chem. Soc. 2015, 137 (34), 10867-10869.
Rogers, C.; Chen, C.; Pedramrazi, Z.; Omrani, A. A.; Tsai, H. Z.; Jung, H. S.; Lin, S.; Crommie, M. F.; Fischer, F. R. Closing the Nanographene Gap: Surface-Assisted Synthesis of Peripentacene from 6,6′-Bipentacene Precursors. Angewandte Chemie—International Edition 2015, 54 (50), 15143-15146.
Mitsudo, K.; Murakami, T.; Shibasaki, T.; Inada, T.; Mandai, H.; Ota, H.; Suga, S. Facile Synthesis of Naphthothiophenone Derivatives and Anthra-Dithiophenedione via Friedel-Crafts Acylation and Their Fundamental Properties. Synlett 2016, 27 (16), 2327-2332.
Imazaki, Y.; Shirakawa, E.; Ueno, R.; Hayashi, T. Ruthenium-Catalyzed Transformation of Aryl and Alkenyl Triflates to Halides. J. Am. Chem. Soc. 2012, 134 (36), 14760-14763.
Org Ziegler, J.; Facchini, P. J. Alkaloid Biosynthesis: Metabolism and Trafficking. Annu Rev Plant Biol 2008, 59, 735-769.
Lichman, B. R. The Scaffold-Forming Steps of Plant Alkaloid Biosynthesis. Nat Prod Rep 2021, 38 (1), 103-129.
Wang, B.; Chai, X.; Zhu, W.; Wang, T.; Wu, Q. A General Approach to Spirolactonized Si-Rhodamines. Chem. Commun. 2014, 50 (92), 14374.
Xu, L.; Chen, Y.; Shen, Z.; Wang, Y.; Li, M. I₂/Fe(NO₃)₃·9H₂O-Catalyzed Oxidative Synthesis of Aryl Carboxylic Acids from Aryl Alkyl Ketones and Secondary Benzylic Alcohols. Tetrahedron Lett. 2018, 59 (49), 4349-4354.
Chakraborti, A. K.; Sharma, L.; Nayak, M. K. Demand-Based Thiolate Anion Generation under Virtually Neutral Conditions: Influence of Steric and Electronic Factors on Chemo- and Regioselective Cleavage of Aryl Alkyl Ethers. J. Org. Chem. 2002, 67 (18), 6406-6414.
Singh, S.; H. Hager, M.; Zhang, C.; R. Griffith, B.; S. Lee, M.; Hallenga, K.; L. Markley, J.; S. Thorson, J. Structural Insight into the Self-Sacrifice Mechanism of Enediyne Resistance. ACS Chem. Biol. 2006, 1 (7), 451-460.
Marchler-Bauer, A.; Bo, Y.; Han, L.; He, J.; Lanczycki, C. J.; Lu, S.; Chitsaz, F.; Derbyshire, M. K.; Geer, R. C.; Gonzales, N. R.; Gwadz, M.; Hurwitz, D. I.; Lu, F.; Marchler, G. H.; Song, J. S.; Thanki, N.; Wang, Z.; Yamashita, R. A.; Zhang, D.; Zheng, C.; Geer, L. Y.; Bryant, S. H. CDD/SPARCLE: Functional Classification of Proteins via Subfamily Domain Architectures. Nucleic Acids Res. 2017, 45 (D1), D200-D203.
Kobe, B.; Gleichmann, T.; Horne, J.; Jennings, I. G.; Scotney, P. D.; the, T. Turn up the HEAT. Structure 1999, 7 (5), R91-R97.
Altschul, S. F.; Madden, T. L.; Schaffer, A. A.; Zhang, J.; Zhang, Z.; Miller, W.; Lipman, D. J. Gapped BLAST and PSI-BLAST: A New Generation of Protein Database Search Programs. Nucleic Acids Res. 1997, 25 (17), 3389-3402.
Jumper, J.; Evans, R.; Pritzel, A.; Green, T.; Figurnov, M.; Ronneberger, O.; Tunyasuvunakool, K.; Bates, R.; Z̆idek, A.; Potapenko, A.; Bridgland, A.; Meyer, C.; Kohl, S. A. A.; Ballard, A. J.; Cowie, A.; Romera-Paredes, B.; Nikolov, S.; Jain, R.; Adler, J.; Back, T.; Petersen, S.; Reiman, D.; Clancy, E.; Zielinski, M.; Steinegger, M.; Pacholska, M.; Berghammer, T.; Bodenstein, S.; Silver, D.; Vinyals, O.; Senior, A. W.; Kavukcuoglu, K.; Kohli, P.; Hassabis, D.; Hassabis, D. Highly Accurate Protein Structure Prediction with AlphaFold. Nature 2021, 596, 583.
Bertoni, M.; Kiefer, F.; Biasini, M.; Bordoli, L.; Schwede, T. Modeling Protein Quaternary Structure of Homo- and Hetero-Oligomers beyond Binary Interactions by Homology. Sci. Rep. 2017, 7 (1), 1-15.
Holm, L.; Ivi Rosenstro, P. Dali Server: Conservation Mapping in 3D. Nucleic Acids Research, Volume 38, Issue suppl_2, 1 Jul. 2010, Pages W545-W549. https://doi.org/10.1093/nar/gkq366.
Caldara-Festin, G.; Jackson, D. R.; Barajas, J. F.; Valentic, T. R.; Patel, A. B.; Aguilar, S.; Nguyen, M. C.; Vo, M.; Khanna, A.; Sasaki, E.; Liu, H. W.; Tsai, S. C.; Smith, J. L. Structural and Functional Analysis of Two Di-Domain Aromatase/Cyclases from Type II Polyketide Synthases. Proc Natl Acad Sci USA 2015, 112 (50), E6844-E6851.
Vaidya, A. S.; Peterson, F. C.; Eckhardt, J.; Xing, Z.; Park, S. Y.; Dejonghe, W.; Takeuchi, J.; Pri-Tal, O.; Faria, J.; Elzinga, D.; Volkman, B. F.; Todoroki, Y.; Mosquna, A.; Okamoto, M.; Cutler, S. R. Click-to-Lead Design of a Picomolar ABA Receptor Antagonist with Potent Activity in Vivo. Proc Natl Acad Sci USA 2021, 118 (38), 1-7.
Sliwiak, J.; Sikorski, M.; Jaskolski, M. PR-10 Proteins as Potential Mediators of Melatonin-Cytokinin Cross-Talk in Plants: Crystallographic Studies of LlPR-10.2B Isoform from Yellow Lupine. FEBS Journal 2018, 285 (10), 1907-1922.
Chen, C. C.; Xue, J.; Peng, W.; Wang, B.; Zhang, L.; Liu, W.; Ko, T. P.; Huang, J. W.; Zhou, S.; Min, J.; Ma, L.; Dai, L.; Guo, R. T.; Yu, X. Structural Insights into Thebaine Synthase 2 Catalysis. Biochem Biophys Res Commun 2020, 529 (2), 156-161.
Arakawa, T.; Sato, Y.; Yamada, M.; Takabe, J.; Moriwaki, Y.; Masamura, N.; Kato, M.; Aoyagi, M.; Kamoi, T.; Terada, T.; Shimizu, K.; Tsuge, N.; Imai, S.; Fushinobu, S. Dissecting the Stereocontrolled Conversion of Short-Lived Sulfenic Acid by Lachrymatory Factor Synthase. ACS Catal. 2020, 10 (1), 9-19.
Neudecker, P.; Schweimer, K.; Nerkamp, J.; Scheurer, S.; Vieths, S.; Sticht, H.; Rösch, P. Allergic Cross-Reactivity Made Visible. Solution Structure of the Major Cherry Allergen Pru Av 1. Journal of Biological Chemistry 2001, 276 (25), 22756-22763.
Elshahawi, S. I.; Ramelot, T. A.; Seetharaman, J.; Chen, J.; Singh, S.; Yang, Y.; Pederson, K.; Kharel, M. K.; Xiao, R.; Lew, S.; Yennamalli, R. M.; Miller, M. D.; Wang, F.; Tong, L.; Montelione, G. T.; Kennedy, M. A.; Bingman, C. A.; Zhu, H.; Phillips, G. N.; Thorson, J. S. Structure-Guided Functional Characterization of Enediyne Self-Sacrifice Resistance Proteins, CalU16 and CalU19. ACS Chem. Biol. 2014, 9 (10), 2347-2358.
Alvarado, S. K.; Miller, M. D.; Bhardwaj, M.; Thorson, J. S.; van Lanen, S. G.; Phillips, G. N. Structural Characterization of DynU16, a START/Bet v1-like Protein Involved in Dynemicin Biosynthesis. Acta Crystallogr F Struct Biol. Commun. 2021, 77, 328-333.
Campanacci, V.; Urvoas, A.; Khodja, L. A.; Aumont-Nicaise, M.; Noiray, M.; Lachkar, S.; Curmi, P. A.; Minard, P.; Gigant, B. Structural Convergence for Tubulin Binding of CPAP and Vinca Domain Microtubule Inhibitors. Proc Natl Acad Sci USA 2022, 119 (19), 1-6.
Tiouajni, M.; Durand, D.; Blondeau, K.; Graille, M.; Urvoas, A.; Valerio-Lepiniec, M.; Guellouz, A.; Aumont-Nicaise, M.; Minard, P.; van Tilbeurgh, H. Structural and Functional Analysis of the Fibronectin-Binding Protein FNE from Streptococcus Equi Spp. Equi. FEBS Journal 2014, 281, 5513-5531.
Xiang, K.; Nagaike, T.; Xiang, S.; Kilic, T.; Beh, M. M.; Manley, J. L.; Tong, L. Crystal Structure of the Human Symplekin-Ssu72-CTD Phosphopeptide Complex. 2010.
Tong, Y.; Charusanti, P.; Zhang, L.; Weber, T.; Lee, S. Y. CRISPR-Cas9 Based Engineering of Actinomycetal Genomes. ACS Synth. Biol. 2015, 4 (9).
Shafir, A.; Buchwald, S. L. Highly Selective Room-Temperature Copper-Catalyzed C—N Coupling Reactions. J Am Chem Soc 2006, 128 (27), 8742-8743.
Kappen, L. S.; Goldberg, I. H. Stabilization of Neocarzinostatin Nonprotein Chromophore Activity by Interaction with Apoprotein and with HeLa Cells. Biochemistry 1980, 19 (21), 4786-4790.
Povirk, L. F.; Goldberg, I. H. Binding of the Nonprotein Chromophore of Neocarzinostatin to Deoxyribonucleic Acid. Biochemistry 1980, 19 (21), 4773-4780.
Liu, W.; Nonaka, K.; Nie, L.; Zhang, J.; Christenson, S. D.; Bae, J.; van Lanen, S. G.; Zazopoulos, E.; Farnet, C. M.; Yang, C. F.; Shen, B. The Neocarzinostatin Biosynthetic Gene Cluster from Streptomyces carzinostaticus ATCC 15944 Involving Two Iterative Type I Polyketide Synthases. Chem. Biol. 2005, 12 (3), 293-302.
Biggins, J. B.; Onwueme, K. C.; Thorson, J. S. Resistance to Enediyne Antitumor Antibiotics by CalC Self-Sacrifice. Science 2003, 301 (5639), 1537-1541.
Kieser, T.; Bibb, M. J.; Buttner, M. J.; Chater, K. F.; Hopwood, D. A. Practical Streptomyces Genetics; John Innes Foundation: Norwich, 2000.
Townsend, C. A.; Davis, S. G.; Christensen, S. B.; Link, J. C.; Lewis, C. P. Methoxymethyl-Directed Aryl Metalation. A Total Synthesis of (±)-Averufin1. J. Am. Chem. Soc. 1981, 103 (23), 6885-6888.
Yu, J.; Zhang, P.; Wu, J.; Shang, Z. Metal-Free C—N Bond-Forming Reaction: Straightforward Synthesis of Anilines, through Cleavage of Aryl C—O Bond and Amide C—N Bond. Tetrahedron Lett. 2013, 54 (24), 3167-3170.
Menashe, N.; Shvo, Y. Hydration of Alkynes in Anhydrous Medium with Formic Acid as Water Donor. J. Org. Chem 1993, 58 (26), 7434-7439.
Altman, R. A.; Buchwald, S. L. 4,7-Dimethoxy-1,10-Phenanthroline: An Excellent Ligand for the Cu-Catalyzed N-Arylation of Imidazoles. Org. Lett. 2006, 8 (13), 2779-2782.
Lam, K. S.; Veitch, J. A.; Forenza, S. Effect of Neutral Resins on the Production of Dynemicins by Micromonospora chersina. J Indust. Micro. 1995, 15, 453-456.
Shevchenko, A.; Wilm, M.; Vorm, O.; Mann, M. Mass Spectrometric Sequencing of Proteins from Silver-Stained Polyacrylamide Gels. Anal. Chem. 1996, 68 (5), 850-858.
Nesvizhskii, A. I.; Keller, A.; Kolker, E.; Aebersold, R. A Statistical Model for Identifying Proteins by Tandem Mass Spectrometry. Anal. Chem. 2003, 75 (17), 4646-4658.
Holm, L. Dali Server: Conservation Mapping in 3D. Nucleic Acids Res 2022, 50, W210-W215. http://ekhidna2.biocenter.helsinki.fi/dali/
Herbst, D. A.; Huitt-Roehl, C. R.; Jakob, R. P.; Kravetz, J. M.; Storm, P. A.; Alley, J. R.; Townsend, C. A.; Maier, T. The Structural Organization of Substrate Loading in Iterative Polyketide Synthases. Nat. Chem. Biol. 2018, 14 (5), 474-479.
Shen, B.; Liu, W.; Nonaka, K. Enediyne Natural Products: Biosynthesis and Prospect Towards Engineering Novel Antitumor Agents. Front. Med. Chem.—Online 2005, 2 (1), 357-369.
Zhen, Y.; Ming, X.; Yu, B.; Otani, T.; Saito, H.; Yamada, Y. A New Macromolecular Antitumor Antibiotic, C-1027. III. Antitumor Activity. J. Anitbiotics 1989, 42, 1294-1298.
Shao, R.-G. Pharmacology and Therapeutic Applications of Enediyne Antitumor Antibiotics. Curr. Mol. Pharmacol. 2010, 1 (1), 50-60.
Meada, H.; Ichimura, H.; Satoh, H.; Ohtsuki, K. Evaluation Of Succinyl Neocarzinostation In Vivo. J. Antibiot. (Tokyo). 1978, 31 (5), 468-472.
Shimoyama, M.; Kimura, K. Effect of Blood, Ascites, and Tumor Cell Density on Cytocidal Action of Neocarzinostatin. Gann. 1979, 70, 165-171.
Kimura, I. Clinical Investigation of Neocarzinostatin in Japan. Recent Results Cancer Res. 1978, 63, 252-260.
Zhen, Y. su; Ming, X. ying; Yu, B.; Otani, T.; Saito, H.; Yamada, Y.; Marunaka, T. A New Macromolecular Antitumor Antibiotic, C-1027. I. Discovery, Taxonomy of Producing Organism, Fermentation and Biological Activity. J. Antibiot. (Tokyo). 1988, 41, 1575-1579.
Lam, K. S.; Hesler, G. A.; Gustavson, D. R.; Crosswell, A. R.; Veitch, J. M.; Forenza, S.; Tomita, K. Kedarcidin, a New Chromoprotein Antitumor Antibiotic: I.
Taxonomy of Producing Organism, Fermentation and Biological Activity. J. Antibiot. (Tokyo). 1991, 44 (5), 472-478.
Leet, J. E.; Schroeder, D. R.; Hofstead, S. J.; Golik, J.; Colson, K. L.; Huang, S.; Klohr, S. E.; Doyle, T. W.; Matson, J. A. Kedarcidin, a New Chromoprotein Antitumor Antibiotic: Structure Elucidation of Kedarcidin Chromophore. J. Am. Chem. Soc 1992, 114, 7946-7948.
Zein, N.; Sinha, A. M.; Mcgahren, W. J.; Ellestad, G. A. Calicheamicin γ1I: An Antitumor Antibiotic That Cleaves Double-Stranded DNA Site Specifically. Science. 1988, 240 (4856), 1198-1201.
Maiese, W. M.; Korshalla, J.; Kuck, N.; Fantini, A. A.; Wildey, M. J.; Thomas, J.; Greenstein, M.; Labeda, D. P. Calicheamicins, A Novel Family of Antitumor Antibiotics: Taxonomy, Fermentation and Biological Properties. J. Antibiot. (Tokyo). 1989, 42, 558-563.
Konishi, M.; Ohkuma, H.; Saitoh, K.; Kawaguchi, H.; Golik, J.; Dubay, G.; Groenewold, G.; Krishnan, B.; Doyle, T. W. Esperamicins, a Novel Class of Potent Antitumor Antibiotics. I. Physicochemical Data and Partial Structure. J. Antibiot. (Tokyo). 1985, 38, 1605-1609.
Batchelder, R.; Wilson, W. R.; Hay, M. P.; Denny, W. A. Oxygen Dependence of the Cytotoxicity of the Enediyne Anti-Tumour Antibiotic Esperamicin A1. Br. J. Cancer 1996, 27, S52-S56.
Konishi, M.; Ohkuma, H.; Matsumoto, K.; Tsuno, T.; Kamei, H.; Miyaki, T.; Oki, T.; Kawaguchi, H.; Vanduyne, G. D.; Clardy, J. Dynemicin A, a Novel Antibiotic with the Anthraquinone and 1,5-Diyn-3-Ene Subunit. J. Antibiot. (Tokyo). 1989, 42 (9), 1449-1452.
Konishi, M.; Ohkuma, H.; Matsumoto, K.; Saitoh, K.; Miyaki, T.; Oki, T.; Kawaguchi, H. Dynemicins, New Antibiotics with the 1,5-Diyn-3-Ene and Anthraquinone Subunit. I. Production, Isolation and Physico-Chemical Properties. J. Antibiot. (Tokyo). 1991, 44, 1300-1305.
Nicolaou, K. C.; Dai, W. M. Chemistry and Biology of the Enediyne Anticancer Antibiotics. Angew. Chemie Int. Ed. English 1991, 30 (11), 1387-1530.
Nicolaou, K. C.; Smith, A. L.; Yue, E. W. Chemistry and Biology of Natural and Designed Enediynes. Proc. Natl. Acad. Sci. U.S.A 1993, 90 (13), 5881-5888.
Jones, R. R.; Bergman, R. G. P-Benzyne. Generation as an Intermediate in a Thermal Isomerization Reaction and Trapping Evidence for the 1,4-Benzenediyl Structure. J. Am. Chem. Soc 1972, 94 (2), 660-661.
Nagata, R.; Yamanaka, H.; Okazaki, E.; Saito, I. Biradical Formation from Acyclic Conjugated Eneyne-Allene System Related to Neocarzinostatin and Esperamicin-Calichemicin. Tetrahedron Lett. 1989, 30 (37), 4995-4998.
Myers, A. G.; Kuo, E. Y.; Finney, N. S.; No, C.; Beckman, M. Thermal Generation of a, 3-Dehydrotoluene from (Z)-1,2,4-Heptatrien-6-Ync. J. Am. Chem. Soc 1989, 111 (20), 8057-8059.
Maeda, H. SMANCS and Polymer-Conjugated Macromolecular Drugs: Advantages in Cancer Chemotherapy. Adv. Drug Deliv. Rev. 2001, 46, 169-185.
Maeda, H. SMANCS/Lipiodol. Gan To Kagaku Ryoho 1994, 21, 907-913.
Hamann, P. R.; Hinman, L. M.; Hollander, I.; Beyer, C. F.; Lindh, D.; Holcomb, R.; Hallett, W.; Tsou, H. R.; Upeslacis, J.; Shochat, D.; Mountain, A.; Flowers, D. A.; Bernstein, I. Gemtuzumab Ozogamicin, a Potent and Selective Anti-CD33 Antibody—Calicheamicin Conjugate for Treatment of Acute Myeloid Leukemia. Bioconjug. Chem. 2002, 13 (1), 47-58.
Boghaert, E. R.; Khandke, K.; Sridharan, L.; Armellino, D.; Dougher, M.; Dijoseph, J. F.; Kunz, A.; Hamann, P. R.; Sridharan, A.; Jones, S.; Discafani, C.; Damle, N. Tumoricidal Effect of Calicheamicin Immuno-Conjugates Using a Passive Targeting Strategy. Int. J. Oncol. 2006, 28 (3), 675-684.
Wynne, J.; Wright, D.; Stock, W. Inotuzumab: From Preclinical Development to Success in B-Cell Acute Lymphoblastic Leukemia. Blood Adv. 2019, 3 (1), 96-104.
Dijoseph, J. F.; Dougher, M. M.; Kalyandrug, L. B.; Armellino, D. C.; Boghaert, E. R.; Hamann, P. R.; Moran, J. K.; Damle, N. K. Antitumor Efficacy of a Combination of CMC-544 (Inotuzumab Ozogamicin), a CD22-Targeted Cytotoxic Immunoconjugate of Calicheamicin, and Rituximab against Non-Hodgkin's B-Cell Lymphoma. Clin. Cancer Res. 2006, 12, 242-249.
Dijoseph, J. F.; Popplewell, A.; Tickle, S.; Ladyman, H.; Lawson, A.; Khandke, K.; Armellino, D. C.; Boghaert, E. R.; Hamann, P. R.; Zinkewich-Peotti, K.; Stephens, S.; Weir, N.; Damle, N. K. Antibody-Targeted Chemotherapy of B-Cell Lymphoma Using Calicheamicin Conjugated to Murine or Humanized Antibody against CD22. Cancer Immunol. Immunother. 2005, 54, 11-24.
Dijoseph, J. F.; Goad, M. E.; Dougher, M. M.; Boghaert, E. R.; Kunz, A.; Hamann, P. R.; Damle, N. K. Potent and Specific Antitumor Efficacy of CMC-544, a CD22-Targeted Immunoconjugate of Calicheamicin, against Systemically Disseminated B-Cell Lymphoma. Clin. Cancer Res. 2004, 10, 8620-8629.
Feng, Y.; Zhen, Y. S.; Dai, Y.; Shang, B. Y.; Zhang, M.; He, H. W.; Li, B.; Shao, R. G. Antitumor Activities of Various Immunoconjugates Composed of Lidamycin and Anti-Type IV Collagenase Monoclonal Antibody. Yao Xue Bao 2007, 42, 704-709.
Li, L.; Huang, Y. H.; Li, Y.; Wang, F. Q.; Shang, B. Y.; Zhen, Y. S. Antitumor Activity of Anti-Type IV Collagenase Monoclonal Antibody and Its Lidamycin Conjugate against Colon Carcinoma. World J. Gastroenterol. 2005, 11, 4478-4483.
Li, J.; Zhen, Y. S.; Yang, Z. Biodistribution of Monoclonal Antibody and Fab Fragment and Antitumor Effect of Their Conjugates on Hepatoma Xenografts. Zhongguo Yi Ke Xue Yuan Xue Bao 1994, 16, 328-333.
Miao, Q. F.; Liu, X. Y.; Shang, B. Y.; Ouyang, Z. G.; Zhen, Y. S. An Enediyne-Energized Single-Domain Antibody-Containing Fusion Protein Shows Potent Antitumor Activity. Anticancer Drugs 2007, 18, 127-137.
Hamann, P. R.; Hinman, L. M.; Beyer, C. F.; Lindh, D.; Upeslacis, J.; Shochat, D.; Mountain, A. Calicheamicin Conjugate with a Fully Humanized Anti-MUC1 Antibody Shows Potent Antitumor Effects in Breast and Ovarian Tumor Xenografts. Bioconjug. Chem. 2005, 16, 354-360.
Hamann, P. R.; Hinman, L. M.; Beyer, C. F.; Greenberger, L. M.; Lin, C.; Lindh, D.; Menendez, A. T.; Wallace, R.; Durr, F. E. Upeslacis, J. An Anti-MUC1 Antibody-Calicheamicin Conjugate for Treatment of Solid Tumors. Choice of Linker and Overcoming Drug Resistance. Bioconjug. Chem. 2005, 16, 346-353.
Boghaert, E. R.; Sridharan, L.; Armellino, D. C.; Khandke, K. M.; Dijoseph, J. F.; Kunz, A.; Dougher, M. M.; Jiang, F.; Kalyandrug, L. B.; Hamann, P. R.; Frost, P.; Damle, N. K. Antibody-Targeted Chemotherapy with the Calicheamicin Conjugate Hu3S193-N-Acetyl Gamma Calicheamicin Dimethyl Hydrazide Targets Lewis^{γ} and Eliminates Lewis^{γ} -Positive Human Carcinoma Cells and Xenografts. Clin. Cancer Res. 2004, 10, 4538-4549.
Knoll, K.; Wrasidlo, W.; Scherberich, J. E.; Gaedicke, G.; Fischer, P. Targeted Therapy of Experimental Renal Cell Carcinoma with a Novel Conjugate of Monoclonal Antibody 138H11 and Calicheamicin γ1I. Cancer Res. 2000, 60, 6089-6094.
Okamoto, K.; Yamaguchi, T.; Otsuji, E.; Yamaoka, N.; Yata, Y.; Tsuruta, H.; Kitamura, K.; Takahashi, T. Targeted Chemotherapy in Mice with Peritoneally Disseminated Gastric Cancer Using Monoclonal Antibody-Drug Conjugate. Cancer Lett. 1998, 122, 231-236.
Otsuji, E.; Yamaguchi, T.; Tsuruta, H.; Yata, Y.; Nishi, H.; Okamoto, K.; Taniguchi, K.; Kato, M.; Kotani, T.; Kitamura, K.; Takahashi, T. Effects of Neocarzinostatin-Chimeric Fab Conjugates on the Growth of Human Pancreatic Carcinoma Xenografts. Br. J. Cancer 1996, 73, 1178-1182.
Nicolaou, K. C.; Hummel, C. W.; Pitsinos, E. N.; Nakada, M.; Smith, A. L.; Shibayama, K.; Saimoto, H. Total Synthesis of Calicheamicin 71I. J. Am. Chem. Soc. 1992, 114 (25), 10082-10084.
Shair, M. D.; Yoon, T.; Danishefsky, S. J. Total Synthesis of (±)-Dynemicin A. Angew. Chemie Int. Ed. Engl. 1995, 34 (16), 1721-1723.
Shair, M. D.; Yoon, T. Y.; Mosny, K. K.; Chou, T. C.; Danishefsky, S. J. The Total Synthesis of Dynemicin A Leading to Development of a Fully Contained Bioreductively Activated Enediyne Prodrug. J. Am. Chem. Soc. 1996, 118 (40), 9509-9525.
Myers, A. G.; Fraley, M. E.; Tom, N. J.; Cohen, S. B.; Madar, D. J. Synthesis of (±)-Dynemicin A and Analogs of Wide Structural Variability: Establishment of the Absolute Configuration of Natural Dynemicin A. Chem. Biol. 1995, 2 (1), 33-43.
Nicolaou, K. C.; Wang, Y.; Lu, M.; Mandal, D.; Pattanayak, M. R.; Yu, R.; Shah, A. A.; Chen, J. S.; Zhang, H.; Crawford, J. J.; Pasunoori, L.; Poudel, Y. B.; Chowdari, N. S.; Pan, C.; Nazeer, A.; Gangwar, S.; Vite, G.; Pitsinos, E. N. Streamlined Total Synthesis of Uncialamycin and Its Application to the Synthesis of Designed Analogues for Biological Investigations. J. Am. Chem. Soc. 2016, 138 (26), 8235-8246.
Nicolaou, K. C.; Das, D.; Lu, Y.; Rout, S.; Pitsinos, E. N.; Lyssikatos, J.; Schammel, A.; Sandoval, J.; Hammond, M.; Aujay, M.; Gavrilyuk, J. Total Synthesis and Biological Evaluation of Tiancimycins A and B, Yangpumicin A, and Related Anthraquinone-Fused Enediyne Antitumor Antibiotics. J. Am. Chem. Soc. 2020, 142 (5), 2549-2561.
Chowdari, N. S.; Pan, C.; Rao, C.; Langley, D. R.; Sivaprakasam, P.; Sufi, B.; Derwin, D.; Wang, Y.; Kwok, E.; Passmore, D.; Rangan, V. S.; Deshpande, S.; Cardarelli, P.; Vite, G.; Gangwar, S. Uncialamycin as a Novel Payload for Antibody Drug Conjugate (ADC) Based Targeted Cancer Therapy. Bioorganic Med. Chem. Lett. 2019, 29 (3), 466-470.
Wang, R.; Li, L.; Zhang, S.; Li, Y.; Wang, X.; Miao, Q.; Zhen, Y. A Novel Enediyne-Integrated Antibody-Drug Conjugate Shows Promising Antitumor Efficacy against CD30+ Lymphomas. Mol. Oncol. 2018, 12 (3), 339-355.
Adhikari, A.; Shen, B.; Rader, C. Challenges and Opportunities to Develop Enediyne Natural Products as Payloads for Antibody-Drug Conjugates. Antib. Ther. 2021, 4 (1), 1-15.
Poudel, Y. B.; Chowdari, N. S.; Cheng, H.; Iwuagwu, C. I.; King, H. D.; Kotapati, S.; Passmore, D.; Rampulla, R.; Mathur, A.; Vite, G.; Gangwar, S. Chemical Modification of Linkers Provides Stable Linker-Payloads for the Generation of Antibody-Drug Conjugates. ACS Med. Chem. Lett. 2020, 11 (11), 2190-2194.
Nicolaou, K. C.; Rigol, S.; Pitsinos, E. N.; Das, D.; Lu, Y.; Rout, S.; Schammel, A. W.; Holte, D.; Lin, B.; Gu, C.; Sarvaiya, H.; Trinidad, J.; Barbour, N.; Valdiosera, A. M.; Sandoval, J.; Lee, C.; Aujay, M.; Fernando, H.; Dhar, A.; Karsunky, H.; Taylor, N.; Pysz, M.; Gavrilyuk, J. Uncialamycin-Based Antibody—Drug Conjugates: Unique Enediyne ADCs Exhibiting Bystander Killing Effect. Proc. Natl. Acad. Sci. U.S.A 2021, 118 (25), 1-7.
Nicolaou C K; Hwang, C. K.; Smith, A. L.; Wendeborn, S. V. Synthesis of Dynemicin A Models. J. Am. Chem. Soc 1990, 112, 7416-7418.
Sugiura, Y.; Shiraki, T.; Konishio, M.; Oki, T. DNA Intercalation and Cleavage of an Antitumor Antibiotic Dynemicin That Contains Anthracycline and Enediyne Cores (Hybrid Antibiotic/Nucleotide Specificity/Minor Groove/Action Mechanism). Proc. Nati. Acad. Sci. USA 1990, 87, 3831-3835.
Langley, D. R.; Doyle, T. W.; Beveridge, D. L. The Dynemicin-DNA Intercalation Complex. A Model Based on Dna Affinity Cleavage and Molecular Dynamics Simulation. J. Am. Chem. Soc. 1991, 113 (12), 4395-4403.
Sugiura, Y.; Shiraki, T.; Konishi, M.; Oki, T. DNA Intercalation and Cleavage of an Antitumor Antibiotic Dynemicin That Contains Anthracycline and Enediyne Cores. Proc. Natl. Acad. Sci. U.S.A 1990, 87 (10), 3831-3835.
Myers, A. G.; Cohen, S. B.; Tom, N. J.; Madar, D. J.; Fraley, M. E. J. Am. Chem. Soc. 1995, 117 (28), 7574-7575.
Myers, A. G.; Kort, M. E.; Cohen, S. B.; Tom, N. J. Enzymatic Activation of DNA Cleavage by Dynemicin A and Synthetic Analogs. Biochemistry 1997, 36 (13), 3903-3908.
Tuttle, T.; Kraka, E.; Cremer, D. Docking, Triggering, and Biological Activity of Dynemicin A in DNA: A Computational Study. J. Am. Chem. Soc. 2005, 127 (26), 9469-9484.
Tuttle, T.; Kraka, E.; Thiel, W.; Cremer, D. A QM/MM Study of the Bergman Reaction of Dynemicin A in the Minor Groove of DNA. J. Phys. Chem. B 2007, 111 (28), 8321-8328.
Bisht, R.; Hoque, M. E.; Chattopadhyay, B. Amide Effects in C—H Activation: Noncovalent Interactions with L-Shaped Ligand for Meta Borylation of Aromatic Amides. Angew. Chemie—Int. Ed. 2018, 57 (48), 15762-15766.
Faigl, F.; Thurner, A.; Molnir, B.; Simig, G.; Volk, B. Manufacturing Synthesis of 5-Substituted Phthalides. Org. Process Res. Dev. 2010, 14 (3), 617-622.
Wang, C.; Dong, G. Direct β-Alkylation of Ketones and Aldehydes via Pd-Catalyzed Redox Cascade. J. Am. Chem. Soc. 2018, 140 (19), 6057-6061.
Borders, D. B., Doyle, T. W., Enediyne Antibiotics as Antitumor Agents, Marcel Dekker Inc., New York, 1995.
Xi, Z., Goldberg, I. H., in Comprehensive Natural Products Chemistry (Eds.: S. D. Barton, K. Nakanishi, O. Meth-Cohn), Pergamon, New York, 1999, pp. 553-592.
Lee, M. D., Dunne, T. S., Siegel, M. M., Chang, C. C., Morton, G. O., Borders, D. B., ‘Calichemicins, a novel family of antitumor antibiotics. 1. Chemistry and partial structure of calichemicin .gamma.1I’, J. Am. Chem. Soc. 1987a, 109, 3464-3466.
Lee, M. D., Dunne, T. S., Chang, C. C., Ellestad, G. A., Siegel, M. M., Morton, G. O., Mcgahren, W. J., Borders, D. B., ‘Calichemicins, a Novel Family of Antitumor Antibiotics. 2. Chemistry and Structure of Calichemicin yl’, J. Am. Chem. Soc. 1987b, 109, 3466-3468.
Konishi, M., Ohkuma, H., Tsuno, T., Oki, T., ‘Crystal and molecular structure of dynemicin A: a novel 1,5-diyn-3-ene antitumor antibiotic’, J. Am. Chem. Soc. 1990, 112, 3715-3716.
Kim, K. H., Kwon, B. M., Myers, A. G., Rees, D. C., ‘Crystal structure of neocarzinostatin, an antitumor protein-chromophore complex’, Science (1979) 1993, 262, 1042-1046.
De Voss, J. J., Townsend, C. A., Ding, W., Morton, G. O., Ellestad, G. A, Tabor, A. B., Schreiber, S. L., ‘Site-specific atom transfer from DNA to a bound ligand defines the geometry of a DNA-calicheamicin .gamma.1I complex’, J. Am. Chem. Soc. 1990, 112, 9669-9670.
Adhikari, A., Teijaro, C. N., Townsend, C. A., Shen, B., in Comprehensive Natural Products III (Eds.: H. W. Liu, T. P. Begley), Elsevier, 2020, pp. 365-414.
Sievers, E. L., Senter, P. D., ‘Antibody-Drug Conjugates in Cancer Therapy’, Annu Rev Med 2013, 64, 15-29.
Norsworthy, K. J., Ko, C.-W., Lee, J. E., Liu, J., John, C. S., Przepiorka, D., Farrell, A. T., Pazdur, R., ‘FDA Approval Summary: Mylotarg for Treatment of Patients with Relapsed or Refractory CD33-Positive Acute Myeloid Leukemia’, Oncologist 2018, 23, 1103-1108.
Appelbaum, F. R., Bernstein, I. D., ‘Gemtuzumab ozogamicin for acute myeloid leukemia’, Blood 2017, 130, 2373-2376.
Fenwarth, L., Fournier, E., Cheok, M., Boyer, T., Gonzales, F., Castaigne, S., Boissel, N., Lambert, J., Dombret, H., Preudhomme, C., Duployez, N., ‘Biomarkers of gemtuzumab ozogamicin response for acute myeloid leukemia treatment’, Int J Mol Sci 2020, 21, 1-21.
Li, X., Zhou, M., Qi, J., Han, Y., ‘Efficacy and Safety of Inotuzumab Ozogamicin (CMC-544) for the Treatment of Relapsed/Refractory Acute Lymphoblastic Leukemia and Non-Hodgkin Lymphoma: A Systematic Review and Meta-Analysis’, Clin Lymphoma Myeloma Leuk 2021, 21, E227-E247.
De Vries, J. F., Zwaan, C. M., De Bie, M., Voerman, J. S. A., Den Boer, M. L., Van Dongen, J. J. M., Van Der Velden, V. H. J., ‘The novel calicheamicin-conjugated CD22 antibody inotuzumab ozogamicin (CMC-544) effectively kills primary pediatric acute lymphoblastic leukemia cells’, Leukemia 2012, 26, 255-264.
Takeshita, A., Yamakage, N., Shinjo, K., Ono, T., Hirano, I., Nakamura, S., Shigeno, K., Tobita, T., Maekawa, M., Kiyoi, H., Naoe, T., Ohnishi, K., Sugimoto, Y., Ohno, R., ‘CMC-544 (inotuzumab ozogamicin), an anti-CD22 immuno-conjugate of calicheamicin, alters the levels of target molecules of malignant B-cells’, Leukemia 2009, 23, 1329-1336.
Tsuchiya, K., Uchida, T., Kobayashi, M., Maeda, H., Konno, T., Yamanaka, H., ‘Tumor-targeted chemotherapy with SMANCS in lipiodol for renal cell carcinoma: longer survival with larger size tumors’, Adult Urology 2000, 55, P495-500.
Meada, H., Konno, T., in Neocarzinostatin (Eds.: H. Maeda, K. Edo, N. Ishida), Spinger, Tokyo, 1997, pp. 227-267.
Beerman, T. A., Gawron, L. S., Shin, S., Shen, B., McHugh, M. M., ‘C-1027, a radiomimetic enediyne anti-cancer drug, preferentially targets hypoxic cells’, Cancer Res 2009, 69, 593-598.
Zhen, Y. Z., Lin, Y. J., Li, Y., Zhen, Y. S., ‘Lidamycin shows highly potent cytotoxic to myeloma cells and inhibits tumor growth in mice’, Acta Pharmacol Sin 2009, 30, 1025-1032.
Kennedy, D. R., Gawron, L. S., Ju, J., Liu, W., Shen, B., Beerman, T. A., ‘Single chemical modifications of the C-1027 enediyne core, a radiomimetic antitumor drug, affect both drug potency and the role of ataxia-telangiectasia mutated in cellular responses to DNA double-strand breaks’, Cancer Res 2007, 67, 773-781.
Schwan, S., Schroder, D., Wegner, H. A., Janek, J., Mollenhauer, D., ‘Substituent Pattern Effects on the Redox Potentials of Quinone-Based Active Materials for Aqueous Redox Flow Batteries’, ChemSusChem 2020, 13, 5480-5488.
Maeda, H., Sawa, T., Konno, T., ‘Mechanism of tumor-targeted delivery of macromolecular drugs, including the EPR effect in solid tumor and clinical overview of the prototype polymeric drug SMANCS’, Journal of Controlled Release 2001, 74, 47-61.
Weissman, K. J., ‘Mutasynthesis—uniting chemistry and genetics for drug discovery’, Trends Biotechnol 2007, 25, 139-142.
Kennedy, J., ‘Mutasynthesis, chemobiosynthesis, and back to semi-synthesis: Combining synthetic chemistry and biosynthetic engineering for diversifying natural products’, Nat Prod Rep 2008, 25, 25-34.
Claridge, C. A., in Basic Biology of New Developments in Biotechnology. Basic Life Sciences (Eds.: A. Hollaender, A. I. Laskin, P. Rogers), Springer, Boston, MA, 1983, pp. 231-269.
Wang, J. B., Pan, H. X., Tang, G. L., ‘Production of doramectin by rational engineering of the avermectin biosynthetic pathway’, Bioorg Med Chem Lett 2011, 21, 3320-3323.
Galm, U., Dessoy, M. A., Schmidt, J., Wessjohnann, L. A., Heide, L., ‘In Vitro and In Vivo Production of New Aminocoumarins by a Combined Biochemical, Genetic, and Synthetic Approach’, Chem Biol 2004, 11, 173-183.
Weist, S., Kittel, C., Bischoff, D., Bister, B., Pfeifer, V., Nicholson, G. J., Wohlleben, W., Süssmuth, R. D., ‘Mutasynthesis of Glycopeptide Antibiotics: Variations of Vancomycin's AB-Ring Amino Acid 3,5-Dihydroxyphenylglycine’, J Am Chem Soc 2004, 126, 5942-5943.
Moss, S. J., Carletti, I., Olano, C., Sheridan, R. M., Ward, M., Math, V., Nur-E-Alam, M., Brana, A. F., Zhang, M. Q., Leadlay, P. F., Mendez, C., Salas, J. A., Wilkinson, B., ‘Biosynthesis of the angiogenesis inhibitor borrelidin: Directed biosynthesis of novel analogues’, Chemical Communications 2006, 2341-2343.
Boddy, C. N., Hotta, K., Tse, M. L., Watts, R. E., Khosla, C., ‘Precursor-directed biosynthesis of epothilone in Escherichia coli’, J Am Chem Soc 2004, 126, 7436-7437.
Pal, P., Alley, J. R., Townsend, C. A., ‘Examining Heterodimerization by Aryl C—N Coupling in Dynemicin Biosynthesis’, ACS Chem Biol 2023a, 18, 304-314.
Pal, P., Wessely, S. M. L., Townsend, C. A., ‘Normal and Aberrant Methyltransferase Activities Give Insights into the Final Steps of Dynemicin A Biosynthesis’, J Am Chem Soc 2023b, 145, 12935-12947.
Ma, G.-L., Tran, H. T., Low, Z. J., Candra, H., Pang, L. M., Cheang, W. W., Fang, M., Liang, Z.-X, ‘Pathway Retrofitting Yields Insights into the Biosynthesis of Anthraquinone-Fused Enediynes’, J Am Chem Soc 2021, 143, 11500-11509.
Gui, C., Kalkreuter, E., Liu, Y. C., Adhikari, A., Teijaro, C. N., Yang, D., Chang, C., Shen, B., ‘Intramolecular C—C Bond Formation Links Anthraquinone and Enediyne Scaffolds in Tiancimycin Biosynthesis’, J Am Chem Soc 2022, 144, 20452-20462.
Bhardwaj, M., Cui, Z., Hankore, E. D., Moonschi, F. H., Esfahani, H. S., Kalkreuter, E., Gui, C., Yang, D., Phillips, G. N., Thorson, J. S., Shen, B., Van Lanen, S. G., ‘A discrete intermediate for the biosynthesis of both the enediyne core and the anthraquinone moiety of enediyne natural products’, Proceedings of the National Academy of Sciences 2023, 120, e2220468120.
Horsman, G. P., Chen, Y., Thorson, J. S., Shen, B., ‘Polyketide synthase chemistry does not direct biosynthetic divergence between 9- and 10-membered enediynes’, Proc Natl Acad Sci USA 2010, 107, 11331-11335.
Belecki, K., Townsend, C. A., ‘Biochemical determination of enzyme-bound metabolites: Preferential accumulation of a programmed octaketide on the enediyne polyketide synthase CalE8’, J Am Chem Soc 2013, 135, 14339-14348.
Yang, D., Ye, F., Teijaro, C. N., Hwang, D., Annaval, T., Adhikari, A., Li, G., Yan, X., Gui, G., Rader, C., Shen, B., ‘Functional Characterization of Cytochrome P450 Hydroxylase YpmL in Yangpumicin A Biosynthesis and Its Application for Anthraquinone-Fused Enediyne Structural Diversification’, Org Lett 2022, 24, 1219-1223.
Wang, B., Chai, X., Zhu, W., Wang, T., Wu, Q., ‘A general approach to spirolactonized Si-rhodamines’, Chemical Communications 2014, 50, 14374-14377.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A compound of formula (I): 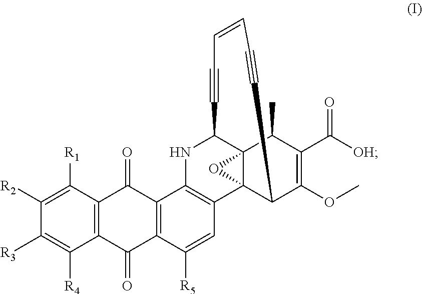
wherein:
R₁ is selected from H, hydroxyl, and —O—(C═O)—CH₃;
R₂ and R₃ are each independently selected from H, hydroxyl, C₁-C₄ alkyl, C₁-C₄ alkoxyl, halogen, carbonyl, carboxyl, acetyl, cyano, mercapto, nitro, —O—(C═O)—CH₃, and —(CH₂)ₙ—NR₆R₇, wherein n is an integer selected from 0, 1, 2, 3, and 4, R₆ and R₇ are each independently selected from H, C₁-C₄ alkyl, and a protecting group;
R₅ is hydroxyl or —O—(C═O)—CH₃;
provided that if R₁, R₄, and R₅ are each hydroxyl, then R₂ and R₃ cannot both be H; and; stereoisomers and pharmaceutically acceptable salts thereof.

2. The compound of claim 1, wherein the compound of formula (I) is a compound of formula (I′): 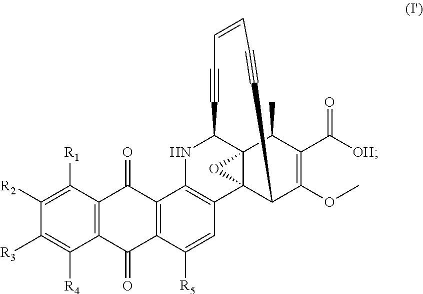
wherein:
R₁ is selected from H, hydroxyl, and —O—(C═O)—CH₃;
R₂ and R₄ are each H;
R₃ is selected from C₁-C₄ alkyl, halogen, —(CH₂)ₙ—NR₆R₇, wherein n is an integer selected from 0, 1, 2, 3, and 4, R₆ and R₇ are each independently selected from H, C₁-C₄ alkyl, and a protecting group;
R₅ is hydroxyl or —O—(C═O)—CH₃; and
stereoisomers and pharmaceutically acceptable salts thereof.

3. The compound of claim 2, wherein the compound is a compound of formula (Ia)-formula (If): 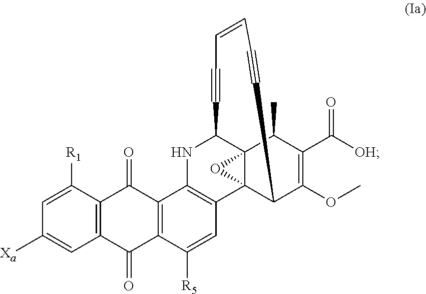
wherein:
Xₐ is halogen or cyano;
R₁ is selected from H, hydroxyl, and —O—(C═O)—CH₃; and
R₅ is hydroxyl or —O—(C═O)—CH₃ 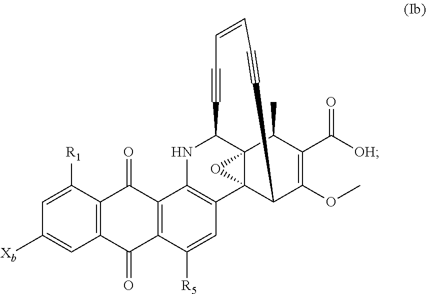
wherein:
X_{b} is C₁-C₄ alkyl;
R₁ is selected from H, hydroxyl, and —O—(C═O)—CH₃; and
R₃ is hydroxyl or —O—(C═O)—CH₃; 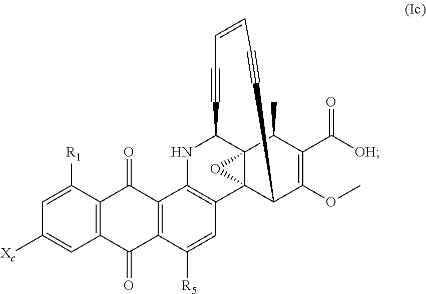
wherein:
X_{c} is selected from Br, cyano, and —(CH₂)ₙ—NR₆R₇, wherein n is an integer selected from 0, 1, 2, 3, and 4, R₆ and R₇ are each independently selected from H, C₁-C₄ alkyl, and a protecting group;
R₁ is selected from H, hydroxyl, and —O—(C═O)—CH₃; and
R₅ is hydroxyl or —O—(C═O)—CH₃; 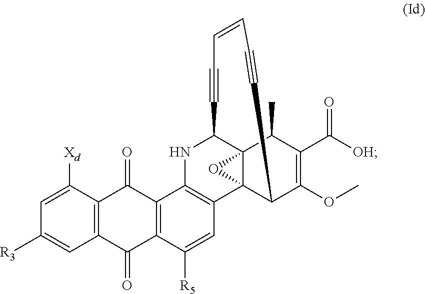
wherein:
X_{d} is H;
R₃ is selected from C₁-C₄ alkyl, halogen, and —(CH₂)ₙ—NR₆R₇, wherein n is an integer selected from 0, 1, 2, 3, and 4, R₆ and R₇ are each independently selected from H, C₁-C₄ alkyl, and a protecting group; and
R₅ is hydroxyl or —O—(C═O)—CH₃; 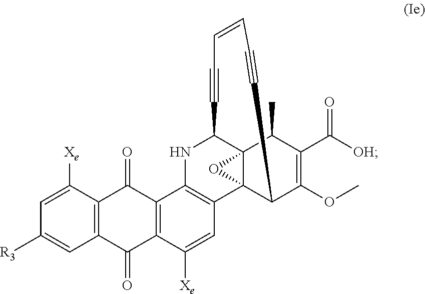
wherein:
each Xₑ is hydroxyl; and
R₃ is selected from C₁-C₄ alkyl, halogen, and —(CH₂)ₙ—NR₆R₇, wherein n is an integer selected from 0, 1, 2, 3, and 4, R₆ and R₇ are each independently selected from H, C₁-C₄ alkyl, and a protecting group; and 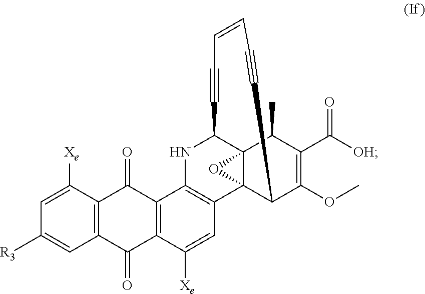
wherein:
each Xₑ is —O—(C═O)—CH₃; and
R₃ is selected from C₁-C₄ alkyl, halogen, and —(CH₂)ₙ—NR₆R₇, wherein n is an integer selected from 0, 1, 2, 3, and 4, R₆ and R₇ are each independently selected from H, C₁-C₄ alkyl, and a protecting group.

4. The compound of claim 3, wherein Xₐ of the compound of formula (Ia) is selected from Br, Cl, F, and cyano.

5. (canceled)

6. The compound of claim 3, wherein X_{b} of a compound of formula (Ib) is methyl or ethyl.

7. (canceled)

8. The compound of claim 7, wherein R₆ and R₇ of the compound of formula (Ic) are each H.

9.-11. (canceled)

12. The compound of claim 2, wherein:
R₃ is F and R₁ and R₅ are each independently OH;
R₃ is F, R₁ is H, and R₅ is OH;
R₃ is F and R₁ and R₅ are each independently —O—(C═O)—CH₃;
R₃ is Cl and R₁ and R₅ are each independently —O—(C═O)—CH₃;
R₃ is methyl and R₁ and R₅ are each independently OH;
R₃ is methyl, R₁ is H, R₅ is OH;
R₃ is methyl, and R₁ and R₅ are each independently —O—(C═O)—CH₃;
R₃ is Cl and R₁ and R₅ are each independently OH;
R₃ is Cl, R₁ is H, and R₅ is OH;
R₃ is Cl, R₁ is H, and R₅ is —O—(C═O)—CH₃;
R₃ is ethyl, R₁ is H, and R₅ is OH;
R₃ is ethyl, R₁ is H, and R₅ is —O—(C═O)—CH₃;
R₃ is NH₂ and R₁ and R₅ are each independently OH; or
R₃ is NH₂, R₁ is H, and R₅ is OH.

13. A pharmaceutical composition comprising a compound of claim 1 and a pharmaceutically acceptable carrier.

14. A conjugate comprising a compound of claim 1 and an antibody.

15. The conjugate of claim 14, wherein the antibody is selected from a CD33 monoclonal antibody (mAB); a CD22 mAB; an mAb directed against type IV collagenase, including MMP-2 and MMP-9; anti-CD19(Fab); an antibody directed against polymorphic epithelial mucin, e.g., hCTM01; an antibody recognizing Lewis^{γ} (Le^{γ}) antigen, e.g., hu3S193; an antibody that recognizes human renal gamma-glutamyltransferase (GGT), e.g., 138H11; an antibody recognizing a tumor-specific antigen, such as the glycoprotein on the cell surface of human colon cancer, e.g., A7; and an antibody recognizing a tumor-specific antigen, such as pancreatic cancer e.g., chA7Fab.

16. A conjugate comprising a compound of claim 1 and a delivery system.

17. The conjugate of claim 16, wherein the delivery system comprises a poly(styrene-co-maleic acid).

18. A compound of formula (II): 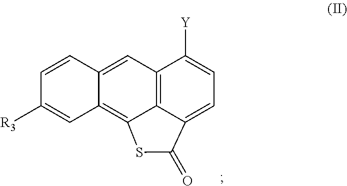
wherein:
Y is selected from halogen, hydroxyl, methoxyl, and —O—S(═O)₂—CF₃ (—OTf); and
R₃ is selected from halogen, C₁-C₄ alkyl, —C(═O)—CH₃, —NH—C(═O)—CH₃, cyano, and —(CH₂)ₙ—NR₆R₇, wherein n is an integer selected from 0, 1, 2, 3, and 4, R₆ and R₇ are each independently selected from H, C₁-C₄ alkyl, and a protecting group.

19. The compound of claim 18, wherein Y is iodine.

20. The compound of claim 18, wherein R₃ is selected from Cl, Br, F, methyl, and ethyl.

21. A method for generating dynemicin or an analogue thereof, the method comprising:
culturing a plurality of mutant Micromonospora lacking ORF15 in growth media supplemented with an iodoanthracene for a period of time and under conditions sufficient to process the iodoanthracene; and
purifying the dynemicin or analogue thereof.

22. The method of claim 21, wherein the iodoanthracene is a modified iodoanthracene.

23. The method of claim 22, wherein the modified iodoanthracene comprises a modification at one of more positions selected from C3, C8, and C9.

24. The method of claim 22, wherein the modified iodoanthracene comprises a modification at one of more positions selected from C8 and C9.

25. The method of claim 22, wherein the modified iodoanthracene is a 9-substituted iodoanthracene.

26. The method of claim 22, wherein the modified iodoanthracene is a compound of formula (II): 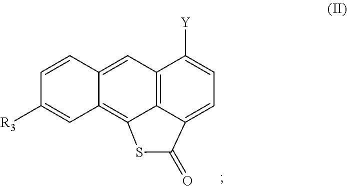
wherein:
Y is selected from halogen, hydroxyl, methoxyl, and —O—S(═O)₂—CF₃ (—OTf); and
R₃ is selected from halogen, C₁-C₄ alkyl, —C(═O)—CH₃, —NH—C(═O)—CH₃, cyano, and —(CH₂)ₙ—NR₆R₇, wherein n is an integer selected from 0, 1, 2, 3, and 4, R₆ and R₇ are each independently selected from H, C₁-C₄ alkyl, and a protecting group.

27. The method of claim 26, wherein Y is iodine.

28. The method of claim 26, wherein R₃ is selected from Cl, Br, F, cyano, methyl, and ethyl.

29. The method of claim 22, wherein the modified iodoanthracene is selected from the group consisting of: 9-fluoro-5-iodoanthracene-γ-thiolactone, 9-chloro-5-iodoanthracene-γ-thiolactone, 9-methyl-5-iodoanthracene-γ-thiolactone, 9-ethyl-5-iodoanthracene-γ-thiolactone, and 9-amino-5-iodoanthracene-γ-thiolactone.

30. The method of claim 22, wherein the modified iodoanthracene is prepared by one or all of:
(a) providing a 5-substituted phthalide;
(b) formation of an anthracene-γ-thiolactone comprising a masking group capable of being converted to an iodine substituent group;
(c) modification of an A-ring substituent group; and
(d) introduction of an iodine group in a C-ring of the anthracene-γ-thiolactone.

31. The method of claim 30, wherein the 5-substituted phthalide is prepared by the method provided in FIG. 21.

32. The method of claim 22, wherein the modified iodoanthracene is prepared by the method provided in FIG. 22.

33. The method of claim 21, wherein the dynemicin analogue is a methyl-, chloro-, ethyl-, fluoro-, cyano-, or an amino-analogue of dynemicin.

34. The method of claim 21, wherein the dynemicin analogue is a compound of formula (I): 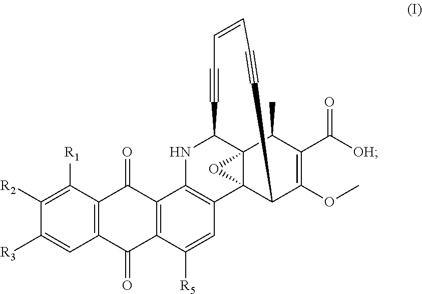
wherein:
R₁ is selected from H, hydroxyl, and —O—(C═O)—CH₃;
R₂ and R₃ are each independently selected from H, hydroxyl, C₁-C₄ alkyl, C₁-C₄ alkoxyl, halogen, carbonyl, carboxyl, acetyl, cyano, mercapto, nitro, —O—(C═O)—CH₃, and —(CH₂)ₙ—NR₆R₇, wherein n is an integer selected from 0, 1, 2, 3, and 4, R₆ and R₇ are each independently selected from H, C₁-C₄ alkyl, and a protecting group;
R₅ is hydroxyl or —O—(C═O)—CH₃;
provided that if R₁, R₄, and R₅ are each hydroxyl, then R₂ and R₃ cannot both be H; and; stereoisomers and pharmaceutically acceptable salts thereof.

35. The method of claim 21, wherein the period of time is about seven or more days.

36. The method of claim 21, wherein the conditions sufficient to process the iodoanthracene include incubation at 28° C. with shaking.

37. The method of claim 21, further comprising generating a mutant Micromonospora lacking ORF15.

38. The method of claim 21, wherein the mutant Micromonospora is Micromonospora chersina.

39. A method for treating cancer, the method comprising administering a therapeutically effective amount of a compound of claim 1 to a subject in need of treatment thereof.

40. The method of claim 39, wherein the cancer is selected from cervical carcinoma; leukemia; melanoma; hepatocellular carcinoma, also known as hepatoma (including advanced or recurrent hepatocellular carcinoma); acute myeloid leukemia (AML) (including first relapse with CD33-positive AML); B-cell acute lymphoblastic leukemia (ALL) (including CD22-positive B-cell ALL); colorectal carcinoma; sarcoma; breast cancer; ovarian carcinoma; gastric (or stomach) cancer; prostate carcinomas; metastasized renal cell carcinoma (RCC) (including metastasized RCC), and pancreatic carcinoma.
